(19) United States
(12) Patent Application Publication
(10) Pub. No.: US 2019/0378982 A1
(43) Pub. Date: Dec. 12, 2019

(54) ORGANIC MIXTURE, COMPOSITION, ORGANIC ELECTRONIC DEVICE AND APPLICATION

(71) Applicant: GUANGZHOU CHINARAY OPTOELECTRONIC MATERIALS LTD., Guangzhou, Guangdong (CN)

(72) Inventors: Ruifeng HE, Guangzhou, Guangdong (CN); Peng SHU, Guangzhou, Guangdong (CN); Yini LI, Guangzhou, Guangdong (CN); Junyou PAN, Guangzhou, Guangdong (US)

(73) Assignee: GUANGZHOU CHINARAY OPTOELECTRONIC MATERIALS LTD., Guangzhou, Guangdong (CN)

(21) Appl. No.: 16/463,341

(22) PCT Filed: Nov. 23, 2017
(86) PCT No.: PCT/CN2017/112715

§ 371 Date: May 22, 2019

(30) Foreign Application Priority Data
Nov. 23, 2016 (CN) 201611047052.X

(51) Int. Cl. H01L 51/00; C07F 5/02; C09K 11/06; C07D 209/86; C07D 487/04

(52) U.S. Cl. CPC H01L 51/008; C07F 5/027; C09K 11/06; C07D 209/86; H01L 51/5012; H01L 51/0054; C07D 487/04; H01L 51/0056; H01L 2251/552; H01L 51/0072

(57) ABSTRACT

An organic mixture, comprising a first organic compound and a second organic compound that forms an exciplex with the first organic compound. The first organic compound is an aromatic compound containing a triphenylboron heterocycle, and the second organic compound is a compound containing an aromatic fused heterocycle. LUMO_{H1}, HOMO_{H1} and E_{T}(H1) are respectively defined as the lowest unoccupied orbital, highest occupied orbital and triplet energy levels of the first organic compound, and LUMO_{H2}, HOMO_{H2} and E_{T}(H2) are respectively defined as the lowest unoccupied orbital, highest occupied orbital and triplet energy levels of the second organic compound, min((LUMO_{H1}−HOMO_{H2}, LUMO_{H2}−HOMO_{H1})≤min(E_{T}(H1), E_{T}(H2))+0.1 eV.

### CROSS-REFERENCE TO RELATED APPLICATIONS

The present application is the national phase of International Application PCT/CN2017/112715, filed on Nov. 23, 2017, which claims priority to Chinese Application No. 201611047052.X, filed on Nov. 23, 2016, both of which are incorporated herein by reference in their entireties.

### TECHNICAL FIELD

The present disclosure relates to the field of organic electronic devices, more particularly, to an organic mixture, a formulation containing the organic mixture, an organic electronic device containing the organic mixture, an organic electronic device prepared using the formulation, and an application of the organic electronic device.

### BACKGROUND

With the properties of light weight, active emitting, wide viewing angle, high contrast, high emitting efficiency, low energy consumption, easy preparation for flexible and large-sized panels, etc., organic light-emitting diodes (OLEDs) are regarded as the most promising next-generation display technology in the industry.

In order to promote the large-scale industrialization of the organic light-emitting diodes, further improving the luminescence properties and lifetime of the organic light-emitting diodes is a key issue that needs to be solved urgently, and high-performance organic optoelectronic material systems will still need to be further developed.

The host material is the key element for obtaining efficient and long-lifetime organic light-emitting diodes. Since the organic light-emitting diodes using phosphorescent materials can achieve nearly 100% internal electroluminescence quantum efficiency, the phosphorescent materials, especially, red and green phosphorescent materials, have become the mainstream material system in the industry. However, the phosphorescent OLEDs have a significant problem of roll-off effect, i.e., the phenomenon that the emitting efficiency decreases rapidly with the increase of current or voltage, due to the charge imbalance in the device, which is particularly disadvantageous for high brightness applications. In order to solve the above problem, Kim et al. (see Kim et al. Adv. Func. Mater. 2013 DOI: 10.1002/adfm.201300547, and Kim et al. Adv. Func. Mater. 2013, DOI: 10.1002/adfm.201300187) obtained the OLEDs with low roll-off and high efficiency by using a co-host that can form an exciplex together with another metal complex as the phosphorescent emitter. However, such devices have problems of short lifetime and poor stability.

### SUMMARY

Based on the above, it is necessary to provide an organic mixture which enables the organic electronic device to have a long lifetime and good stability.

In addition, a formulation and an electronic device comprising the organic mixture, and an application thereof are also provided.

An organic mixture comprising a first organic compound and a second organic compound that forms an exciplex with the first organic compound is provided, wherein, the first organic compound is an aromatic compound containing a triphenylboron heterocycle, the second organic compound is a compound containing an aromatic fused heterocycle, and LUMO_{H1} is defined as the lowest unoccupied molecular orbital energy level of the first organic compound, HOMO_{H1} is defined as the highest occupied molecular orbital energy level of the first organic compound, E_{T}(H1) is defined as the triplet excited state energy level of the first organic compound, and LUMO_{H2} is defined as the lowest unoccupied molecular orbital energy level of the second organic compound, HOMO_{H2} is defined as the highest occupied molecular orbital energy level of the second organic compound, E_{T}(H2) is defined as the triplet excited state energy level of the second organic compound, wherein, min((LUMO_{H1}−HOMO_{H2}, LUMO_{H2}−HOMO_{H1})≤min(E_{T}(H1), E_{T}(H2))+0.1 eV.

A formulation comprising the above organic mixture and an organic solvent is further provided.

An organic electronic device is further provided, comprising a functional layer whose material comprises one of the above organic mixture and the above formulation.

An application of the above organic electronic device in display equipments, lighting equipments, light sources, or sensors is further provided.

The details of one or more embodiments of the present disclosure are set forth in the accompanying drawings and the description below. Other features, objects and advantages of the present disclosure will become apparent from the description, the accompanying drawings, and the claims.

### DETAILED DESCRIPTION OF THE EMBODIMENTS

In order to facilitate the understanding of the present disclosure, the present disclosure will be described more fully hereinafter with reference to the related accompanying drawings.

The organic mixture of an embodiment can be used as a material for the functional layer of the organic electronic device. The organic electronic device may be one selected from the group consisting of an organic light-emitting diode (OLED), an organic photovoltaic cell (OPV), an organic light-emitting electrochemical cell (OLEEC), an organic field effect transistor (OFET), an organic light-emitting field effect transistor, an organic laser, an organic spintronic device, an organic sensor and an organic plasmon emitting diode. These organic electronic devices may be applied in display equipments, lighting equipments, light sources, or sensors. For example, the organic mixture is used as the material of the light-emitting layer in the organic light-emitting diode.

Wherein, the organic mixture comprises a first organic compound and a second organic compound that forms an exciplex with the first organic compound, the first organic compound is an aromatic compound containing a triphenylboron heterocycle, the second organic compound is a compound containing an aromatic fused heterocycle, and LUMO_{H1} is defined as the lowest unoccupied molecular orbital energy level of the first organic compound, HOMO_{H1} is defined as the highest occupied molecular orbital energy level of the first organic compound, E_{T}(H1) is defined as the triplet excited state energy level of the first organic compound, and LUMO_{H2} is defined as the lowest unoccupied molecular orbital energy level of the second organic compound, HOMO_{H2} is defined as the highest occupied molecular orbital energy level of the second organic compound, E_{T}(H2) is defined as the triplet excited state energy level of the second organic compound.

Wherein, min((LUMO_{H1}−HOMO_{H2}, LUMO_{H2}−HOMO_{H1})≤min(E_{T}(H1), E_{T}(H2))+0.1 eV.

Further, in the organic mixture, the first organic compound and the second organic compound form a type II heterojunction structure.

Further, min((LUMO_{H1}−HOMO_{H2}, LUMO_{H2}−HOMO_{H1})≤min(E_{T}(H1), E_{T}(H2));

further, min((LUMO_{H1}−HOMO_{H2}, LUMO_{H2}−HOMO_{H1})≤min(E_{T}(H1), E_{T}(H2))−0.05 eV;

further, min((LUMO_{H1}−HOMO_{H2}, LUMO_{H2}−HOMO_{H1})≤min(E_{T}(H1), E_{T}(H2))−0.1 eV;

further, min((LUMO_{H1}−HOMO_{H2}, LUMO_{H2}−HOMO_{H1})≤min(E_{T}(H1), E_{T}(H2))−0.15 eV;

and still further, min((LUMO_{H1}−HOMO_{H2}, LUMO_{H2}−HOMO_{H1})≤min(E_{T}(H1), E_{T}(H2))−0.2 eV.

In the present embodiment, the triplet excited state energy level E_{T}, HOMO and LUMO play a key role on the energy level structure of the organic material. The determinations of these energy levels are described below.

The HOMO and LUMO energy levels can be measured by photoelectric effect, such as XPS (X-ray Photoelectron Spectroscopy) and UPS (Ultroviolet Photoelectron Spectroscopy), or by Cyclic Voltammetry (hereinafter referred to as CV). Recently, quantum chemistry method such as density functional theory (hereinafter referred to as DFT), has also become a feasible method for calculating molecular orbital energy levels.

The triplet excited state energy level E_{T} of organic materials can be measured by low temperature time-resolved luminescence spectroscopy, or by quantum simulation calculation (e.g., by Time-dependent DFT), such as by the commercial software Gaussian 09W (Gaussian Inc.) in which the specific simulation method may refer to WO2011141110, or by methods as described in the embodiments below.

It should be noted that, the absolute values of HOMO, LUMO, E_{T} depend on the measurement method or calculation method used, even for the same method, different evaluation methods will also lead to different results, for example, different HOMO/LUMO value may be obtained at starting point and peak point on the CV curve. Therefore, reasonable and meaningful comparisons should be made using the same measurement method and the same evaluation method.

The values of HOMO, LUMO and E_{T} in the present embodiment are obtained based on the simulation of Time-dependent DFT. It should be noted that the acquisition of HOMO, LUMO and E_{T} is not limited to the method, and they may be obtained by other measurement methods or calculation methods.

A possible advantage of the organic mixture of the present embodiment is that the excited state of the system will preferentially occupy the exciplex with the lowest energy to facilitate the energy transfer of the triplet excited state of the first organic compound or the second organic compound to the exciplex, so as to improve the concentration of the exciplex.

The organic mixture of the present embodiment can be used as a host material.

Further, (HOMO−1) is defined as the second highest occupied molecular orbital energy level, and (HOMO−2) is defined as the third highest occupied molecular orbital energy level, and so on. (LUMO+1) is defined as the second lowest unoccupied molecular orbital energy level, and (LUMO+2) is defined as the third lowest occupied molecular orbital energy level, and so on. Therefore, in the above organic mixture, min((LUMO_{H1}−HOMO_{H2}), (LUMO_{H2}−HOMO_{H1})) is less than or equal to the energy level of the triplet excited state of the first organic compound, and min((LUMO_{H1}−HOMO_{H2}), (LUMO_{H2}−HOMO_{H1})) is less than or equal to the energy level of the triplet excited state of the second organic compound. The energy to form an exciplex between the first organic compound and the second organic compound depends on the value of min((LUMO_{H1}−HOMO_{H2}), (LUMO_{H2}−HOMO_{H1})).

Specifically, at least one of the first organic compound and the second organic compound satisfies ((HOMO−(HOMO−1))≥0.2 eV, further ((HOMO−(HOMO−1))≥0.25 eV, further ((HOMO−(HOMO−1))≥0.3 eV, further ((HOMO−(HOMO−1))≥0.35 eV, further ((HOMO−(HOMO−1))≥0.4 eV, and still further ((HOMO)−(HOMO−1))≥0.45 eV.

Further, in the above organic mixture, the second organic compound satisfies ((HOMO−(HOMO−1))≥0.2 eV, further ((HOMO−(HOMO−1))≥0.25 eV, further ((HOMO−(HOMO−1))≥0.3 eV, further ((HOMO−(HOMO−1))≥0.35 eV, further ((HOMO−(HOMO−1))≥0.4 eV, and still further ((HOMO−(HOMO−1))≥0.45 eV.

Wherein, in the above organic mixture, at least one of the first organic compound and the second organic compound satisfies ((LUMO+1)−LUMO)≥0.1 eV, further ((LUMO+1)−LUMO)≥0.15 eV, further, ((LUMO+1)−LUMO)≥0.20 eV, further ((LUMO+1)−LUMO)≥0.25 eV, still further ((LUMO+1)−LUMO)≥0.30 eV.

Further in the above organic mixture, the first organic compound satisfies ((LUMO+1)−LUMO)≥20.1 eV, further ((LUMO+1)−LUMO)≥20.15 eV, further, ((LUMO+1)−LUMO)≥20.20 eV, further ((LUMO+1)−LUMO)≥20.25 eV, still further ((LUMO+1)−LUMO)≥20.30 eV.

Specifically, the molar ratio of the first organic compound to the second organic compound is 2:8 to 8:2; further, the molar ratio of the first organic compound to the second organic compound is 3:7 to 7:3; and still further, the molar ratio of the first organic compound to the second organic compound is 4:6 to 6:4.

Specifically, the difference between the molar mass of the first organic compound and the second organic compound does not exceed 100 g/mmol. Further, the difference between the molar mass of the first organic compound and the second organic compound does not exceed 60 g/mmol; and still further, the difference between the molar mass of the first organic compound and the second organic compound does not exceed 30 g/mmol.

Specifically, the difference between the sublimation temperature of the first organic compound and the second organic compound does not exceed 30 K. Further, the difference between the sublimation temperature of the first organic compound and the second organic compound does not exceed 20 K; and still further, the difference between the sublimation temperature of the first organic compound and the second organic compound does not exceed 10 K.

Specifically, at least one of the first organic compound and the second organic compound has a glass transition temperature T_{g} of 100° C.; further, at least one of the first organic compound and the second organic compound has a glass transition temperature T_{g} of 120° C.; further, at least one of the first organic compound and the second organic compound has a glass transition temperature T_{g} of 140° C.; further, at least one of the first organic compound and the second organic compound has a glass transition temperature T_{g} of 160° C.; further, at least one of the first organic compound and the second organic compound has a glass transition temperature T_{g} of 180° C.

Further, a part of hydrogen atoms of at least one of the first organic compound and the second organic compound are substituted by deuterium; further, 10% of hydrogen atoms of at least one of the first organic compound and the second organic compound are substituted by deuterium; further, 20% of hydrogen atoms of at least one of the first organic compound and the second organic compound are substituted by deuterium; further, 30% of hydrogen atoms of at least one of the first organic compound and the second organic compound are substituted by deuterium; still further, 40% of hydrogen atoms of at least one of the first organic compound and the second organic compound are substituted by deuterium.

Further, both of the first organic compound and the second organic compound are a small molecular material. Wherein, the “small molecule” referred to herein has no repeating structure and is not a polymer, oligomer, dendrimer, or blend; and the molar mass of the small molecule is 3000 g/mmol or less; further, the molar mass of the small molecule is 2000 g/mmol or less; and still further, the molar mass of the small molecule is 1500 g/mmol or less.

When the above organic mixture is used to prepare an OLED device by evaporation, the molar masses of the first organic compound and the second organic compound are respectively 1000 g/mmol or less; further, the molar masses of the first organic compound and the second organic compound are respectively 900 g/mmol or less; further, the molar masses of the first organic compound and the second organic compound are respectively 850 g/mmol or less; further, the molar masses of the first organic compound and the second organic compound are respectively 800 g/mmol or less; and still further, the molar masses of the first organic compound and the second organic compound are respectively 700 g/mmol or less.

In the present embodiment, the first organic compound has the following structural formula:

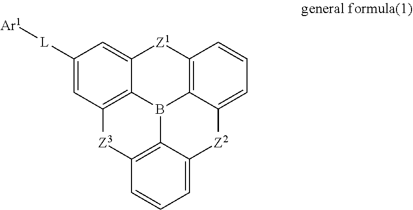

in the general formula (1), -L- is one selected from a single bond, a double bond and a triple bond, or L is one selected from an aromatic group with a ring atom number of 5 to 30 and a heteroaromatic group with a ring atom number of 5 to 30.

Further, -L- is one selected from a single bond, a double bond and a triple bond, or L is one selected from an aromatic group with a ring atom number of 5 to 20 and a heteroaromatic group with a ring atom number of 5 to 20; and still further, -L- is one selected from a single bond, a double bond and a triple bond, or L is one selected from an aromatic group with a ring atom number of 5 to 15 and a heteroaromatic group with a ring atom number of 5 to 15.

The aromatic group refers to a hydrocarbyl comprising at least one aromatic ring, that is, the aromatic group includes monocyclic aromatic group and polycyclic aromatic group. The heteroaromatic group refers to a hydrocarbyl comprising at least one heteroaromatic ring (containing heteroatoms), that is, the heteroaromatic group includes monocyclic heteroaromatic group and polycyclic heteroaromatic group. Such polycyclic aromatic groups and polycyclic heteroaromatic groups may have two or more rings, wherein two carbon atoms are shared by two adjacent rings, i.e., fused ring. At least one of the polycyclic aromatic groups is an aromatic ring, and at least one of the polycyclic heteroaromatic groups is a heteroaromatic ring.

It should be noted that the aromatic group referred to herein is not limited to an aromatic group, and the heteroaromatic group is not limited to a heteroaromatic group, wherein, a plurality of aromatic groups or heteroaromatic groups may be interrupted by short non-aromatic units (<10% of non-H atoms, further less than 5% of non-H atoms, such as C, N or O atoms). Therefore, systems such as 9,9′-spirobifluorene, 9,9-diarylfluorene, triarylamine, diaryl ether and the like are also considered to be fused ring aromatic groups.

Specifically, the aromatic group is one selected from the group consisting of benzene, naphthalene, anthracene, phenanthrene, perylene, tetracene, pyrene, benzopyrene, triphenylene, acenaphthene, fluorene, derivatives of benzene, derivatives of naphthalene, derivatives of anthracene, derivatives of phenanthrene, derivatives of perylene, derivatives of tetracene, derivatives of pyrene, derivatives of benzopyrene, derivatives of triphenylene, derivatives of acenaphthene, and derivatives of fluorene.

Specifically, the heteroaromatic group is one selected from the group consisting of furan, derivatives of furan, benzofuran, thiophene, benzothiophene, pyrrole, pyrazole, triazole, imidazole, oxazole, oxadiazole, thiazole, tetrazole, indole, carbazole, pyrroloimidazole, pyrrolopyrrole, thienopyrrole, thienothiophene, furopyrrole, furofuran, thienofuran, benzisoxazole, benzisothiazole, benzimidazole, pyridine, pyrazine, pyridazine, pyrimidine, triazine, quinoline, isoquinoline, cinnoline, quinoxaline, phenanthridine, perimidine, quinazoline, quinazolinone, derivatives of furan, derivatives of benzofuran, derivatives of thiophene, benzothiophene, derivatives of pyrrole, derivatives of pyrazole, derivatives of triazole, derivatives of imidazole, derivatives of oxazole, derivatives of oxadiazole, derivatives of thiazole, derivatives of tetrazole, derivatives of indole, derivatives of carbazole, derivatives of pyrroloimidazole, derivatives of pyrrolopyrrole, derivatives of thienopyrrole, derivatives of thienothiophene, derivatives of furopyrrole, derivatives of furofuran, derivatives of thienofuran, derivatives of benzisoxazole, derivatives of benzisothiazole, derivatives of benzimidazole, derivatives of pyridine, derivatives of pyrazine, derivatives of pyridazine, derivatives of pyrimidine, derivatives of triazine, derivatives of quinoline, derivatives of isoquinoline, derivatives of cinnoline, derivatives of quinoxaline, derivatives of phenanthridine, derivatives of perimidine, derivatives of quinazoline, and derivatives of quinazolinone.

Further, in the general formula (1), L is one selected from the group consisting of

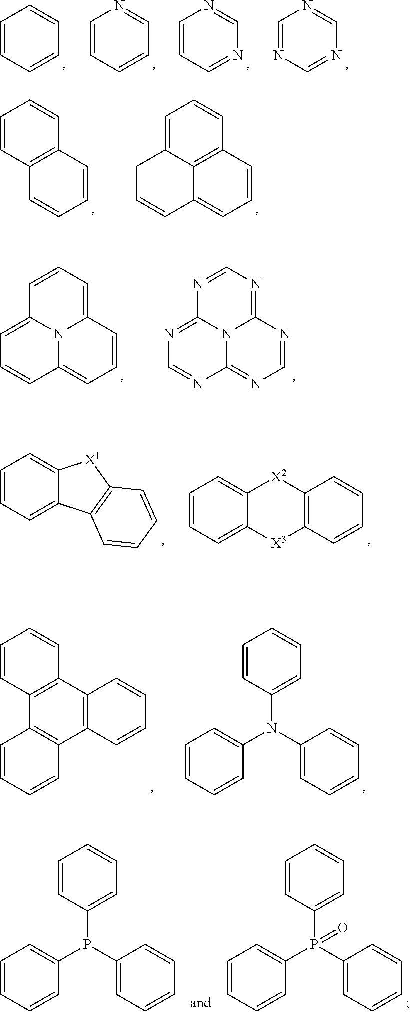

wherein, C—X¹—C, C—X²—C and C—X³—C are each independently selected from the group consisting of C—N(R)—C, C—C(R)₂—C, C—Si(R)₂—C, C—O—C, C—C═N(R)—C, C—C═C(R)₂—C, C—P(R)—C, C—P(═O)R—C, C—S—C, C—S═O—C, C—SO₂—C and C—C, and at most one of C—X²—C and C—X³—C is C—C.

Wherein, in the general formula (1), Ar¹ is one selected from the group consisting of an aromatic group with a ring atom number of 5 to 60, and a heteroaromatic group with a ring atom number of 5 to 60. Further, Ar¹ is one selected from the group consisting of an aromatic group with a ring atom number of 5 to 40, and a heteroaromatic group with a ring atom number of 5 to 40; And still further, Ar¹ is one selected from the group consisting of an aromatic group with a ring atom number of 5 to 30, and a heteroaromatic group with a ring atom number of 5 to 30.

Wherein, in the general formula (1), —Z¹—, —Z²— and —Z³— are each independently selected from the group consisting of none, —N(R)—, —C(R)₂—, —Si(R)₂—, —O—, —C═N(R)—, —C═C(R)₂—, —P(R)—, —P(═O)R—, —S—, —S═O— and —SO₂—, and at most two of —Z¹—, —Z²— and —Z³— are none; R is one selected from the group consisting of H, D, F, CN, alkenyl, alkynyl, nitrile group, amino, nitro, acyl, alkoxy, carbonyl, sulfonyl, C_{1˜30} alkyl, C_{3˜30} cycloalkyl, an aromatic hydrocarbyl with a ring atom number of 5 to 60, and an aromatic heterocyclic group with a ring atom number of 5 to 60.

Further, in the general formula (1), the structural formula of Ar¹ is one selected from the group consisting of

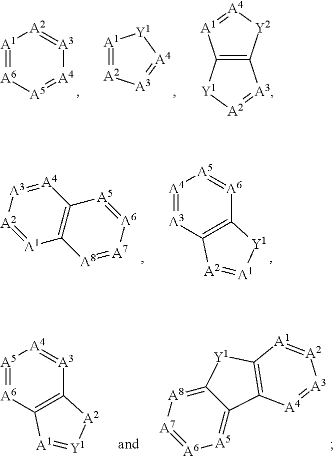

wherein, A¹, A², A³, A⁴, A⁵, A⁶, A⁷ and A⁸ are each independently selected from CR³ and N;

Y¹ and Y² are each independently selected from the group consisting of CR⁴R⁵, SiR⁴R⁵, NR³, C(═O), S and O;

R³, R⁴ and R⁵ are each independently selected from the group consisting of H, D, a linear alkyl with a carbon atom number of 1 to 20, a branched alkyl with a carbon atom number of 1 to 20, a cyclic alkyl with a carbon atom number of 1 to 20, an alkoxy with a carbon atom number of 1 to 20, a thioalkoxy with a carbon atom number of 1 to 20, a silyl with a carbon atom number of 1 to 20, a ketone group with a carbon atom number of 1 to 20, an alkoxycarbonyl group with a carbon atom number of 1 to 20, an aryloxycarbonyl with a carbon atom number of 7 to 20, cyano, carbamoyl (—C(═O)NH₂), haloformyl (C(═O)—X, wherein X represents a halogen atom), formyl(—C(═O)—H), isocyano, an isocyanate group, a thiocyanate group, an isothiocyanate group, hydroxyl, nitro, CF₃, Cl, Br, F, a crosslinkable group, an aryl containing 5 to 40 ring atoms, a heteroaryl containing 5 to 40 ring atoms, an aryloxy containing 5 to 40 ring atoms and a heteroaryloxy containing 5 to 40 ring atoms. Wherein, the crosslinkable group refers to a functional group containing unsaturated bonds, such as alkenyl, alkynyl, etc.

Further, in the general formula (1), Ar¹ is one selected from the group consisting of

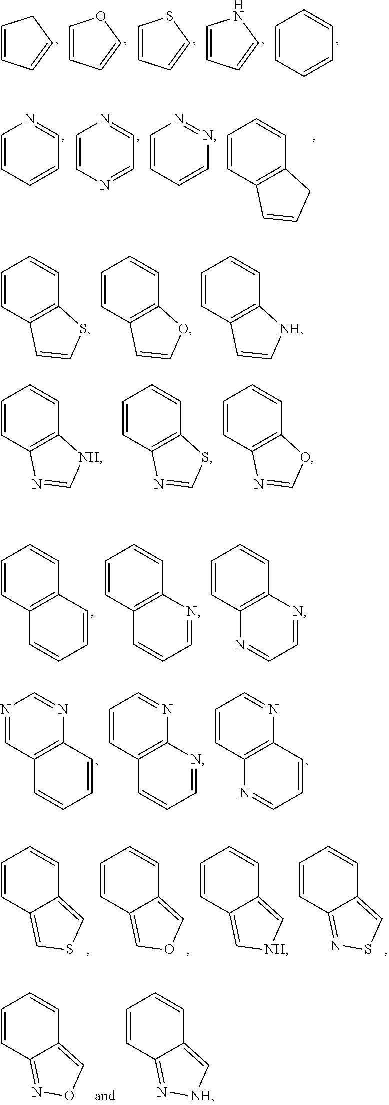

wherein, H on the ring may be arbitrarily substituted. Here, that H on the ring may be arbitrarily substituted means that Ar¹ is further selected from the group consisting of

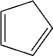

with hydrogen atoms substituted,

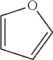

with hydrogen atoms substituted,

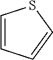

with hydrogen atoms substituted,

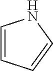

with hydrogen atoms substituted,

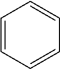

with hydrogen atoms substituted,

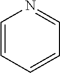

with hydrogen atoms substituted,

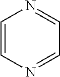

with hydrogen atoms substituted,

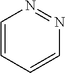

with hydrogen atoms substituted,

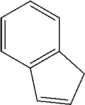

with hydrogen atoms substituted,

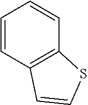

with hydrogen atoms substituted,

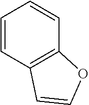

with hydrogen atoms substituted,

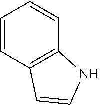

with hydrogen atoms substituted,

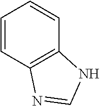

with hydrogen atoms substituted,

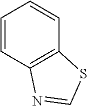

with hydrogen atoms substituted,

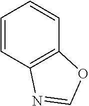

with hydrogen atoms substituted,

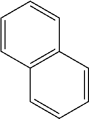

with hydrogen atoms substituted,

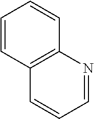

with hydrogen atoms substituted,

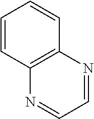

with hydrogen atoms substituted,

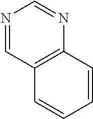

with hydrogen atoms substituted,

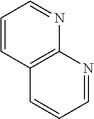

with hydrogen atoms substituted,

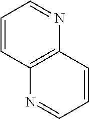

with hydrogen atoms substituted,

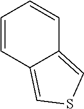

with hydrogen atoms substituted,

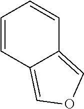

with hydrogen atoms substituted,

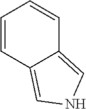

with hydrogen atoms substituted,

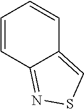

with hydrogen atoms substituted,

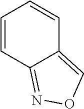

with hydrogen atoms substituted and

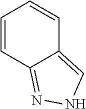

with hydrogen atoms substituted.

Alternatively, in the general formula (1), Ar¹ is one selected from

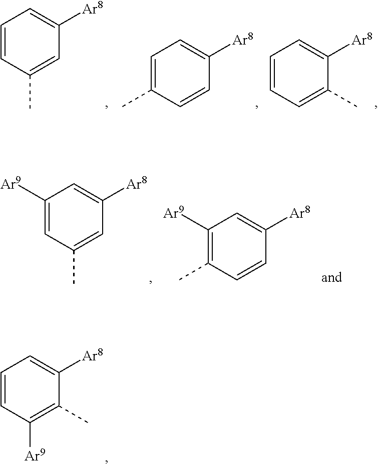

wherein, Ar⁸ and Ar⁹ are each independently selected from the group consisting of an aromatic group with a ring atom number of 5 to 50, and a heteroaromatic group with a ring atom number of 5 to 50.

Specifically, the first organic compound is one selected from the group consisting of

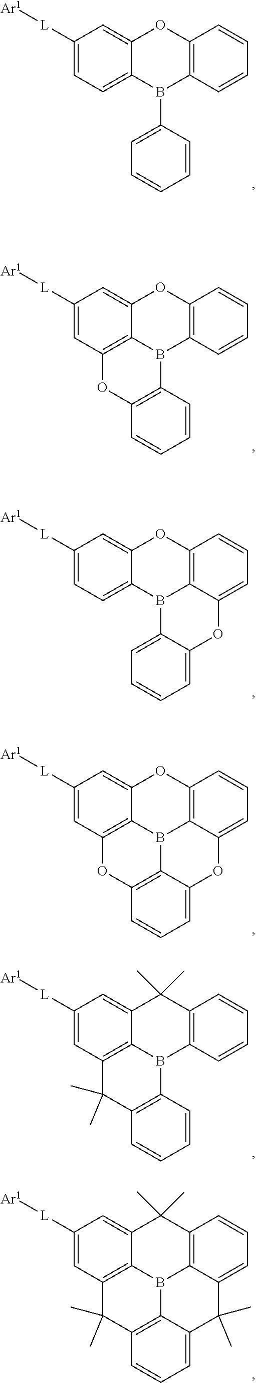

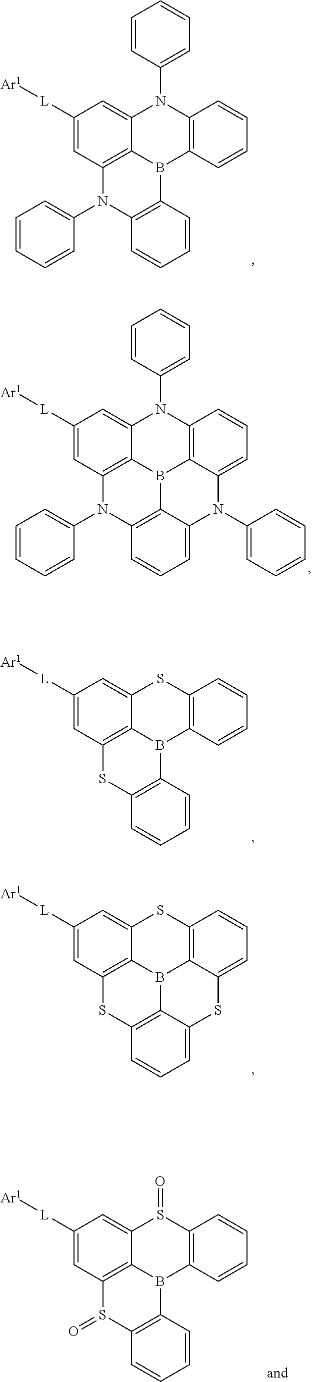

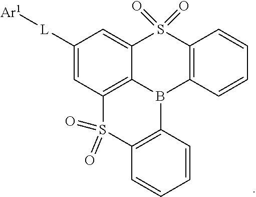

Further, in the general formula (1), Ar¹ is one selected from the group consisting of an aromatic group with a ring atom number of 5 to 60 containing an electron-accepting group, and a heteroaromatic group with a ring atom number of 5 to 60 containing an electron-accepting group.

Further, the electron-accepting group is one selected from the group consisting of F, CN,

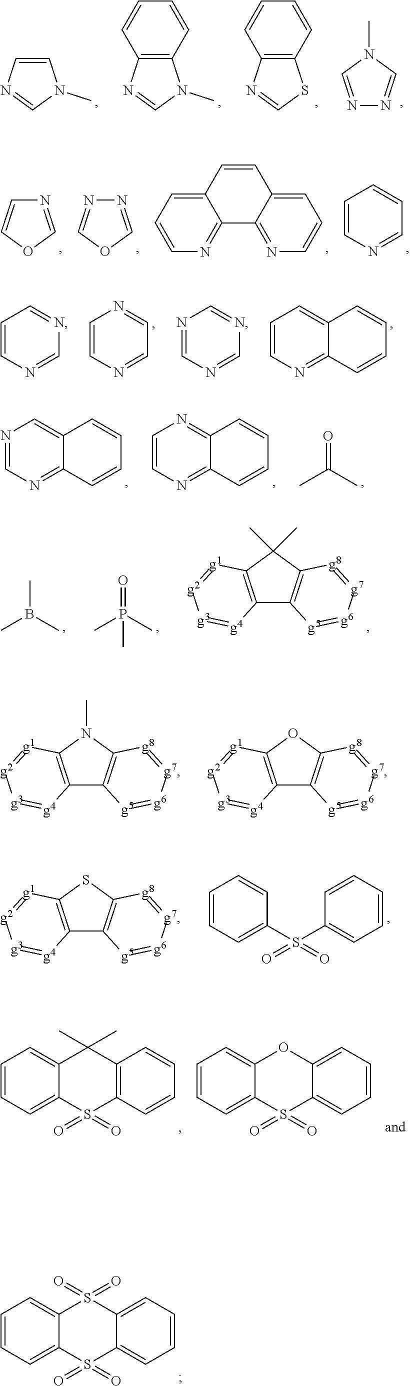

wherein, g¹, g², g³, g⁴, g⁵, g⁶, g⁷ and g⁸ are each independently selected from C and N, and at least one of g¹, g², g³, g⁴, g⁵, g⁶, g⁷ and g⁸ is N.

Further, the electron-accepting group is one selected from the group consisting of CN,

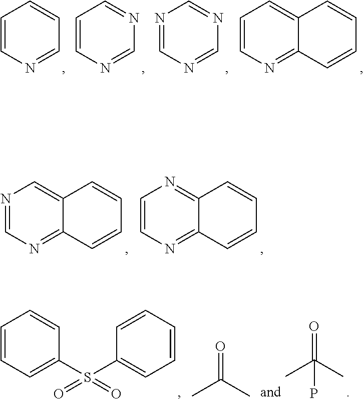

Further, the first organic compound satisfies ((LUMO+1)−LUMO)≥20.1 eV; further the first organic compound satisfies ((LUMO+1)−LUMO)≥20.15 eV; further, the first organic compound satisfies ((LUMO+1)−LUMO)≥20.20 eV; further, the first organic compound satisfies ((LUMO+1)−LUMO)≥0.25 eV; and still further, the first organic compound satisfies ((LUMO+1)−LUMO)≥20.30 eV.

Further, the first organic compound has a glass transition temperature T_{g}≥100° C.; further, the first organic compound has a glass transition temperature T_{g}≥120° C.; further, the first organic compound has a glass transition temperature T_{g}≥140° C.; further, the first organic compound has a glass transition temperature T_{g}≥160° C.; and still further, the first organic compound has a glass transition temperature T_{g}≥180° C.;

In the present embodiment, the structural formula of the second organic compound is one selected from

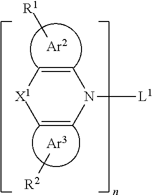

general formula (2),

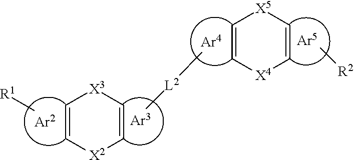

general formula (3),

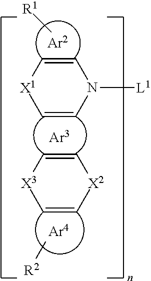

general formula (4), and

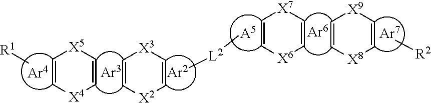

general formula (5).

Wherein, in the general formula (2) and general formula (4), L¹ is one selected from the group consisting of an aromatic group with a ring atom number of 5 to 60, and a heteroaromatic group with a ring atom number of 5 to 60. Further, L¹ is one selected from the group consisting of an aromatic group with a ring atom number of 5 to 50, and a heteroaromatic group with a ring atom number of 5 to 50; further, L¹ is one selected from the group consisting of an aromatic group with a ring atom number of 5 to 40, and a heteroaromatic group with a ring atom number of 5 to 40; and further, L¹ is one selected from the group consisting of an aromatic group with a ring atom number of 5 to 30, and a heteroaromatic group with a ring atom number of 5 to 30.

In the general formulas (3) and (5), -L²- is a single bond, or L² is one selected from the group consisting of an aromatic group with a ring atom number of 5 to 30, and a heteroaromatic group with a ring atom number of 5 to 30. Further, -L²- is a single bond, or L² is one selected from the group consisting of an aromatic group with a ring atom number of 5 to 25, and a heteroaromatic group with a ring atom number of 5 to 25; further, -L²- is a single bond, or L² is one selected from the group consisting of an aromatic group with a ring atom number of 5 to 20, and a heteroaromatic group with a ring atom number of 5 to 20; and further, -L²- is a single bond, or L² is one selected from the group consisting of an aromatic group with a ring atom number of 5 to 15, and a heteroaromatic group with a ring atom number of 5 to 15.

In the general formulas (2) to (5),

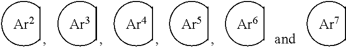

are each independently selected from the group consisting of an aromatic group with a ring atom number of 5 to 30, and a heteroaromatic group with a ring atom number of 5 to 30; further,

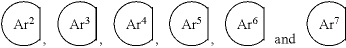

are each independently selected from the group consisting of an aromatic group with a ring atom number of 5 to 25, and a heteroaromatic group with a ring atom number of 5 to 25; further

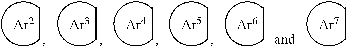

are each independently selected from the group consisting of an aromatic group with a ring atom number of 5 to 20, and a heteroaromatic group with a ring atom number of 5 to 20; and further,

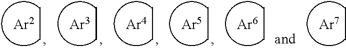

and are each independently selected from the group consisting of an aromatic group with a ring atom number of 5 to 15, and a heteroaromatic group with a ring atom number of 5 to 15.

Further, in the general formulas (2) to (5), the structural formulas of

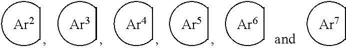

are each independently selected from one of

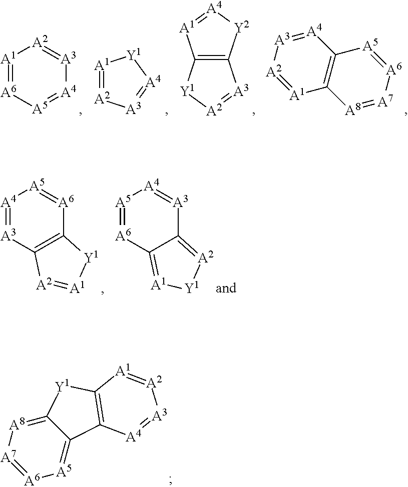

wherein, A¹, A², A³, A⁴, A⁵, A⁶, A⁷ and A⁸ are each independently one selected from CR³ and N;

Y¹ and Y² are each independently selected from the group consisting of CR⁴R⁵, SiR⁴R⁵, NR³, C(═O), S and O;

R³, R⁴ and R⁵ are each independently selected from the group consisting of H, D, a linear alkyl with a carbon atom number of 1 to 20, an alkoxy with a carbon atom number of 1 to 20 and a thioalkoxy with a carbon atom number of 1 to 20, a branched alkyl with a carbon atom number of 1 to 20, a cyclic alkyl with a carbon atom number of 1 to 20, and a silyl with a carbon atom number of 1 to 20, a ketone group with a carbon atom number of 1 to 20, an alkoxycarbonyl group with a carbon atom number of 1 to 20, an aryloxycarbonyl with a carbon atom number of 7 to 20, cyano, carbamoyl (—C(═O)NH₂), haloformyl (C(═O)—X, wherein X represents a halogen atom), formyl(—C(═O)—H), isocyano, an isocyanate group, a thiocyanate group, an isothiocyanate group, hydroxyl, nitro, CF₃, Cl, Br, F, a crosslinkable group, an aryl containing 5 to 40 ring atoms, a heteroaryl containing 5 to 40 ring atoms, an aryloxy containing 5 to 40 ring atoms and a heteroaryloxy containing 5 to 40 ring atoms. The crosslinkable group refers to a functional group containing unsaturated bonds, such as alkenyl, alkynyl, etc.

Still further, in the general formulas (2) to (5),

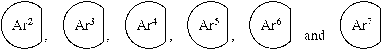

are each independently selected from the group consisting of

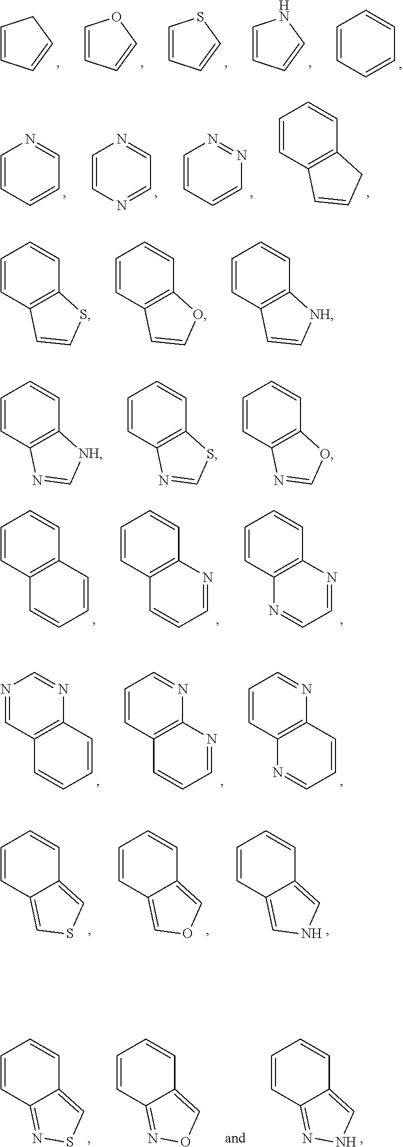

wherein, H on the ring may be arbitrarily substituted. Wherein, that H on the ring may be arbitrarily substituted means that Ar¹ is further selected from the group consisting of

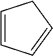

with hydrogen atoms substituted,

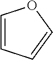

with hydrogen atoms substituted,

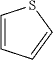

with hydrogen atoms substituted,

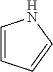

with hydrogen atoms substituted,

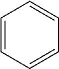

with hydrogen atoms substituted,

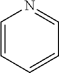

with hydrogen atoms substituted,

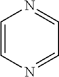

with hydrogen atoms substituted,

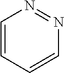

with hydrogen atoms substituted,

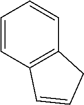

with hydrogen atoms substituted,

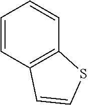

with hydrogen atoms substituted,

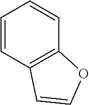

with hydrogen atoms substituted,

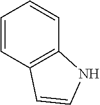

with hydrogen atoms substituted,

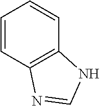

with hydrogen atoms substituted,

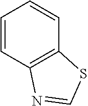

with hydrogen atoms substituted,

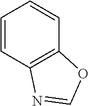

with hydrogen atoms substituted, with

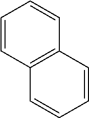

hydrogen atoms substituted,

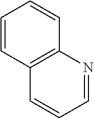

with hydrogen atoms substituted,

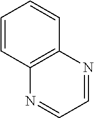

with hydrogen atoms substituted,

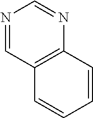

with hydrogen atoms substituted,

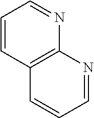

with hydrogen atoms substituted,

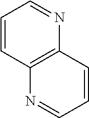

with hydrogen atoms substituted,

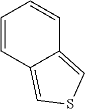

with hydrogen atoms substituted,

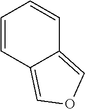

with hydrogen atoms substituted,

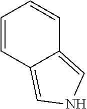

with hydrogen atoms substituted,

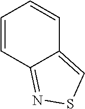

with hydrogen atoms substituted,

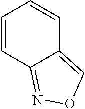

with hydrogen atoms substituted and

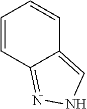

with hydrogen atoms substituted.

In the general formulas (2) and (4), —X¹— is a single bond, or X¹ is one selected from the group consisting of N(R), C(R)₂, Si(R)₂, O, C═N(R), C═C(R)₂, P(R), P(═O)R, S, S═O and SO₂. Further, —X¹— in the general formulas (2) to (4) is a single bond, or X¹ is one selected from the group consisting of N(R), C(R)₂, O and S.

In the general formulas (3) to (5), —X²—, —X³—, —X⁴—, —X⁵—, —X⁶—, —X⁷—, —X¹— and —X⁹— are each independently selected from the group consisting of a single bond, —N(R)—, —C(R)₂—, —Si(R)₂—, —O—, —(C═N(R⁶))—, —(C═C(R⁶)₂)—, —P(R⁶)—, —(P(═O)R⁶)—, —S—, —(S═O)— and —(SO₂)—, and at most one of —X²— and —X³— is a single bond, at most one of —X⁴— and —X¹— is a single bond, at most one of —X⁶— and —X⁷— is a single bond, and at most one of —X¹— and —X⁹— is a single bond. In one embodiment, in the general formulas (3) to (5), one of —X²— and —X³— is a single bond, and the other is one selected from —N(R)—, —C(R)₂—, —O— and —S—; one of —X⁴— and —X⁵— is a single bond, and the other is one selected from —N(R⁶)—, —C(R⁶)₂—, —O— and —S—; one of —X⁶— and —X⁷— is a single bond, and the other is one selected from —N(R⁶)—, —C(R⁶)₂—, —O— and —S—; and one of —X⁸— and —X⁹— is a single bond, and the other is one selected from —N(R⁶)—, —C(R⁶)₂—, —O— and —S—.

R¹, R² and R⁶ are each independently selected from the group consisting of H, D, F, CN, alkenyl, alkynyl, nitrile group, amino, nitro, acyl, alkoxy, carbonyl, sulfonyl, C_{1˜30} alkyl, C_{3˜30} cycloalkyl, an aromatic hydrocarbyl with a ring atom number of 5 to 60, and an aromatic heterocyclic group with a ring atom number of 5 to 60. Wherein, R¹ and R² are attached to any carbon atom on the fused ring.

Further, R¹, R² and R⁶ are each independently selected from the group consisting of methyl, benzene, diphenyl, naphthalene, anthracene, phenanthrene, pyrene, pyridine, pyrimidine, triazine, fluorene, dibenzothiophene, silafluorene, carbazole, thiophene, furan, thiazole, triphenylamine, triphenylphosphine oxide, tetraphenyl silicane, spirofluorene, and spirosilafluorene; Furthermore, R¹, R² and R⁶ are each independently selected from the group consisting of benzene, diphenyl, pyridine, pyrimidine, triazine and carbazole.

In the general formulas (2) and (4), n is any integer selected from 1 to 4. Further, n is any integer selected from 1 to 3; and still further, n is any integer selected from 1 to 2.

In an embodiment, in the general formulas (1) to (5), L, L¹ and L² are each independently selected from the following groups and the following groups with hydrogen atom substituted:

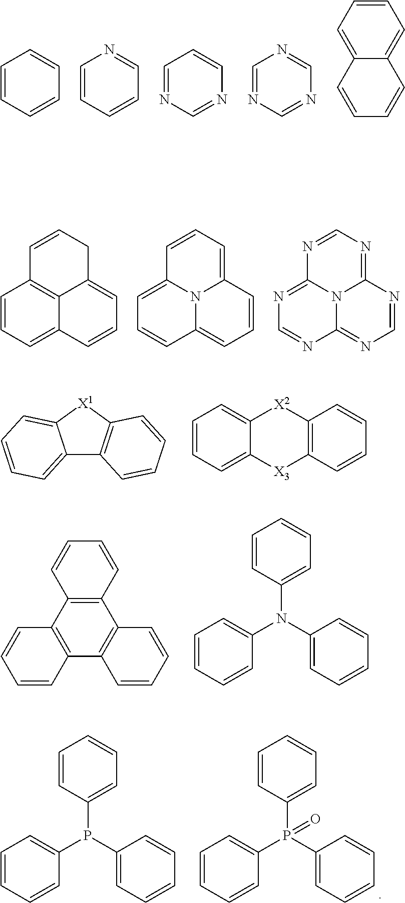

Alternatively, in the general formulas (1) to (5), L, L¹ and L² are each independently selected from the following groups and the following groups with hydrogen atom substituted (where n has the same meaning as described above):

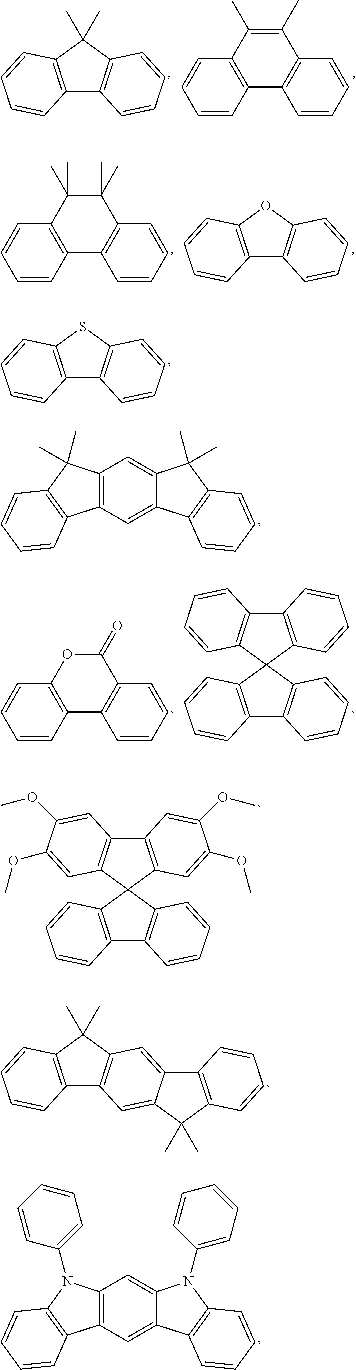

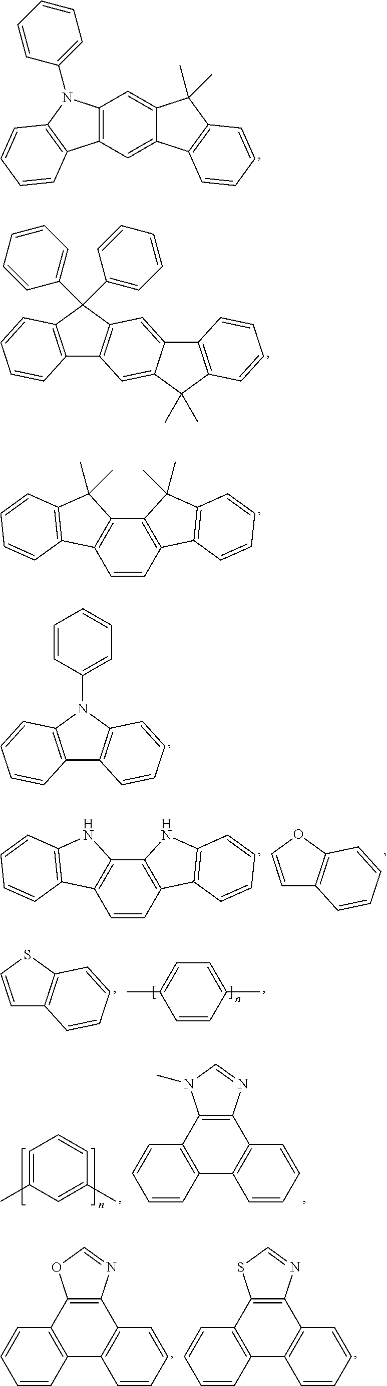

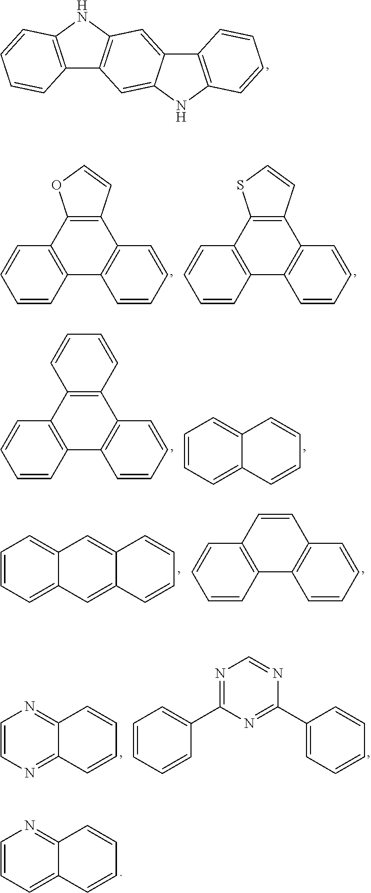

Further, the second organic compound of general formula (2) is one selected from the following structural formulas:

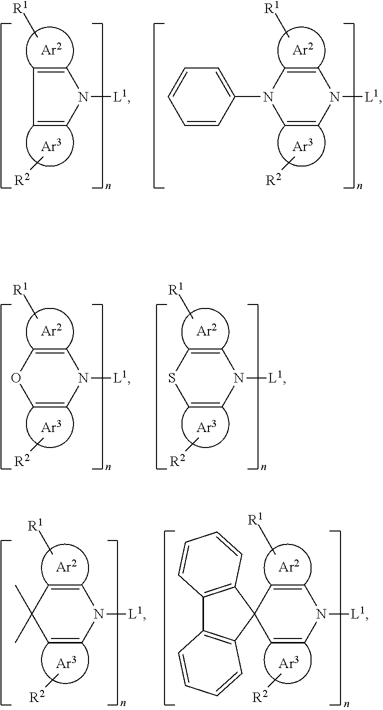

wherein,

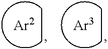

R¹, R², L¹ and n have the same definitions as those in the general formula (2).

Further, the second organic compound of general formula (3) is one selected from the following structural formulas:

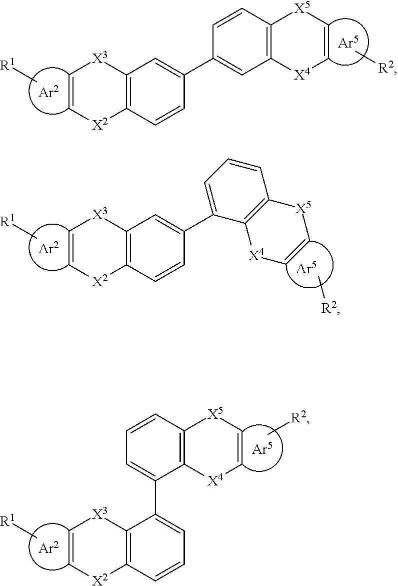

wherein,

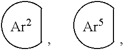

—X²—, —X³—, —X⁴—, —X⁵—, R¹ and R² have the same definitions as those in the general formula (3).

Further, the second organic compound of general formula (4) is one selected from the following structural formulas:

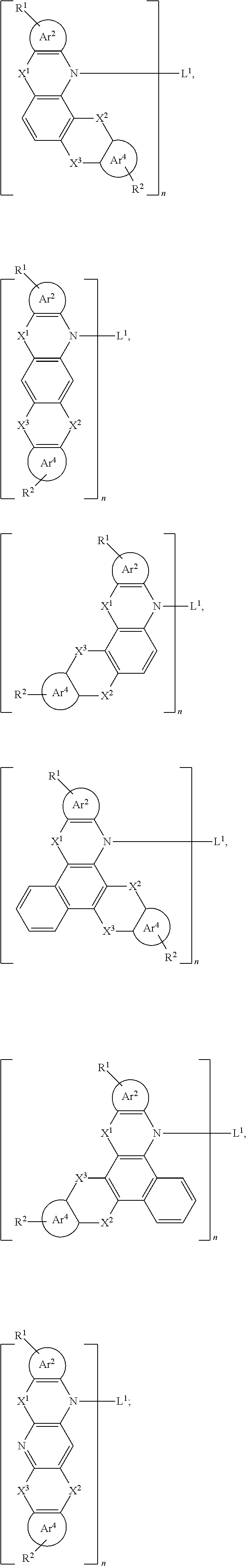

wherein,

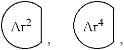

—X¹—, —X²—, —X³—, R¹, R², L¹ and n have the same definitions as those in the general formula (4).

Further, the second organic compound of general formula (5) is one selected from the following structural formulas:

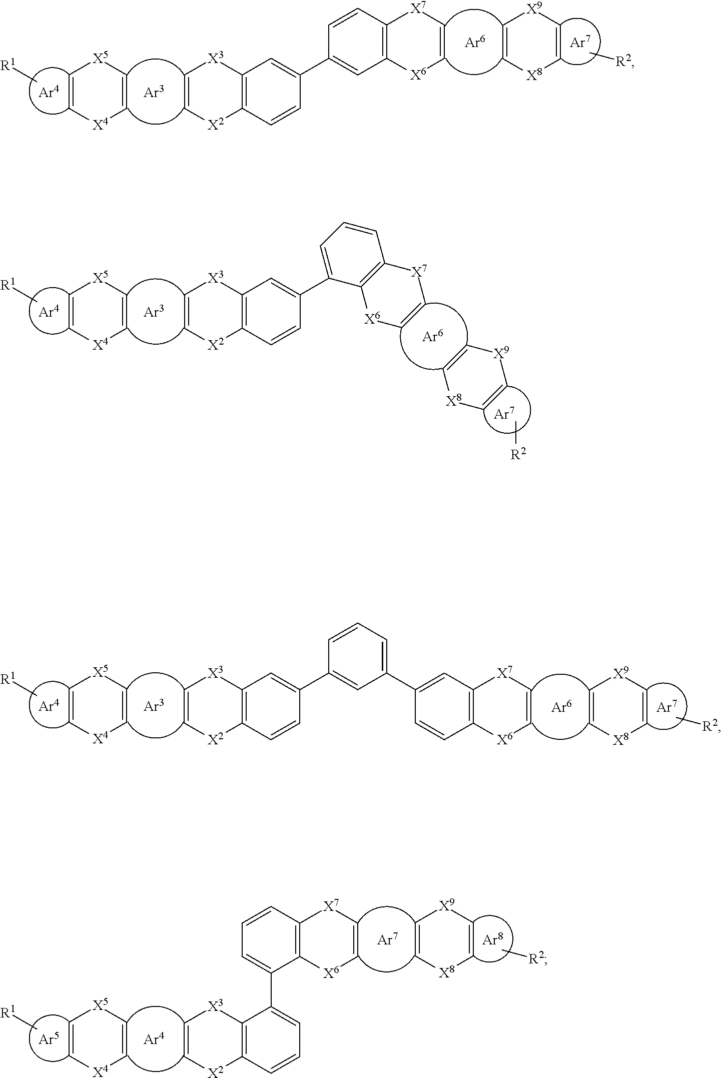

wherein,

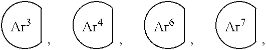

—X²—, —X³—, —X⁴—, —X⁵—, —X⁶—, —X⁷—, —X⁸—, —X⁹—, R¹ and R² have the same definitions as those in the general formula (5).

Specifically, the first organic compound satisfying the general formula (1) includes, but is not limited to, the following compounds:

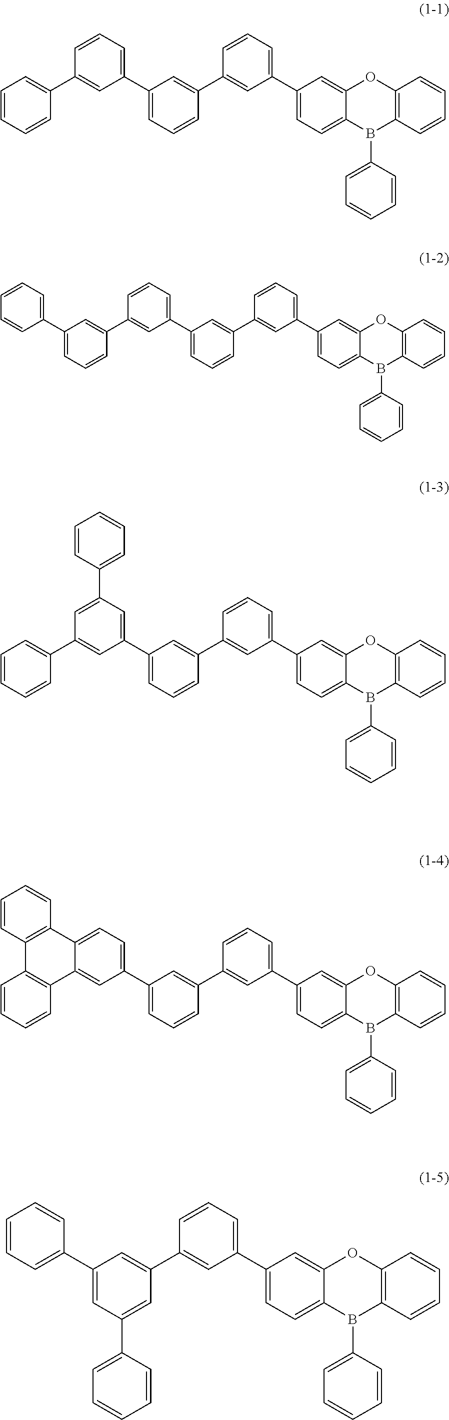

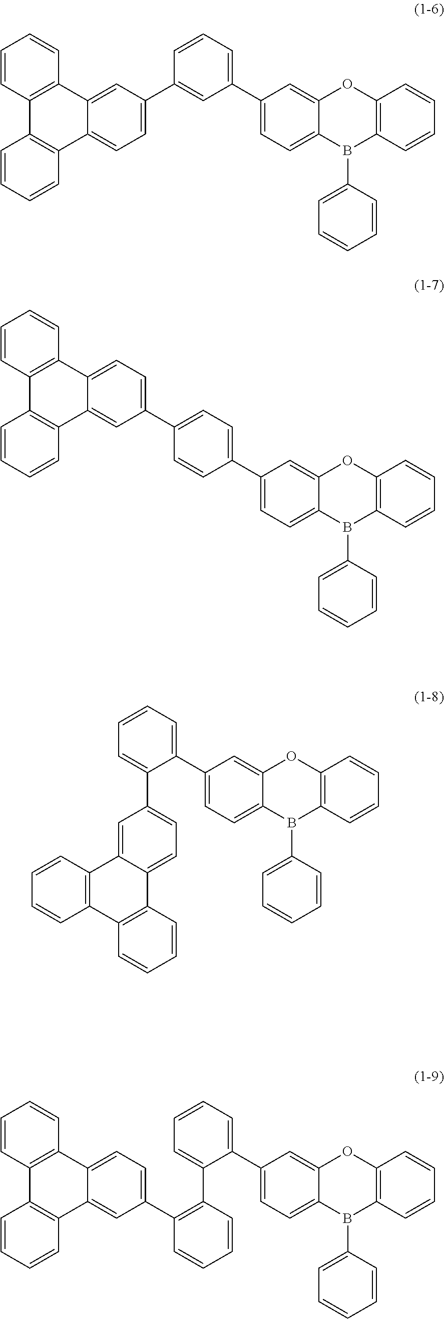

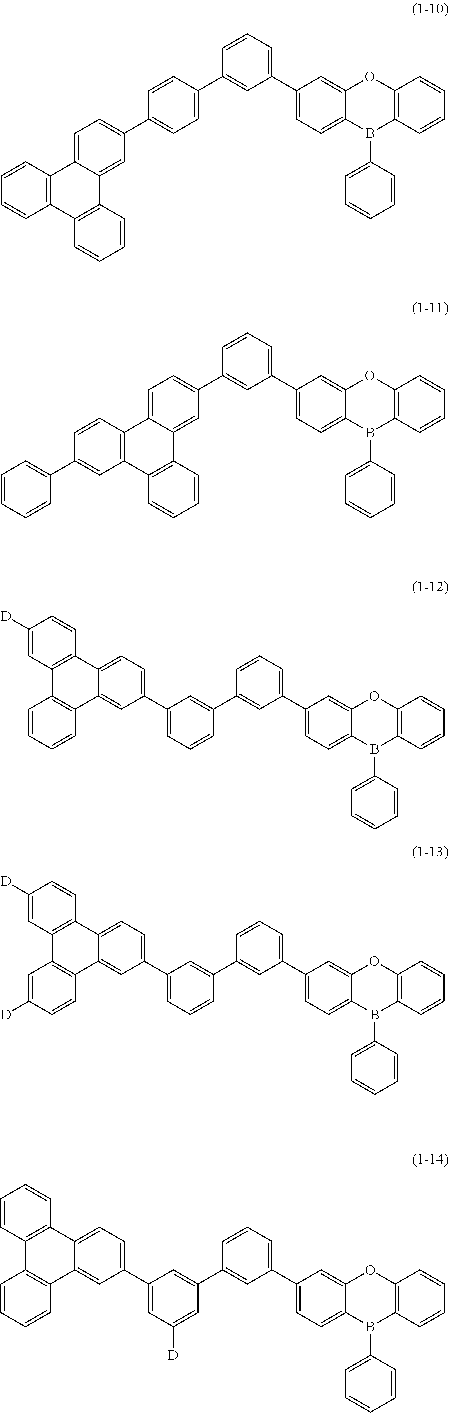

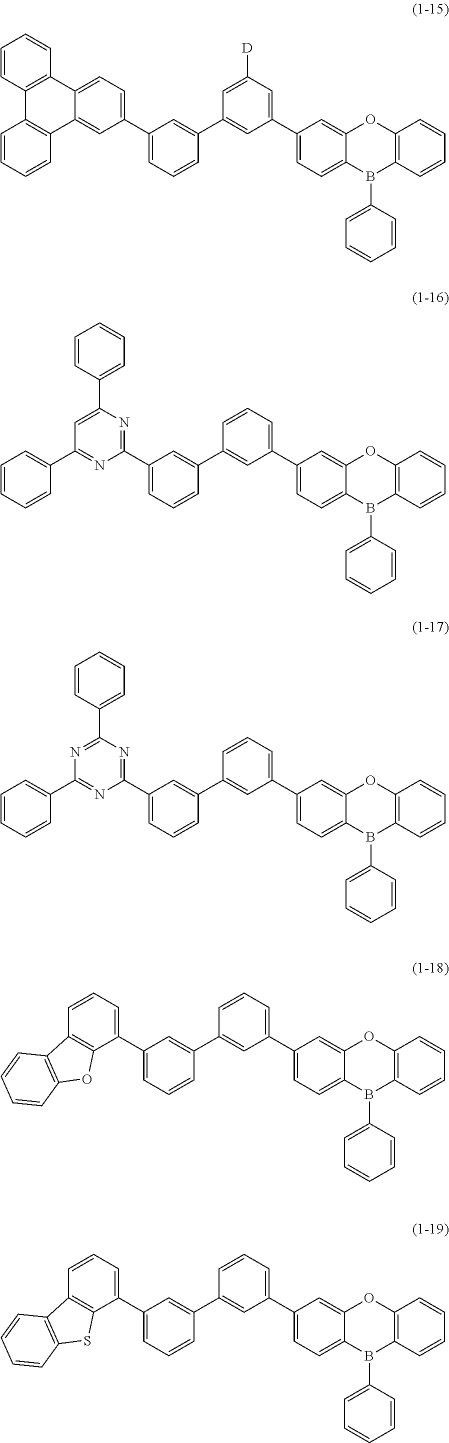

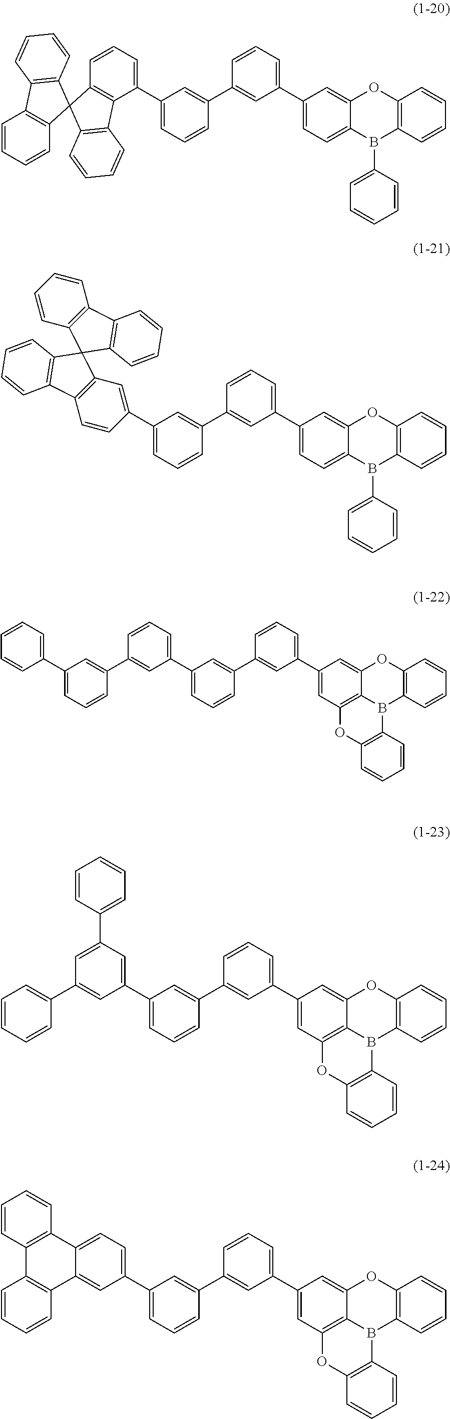

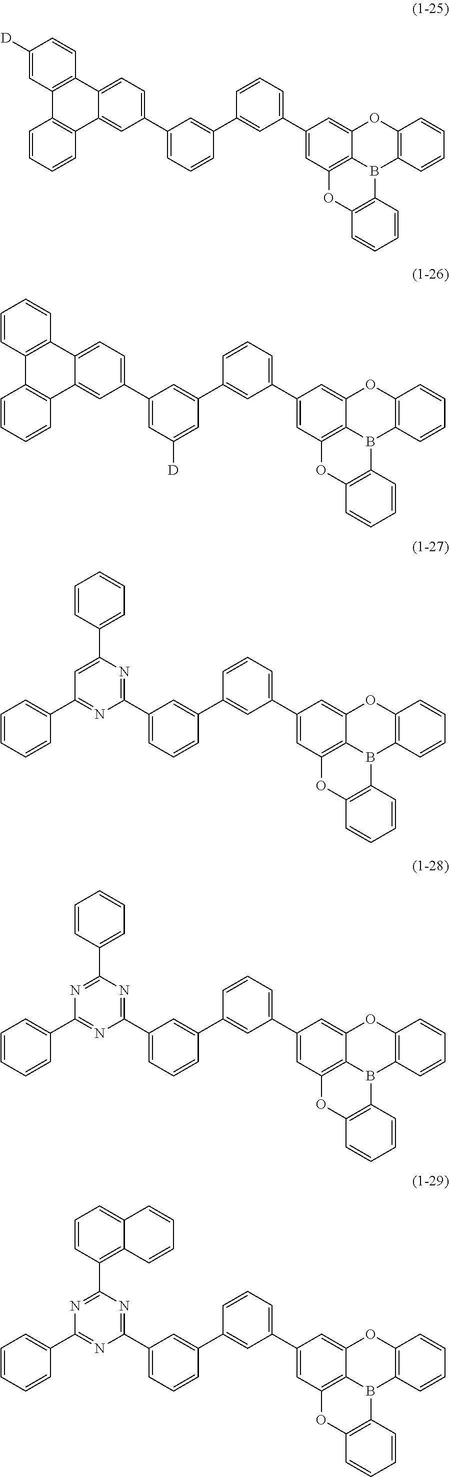

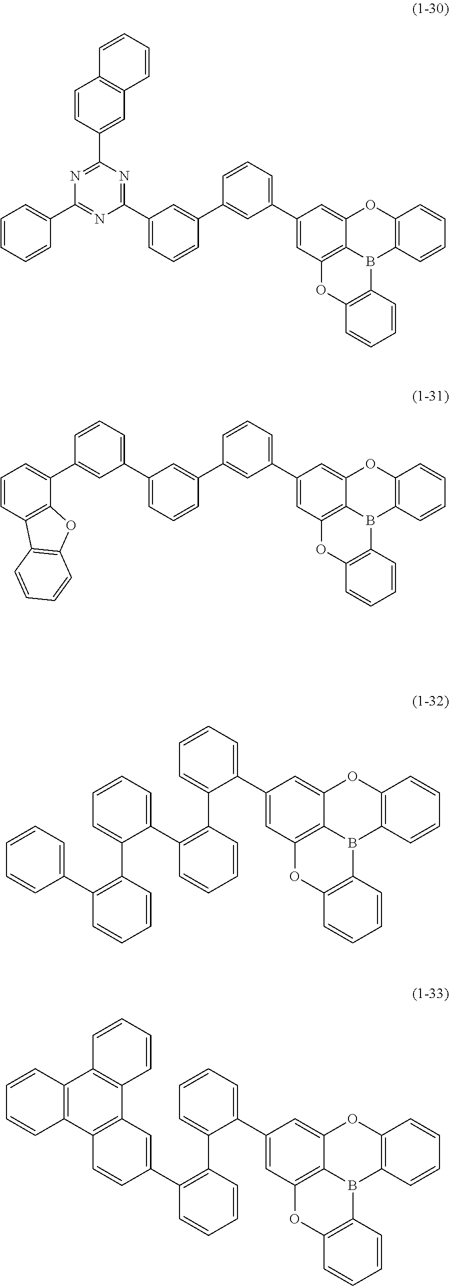

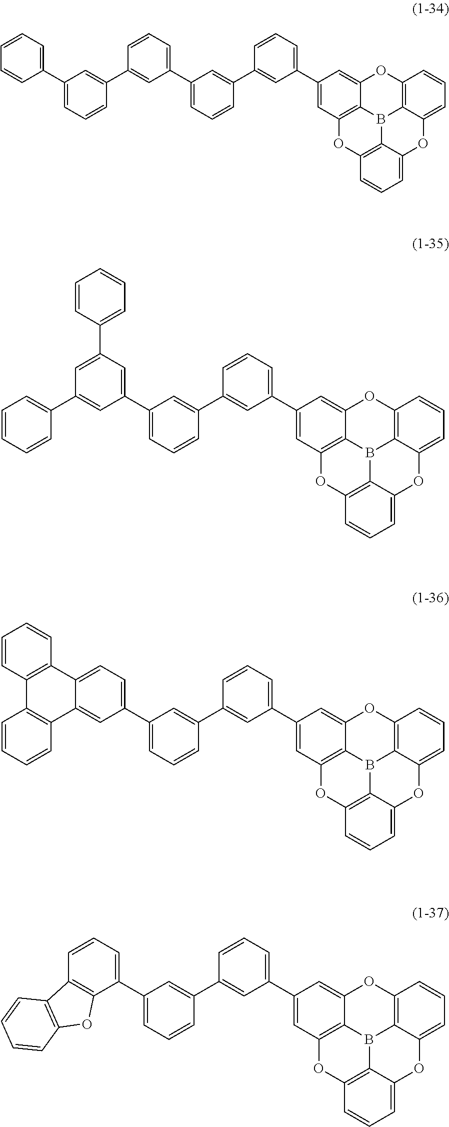

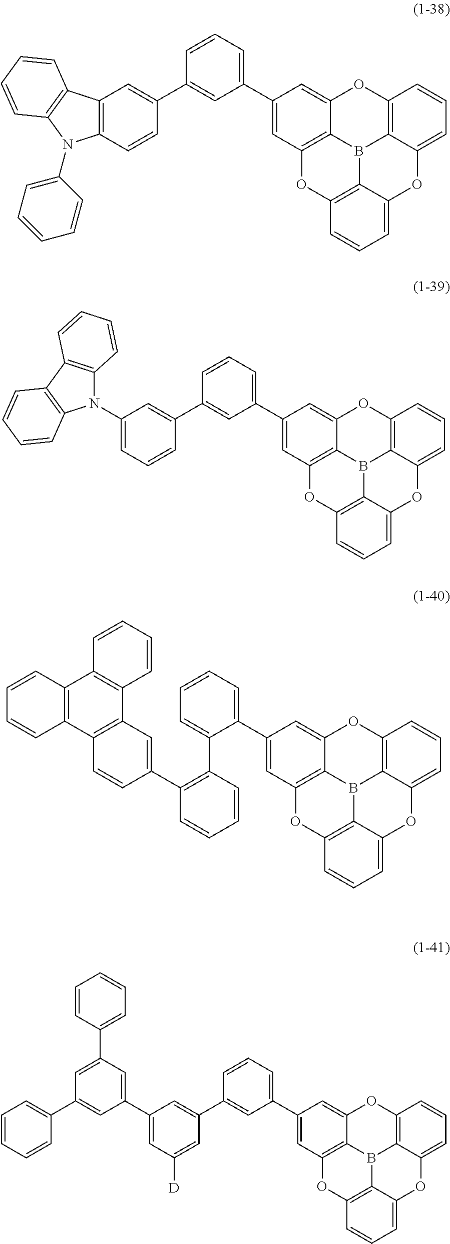

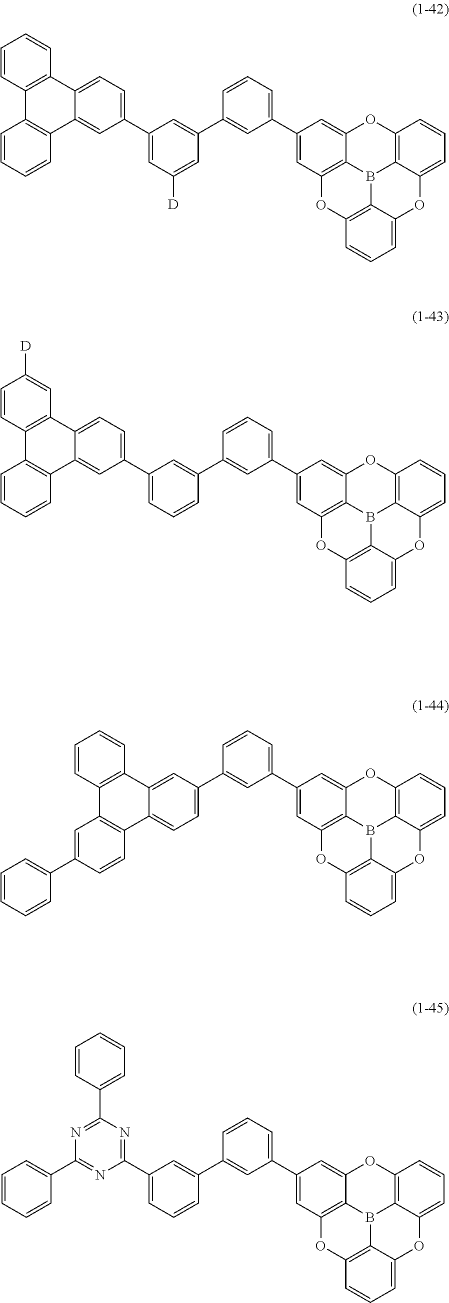

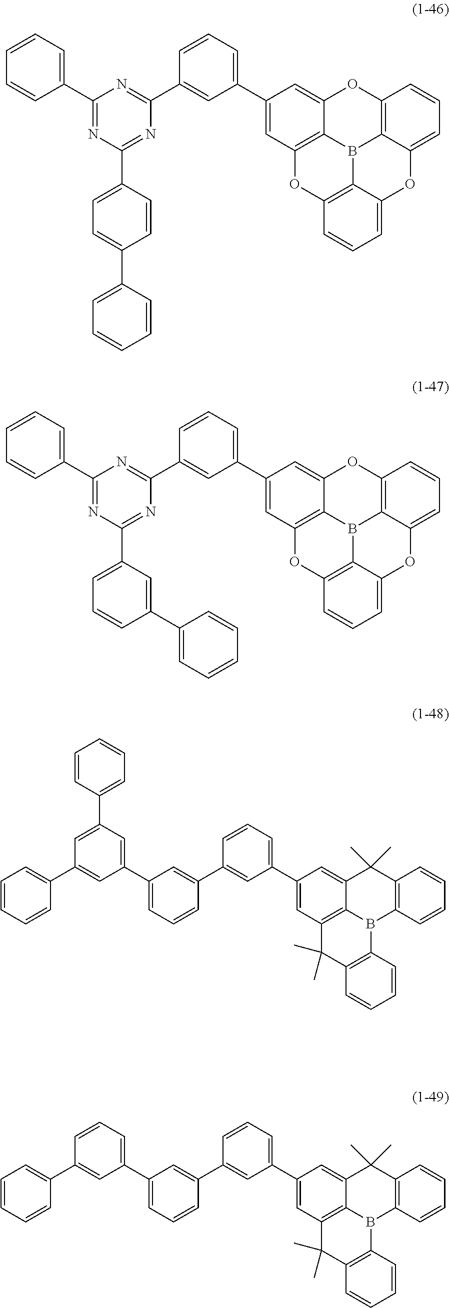

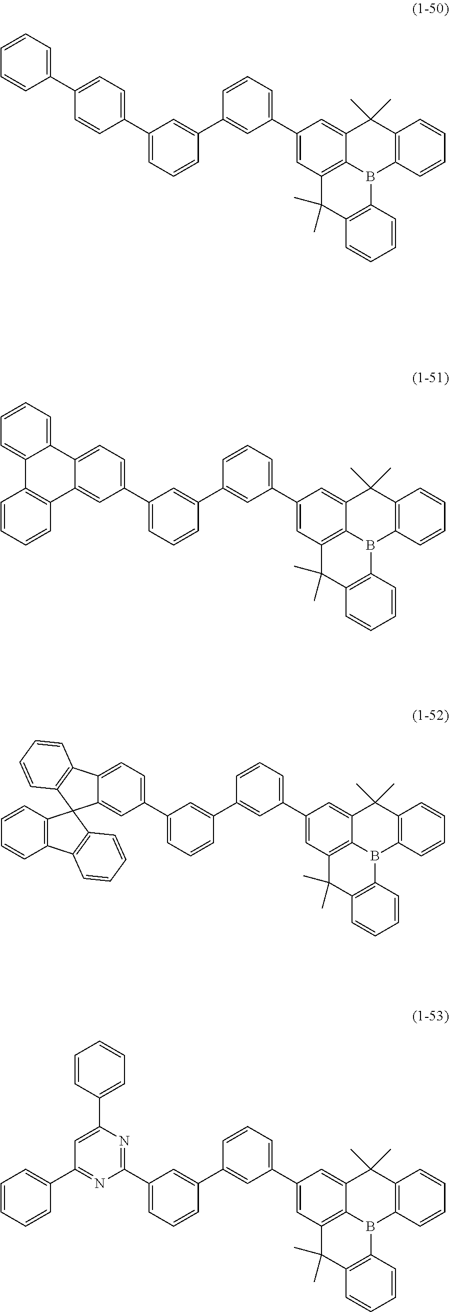

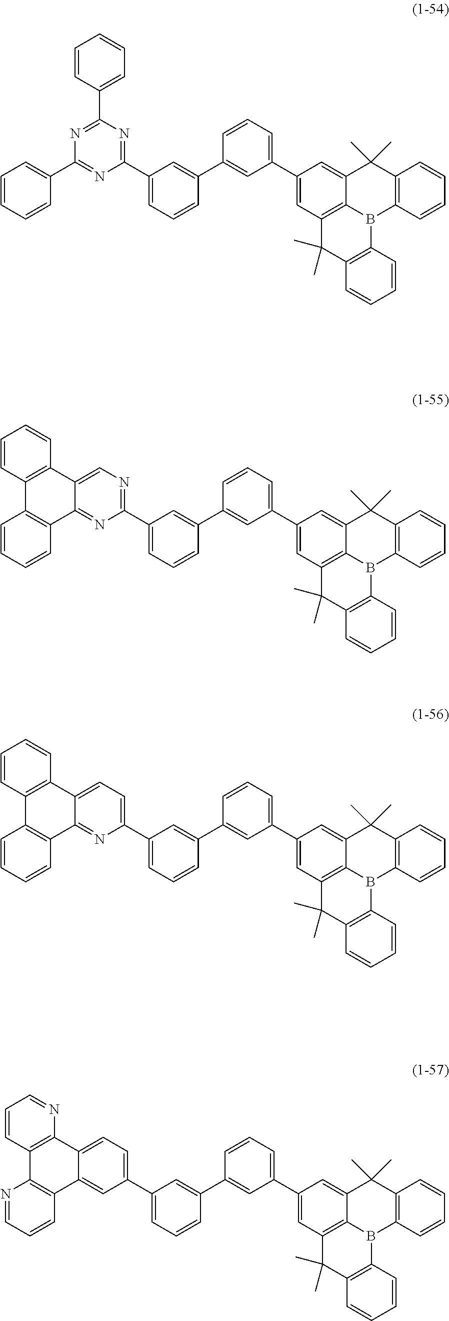

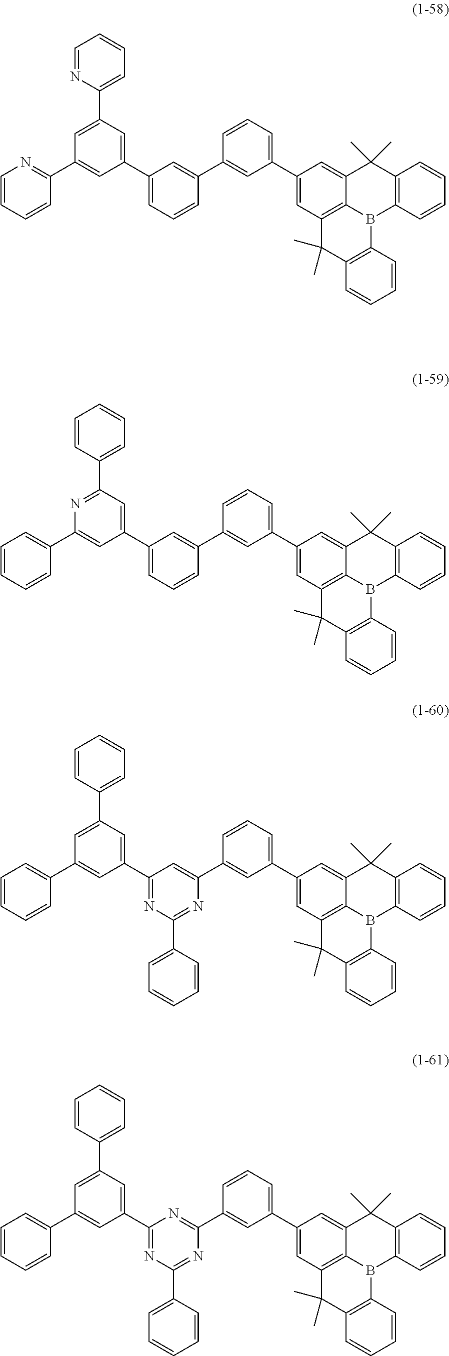

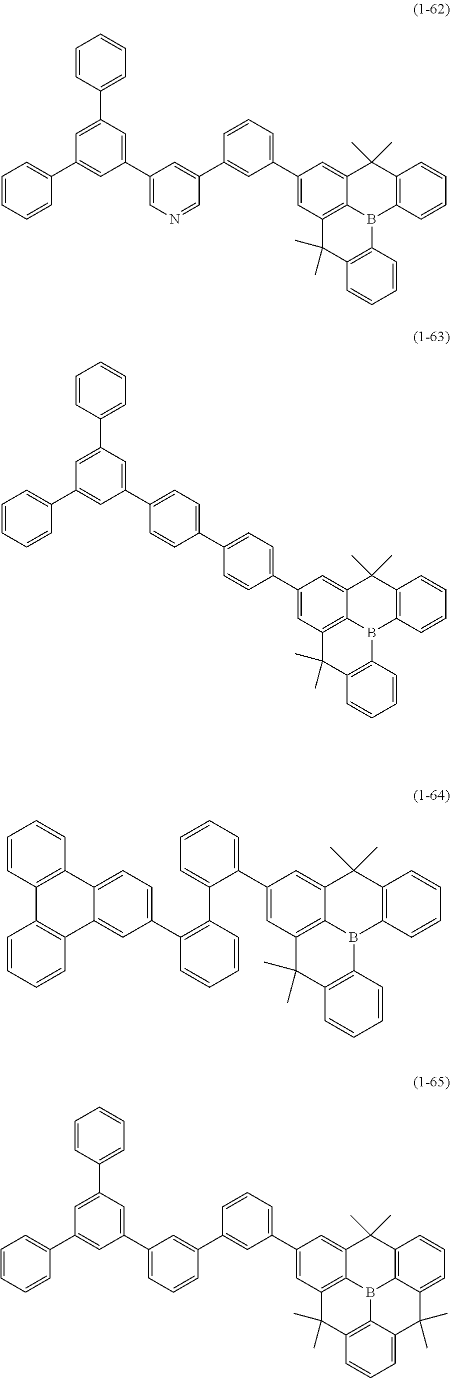

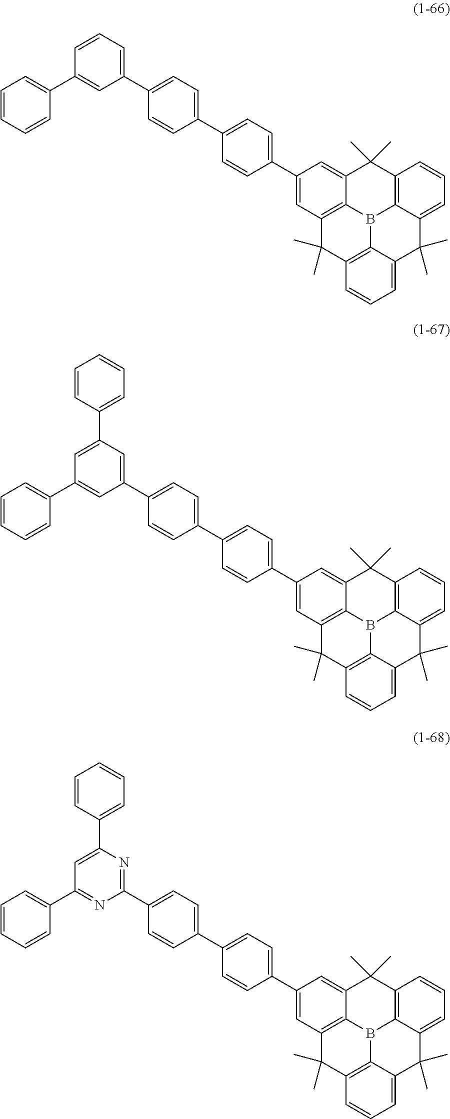

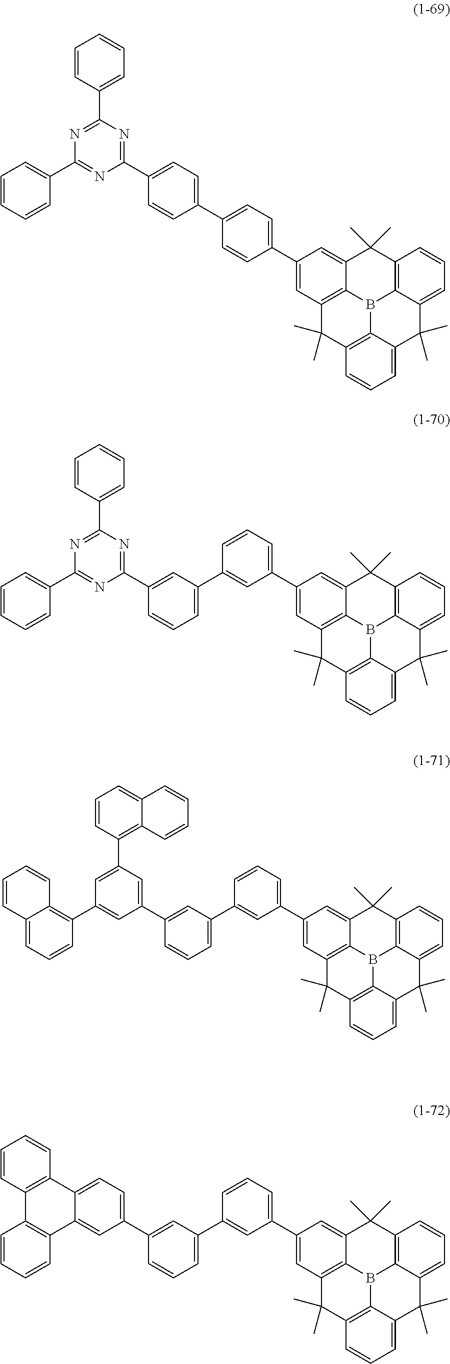

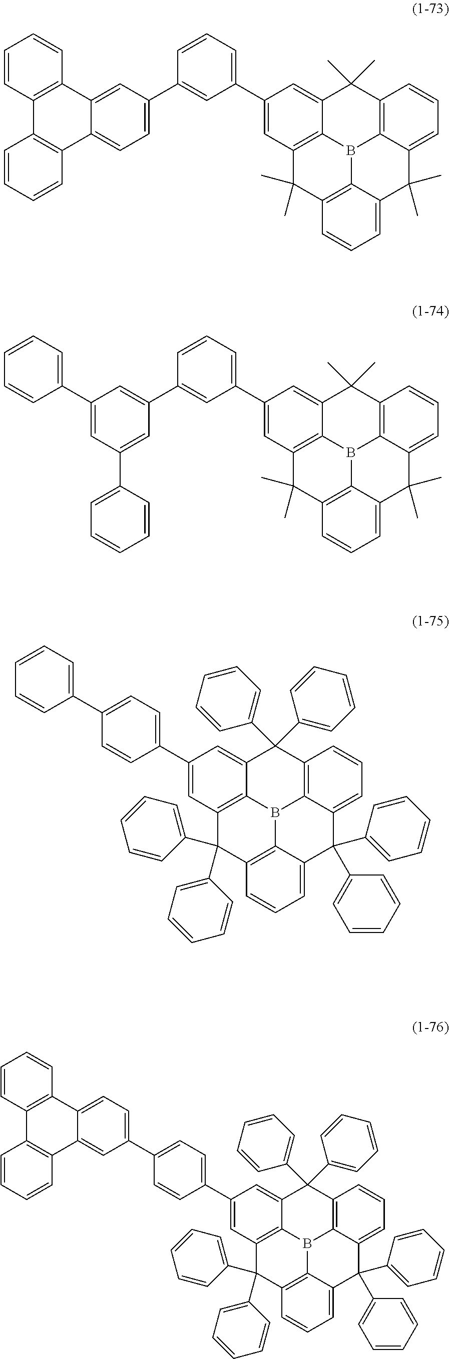

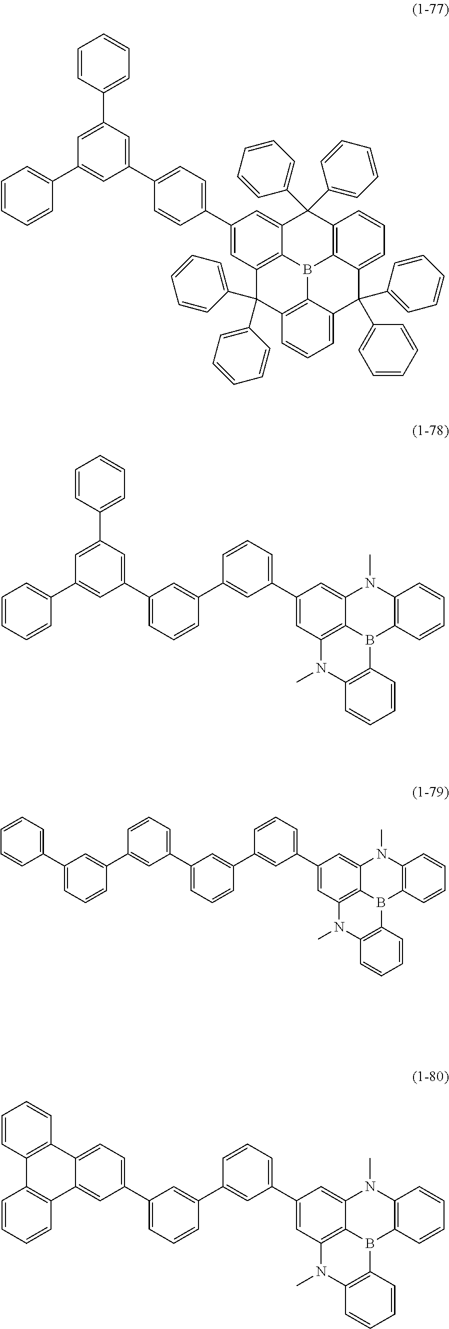

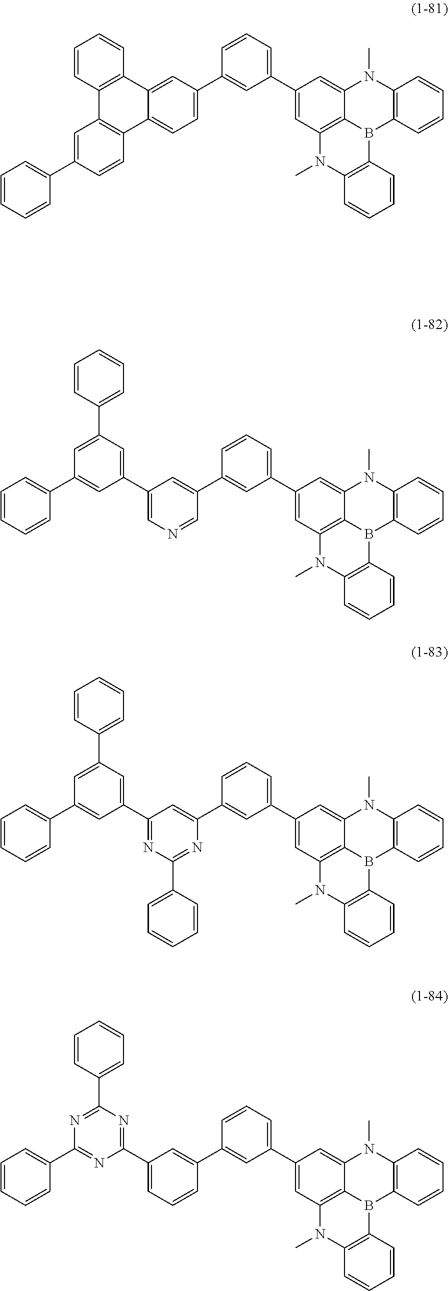

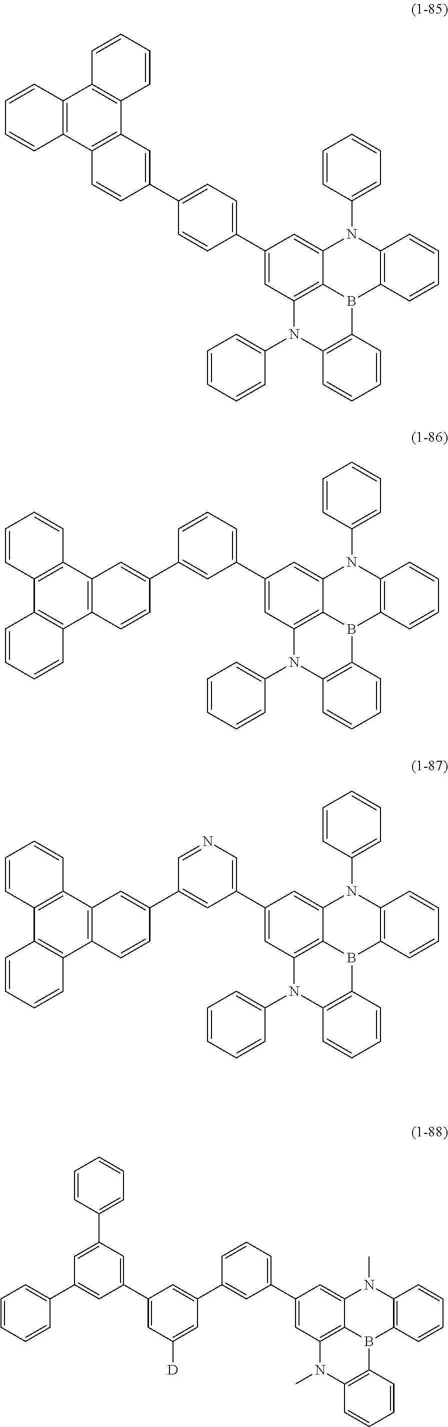

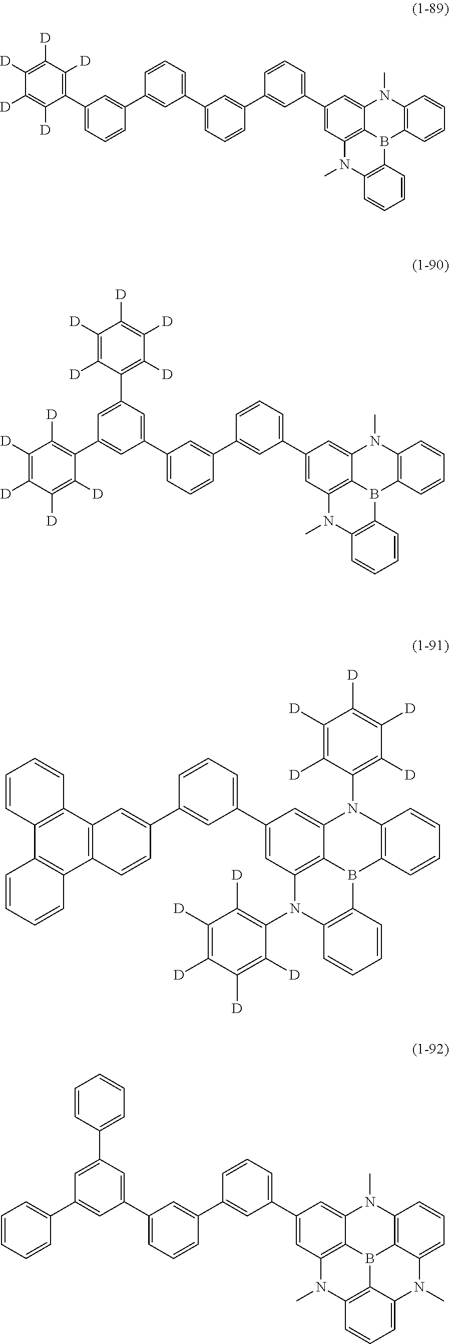

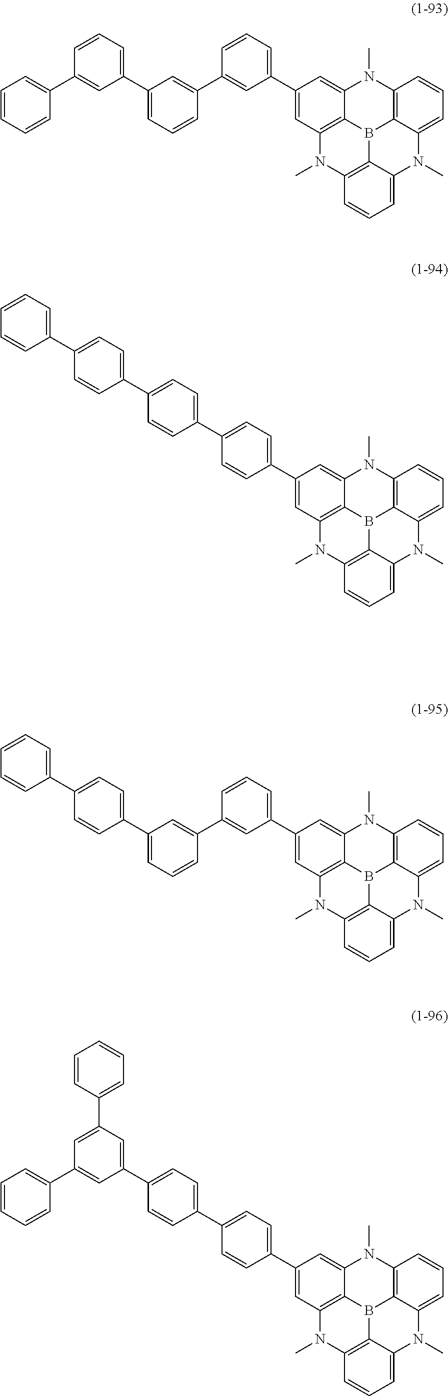

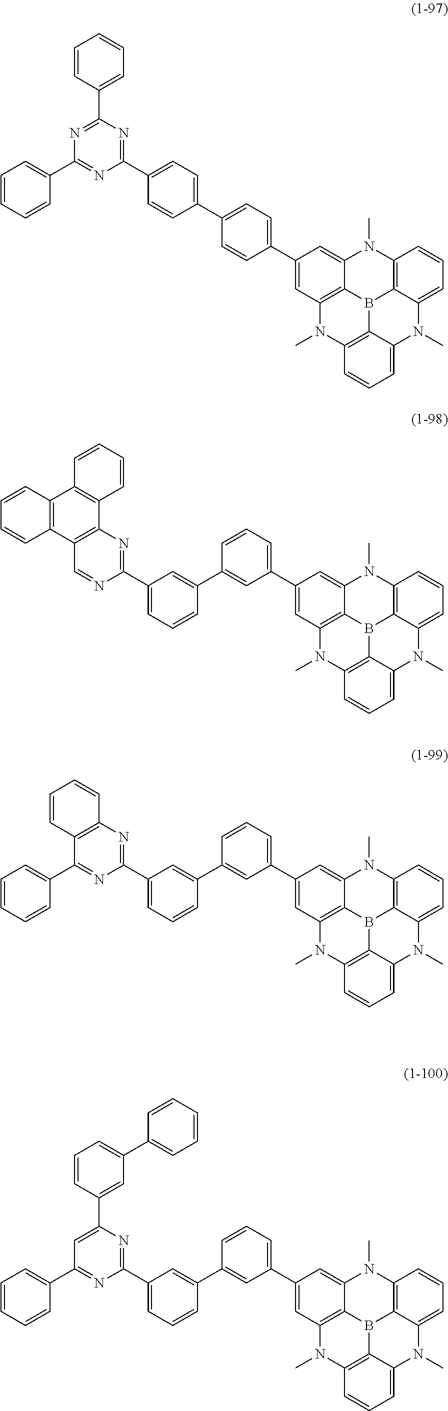

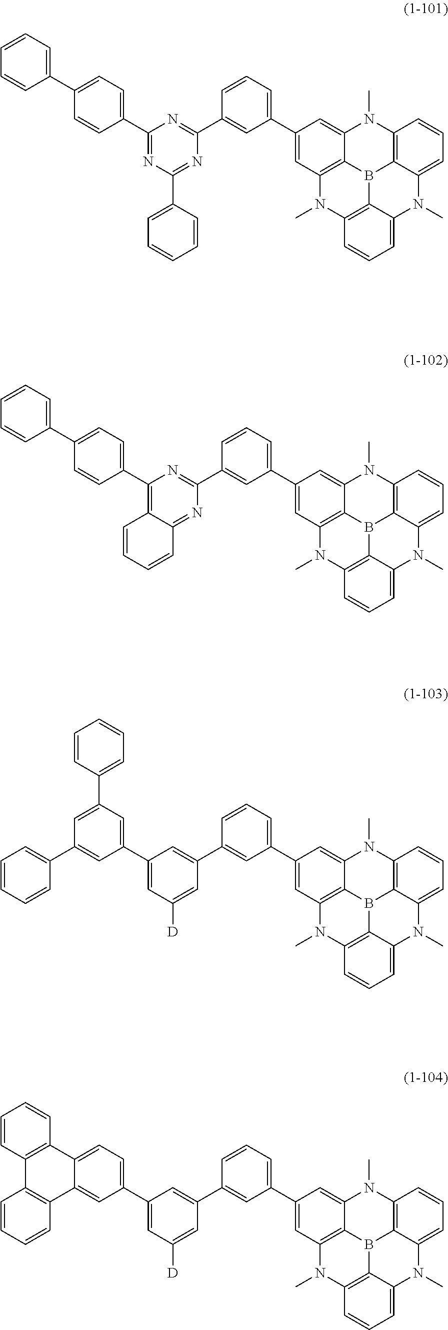

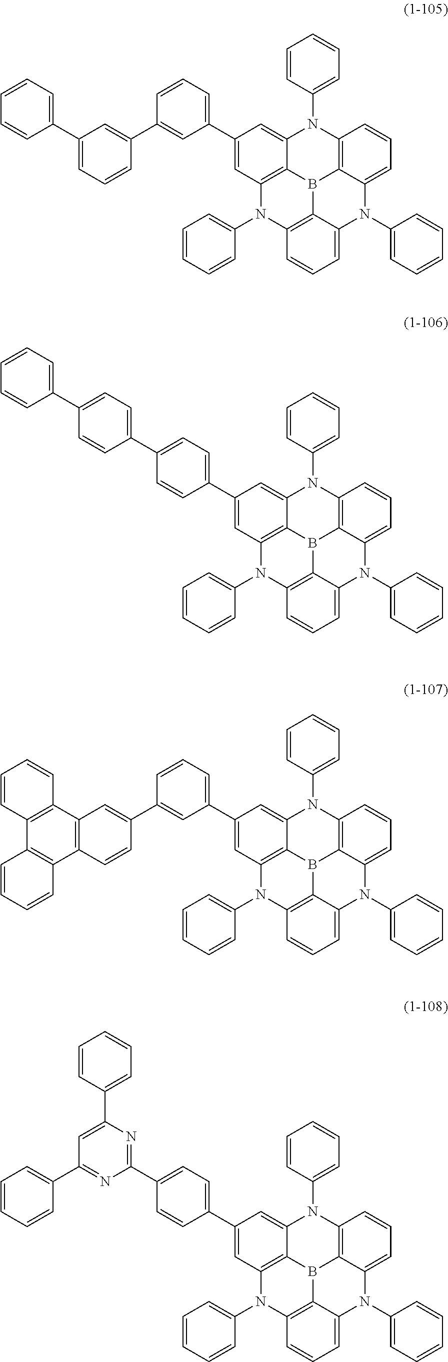

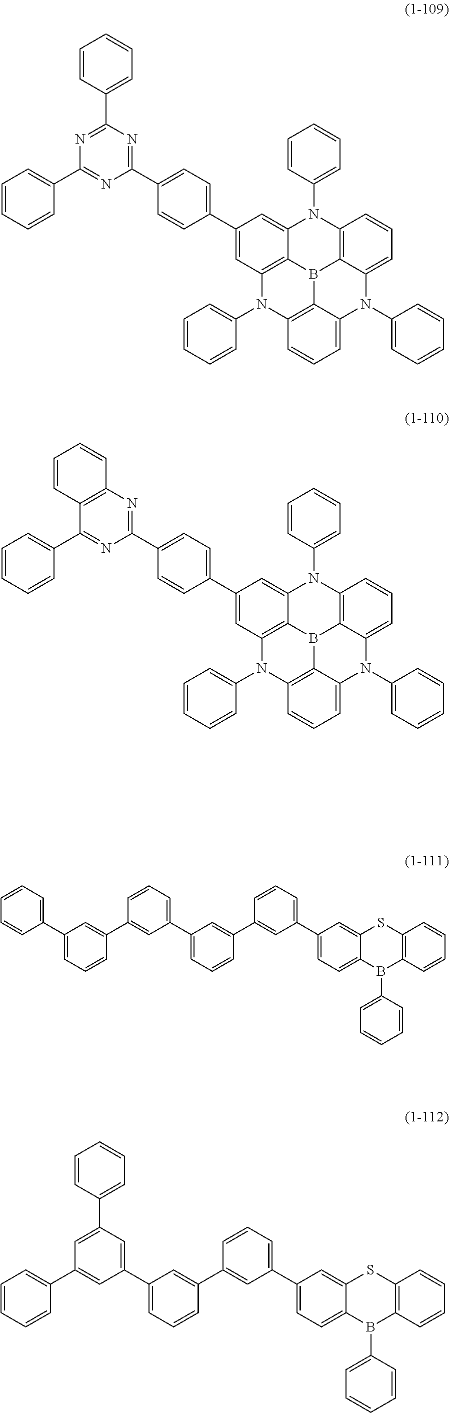

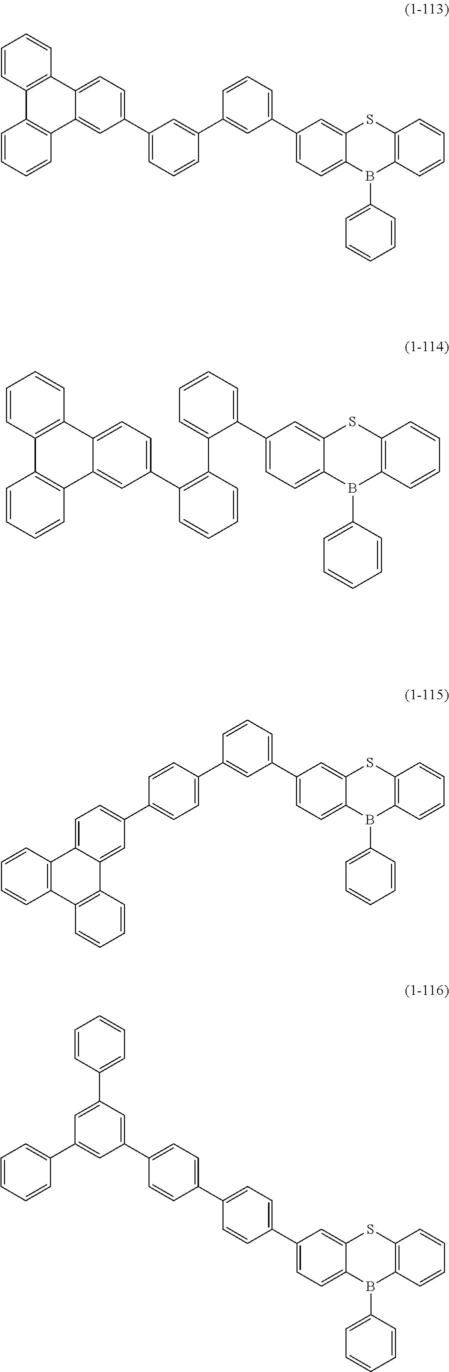

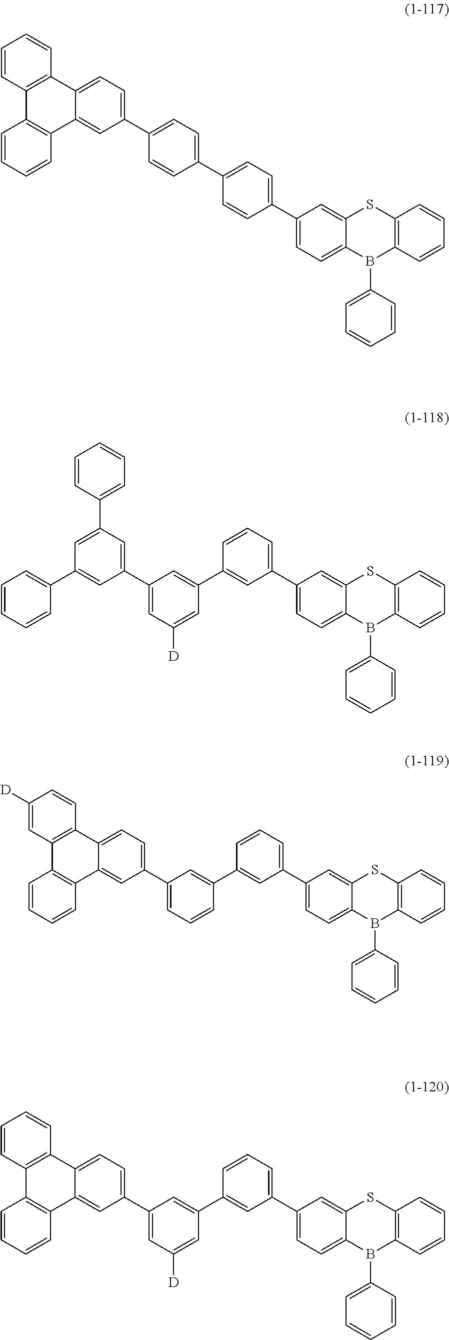

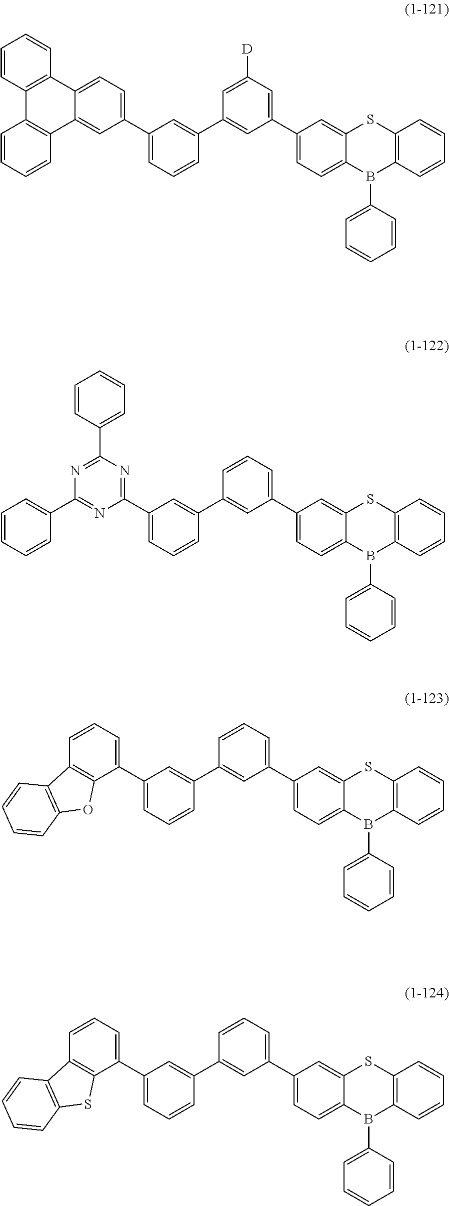

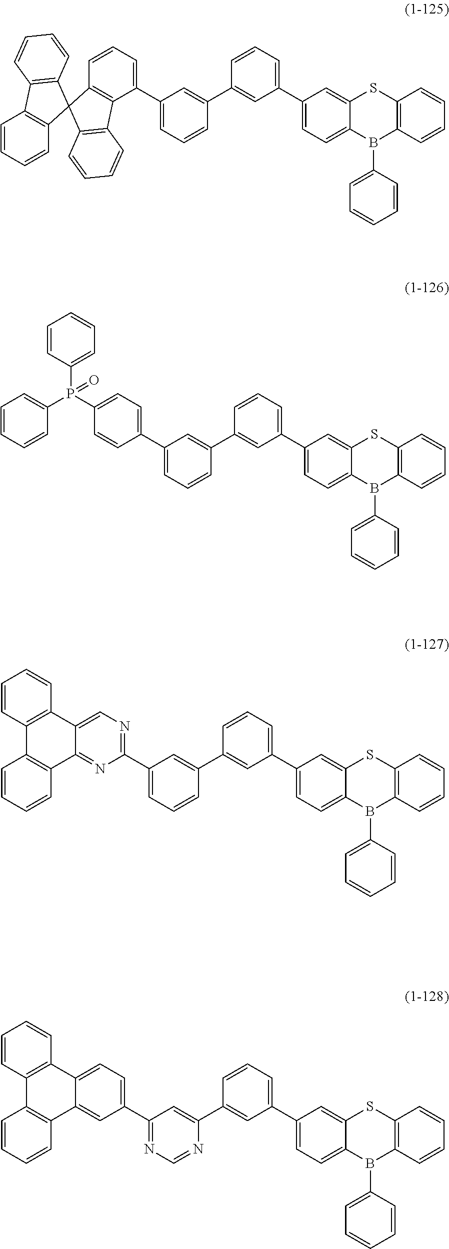

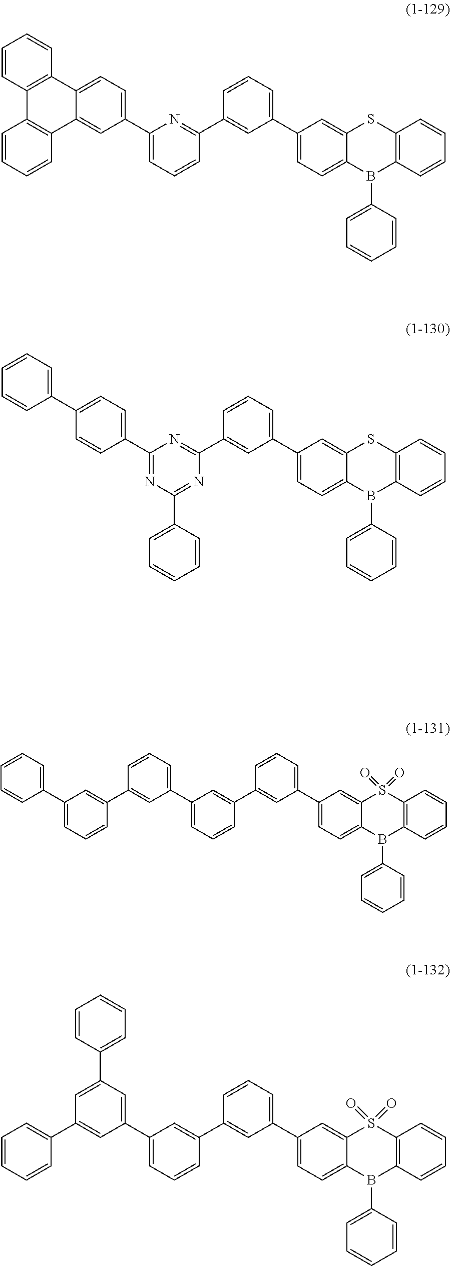

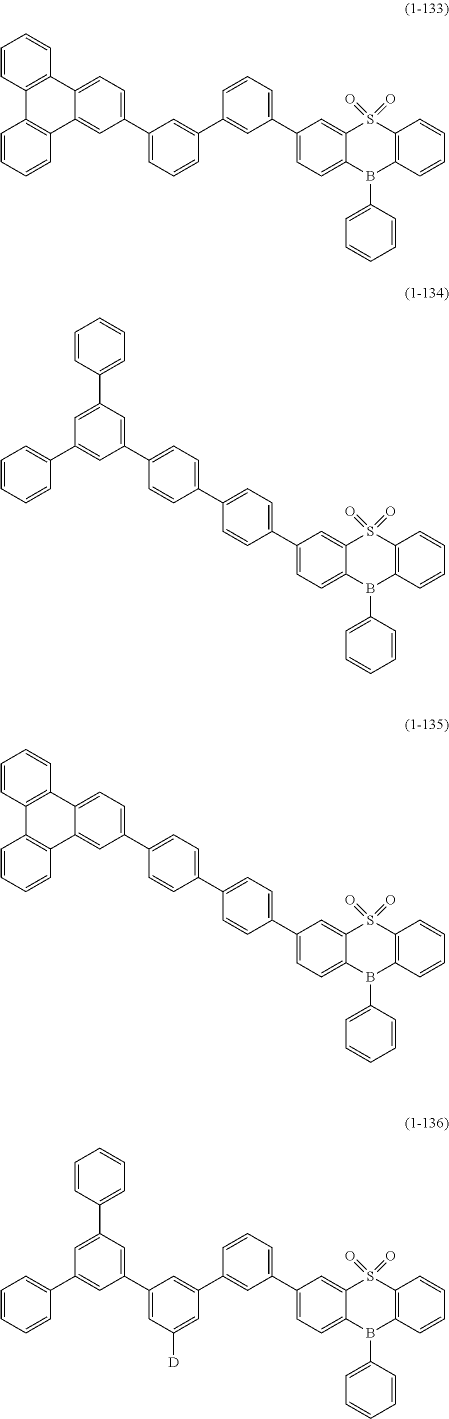

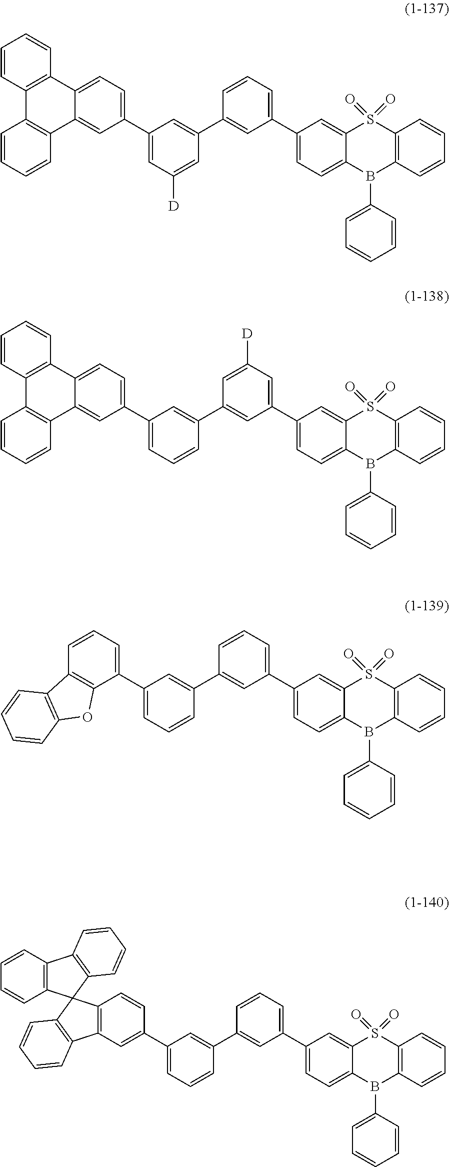

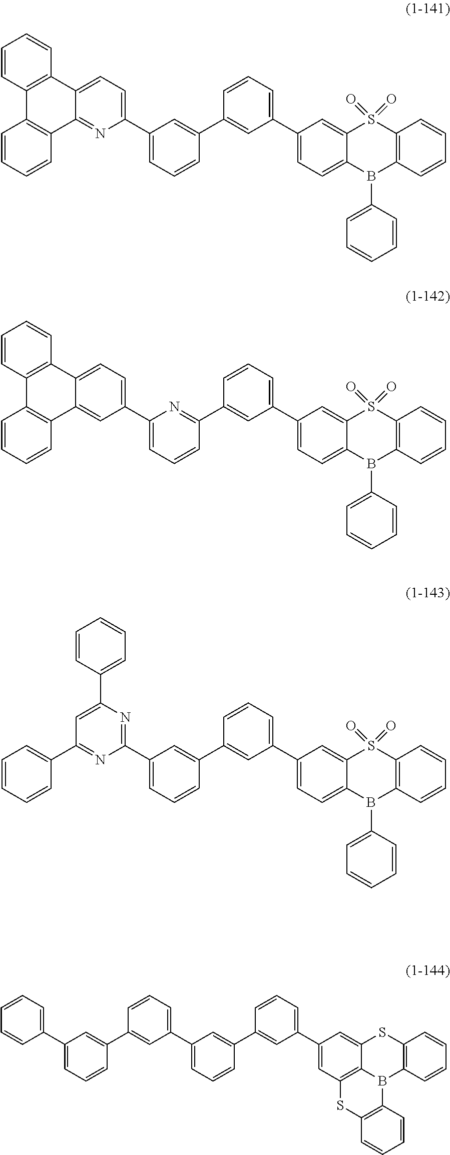

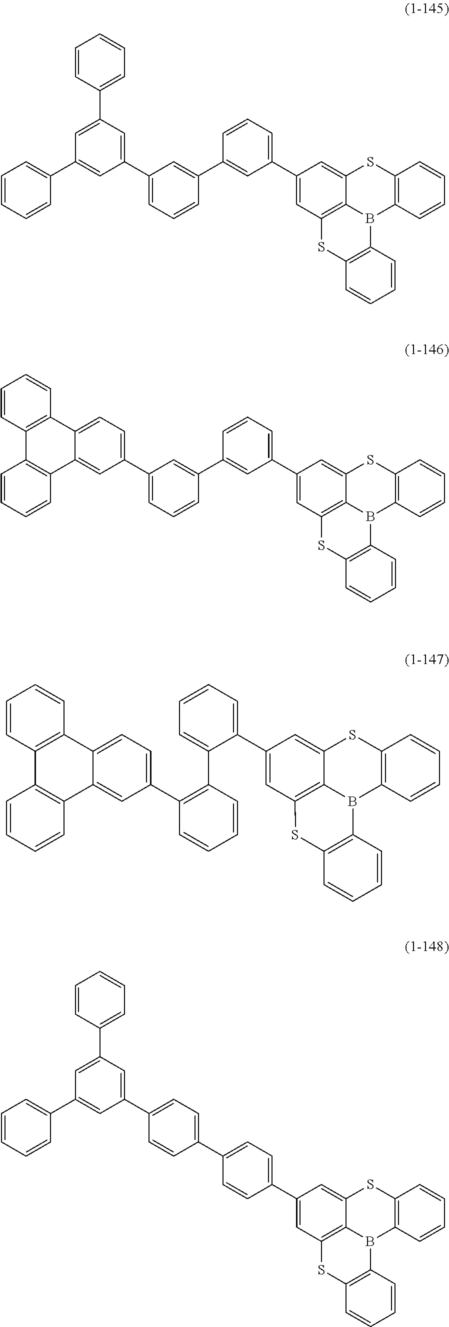

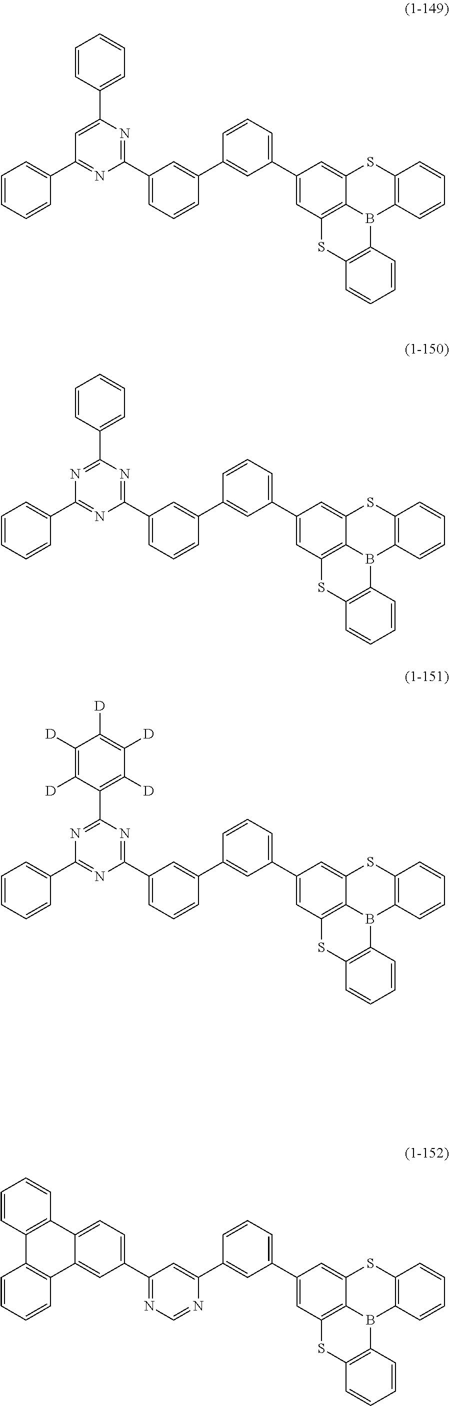

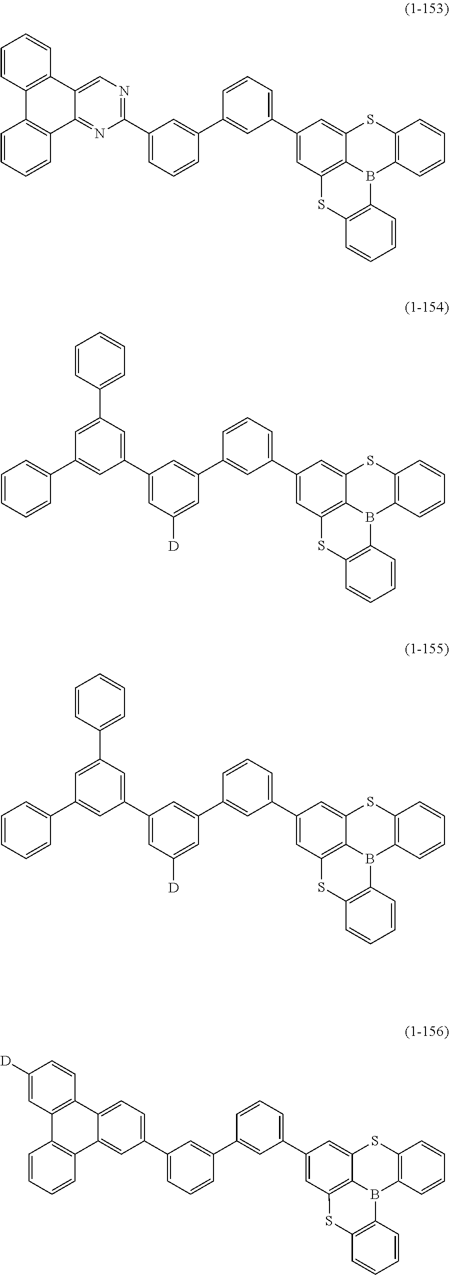

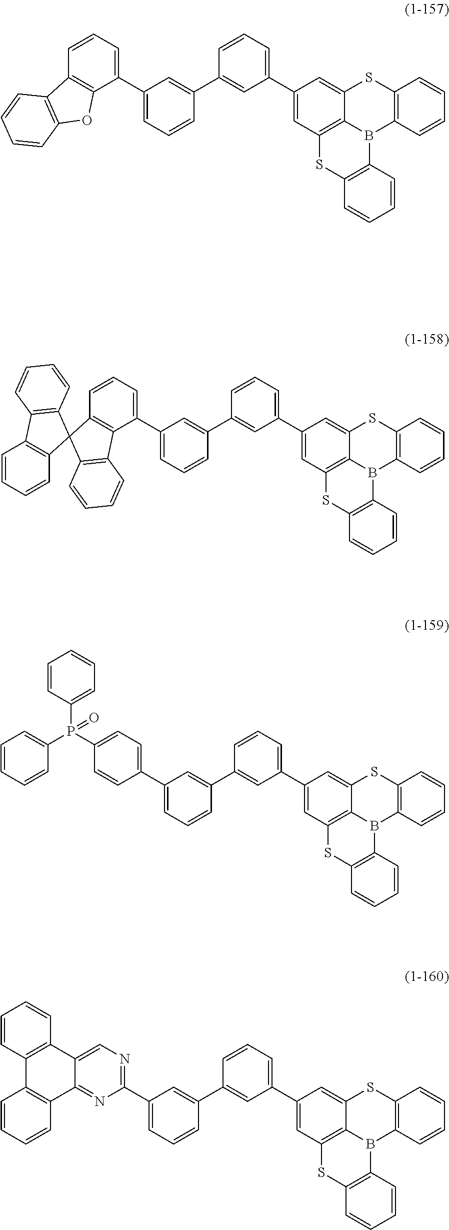

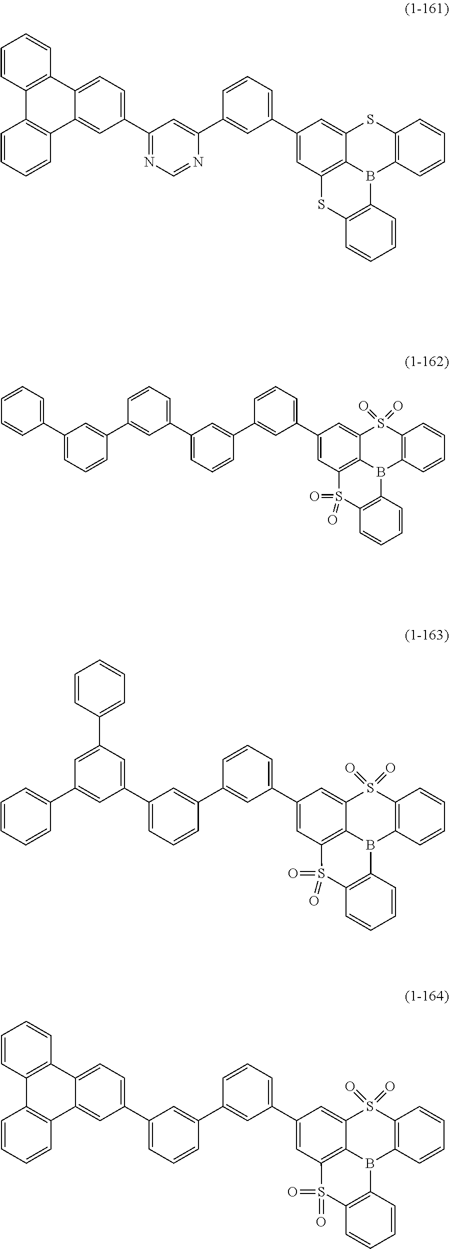

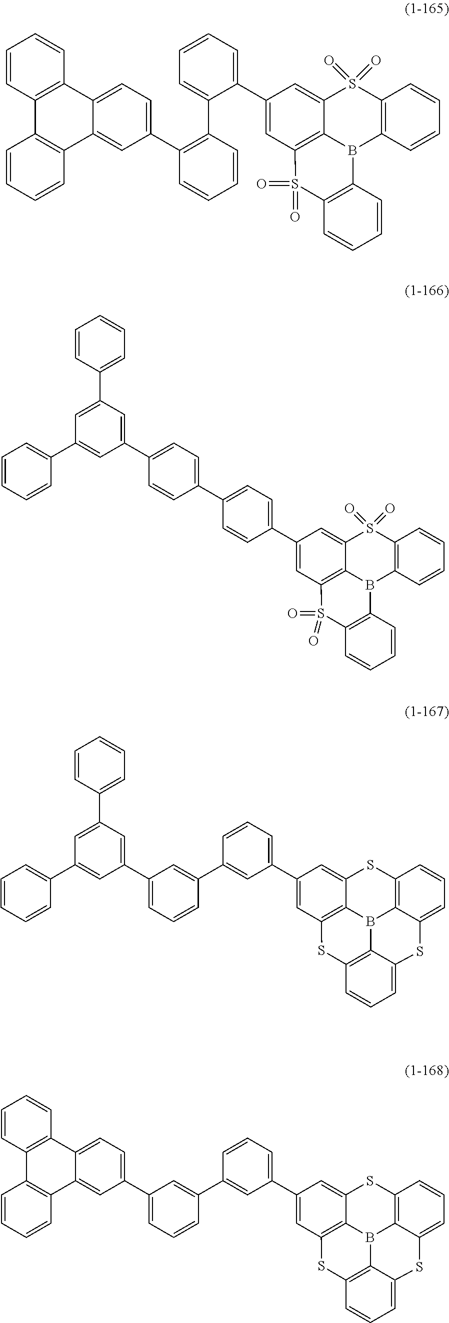

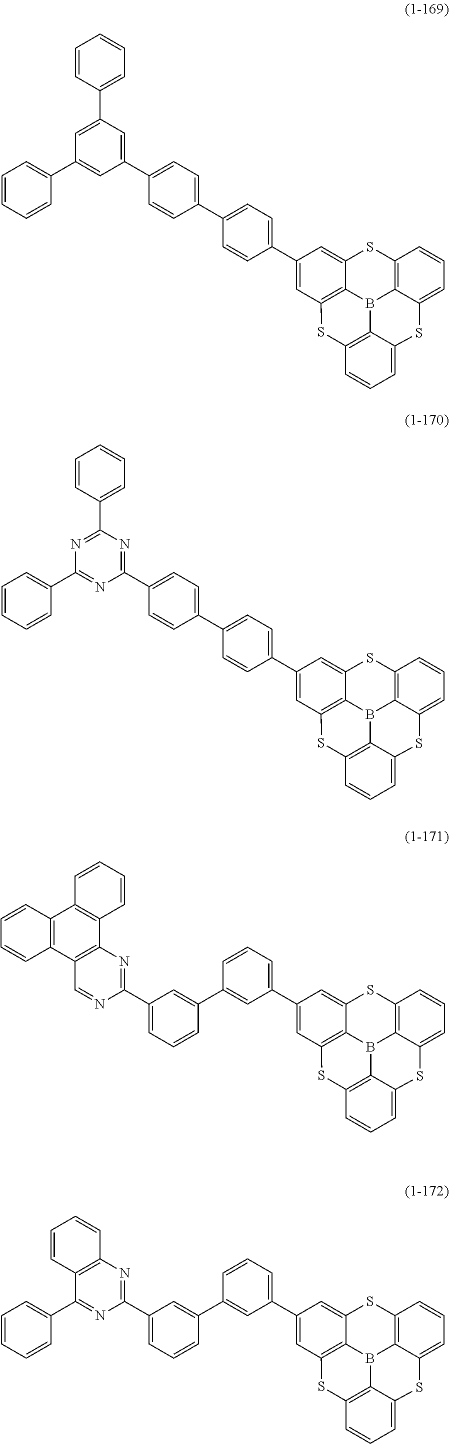

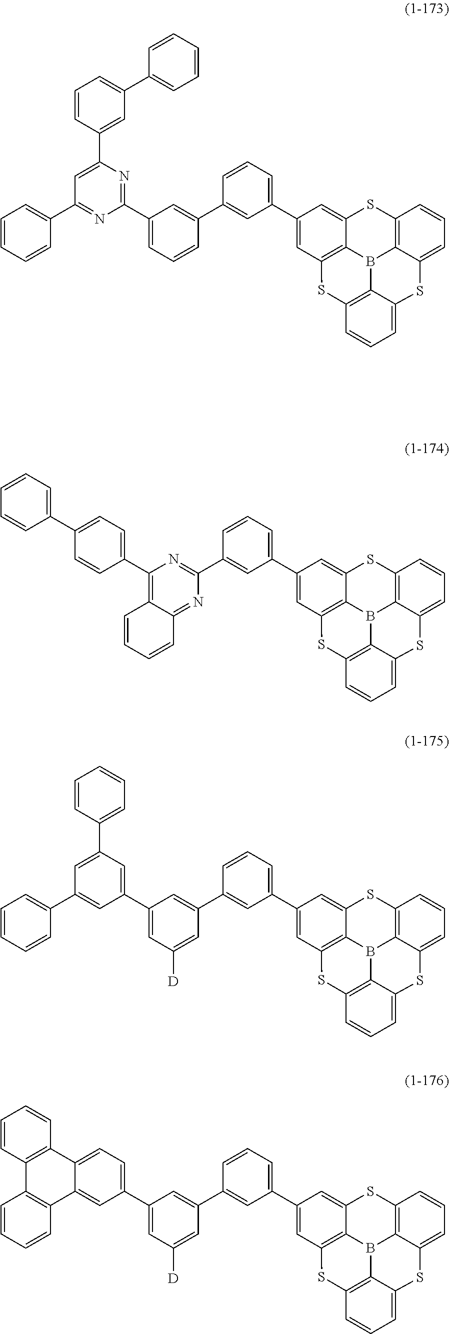

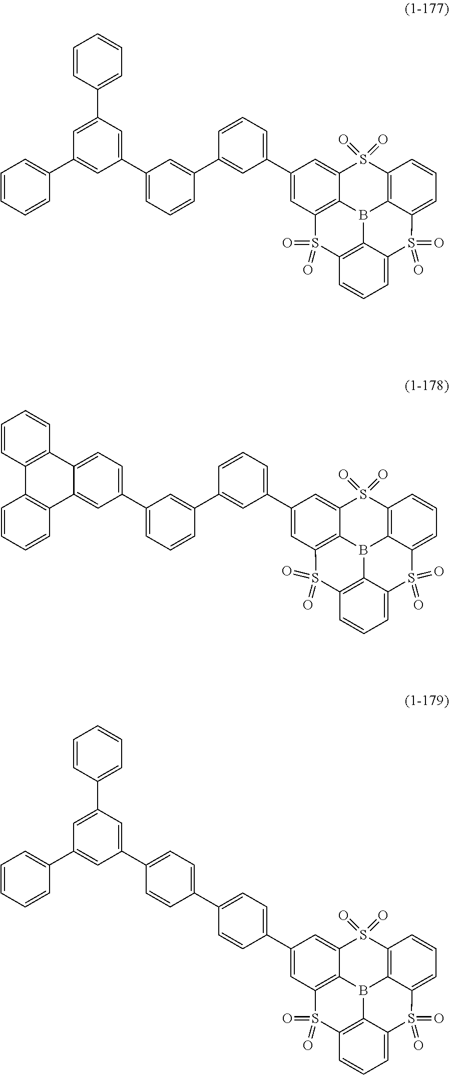

Specifically, the second organic compound satisfying the general formula (2) includes, but is not limited to, the following compounds:

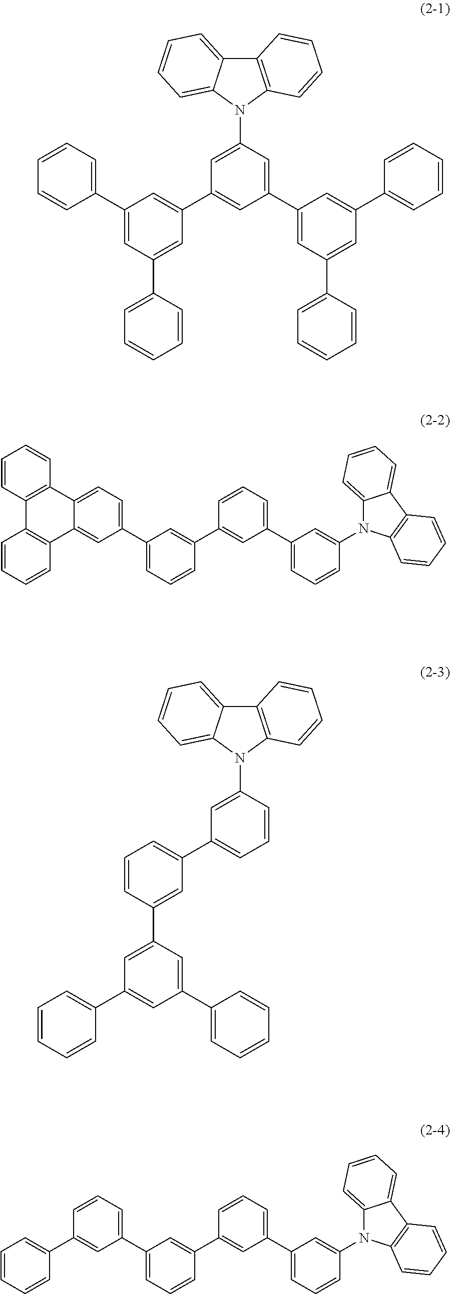

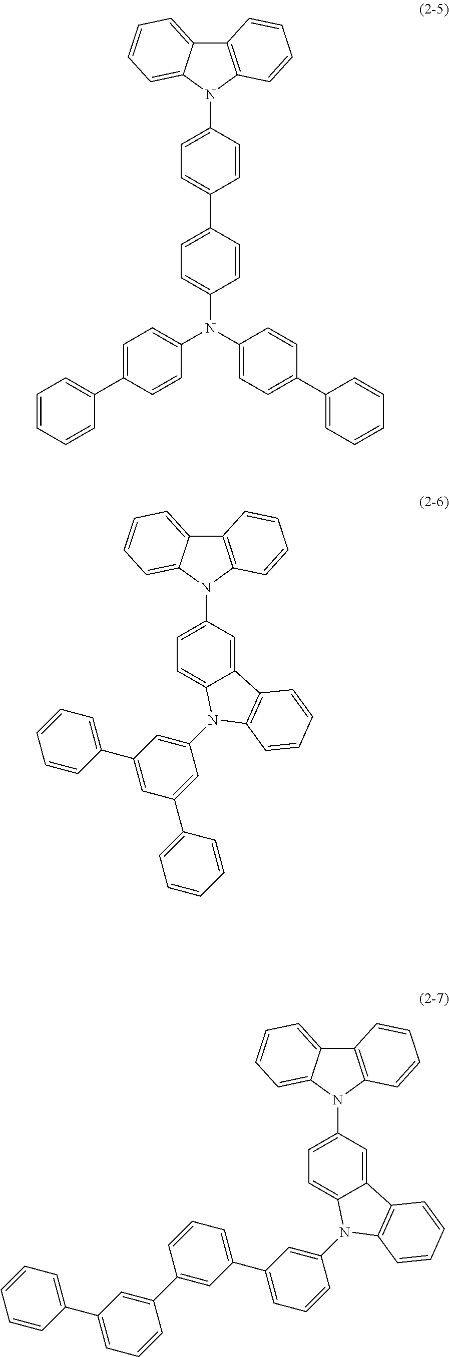

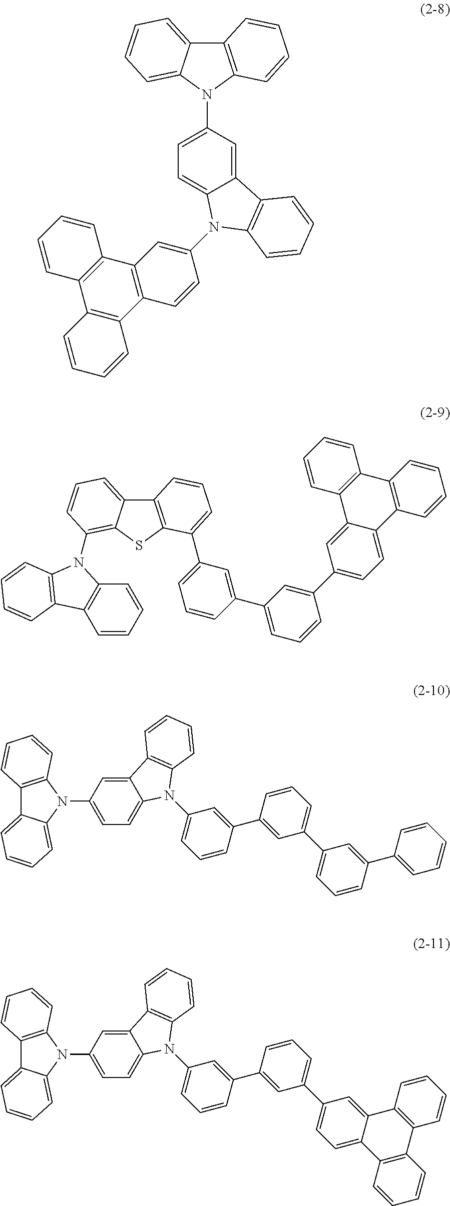

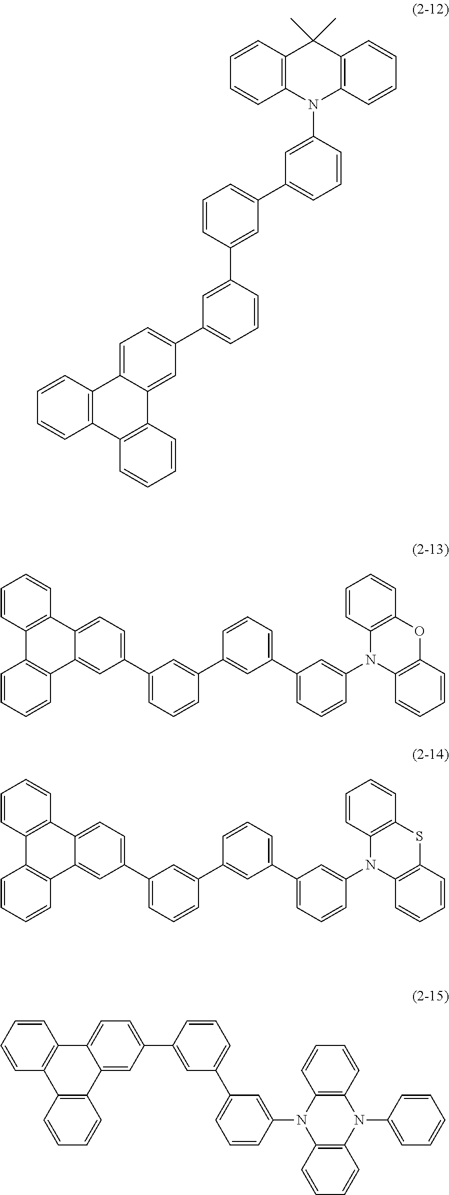

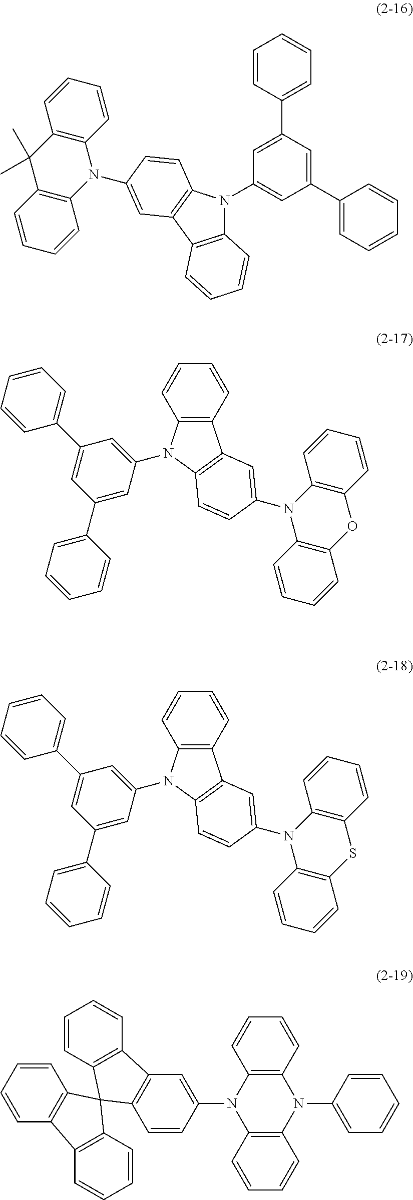

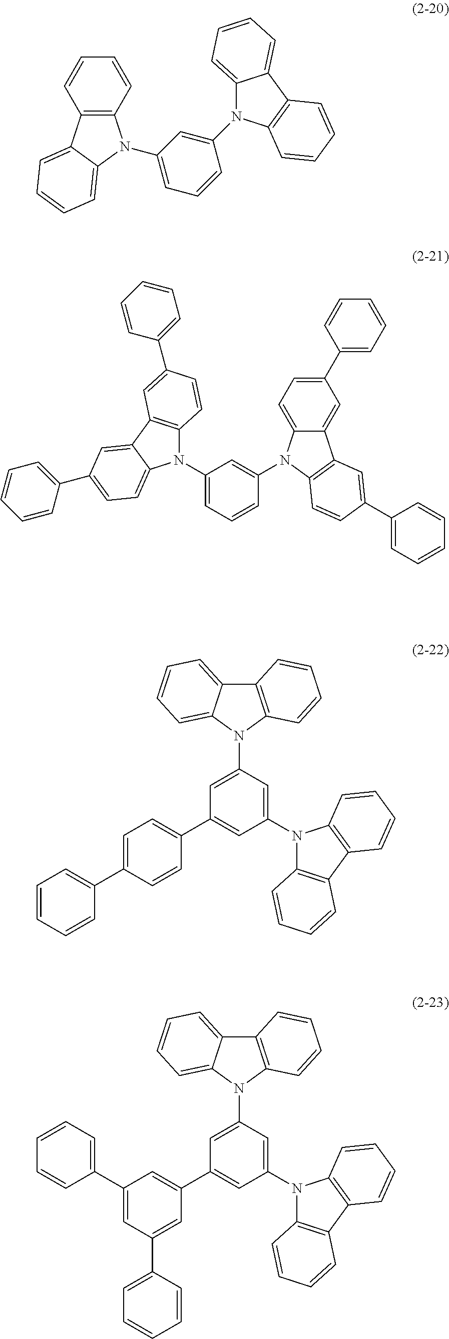

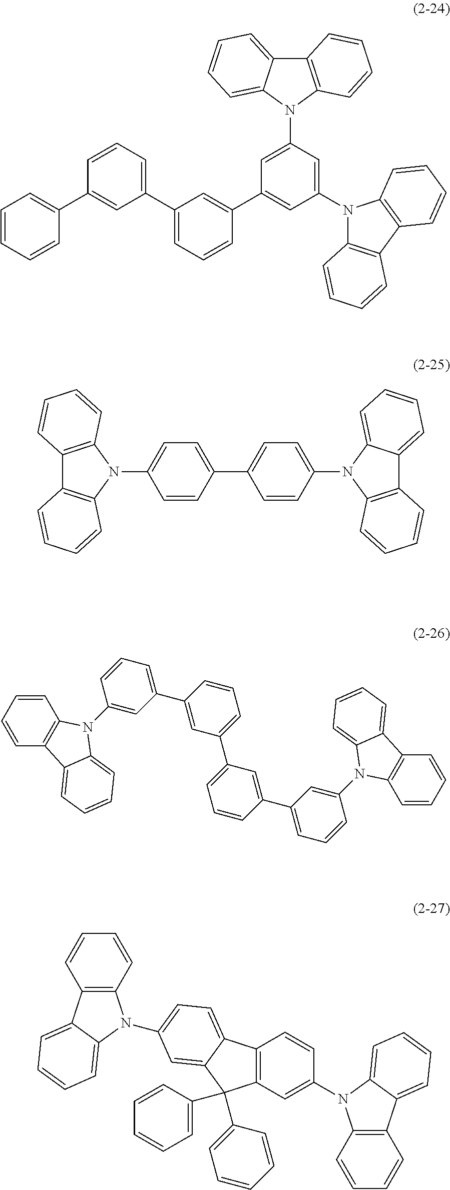

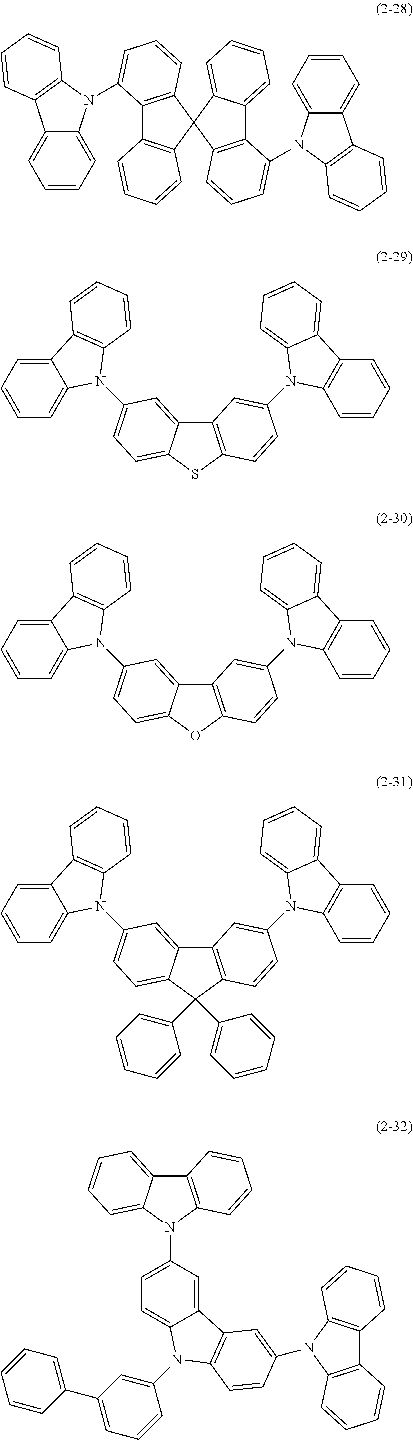

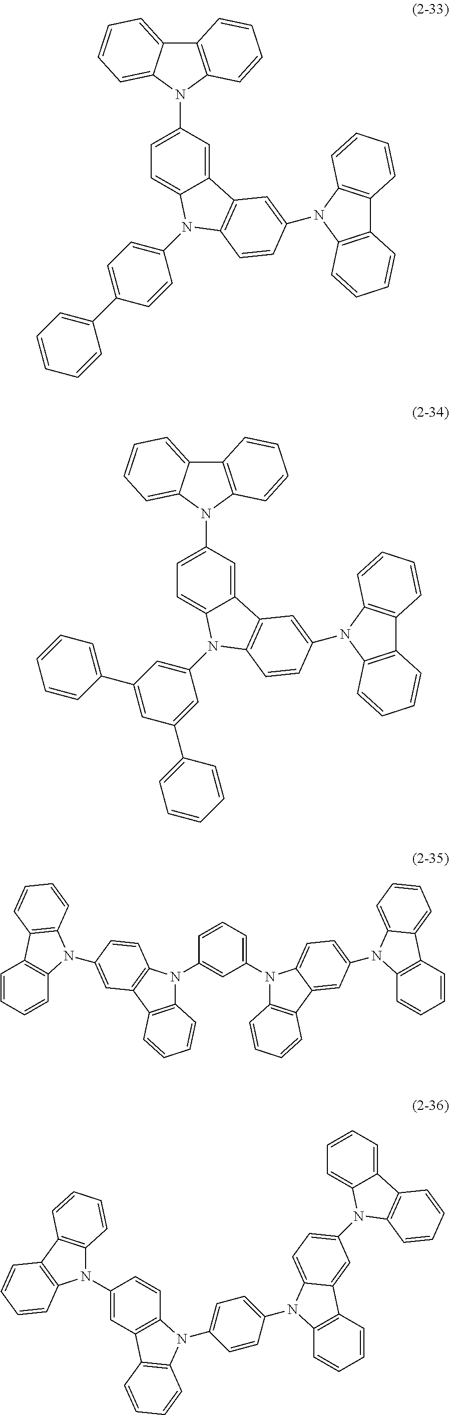

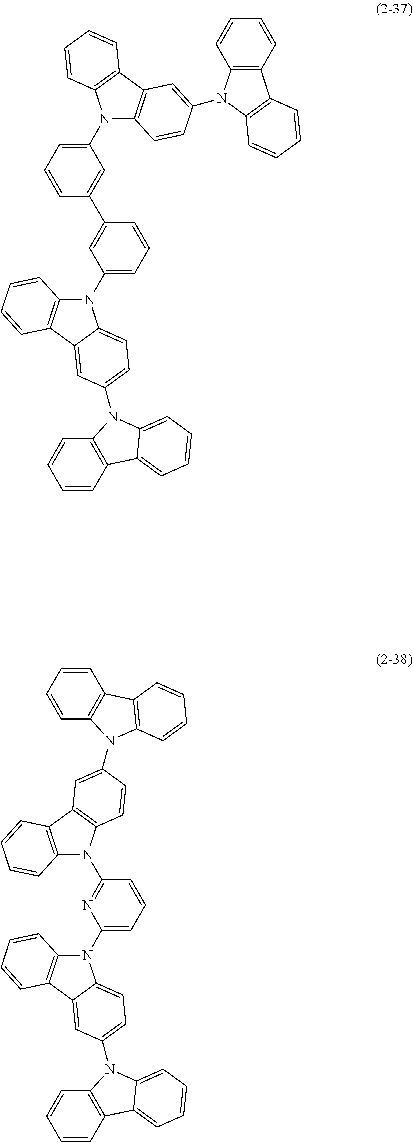

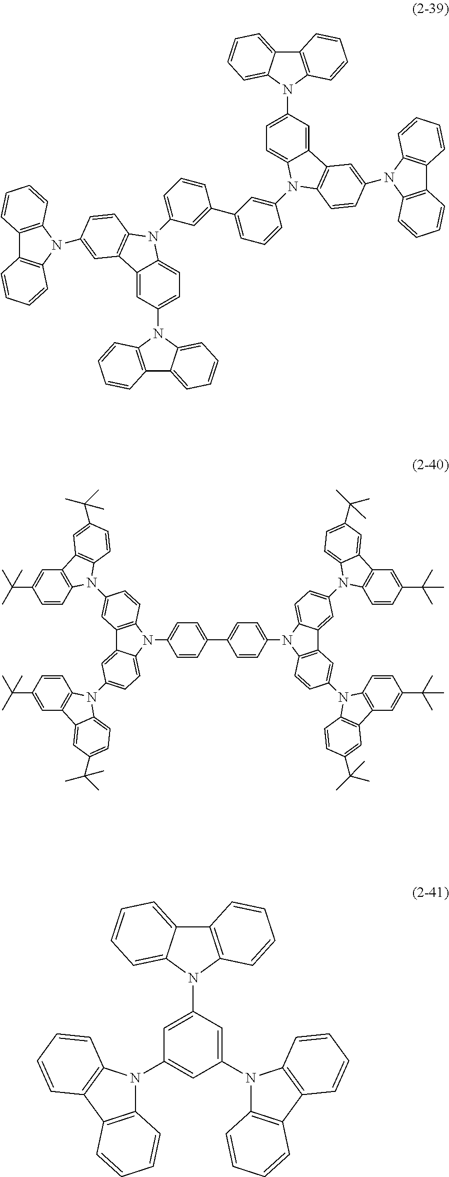

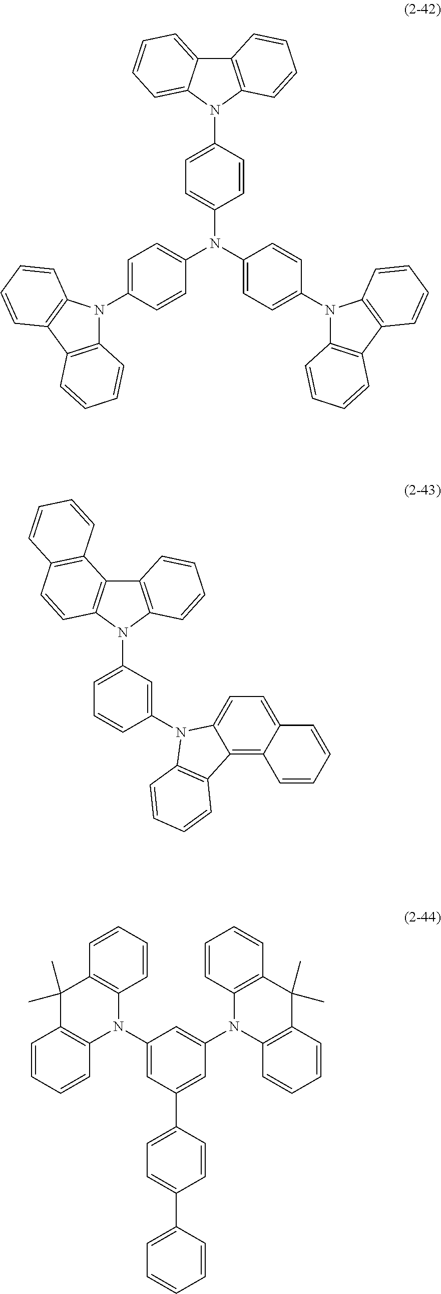

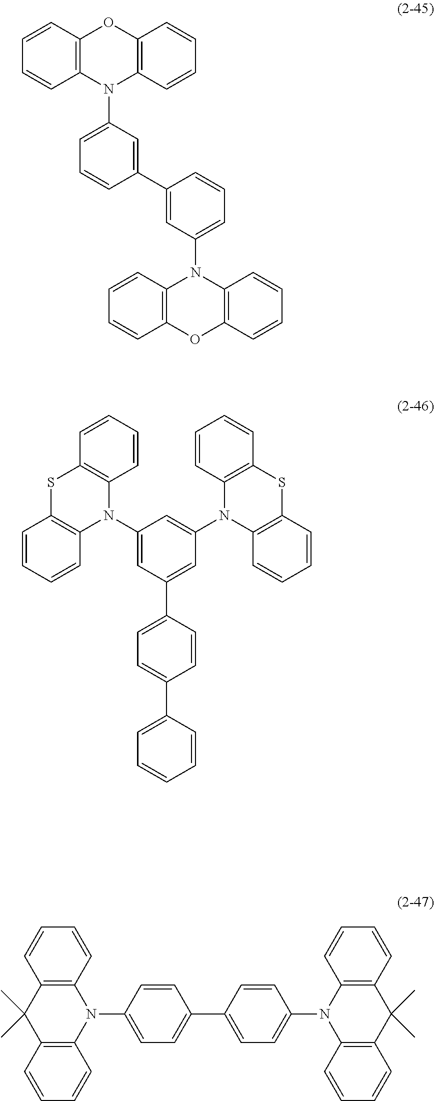

Specifically, the second organic compound satisfying the general formula (3) includes, but is not limited to, the following compounds:

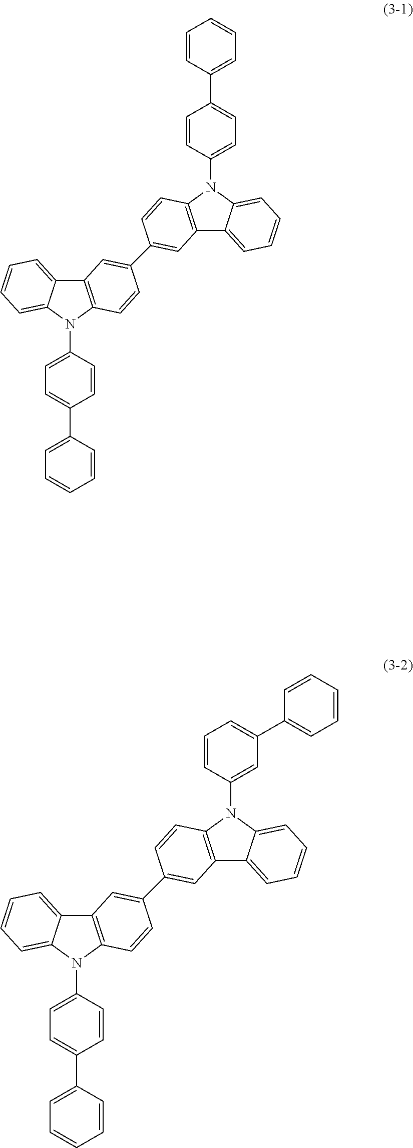

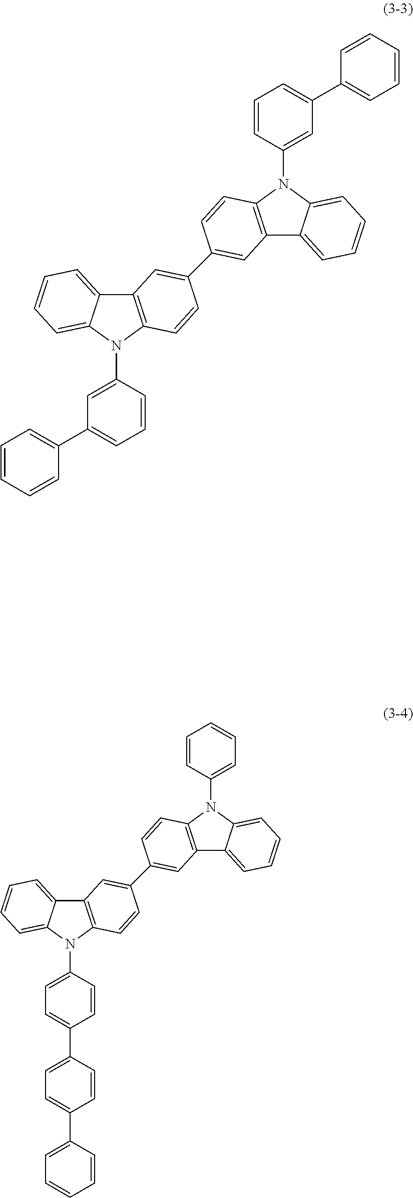

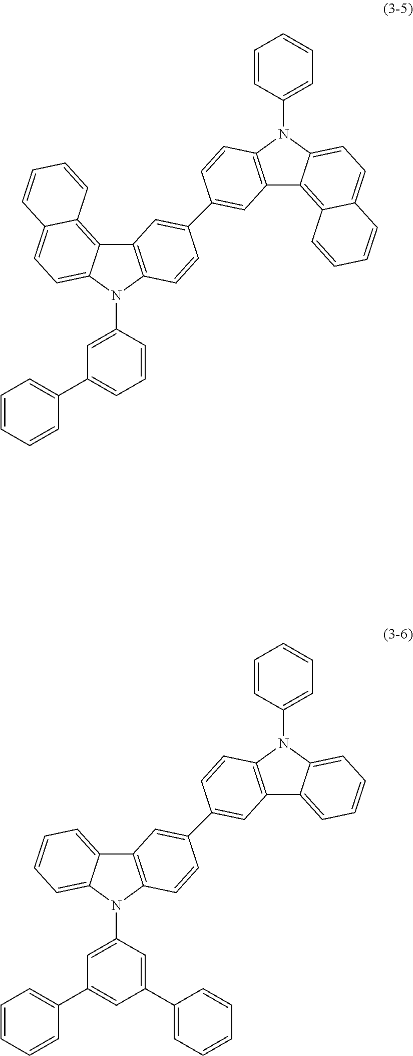

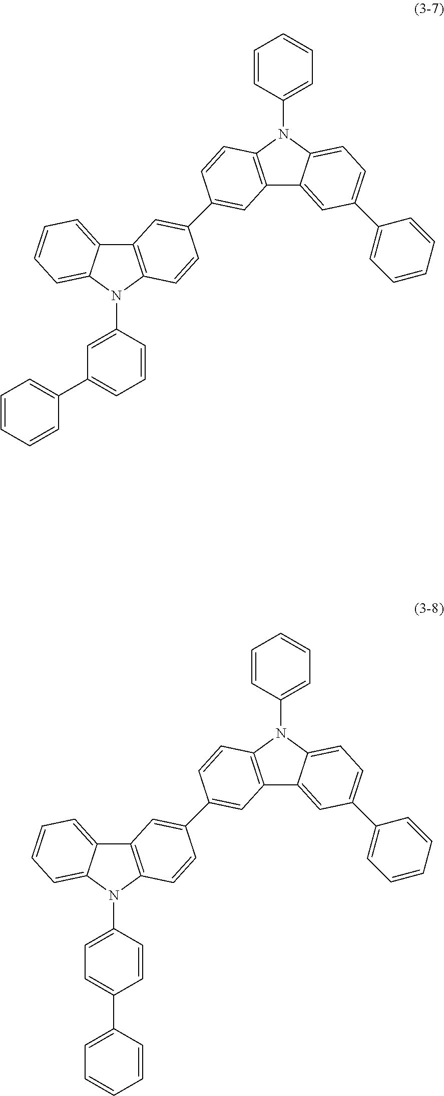

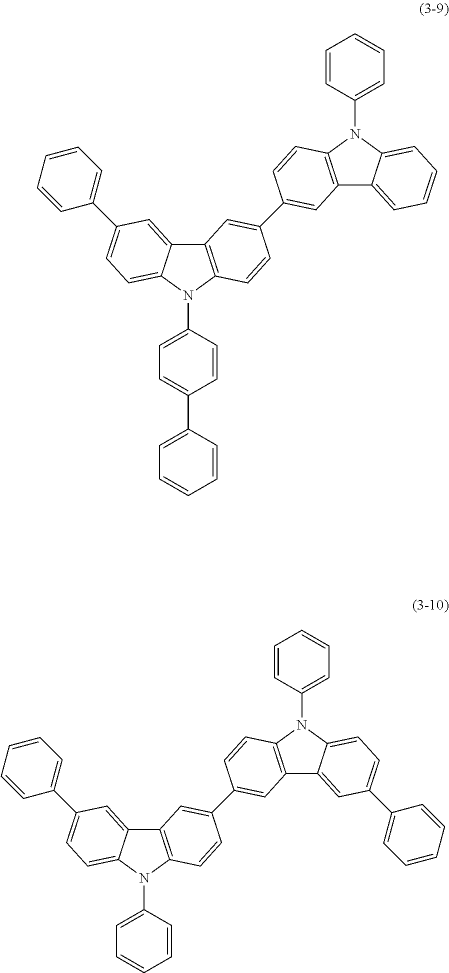

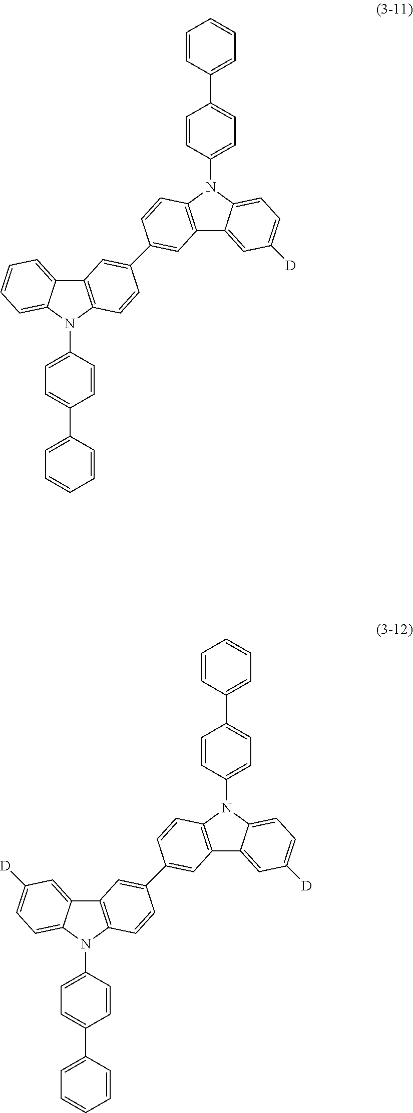

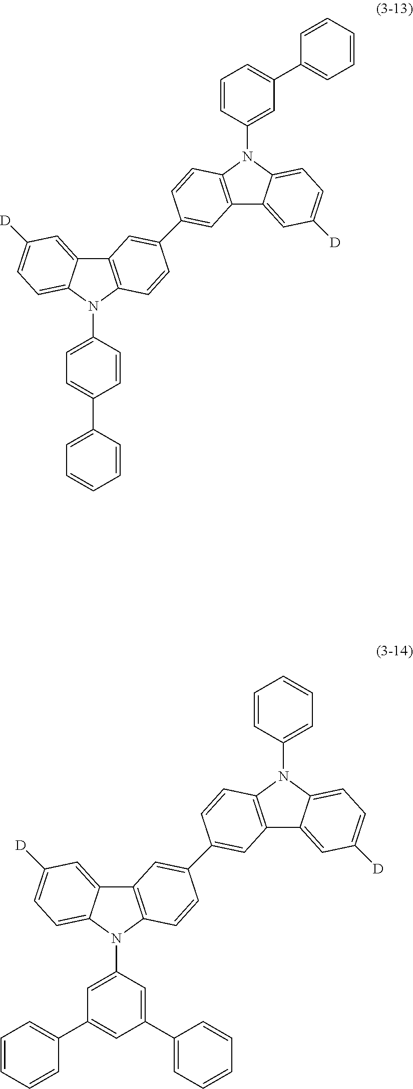

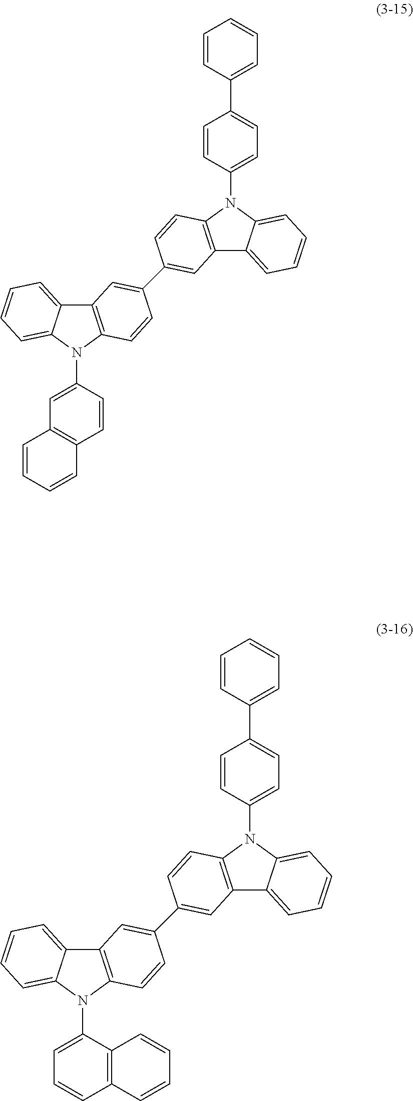

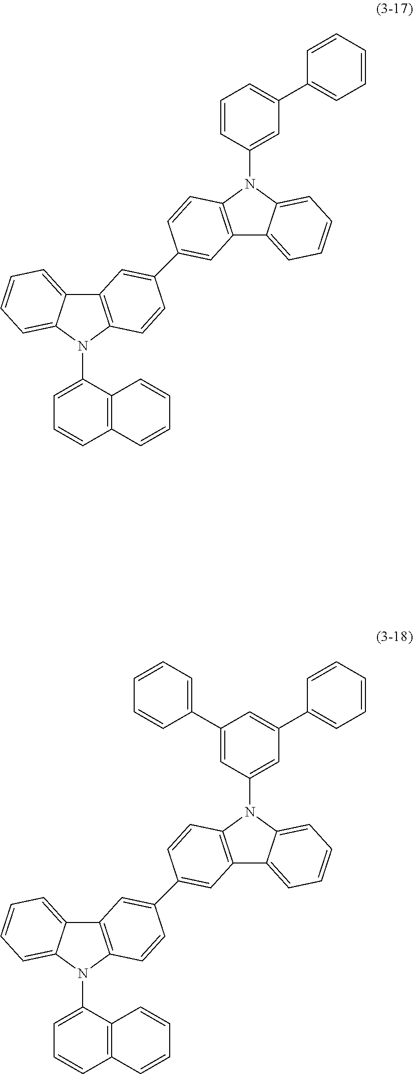

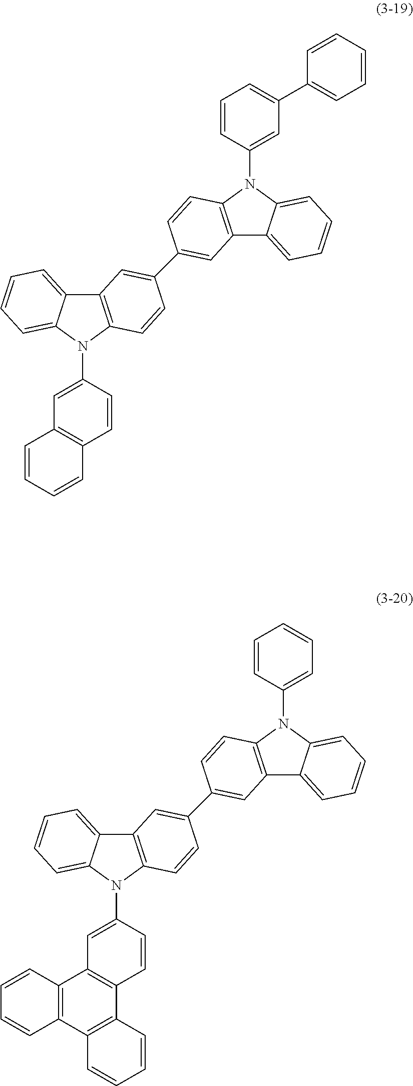

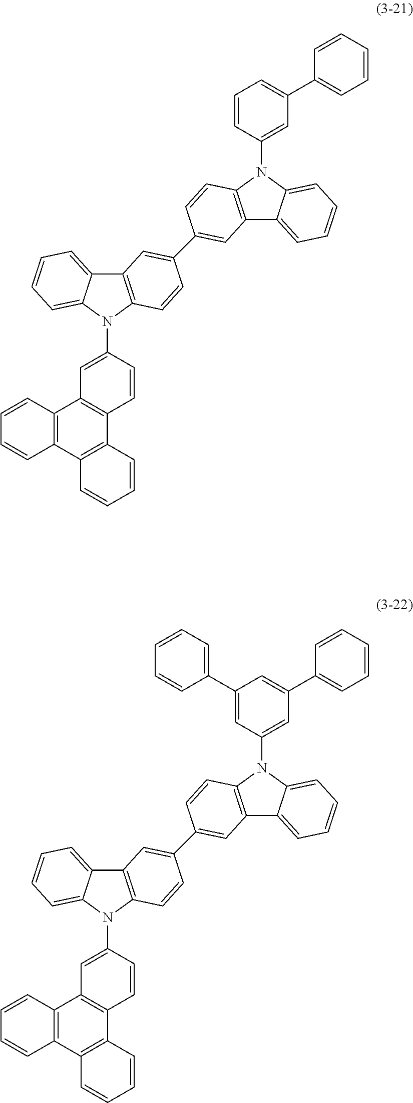

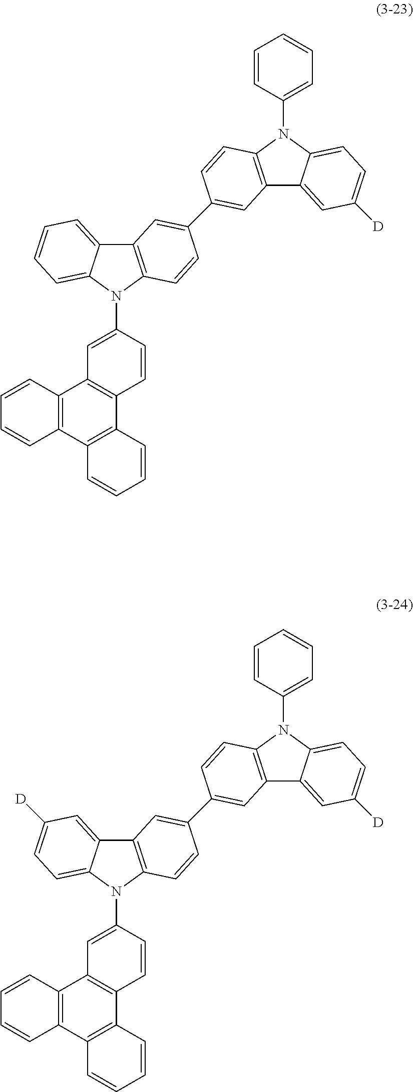

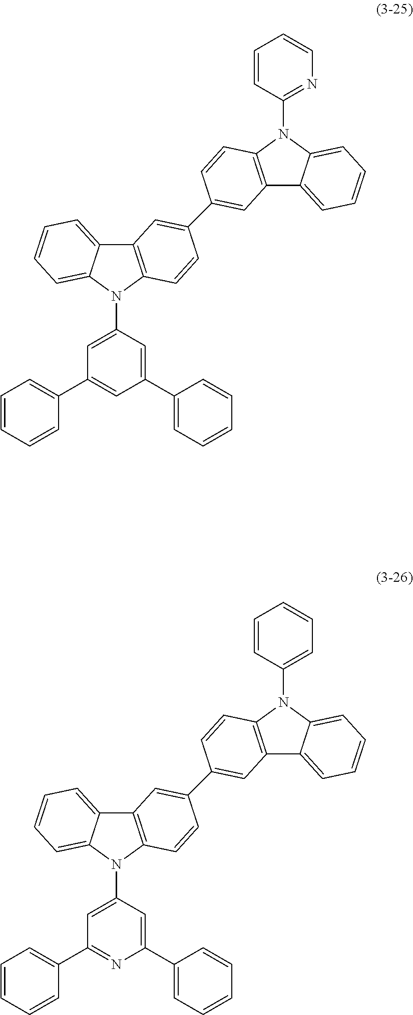

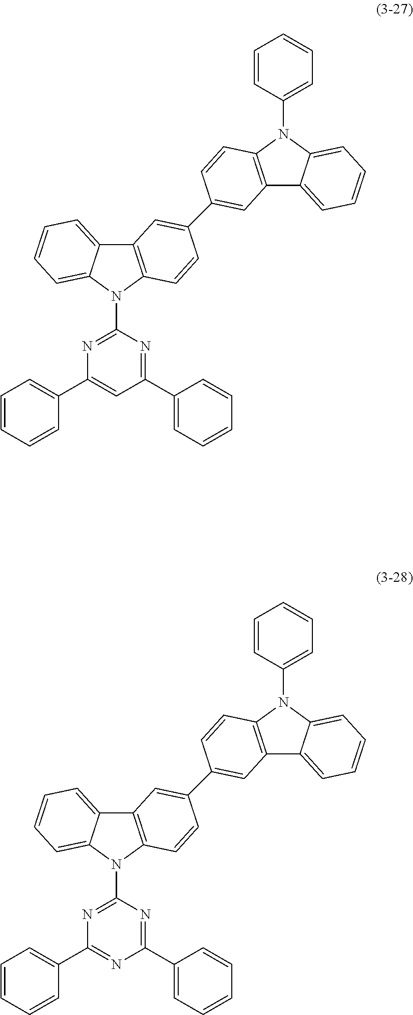

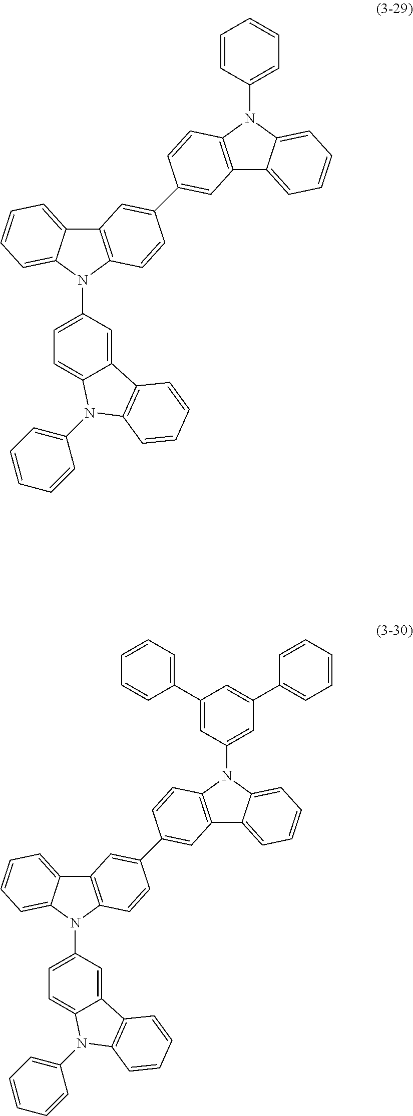

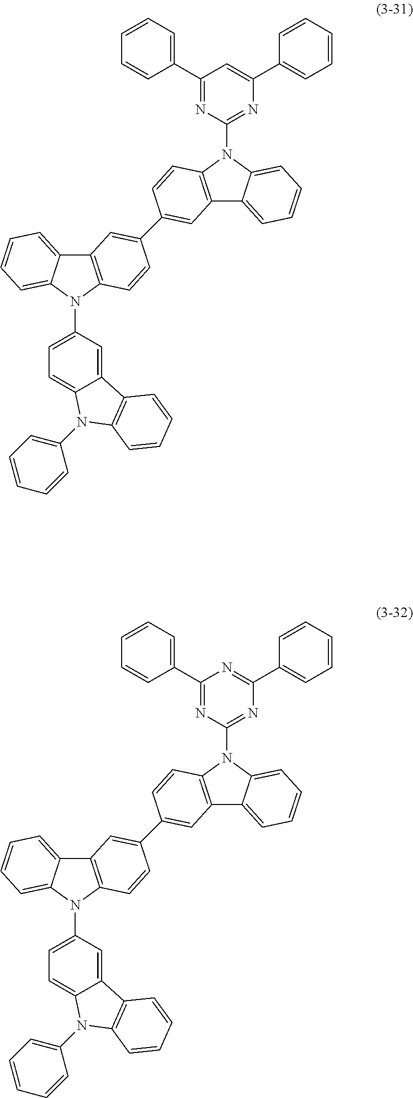

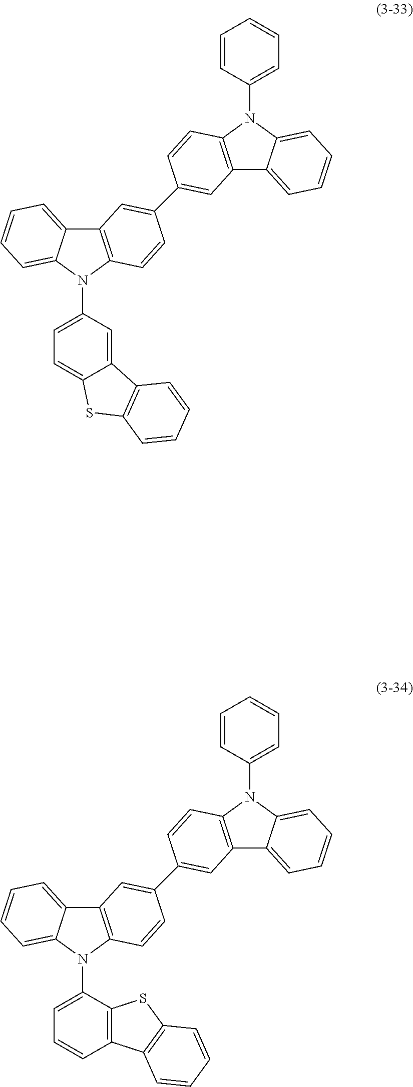

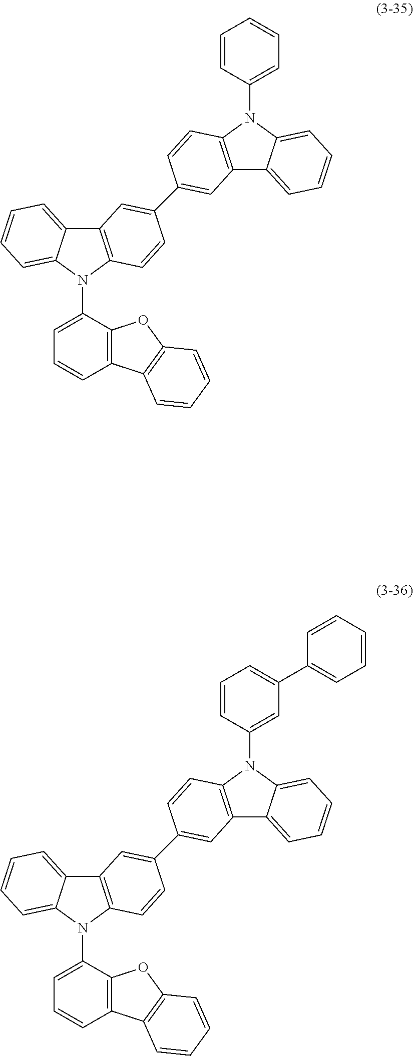

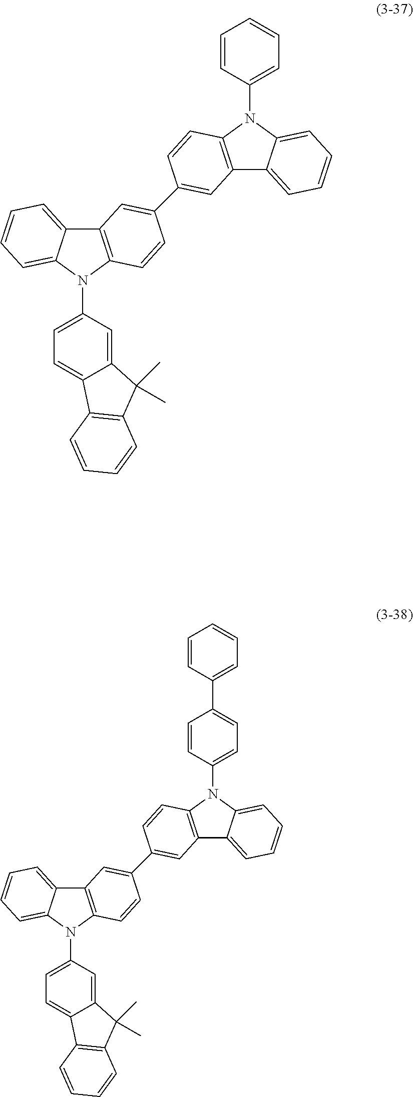

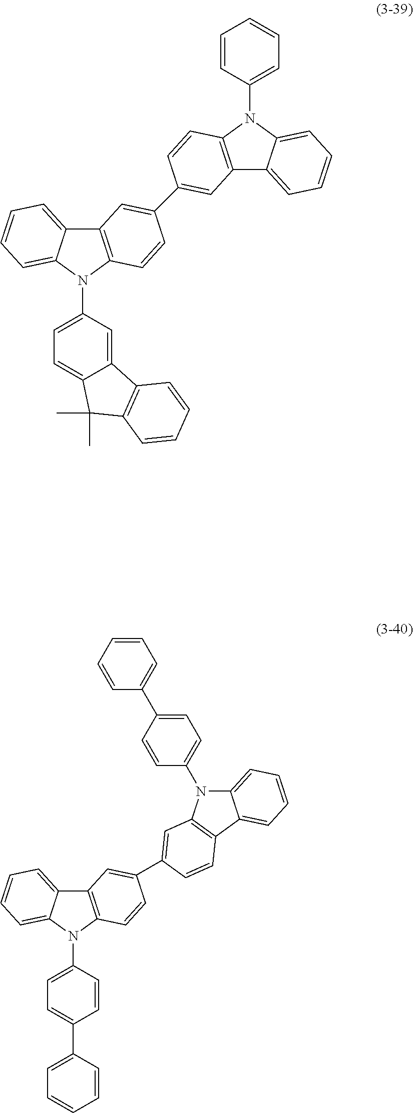

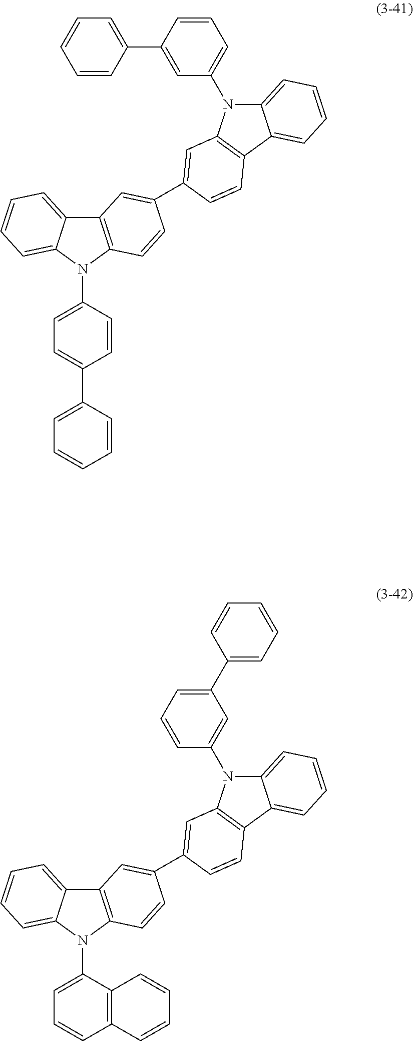

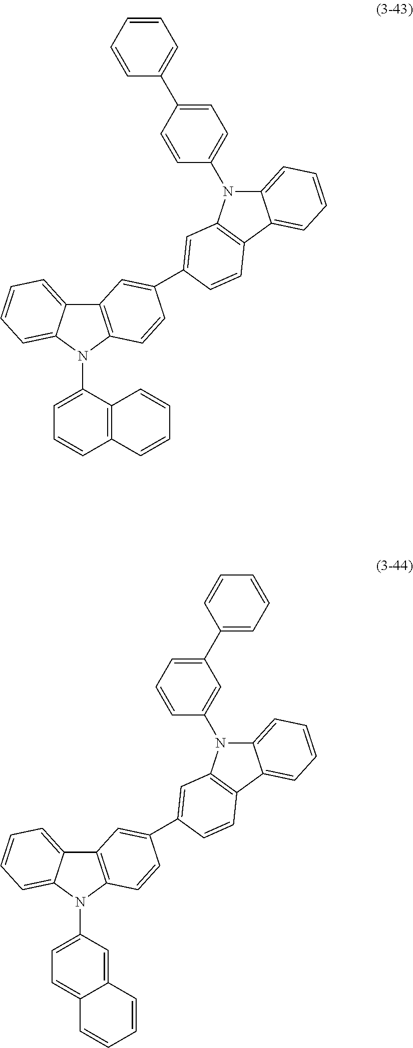

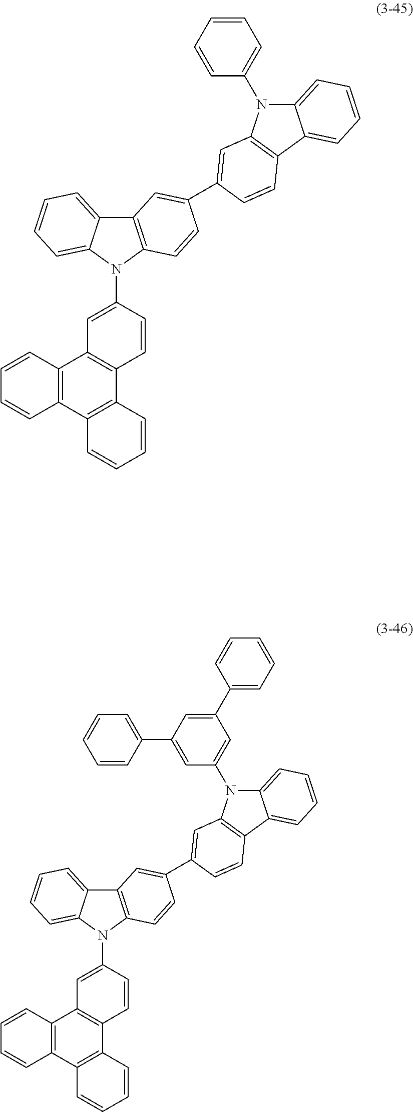

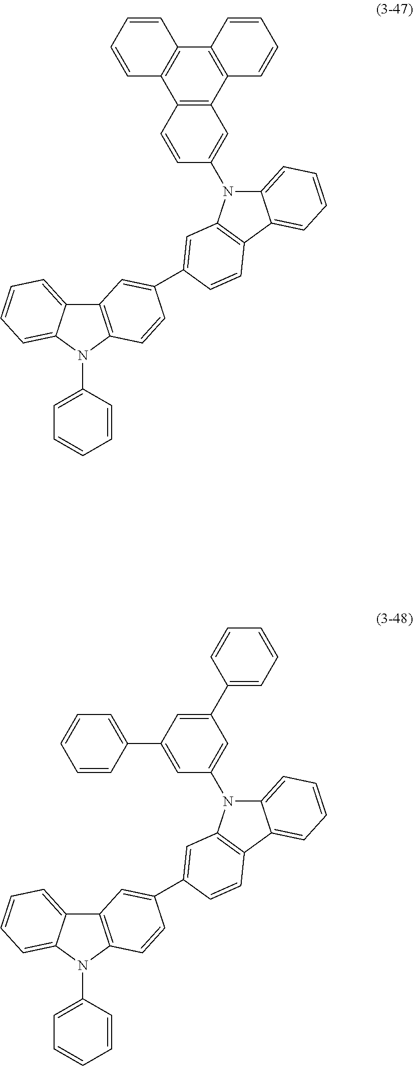

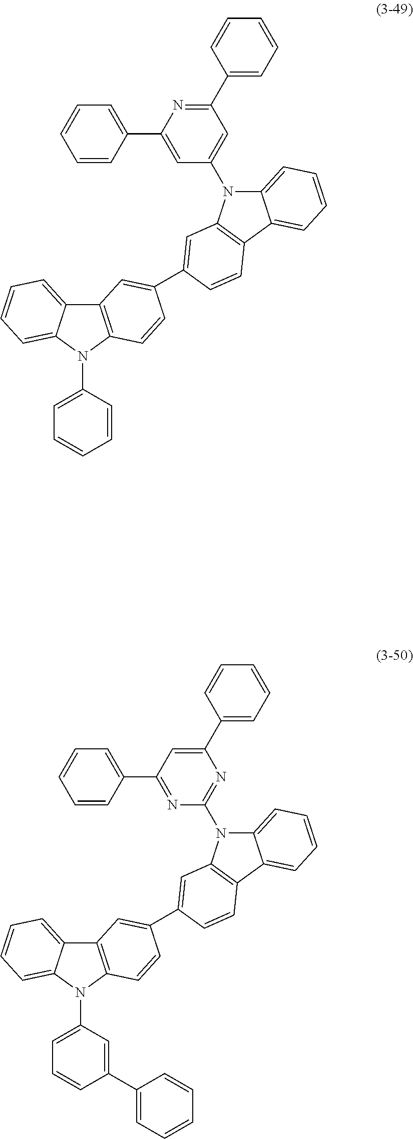

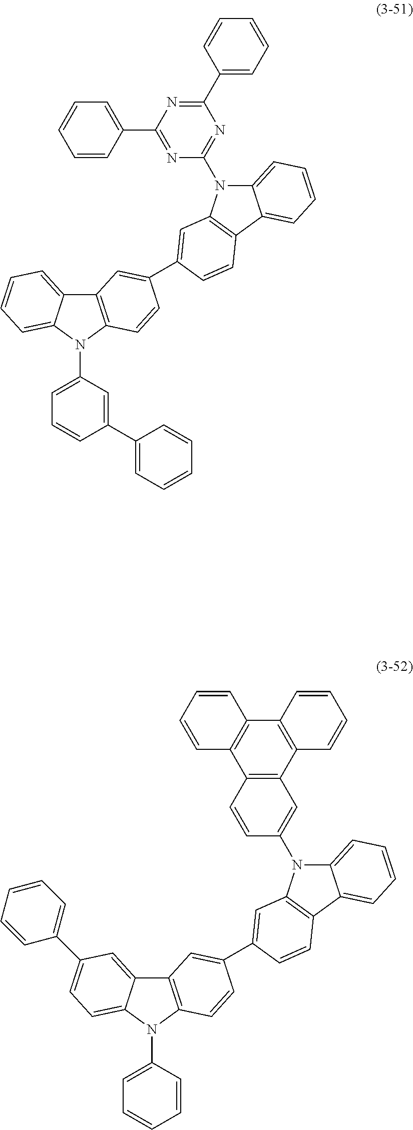

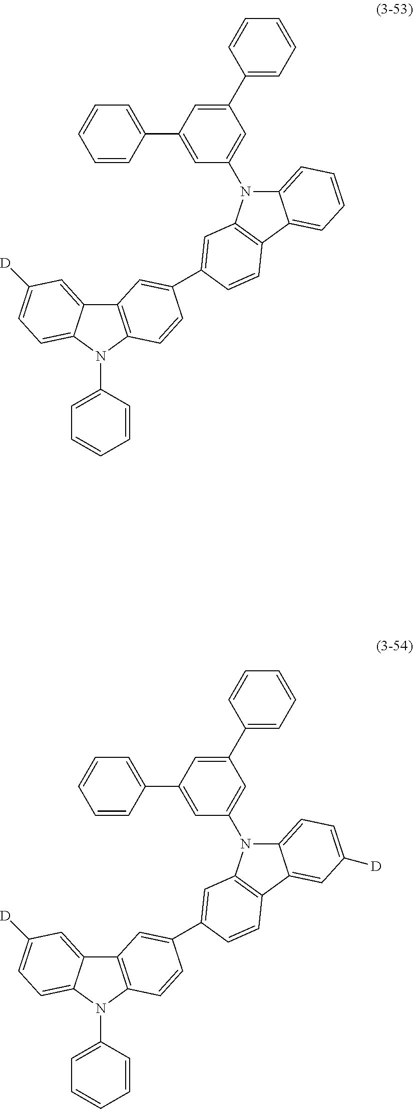

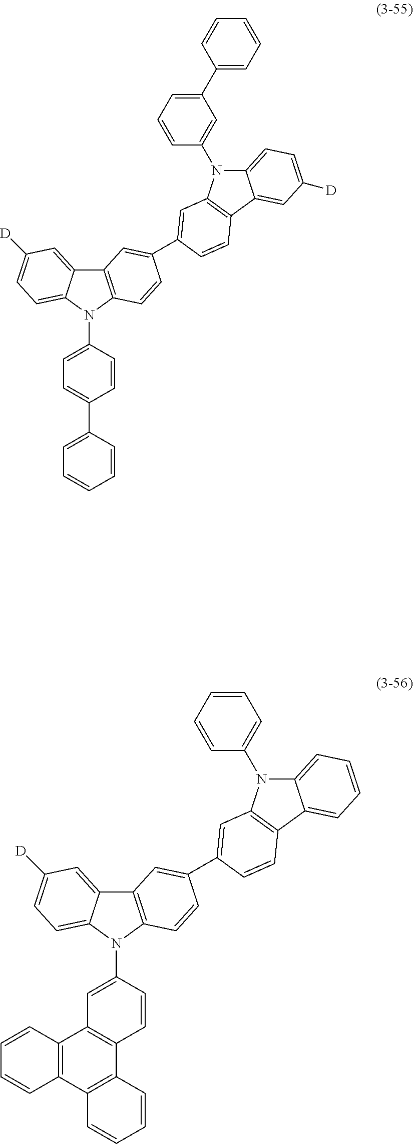

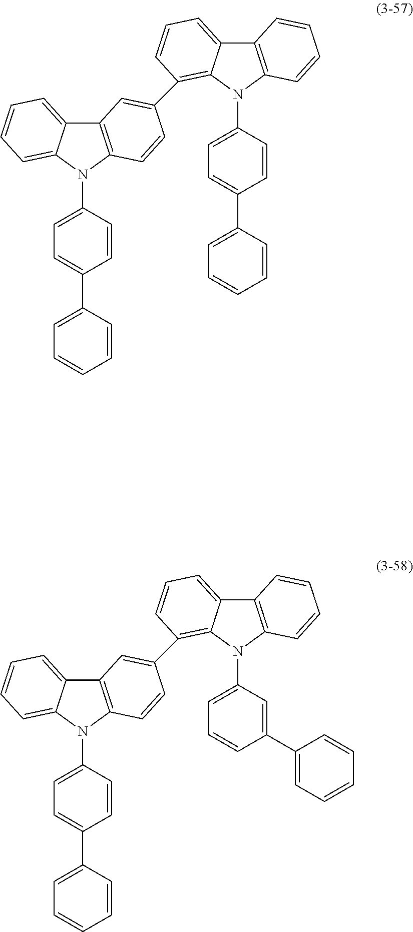

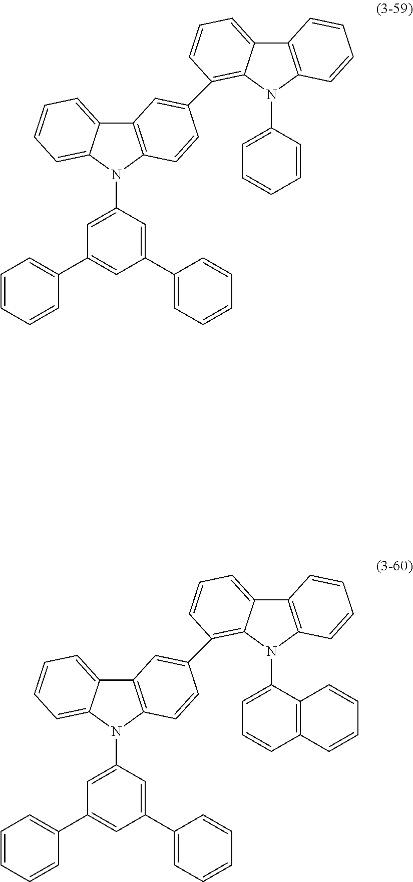

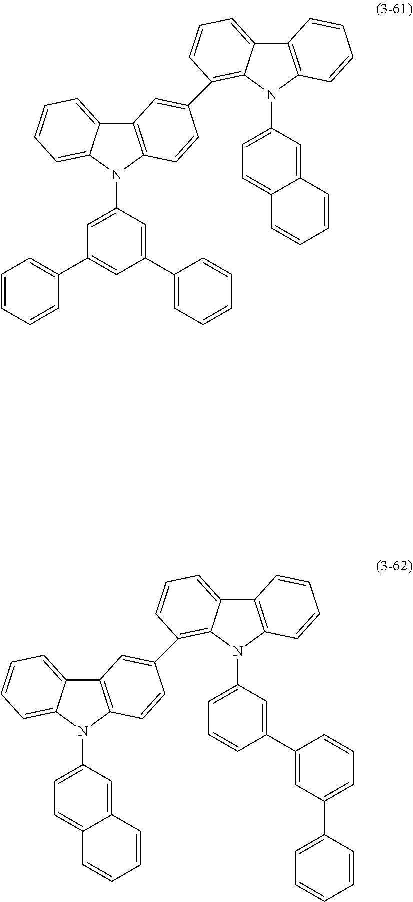

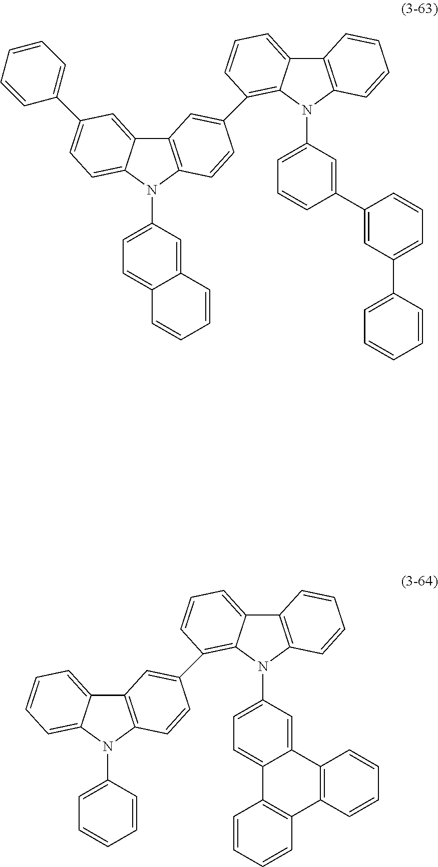

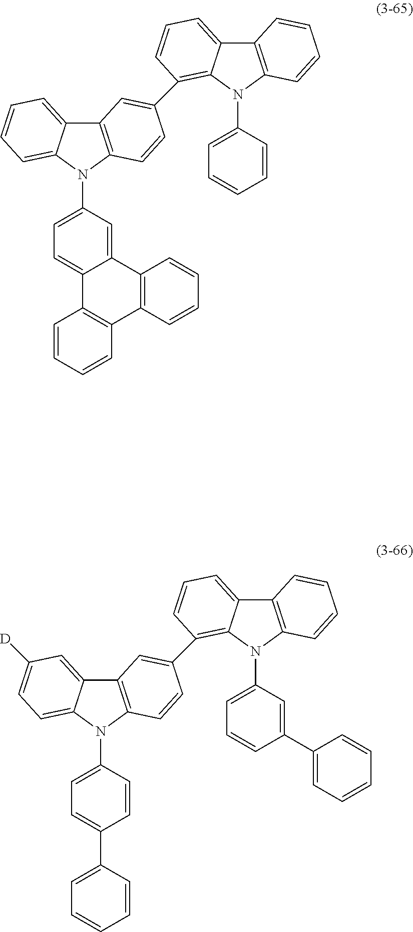

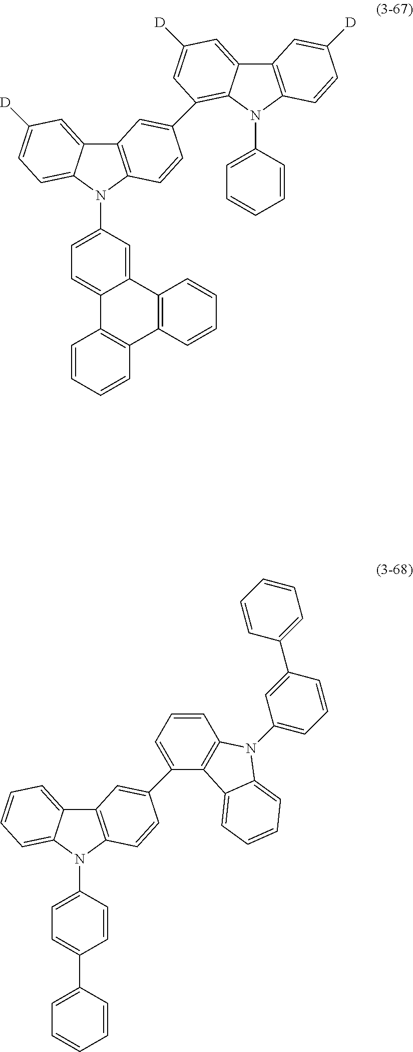

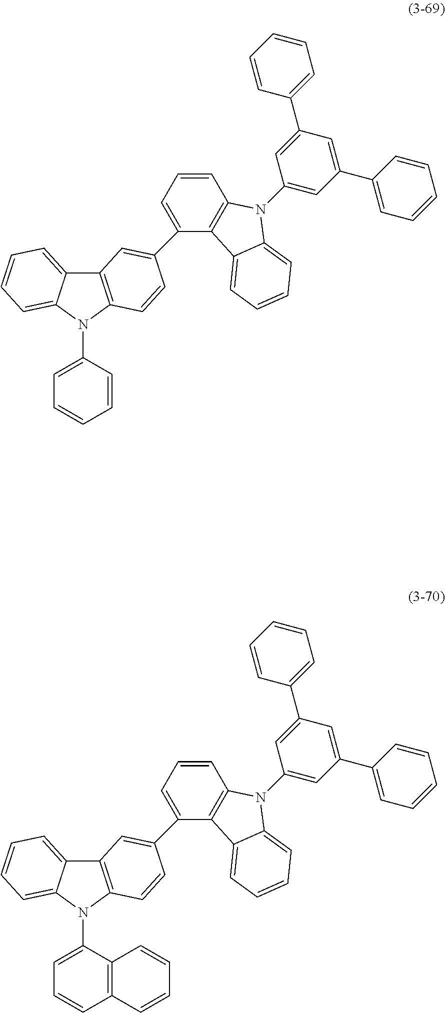

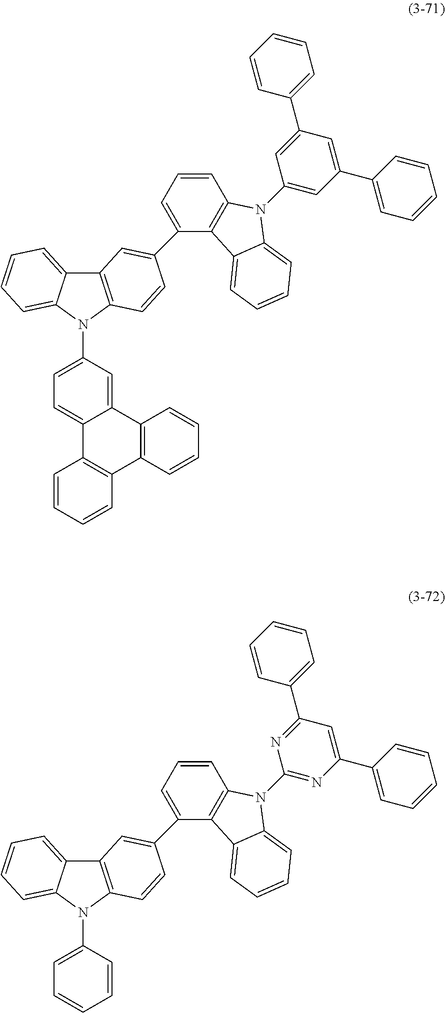

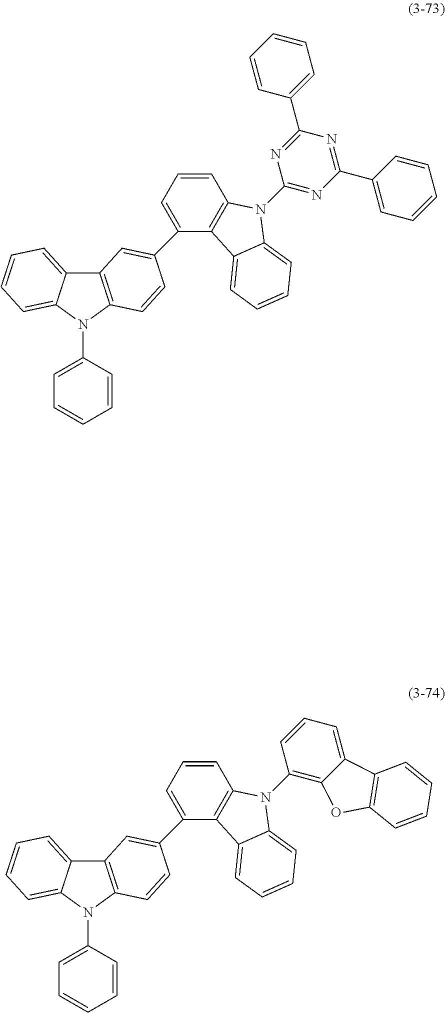

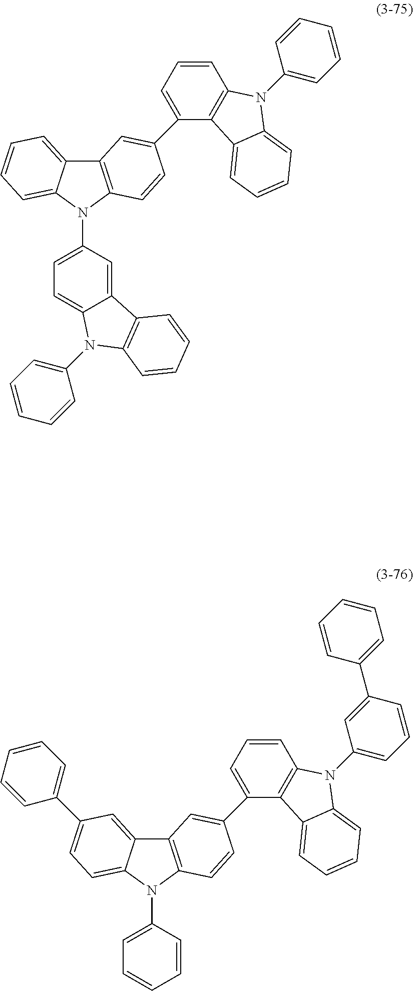

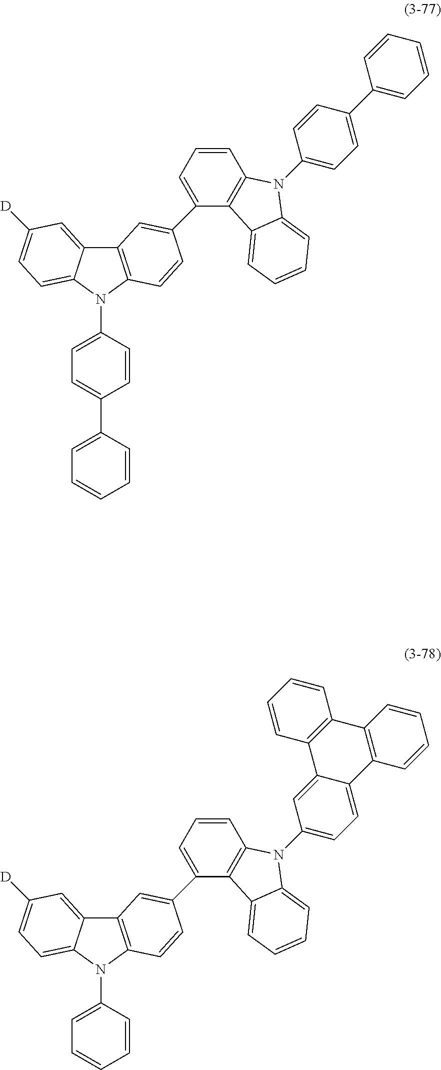

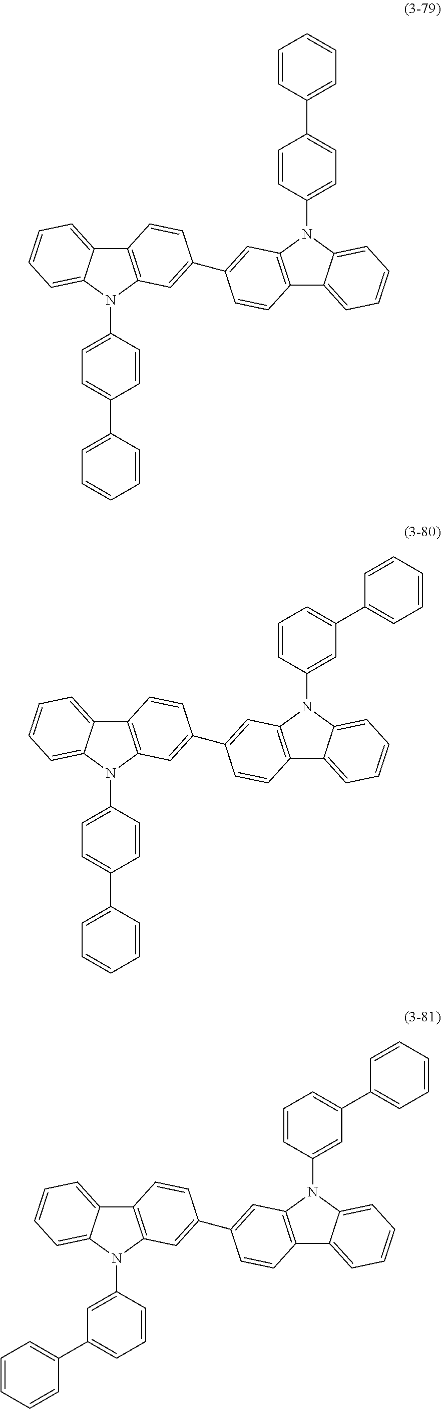

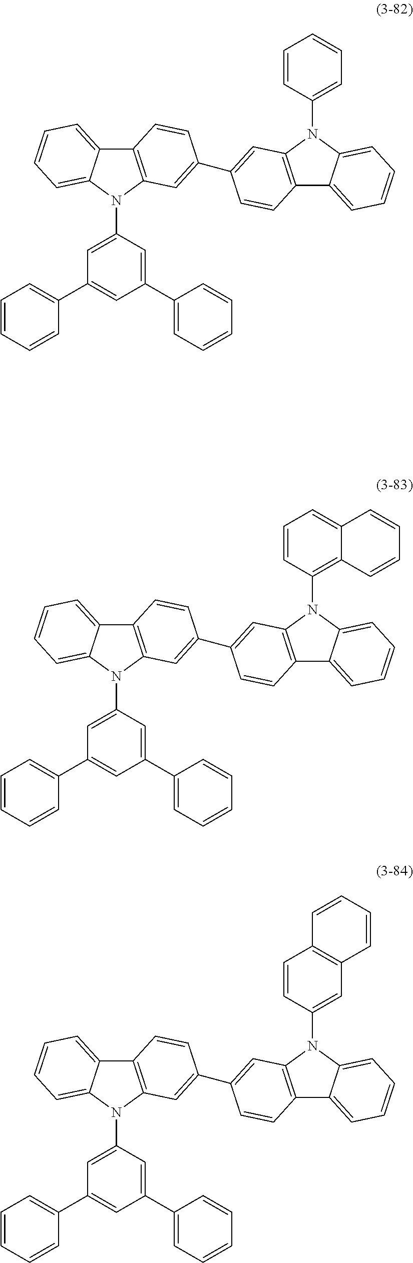

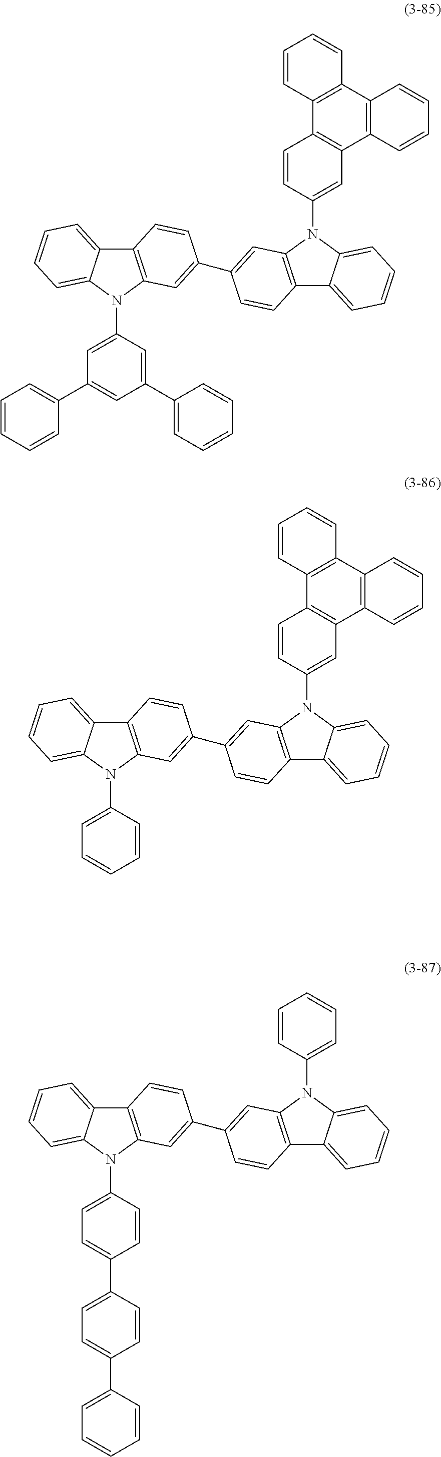

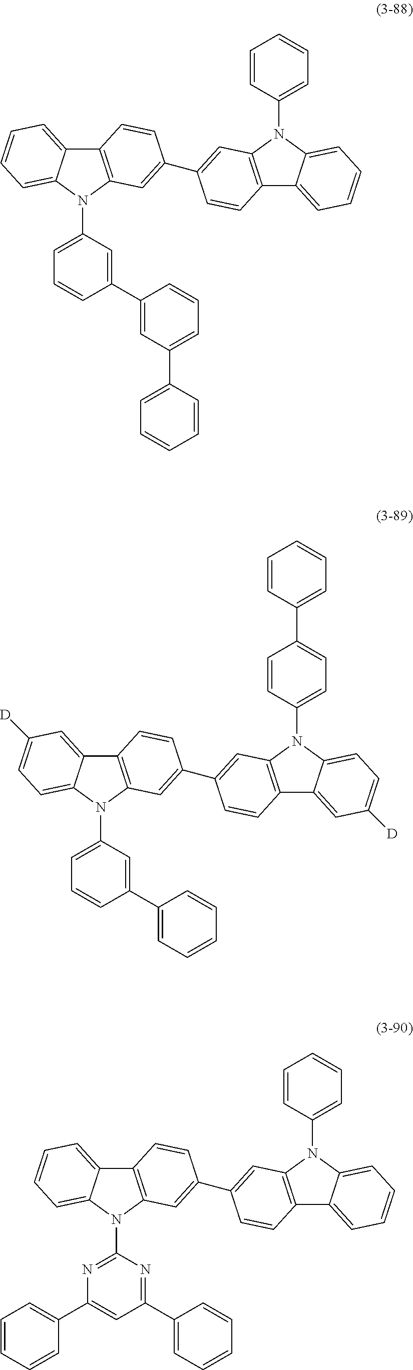

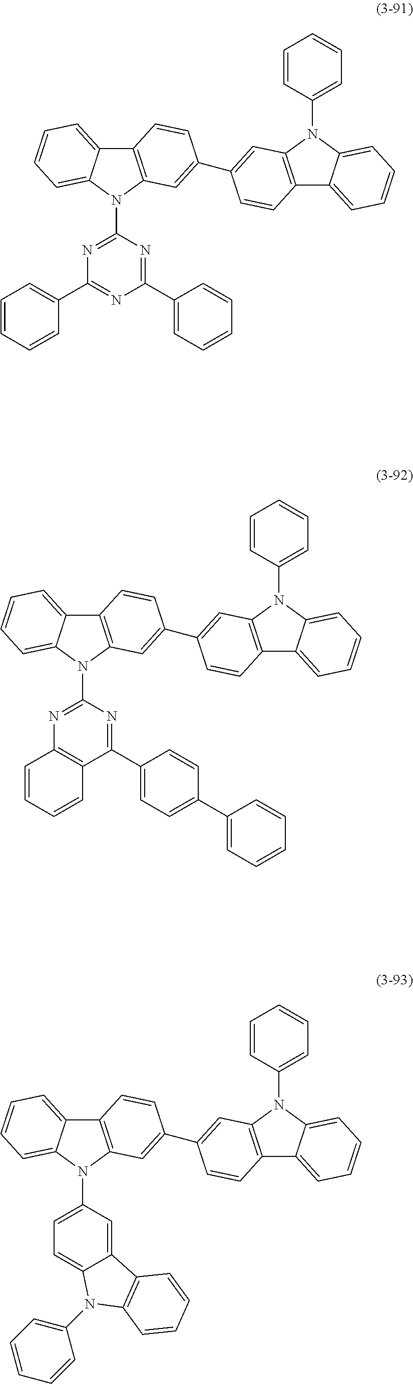

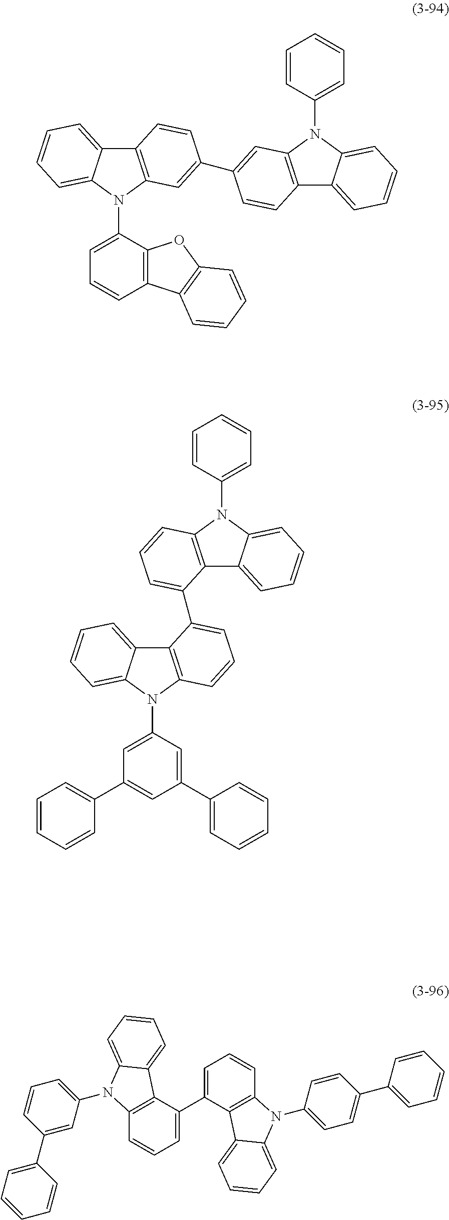

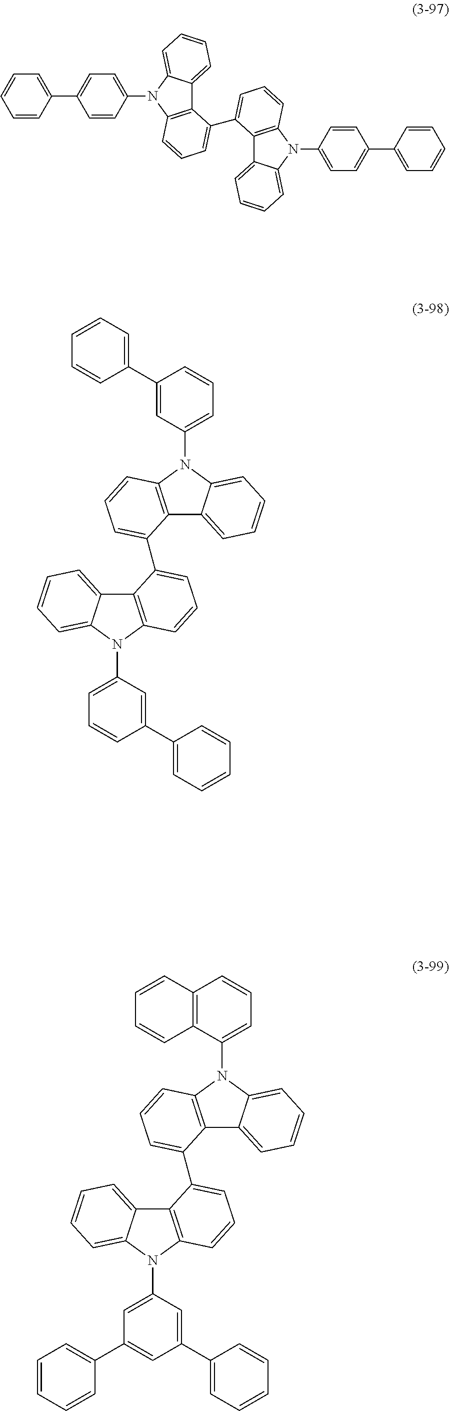

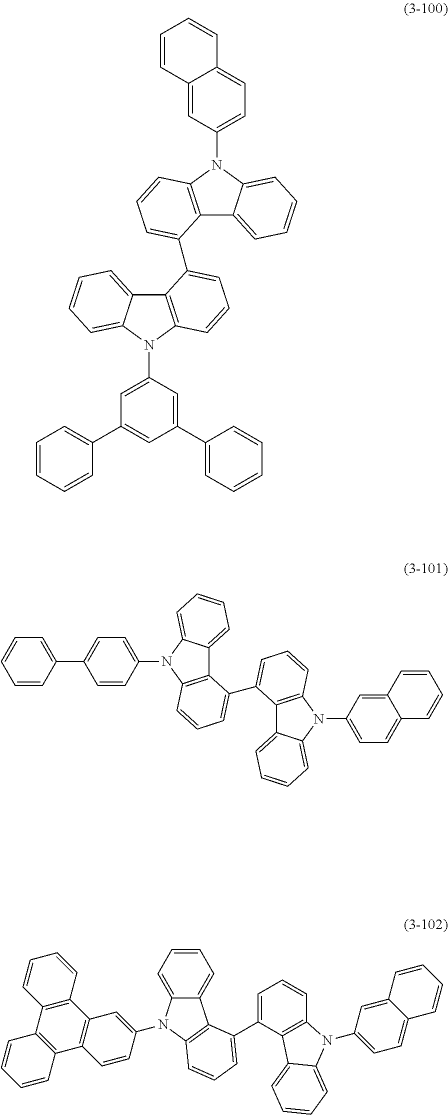

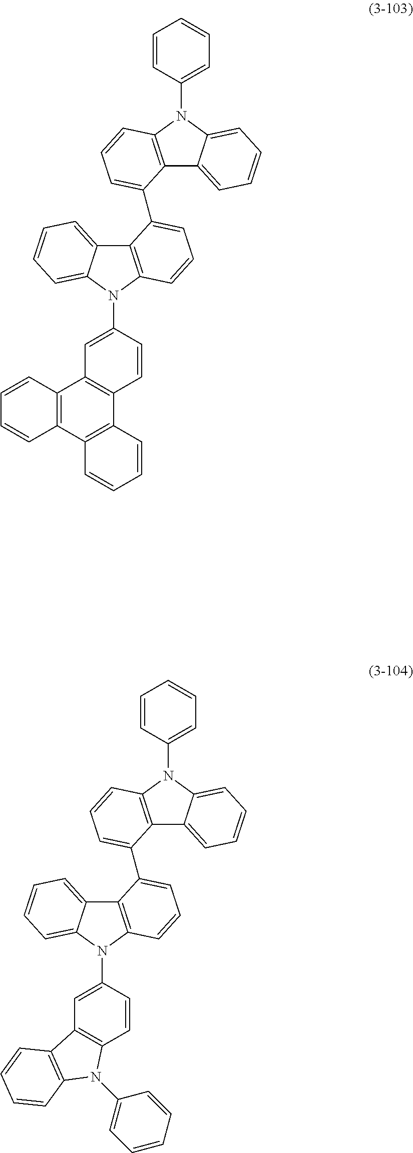

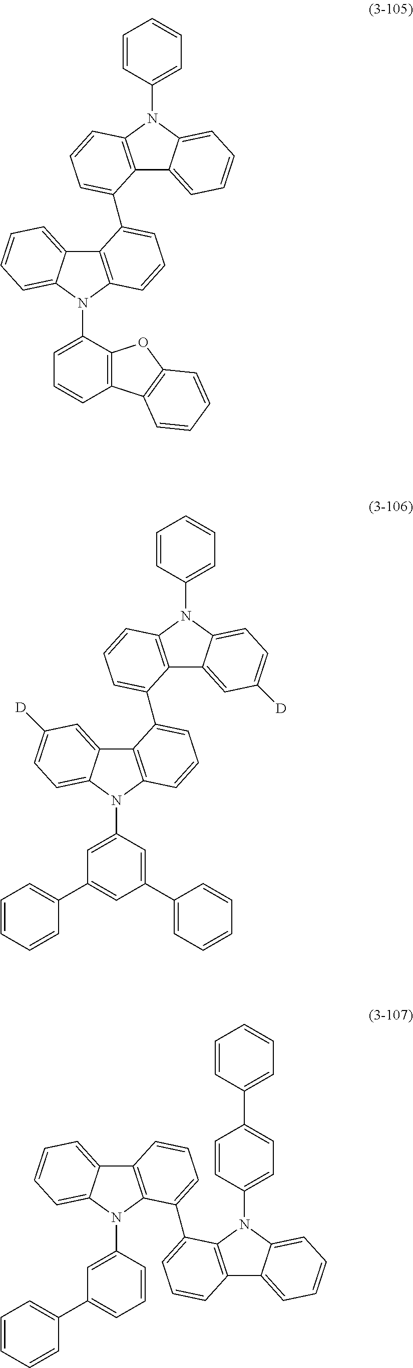

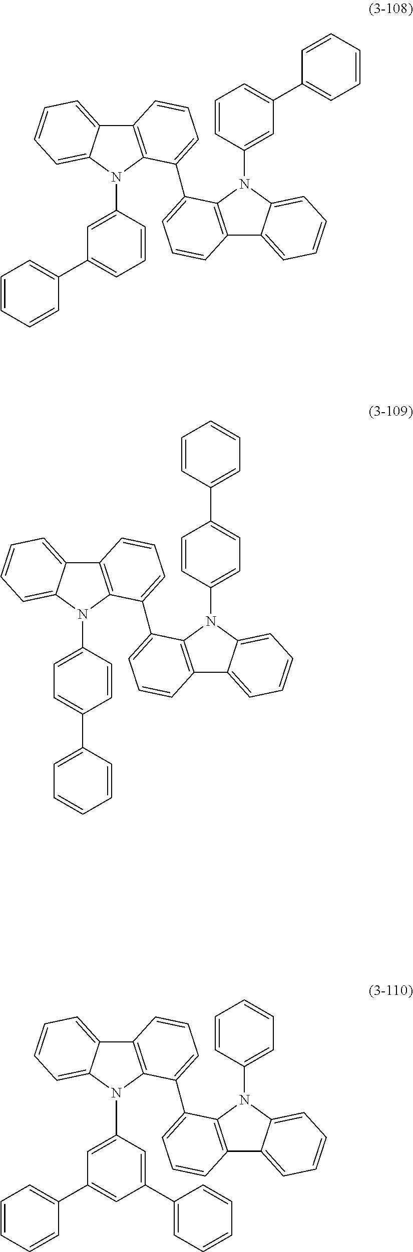

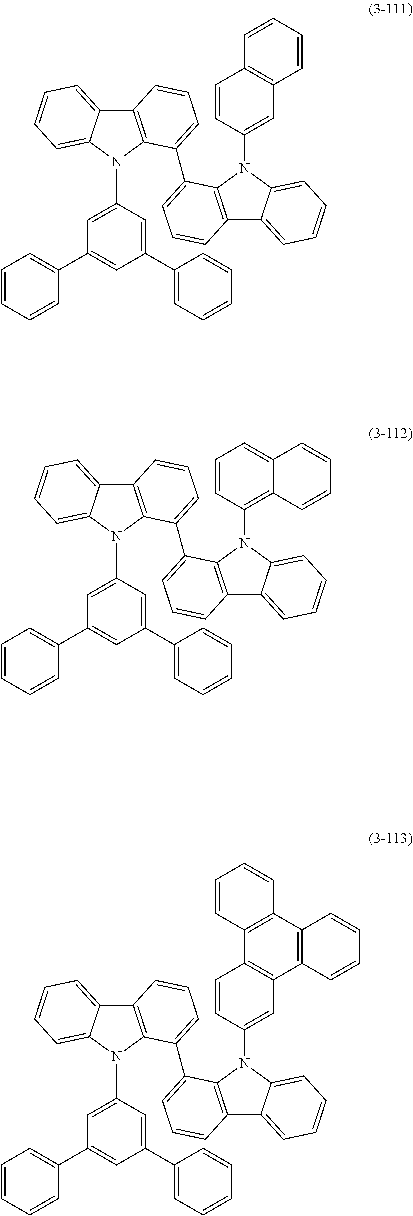

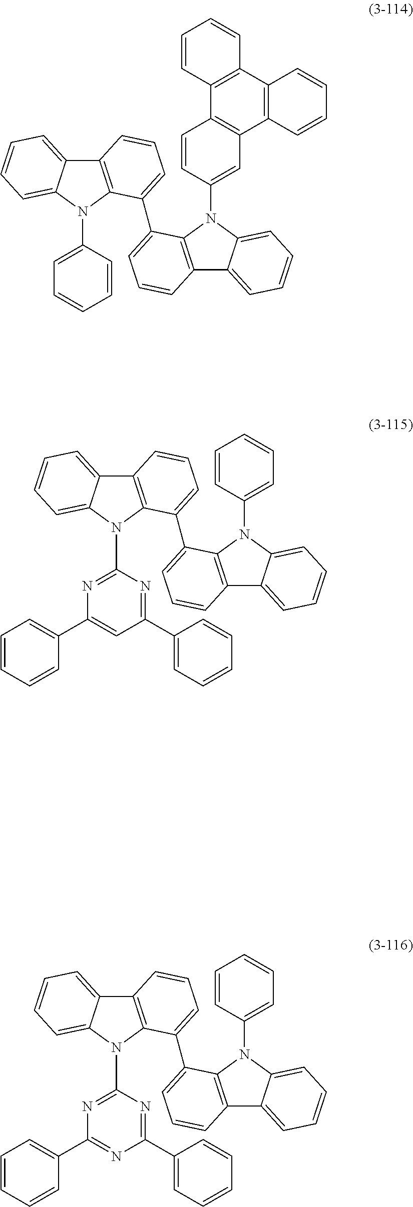

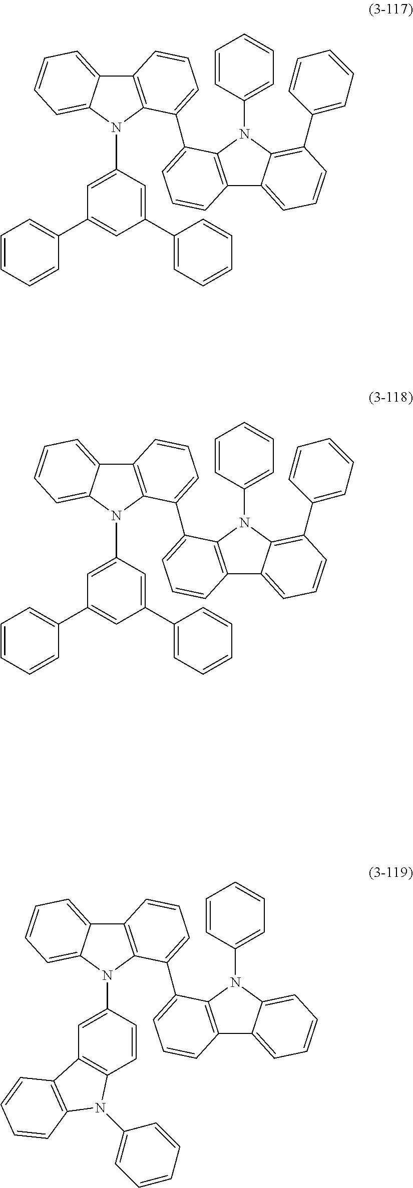

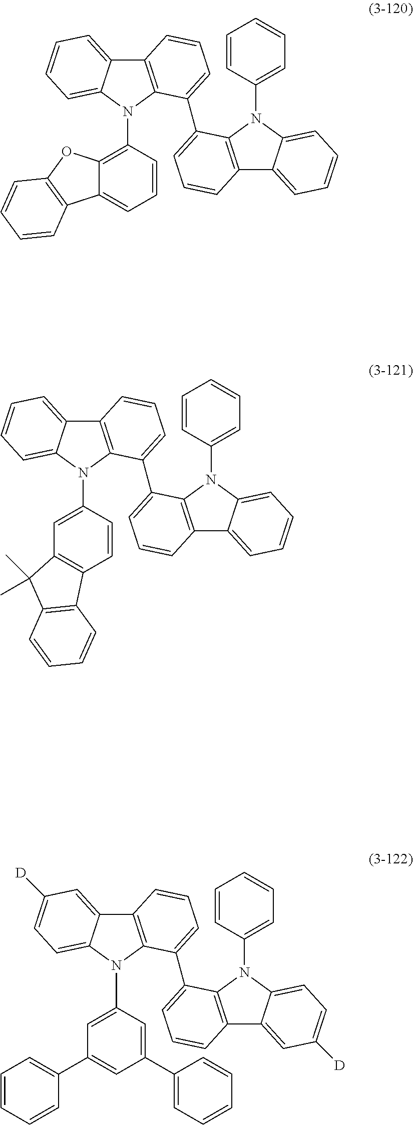

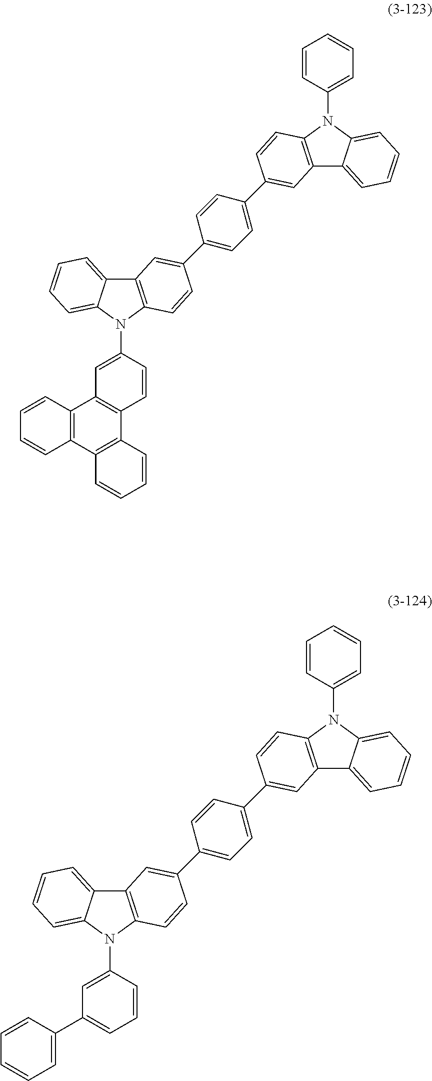

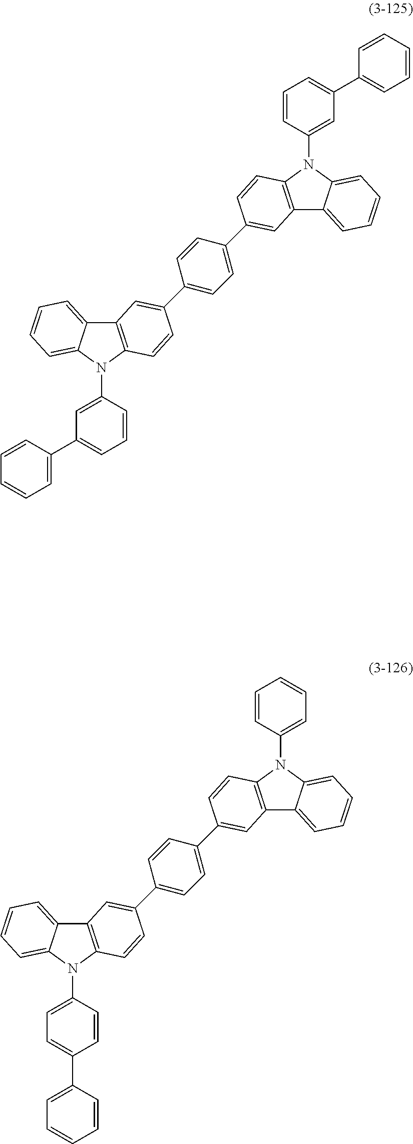

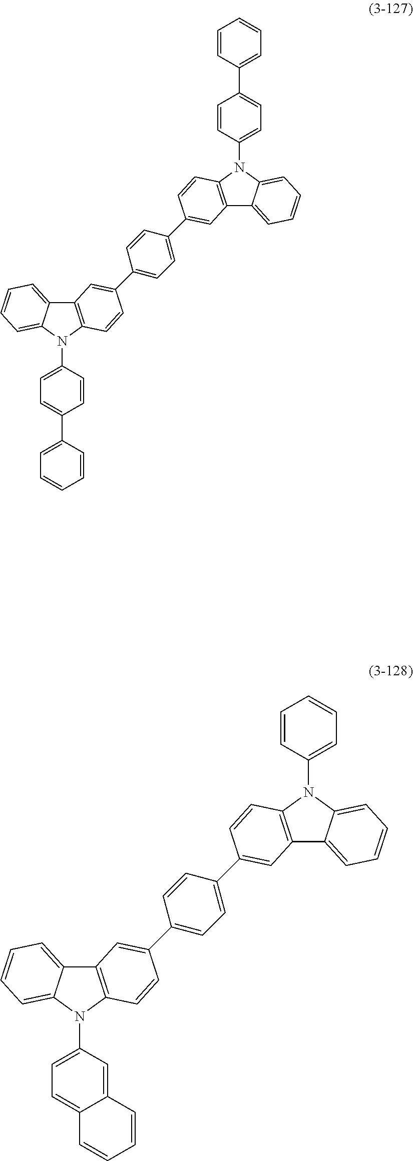

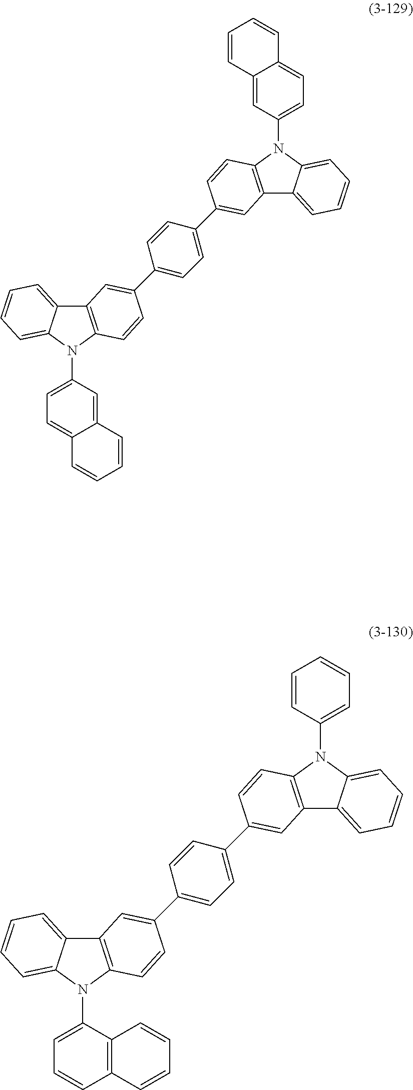

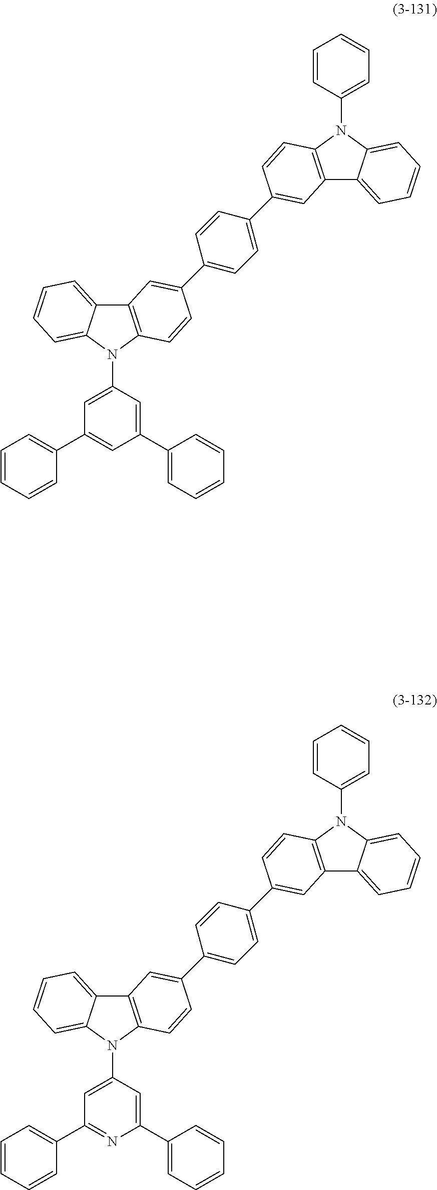

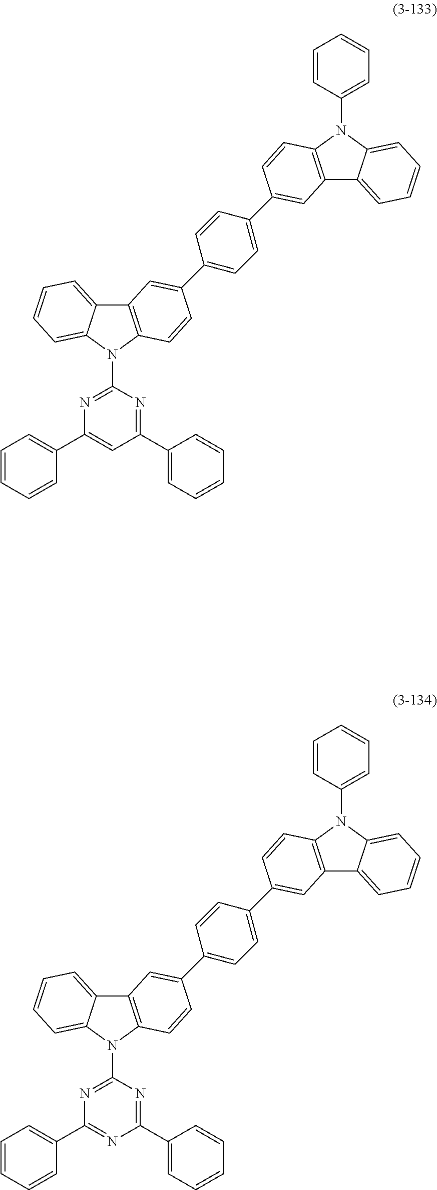

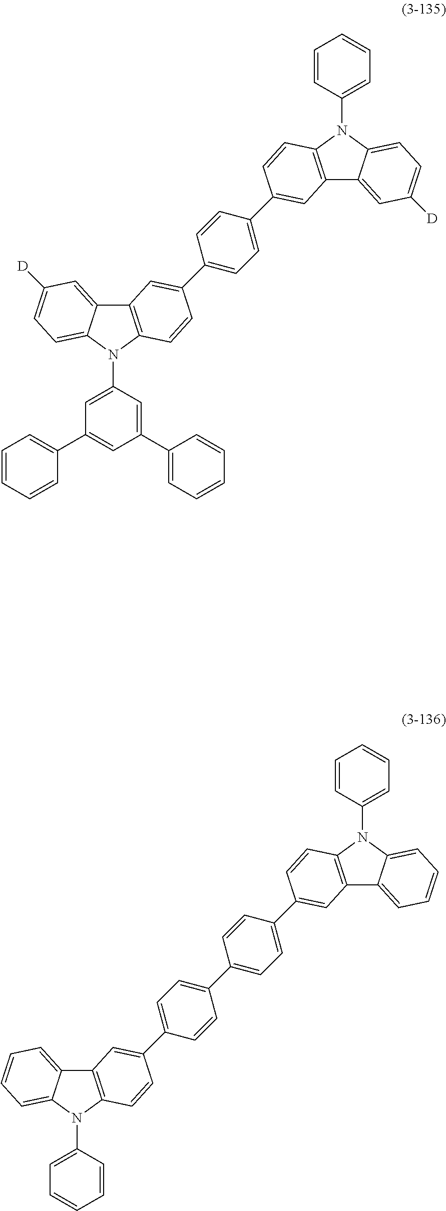

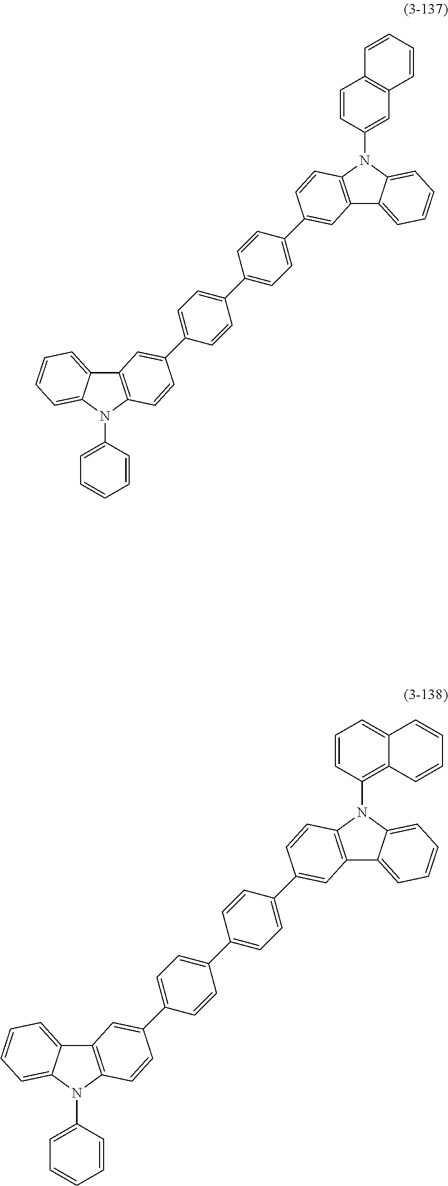

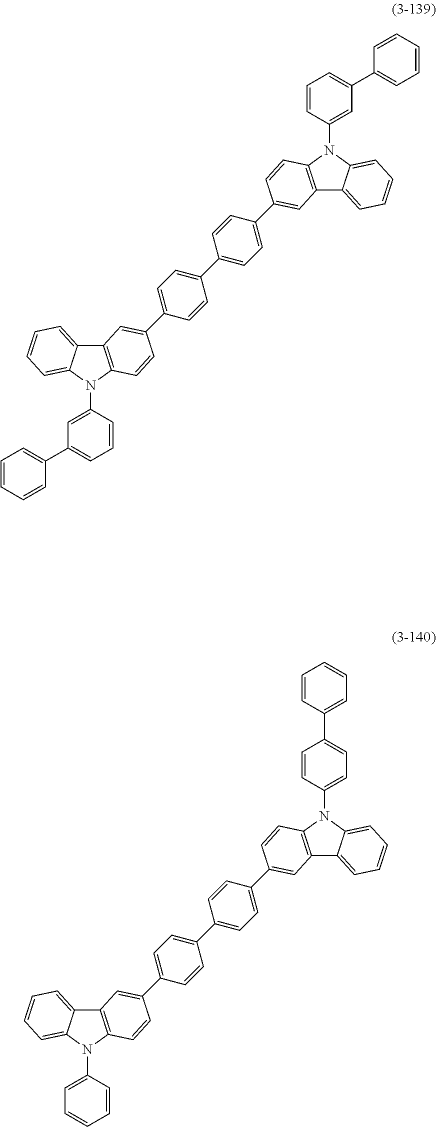

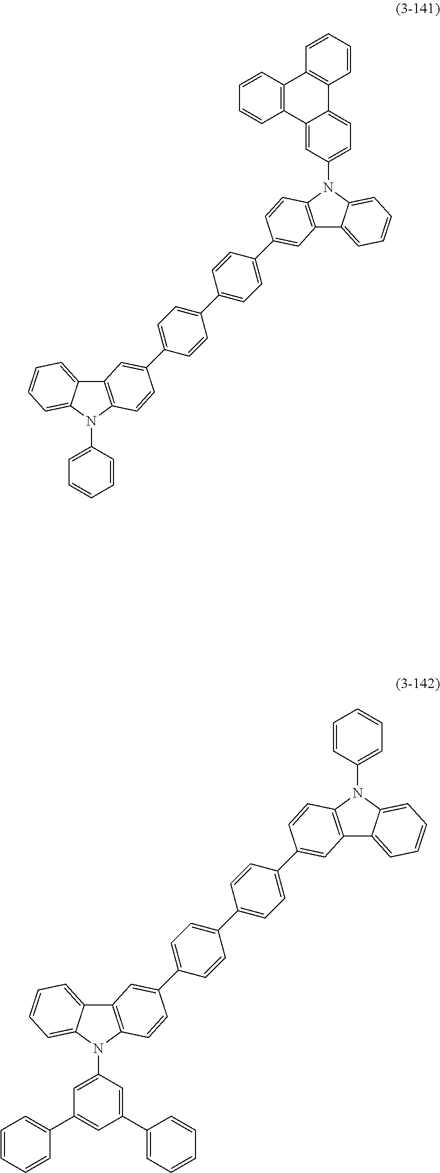

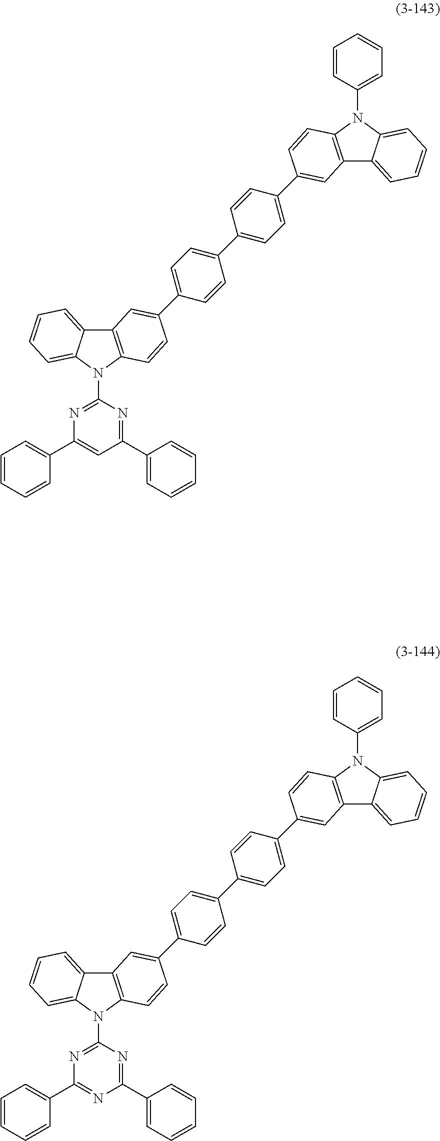

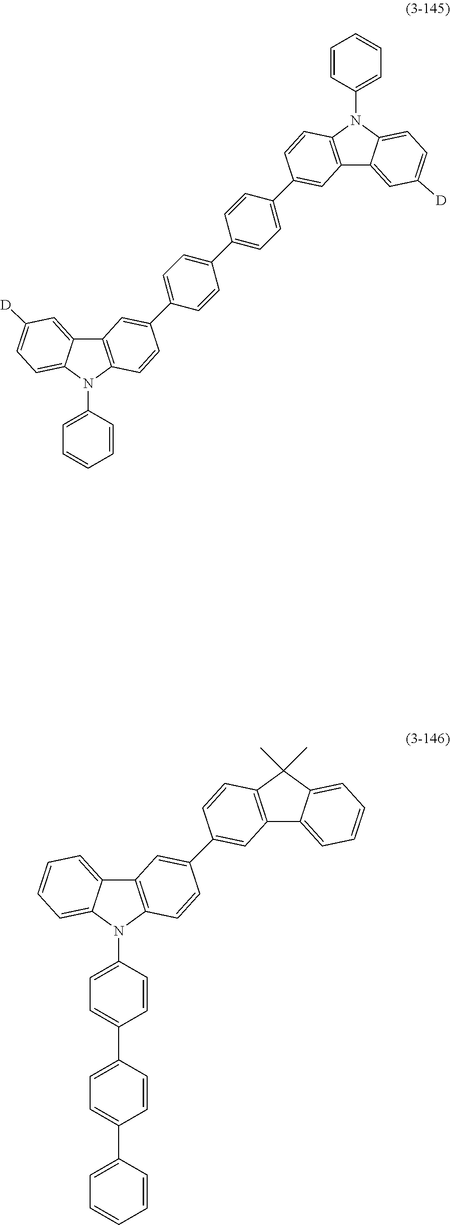

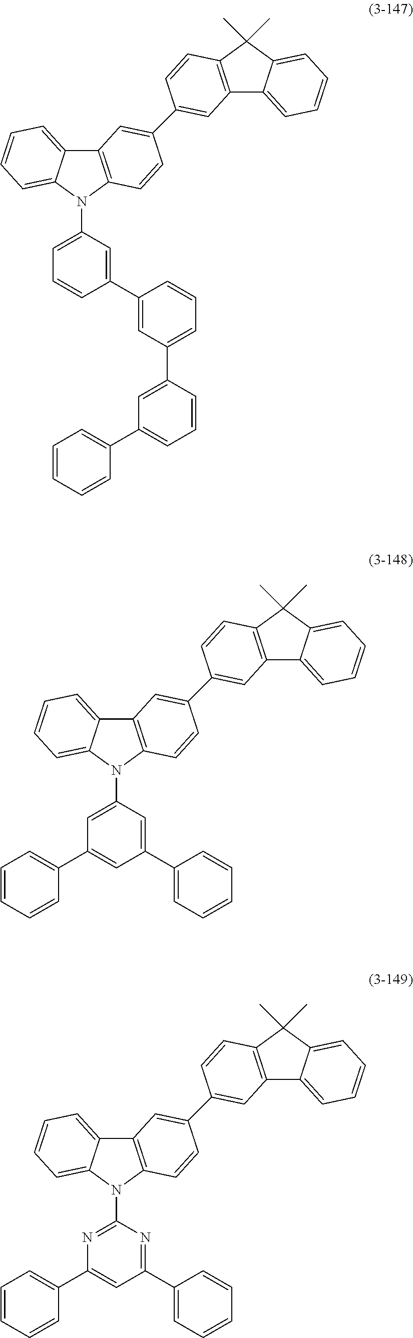

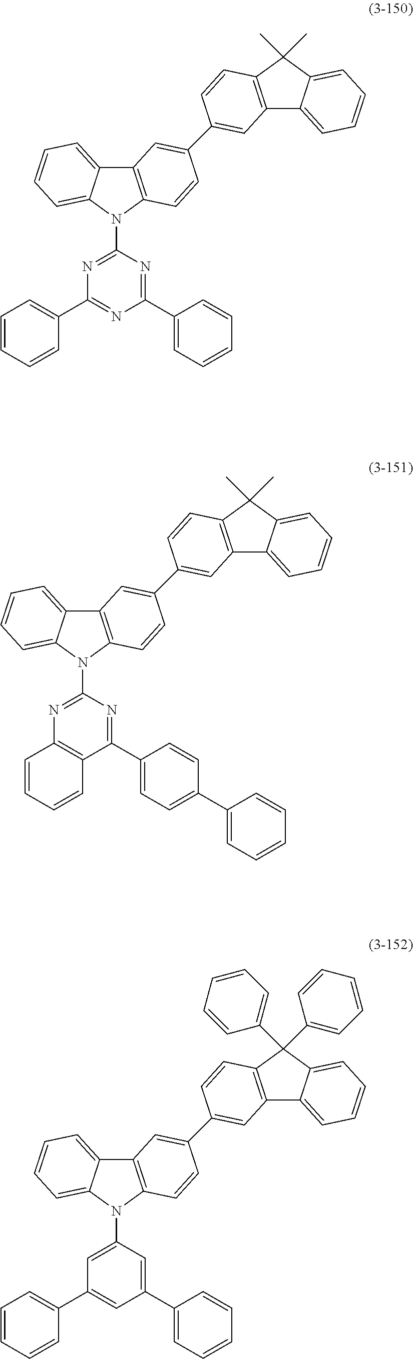

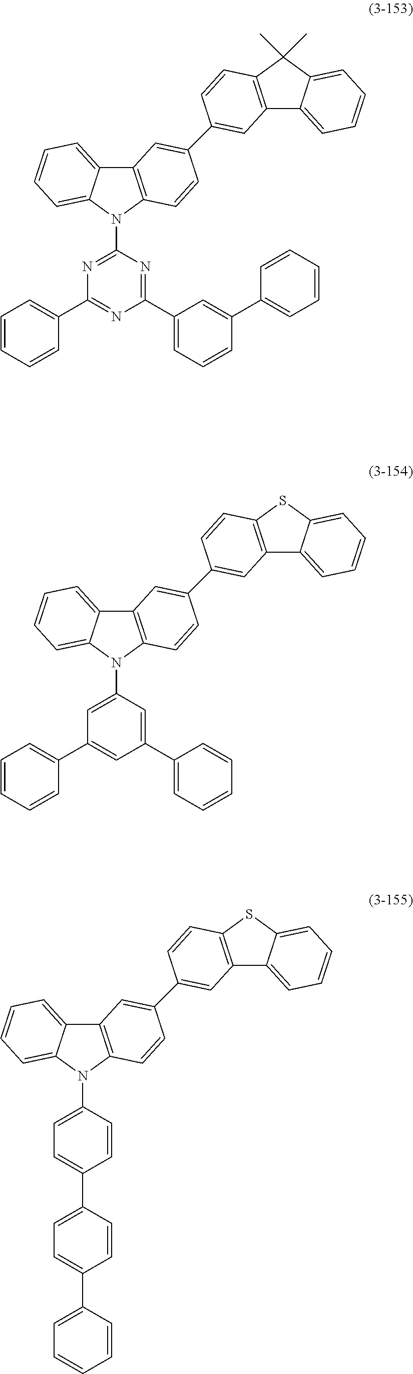

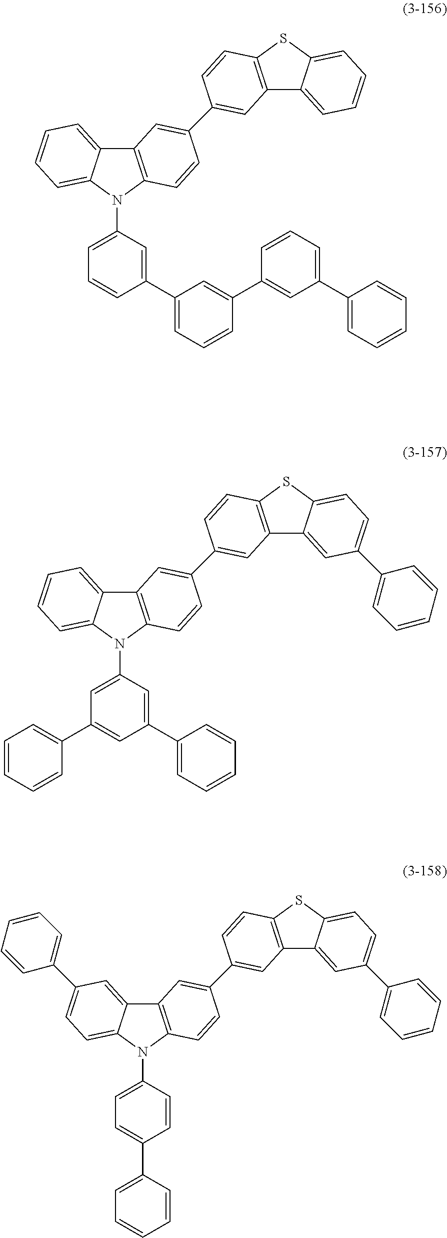

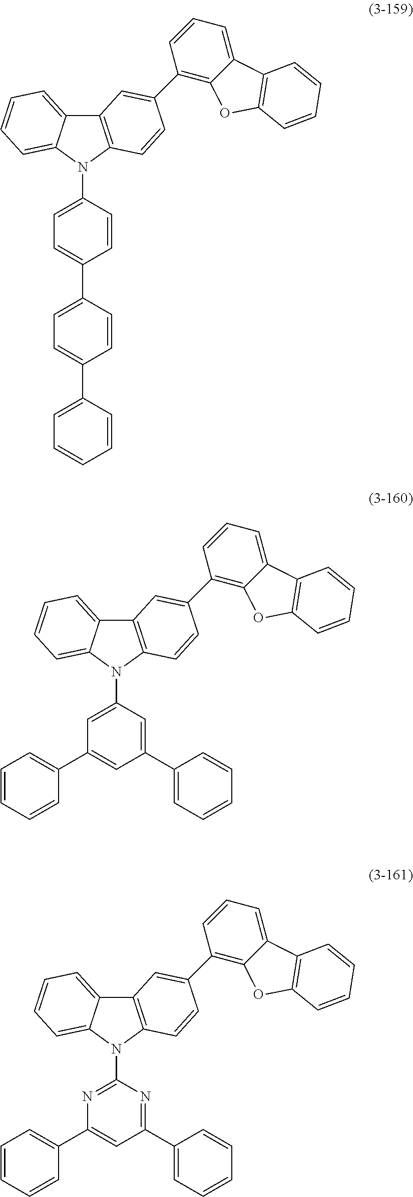

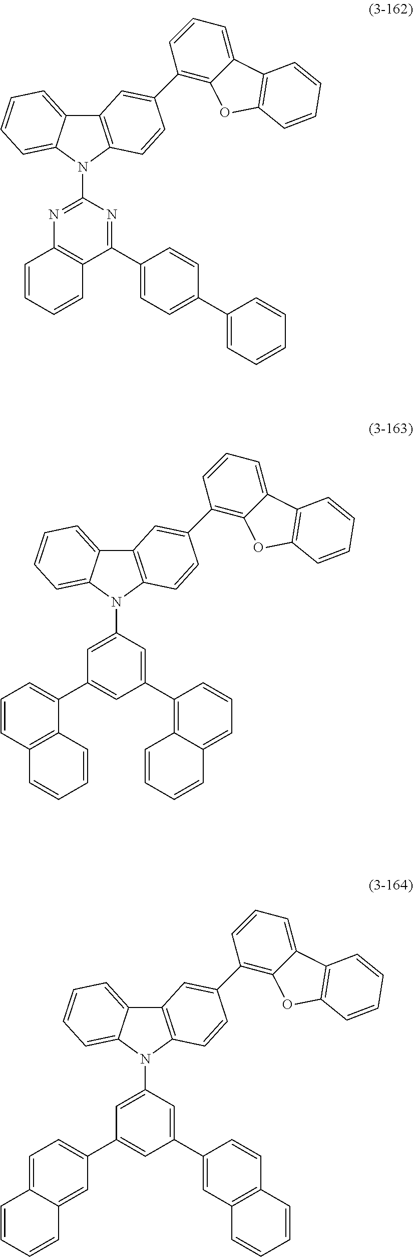

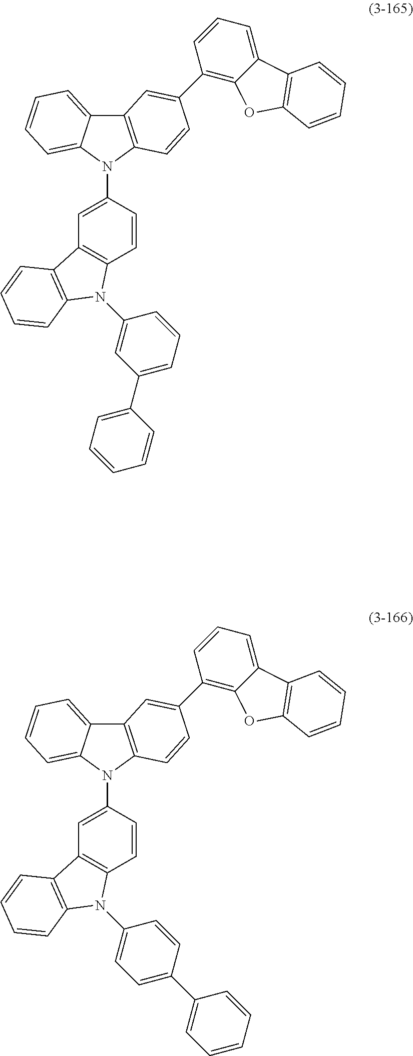

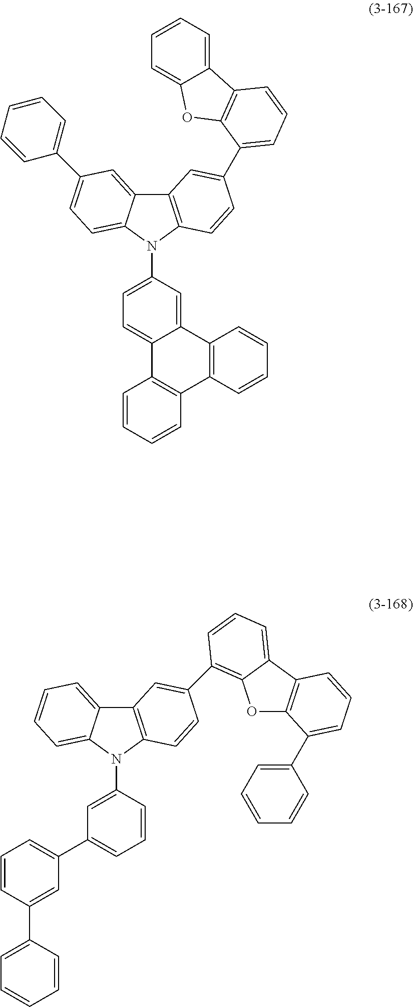

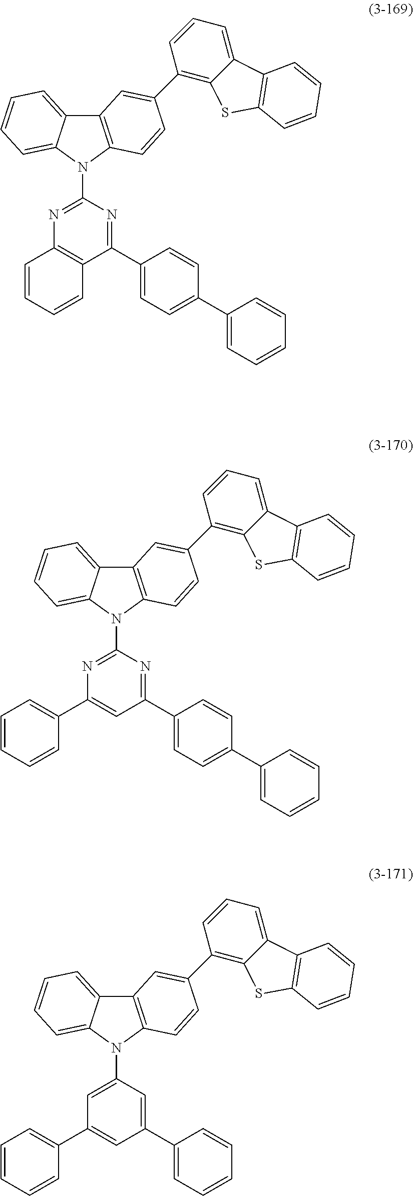

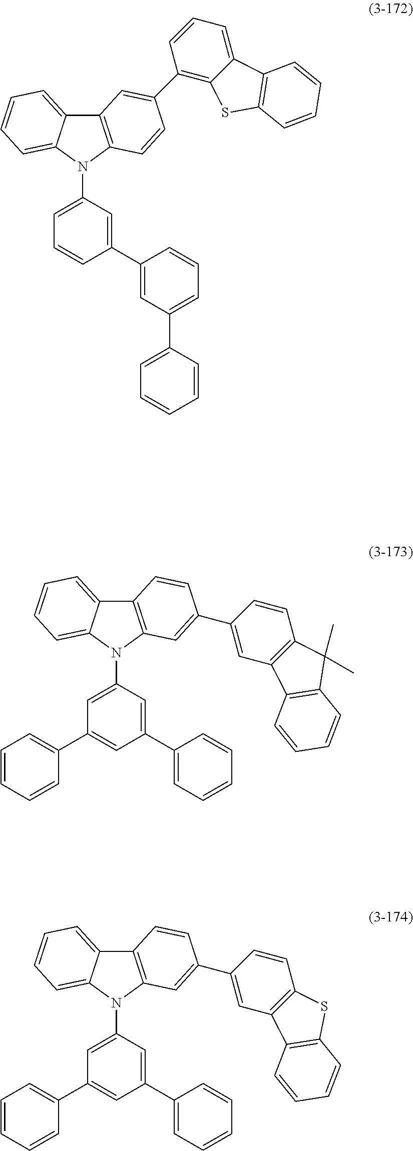

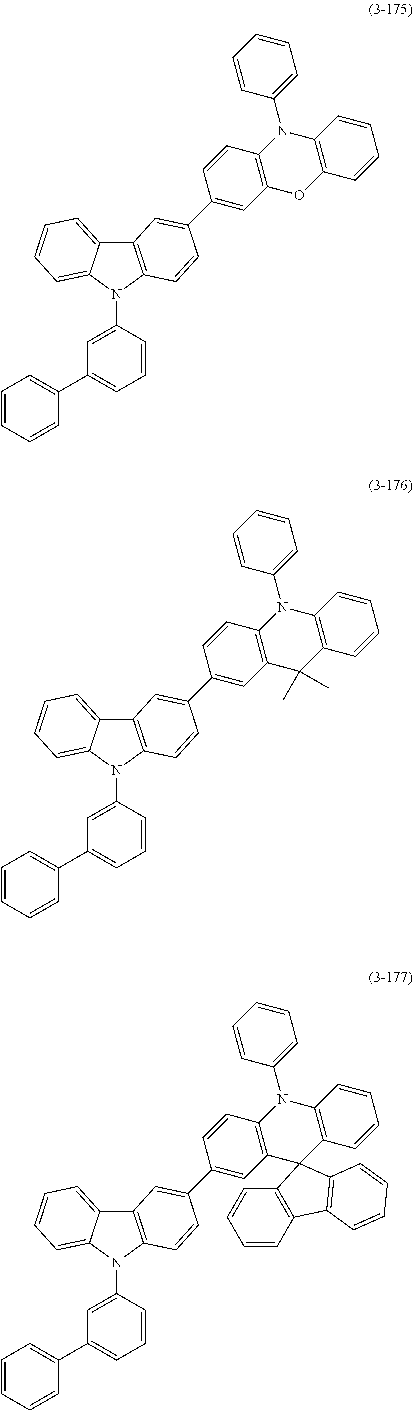

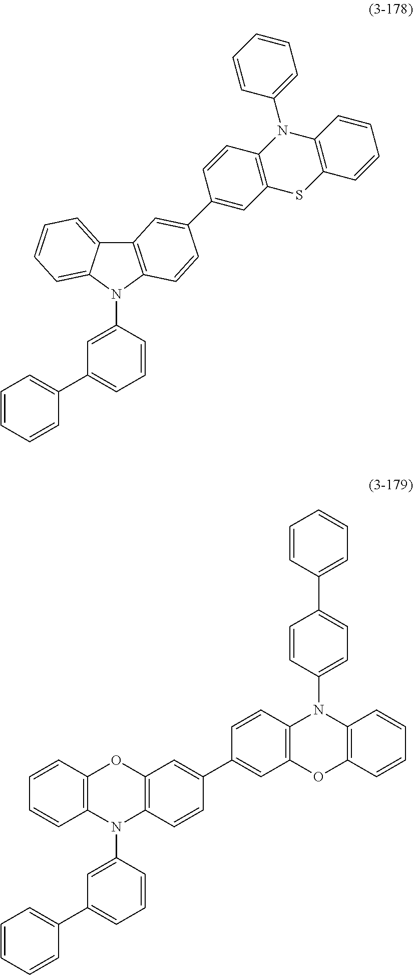

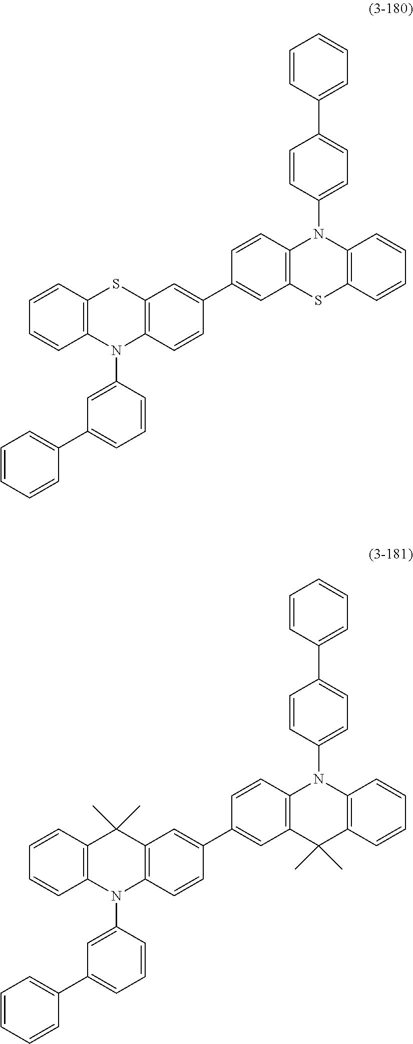

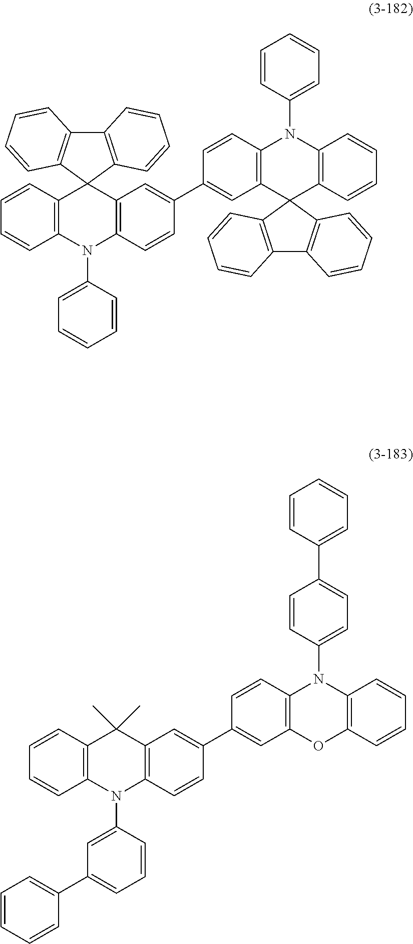

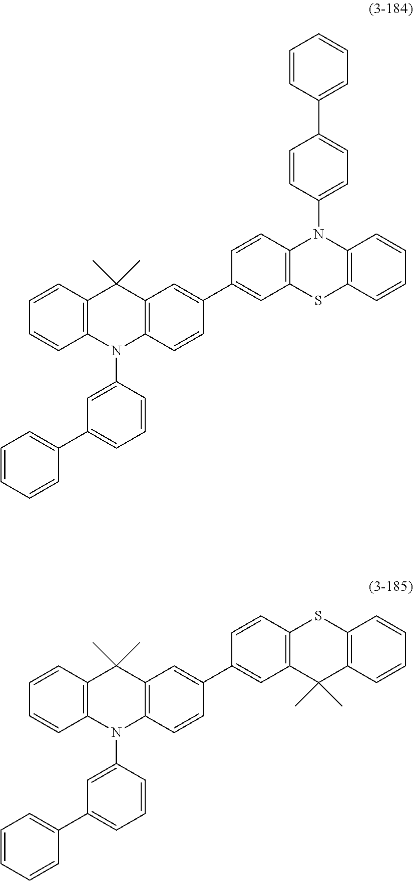

Specifically, the second organic compound satisfying the general formula (4) includes, but is not limited to, the following compounds:

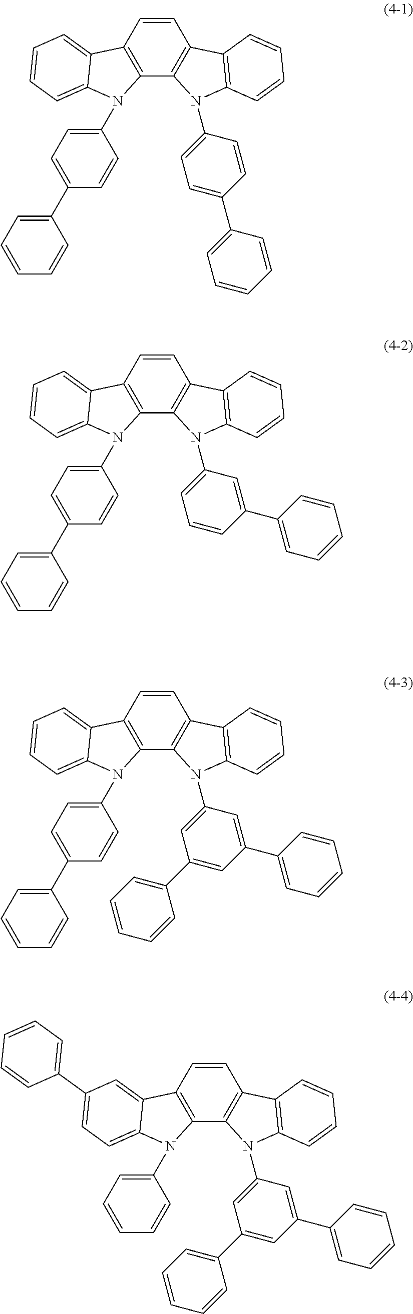

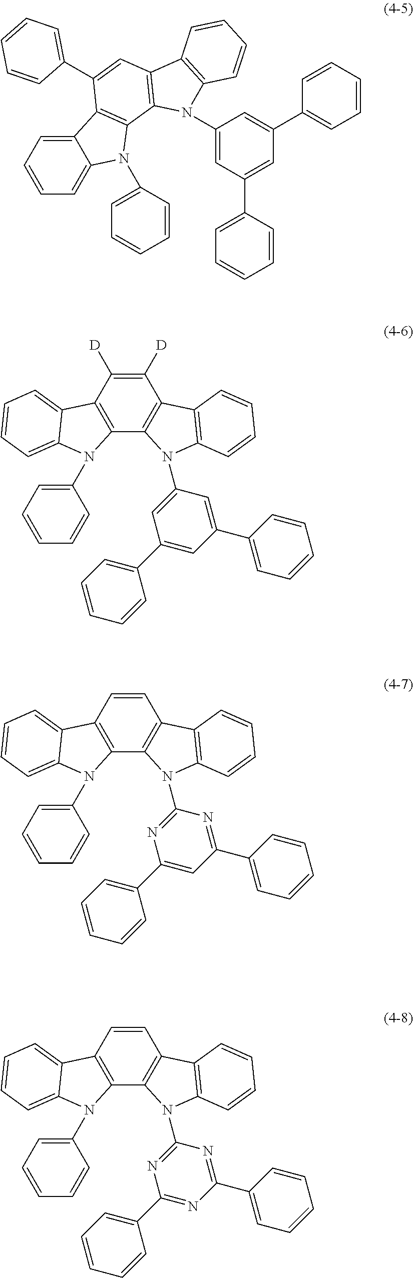

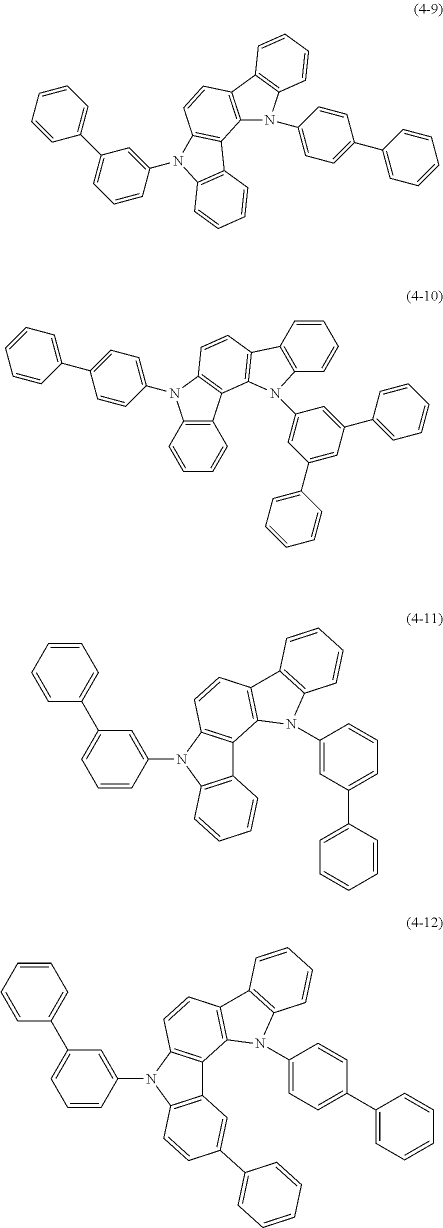

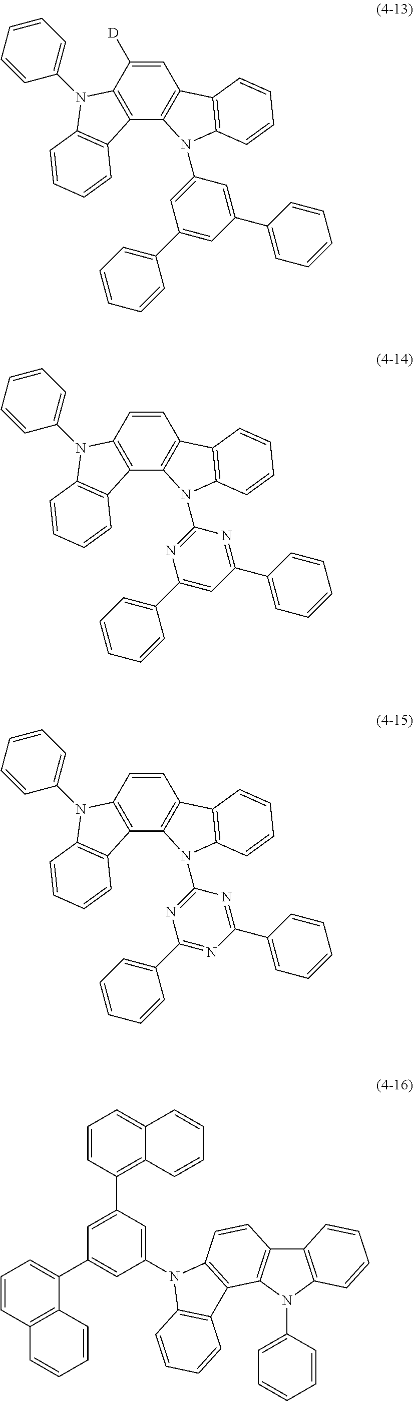

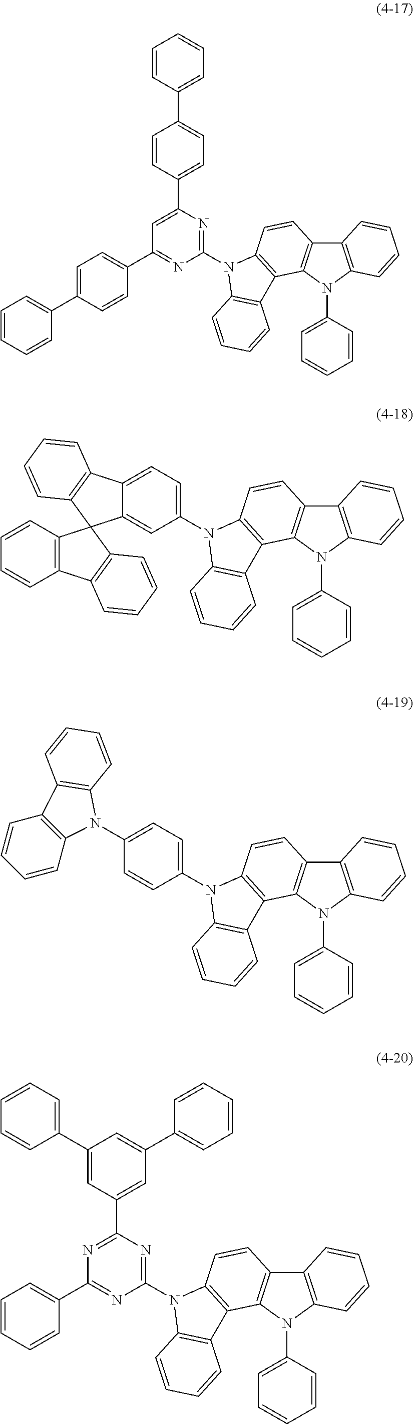

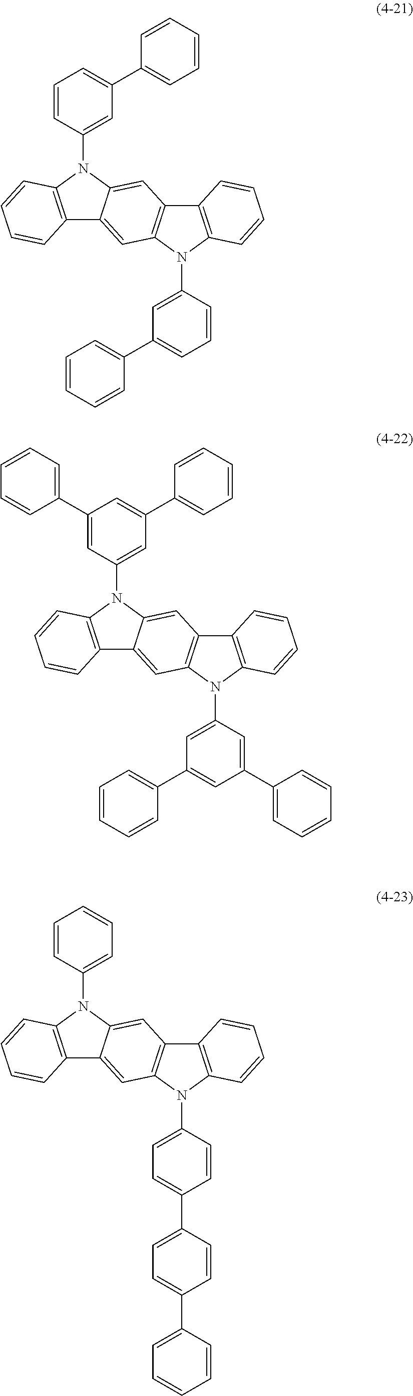

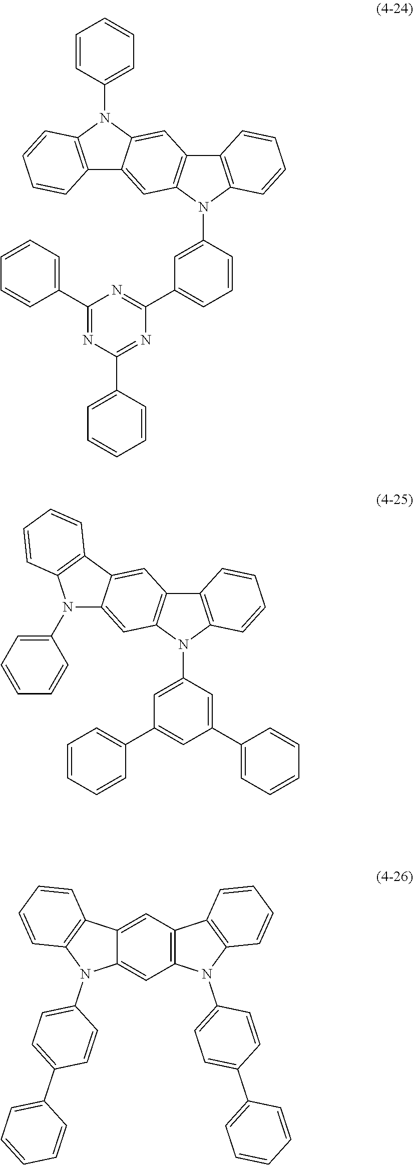

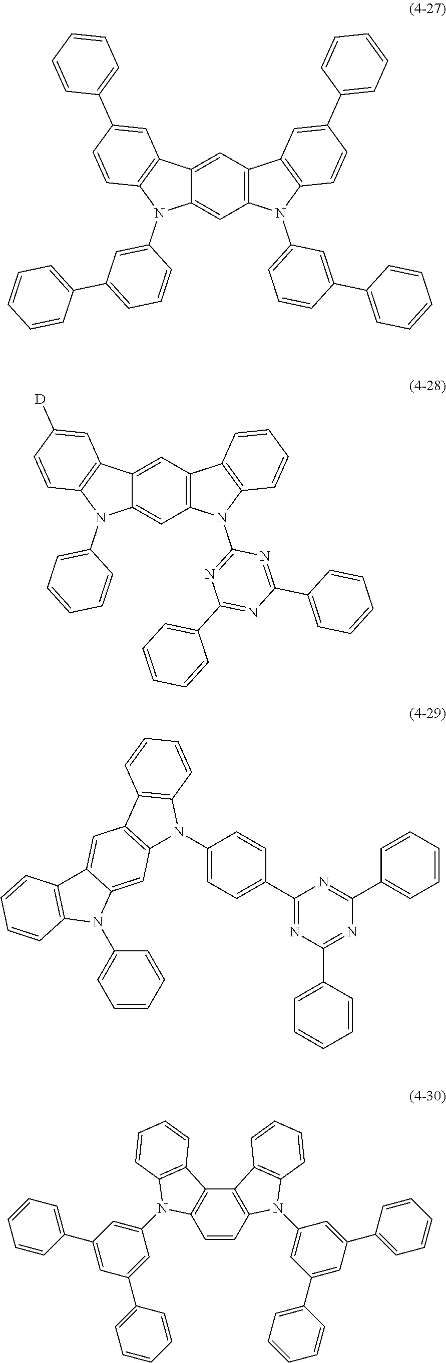

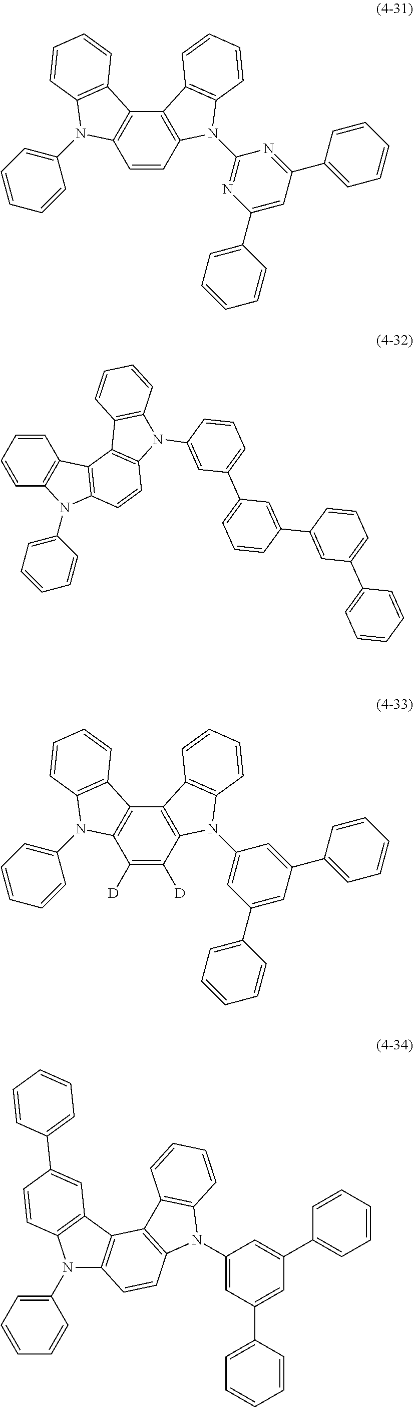

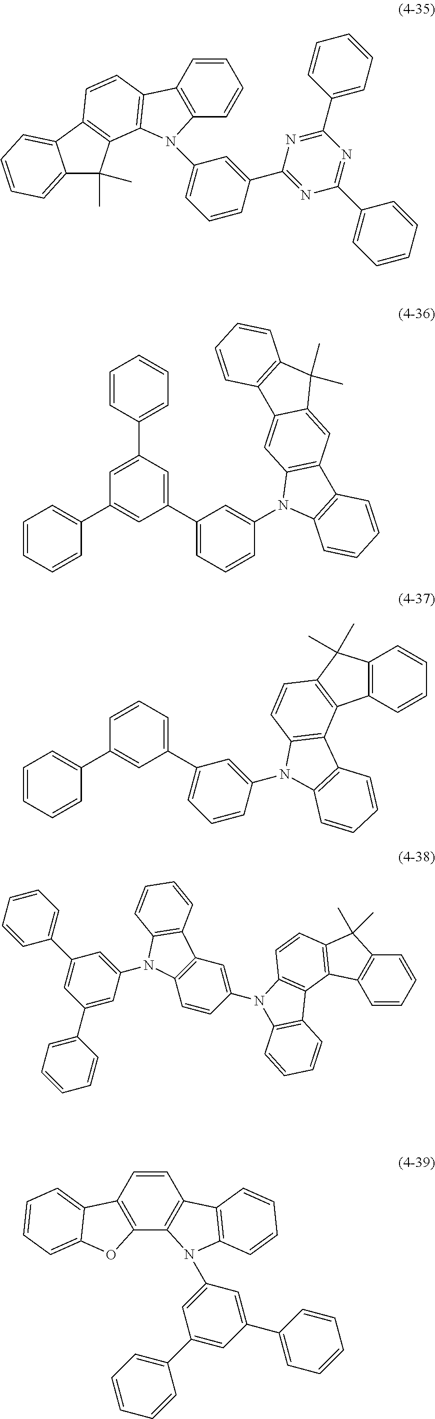

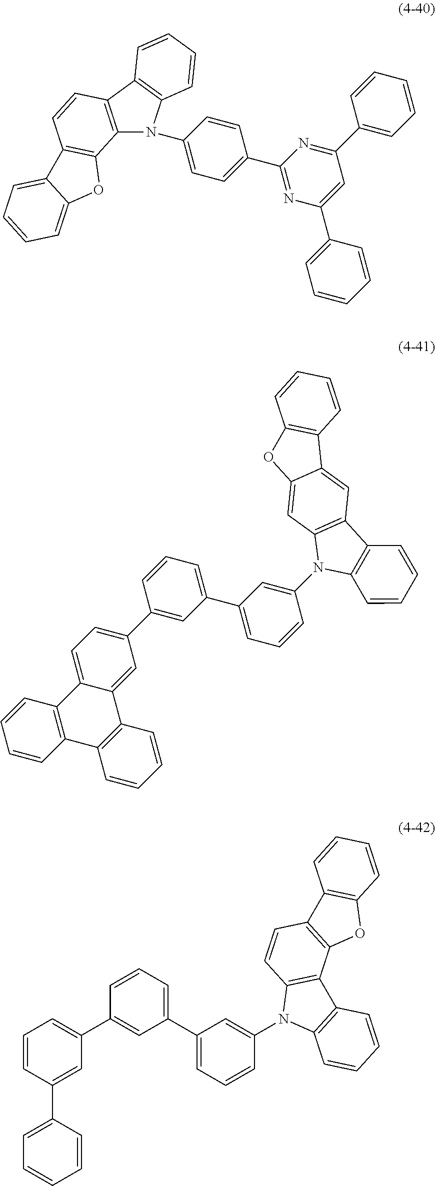

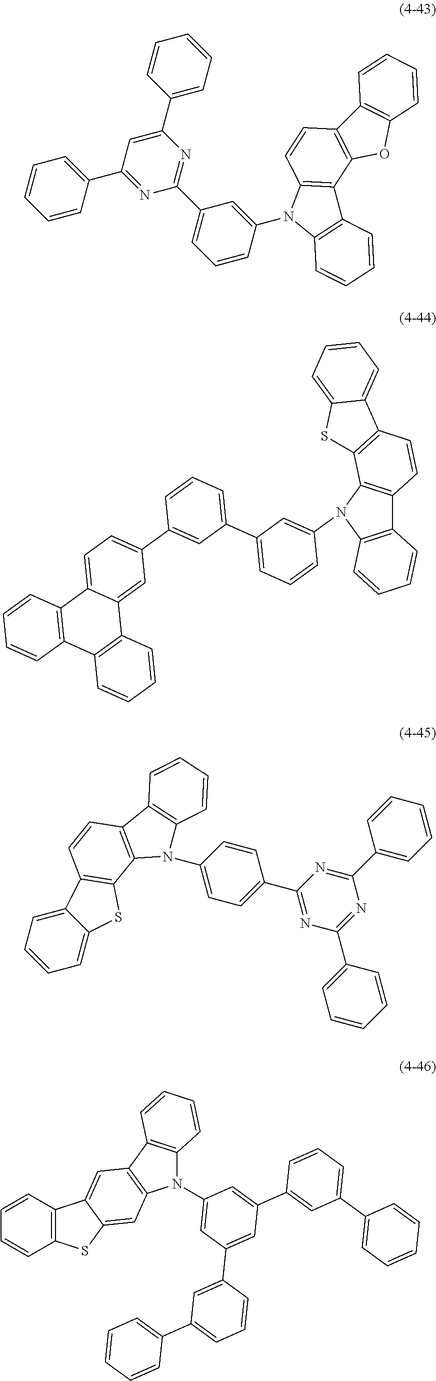

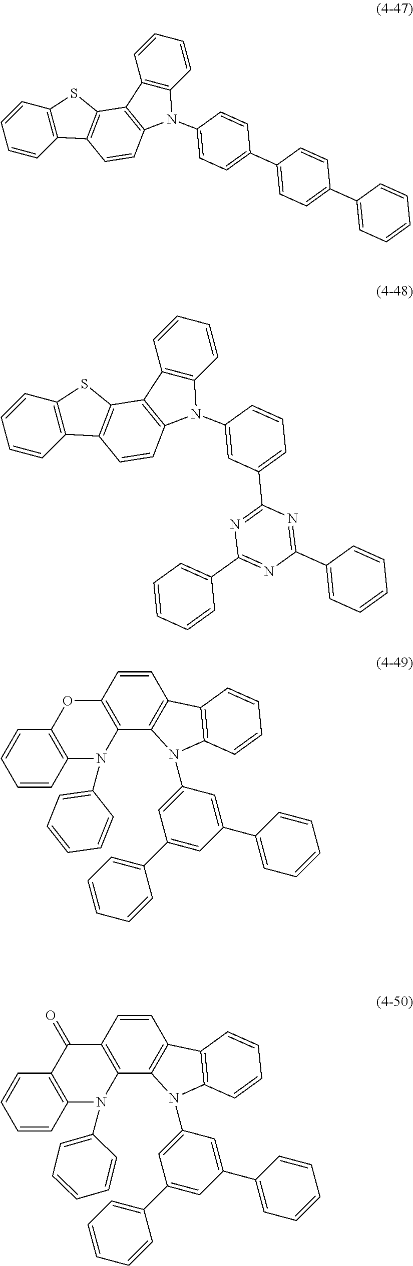

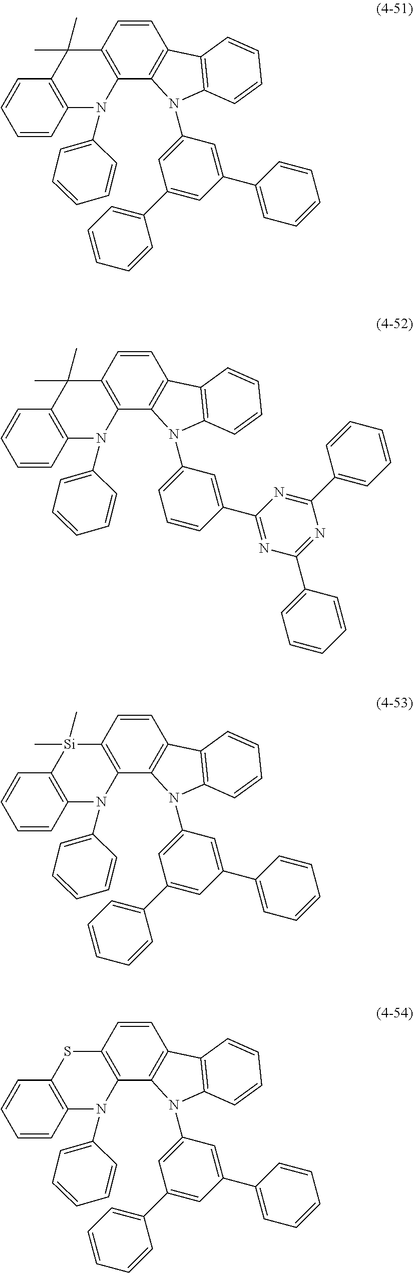

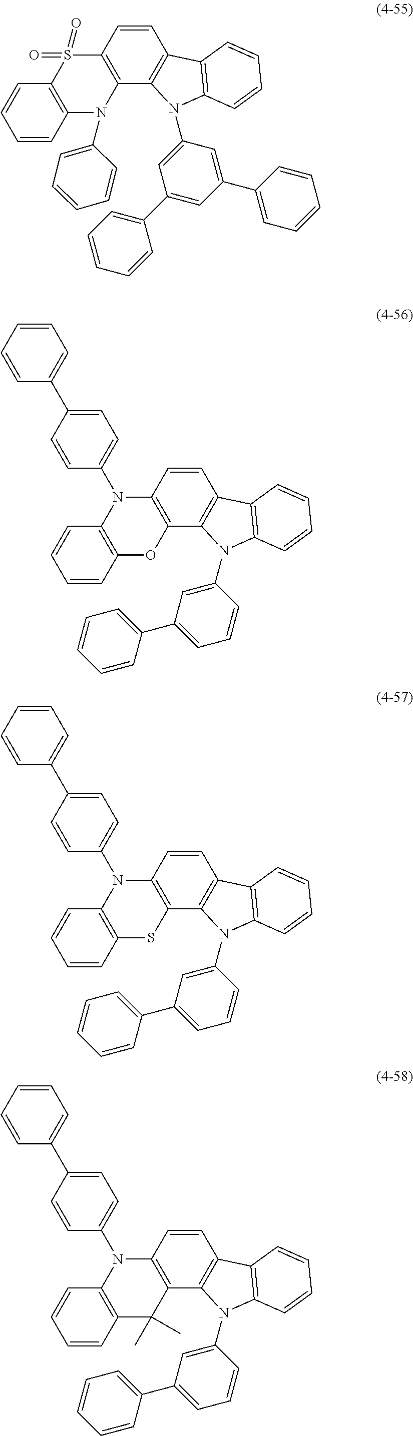

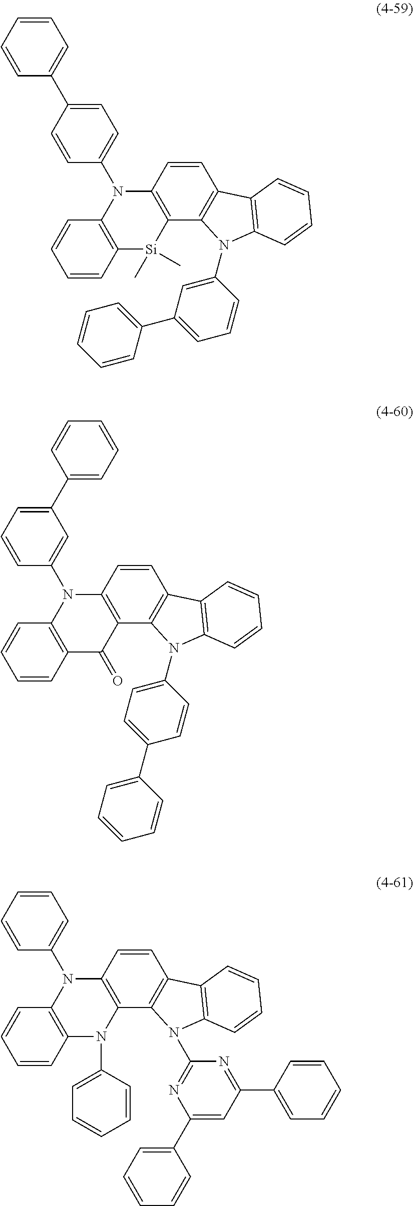

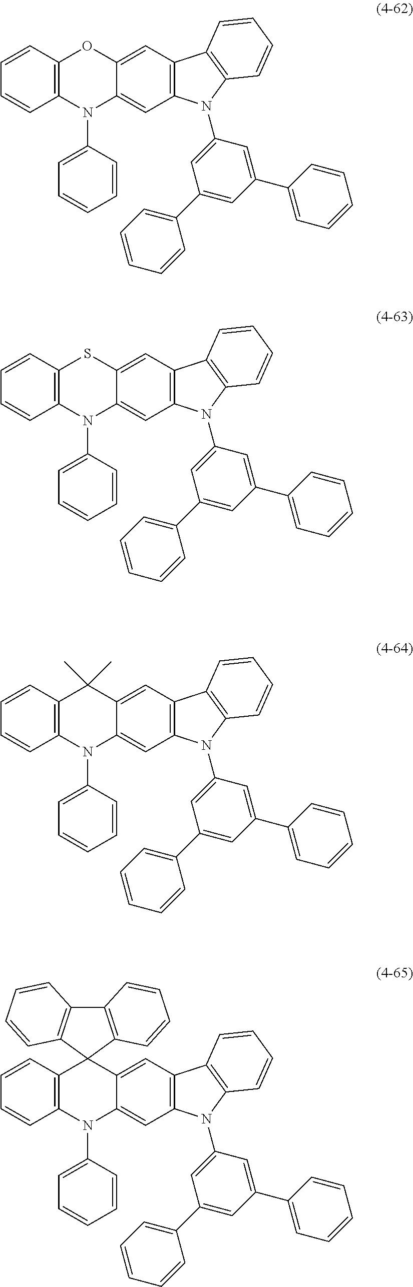

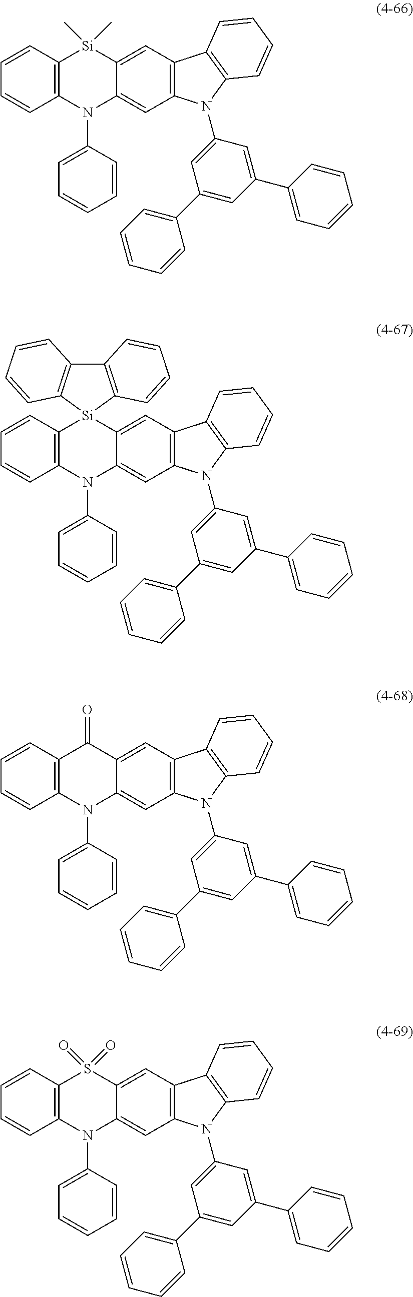

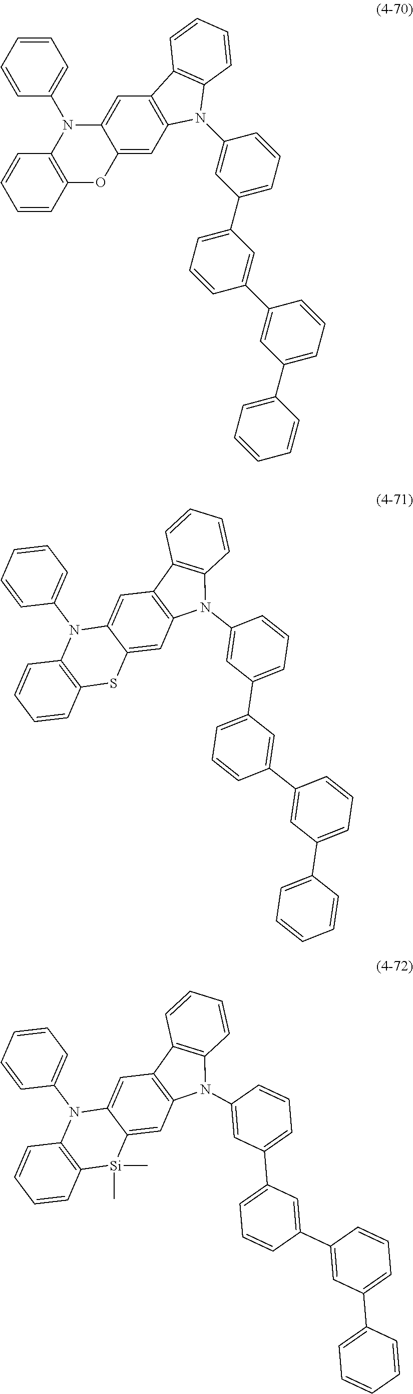

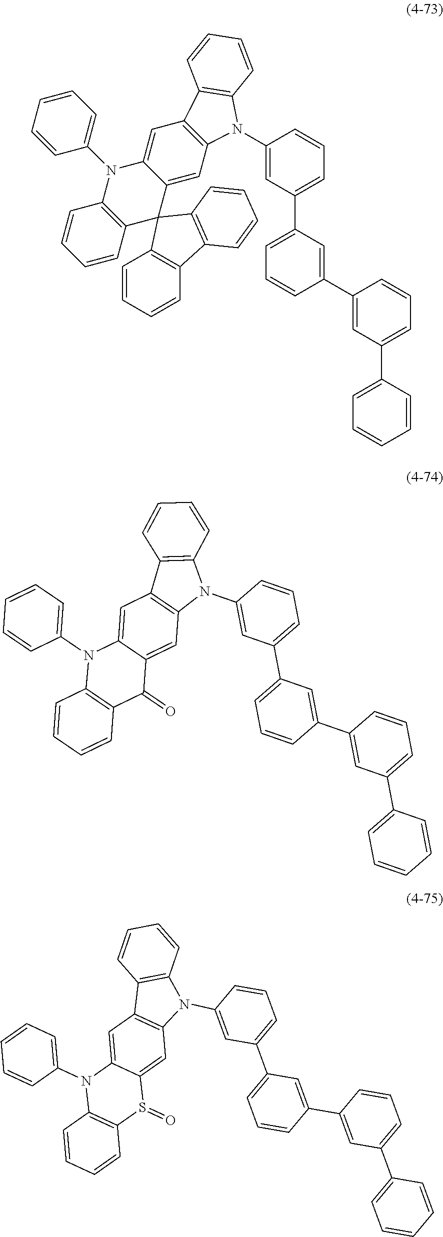

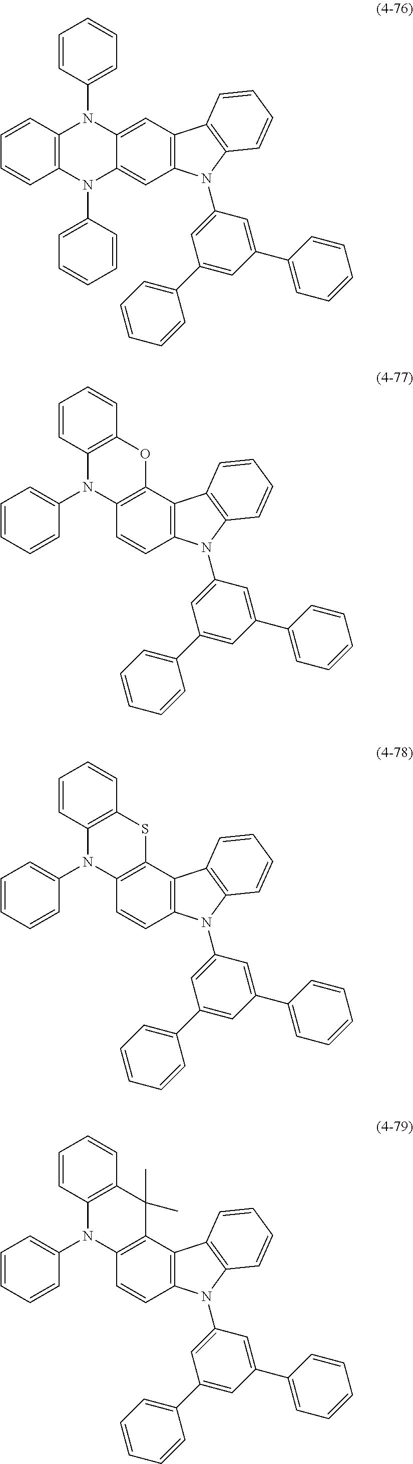

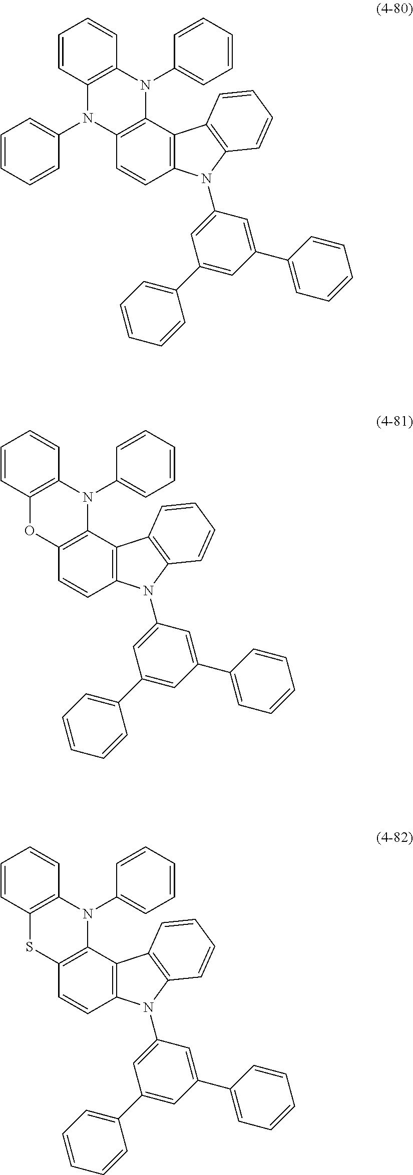

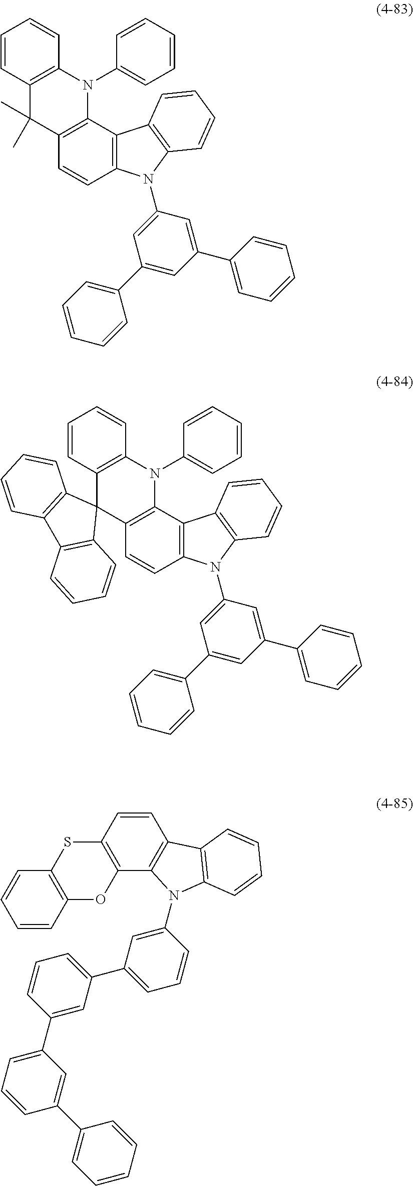

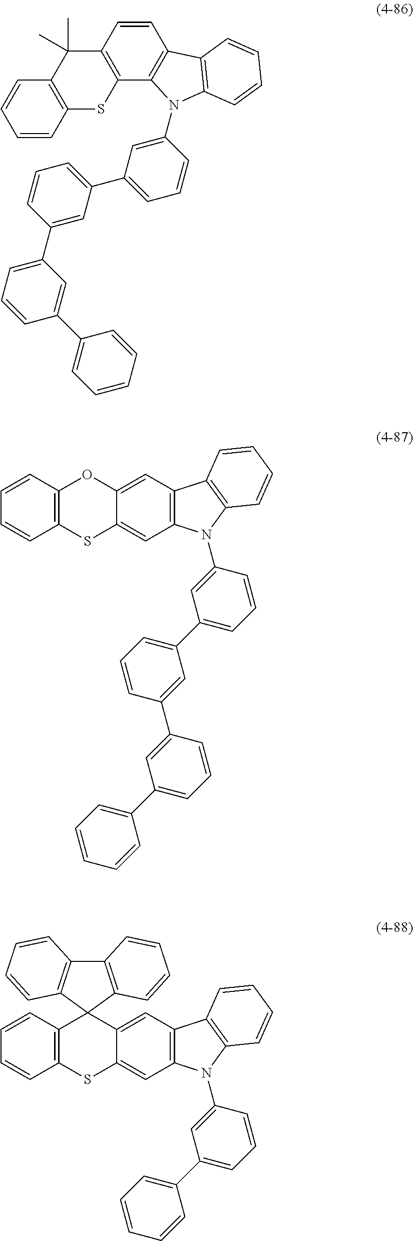

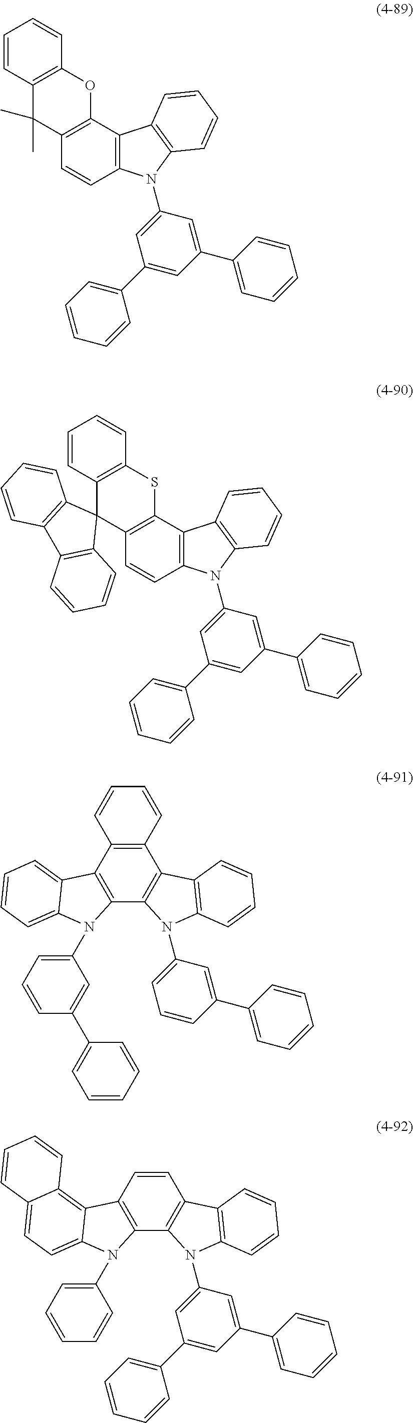

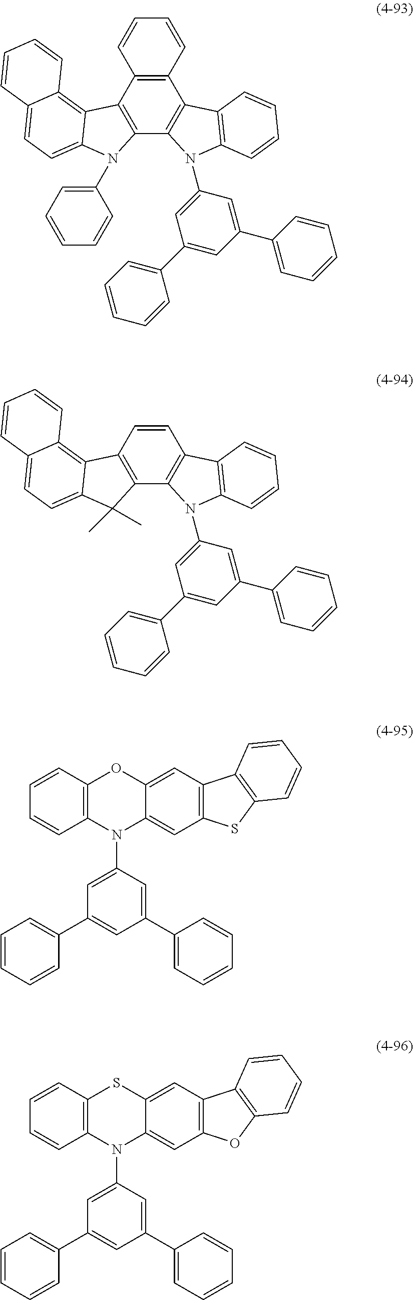

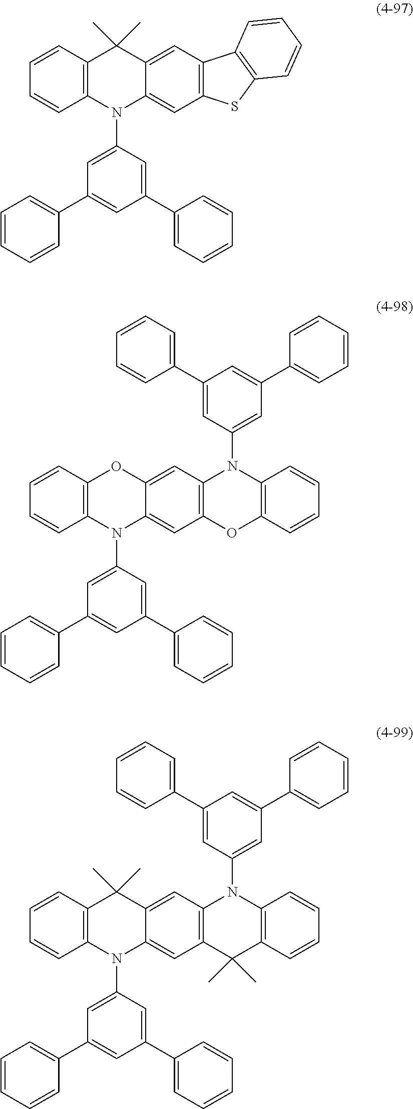

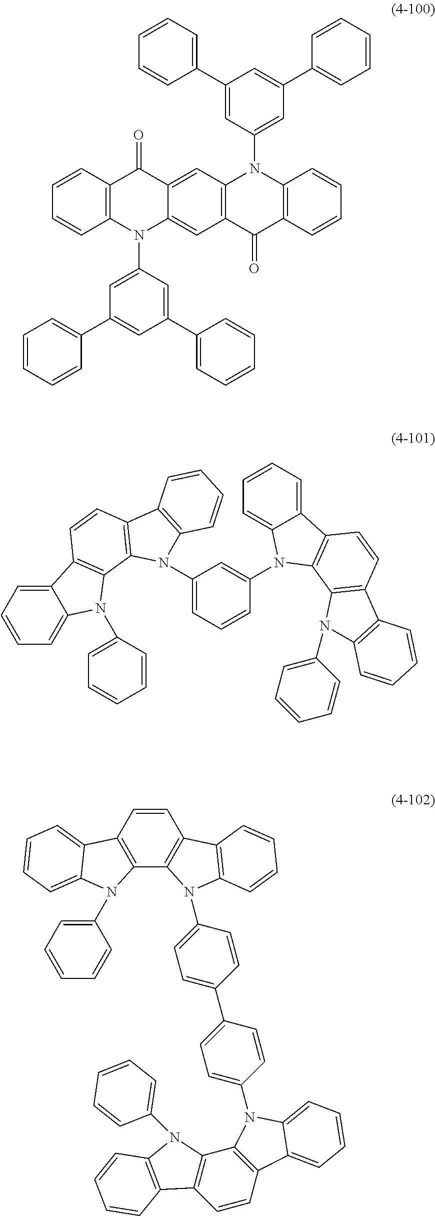

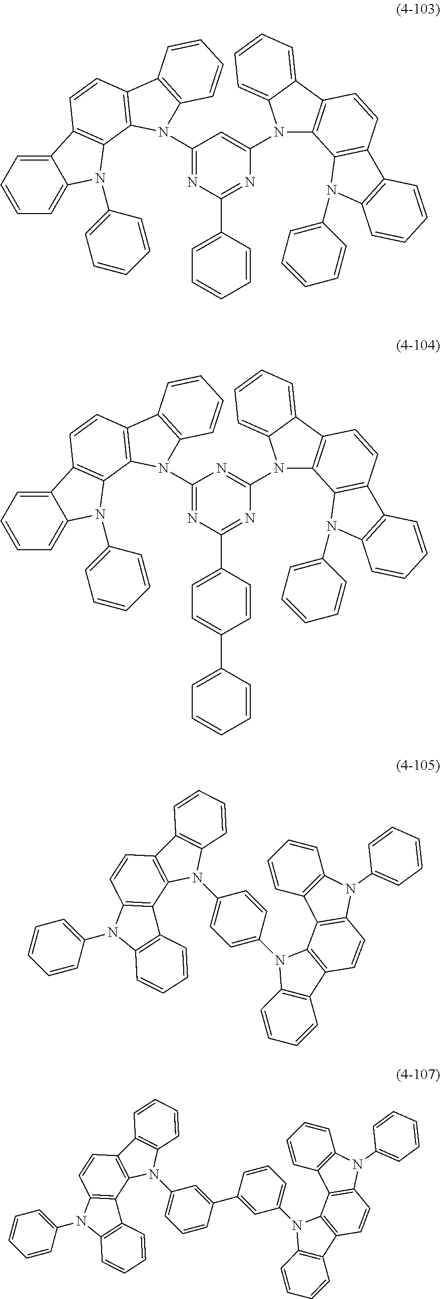

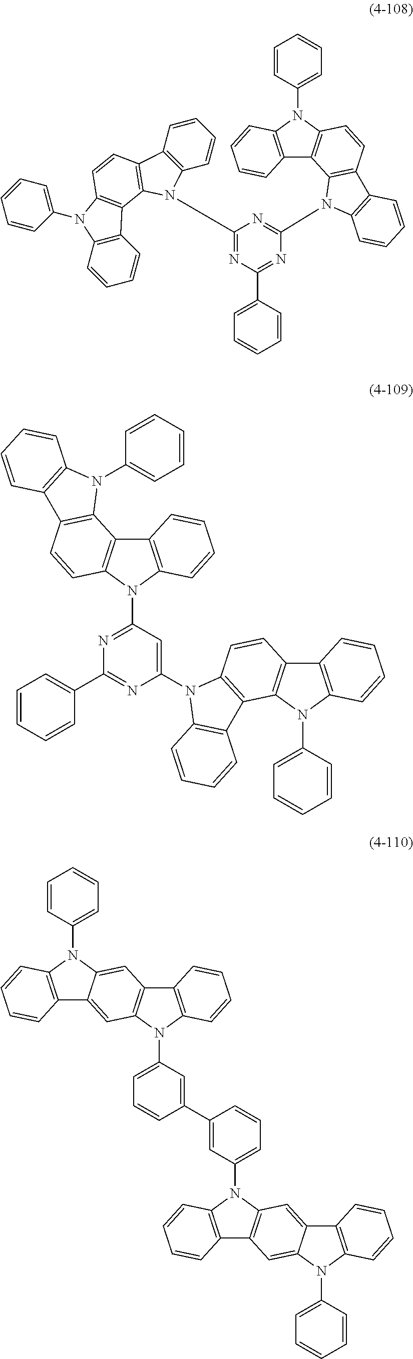

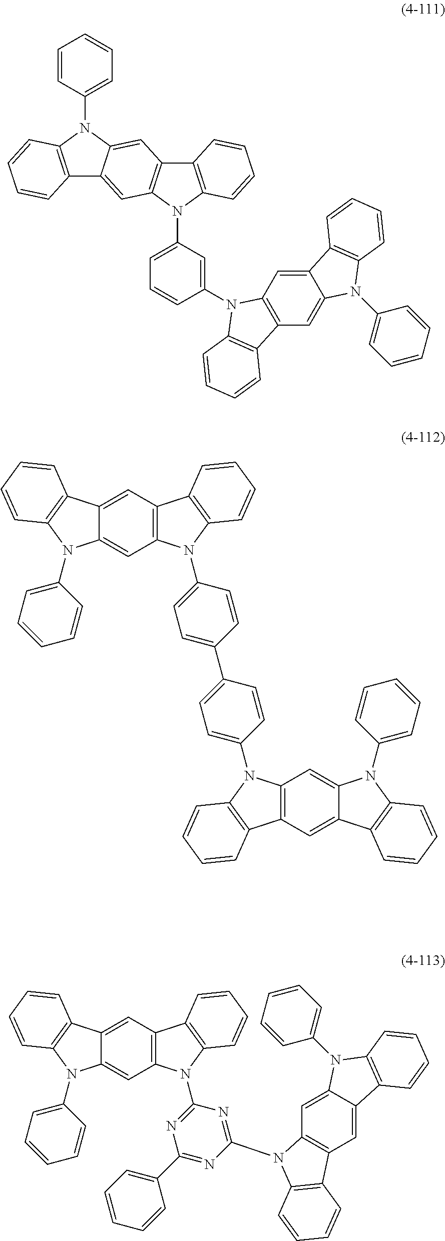

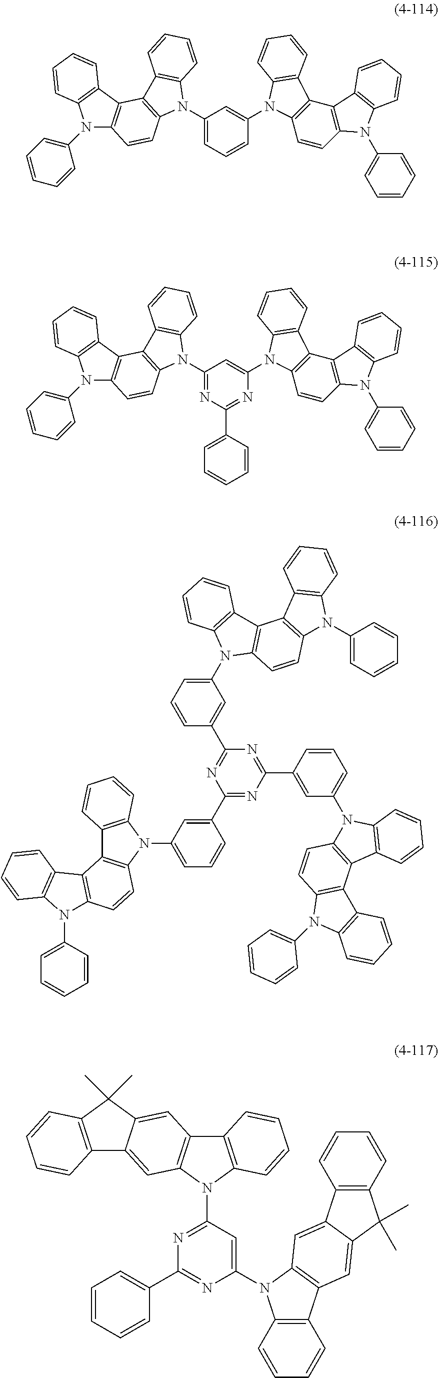

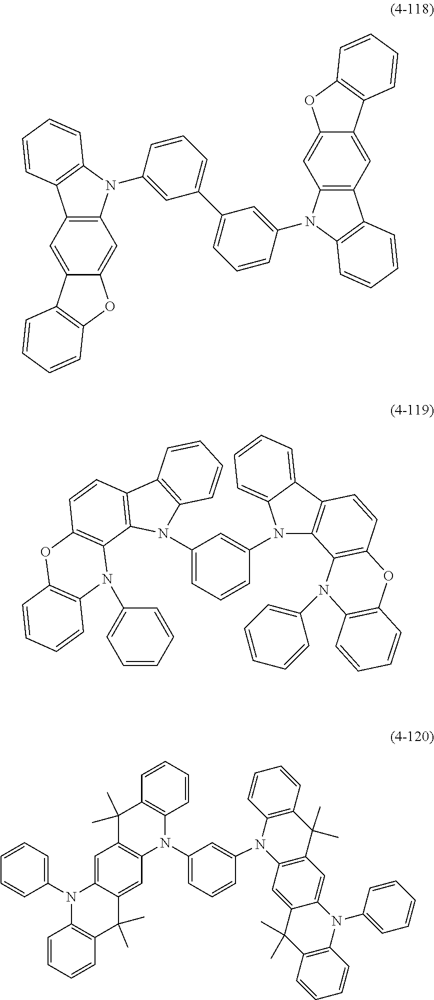

Specifically, the second organic compound satisfying the general formula (5) includes, but is not limited to, the following compounds:

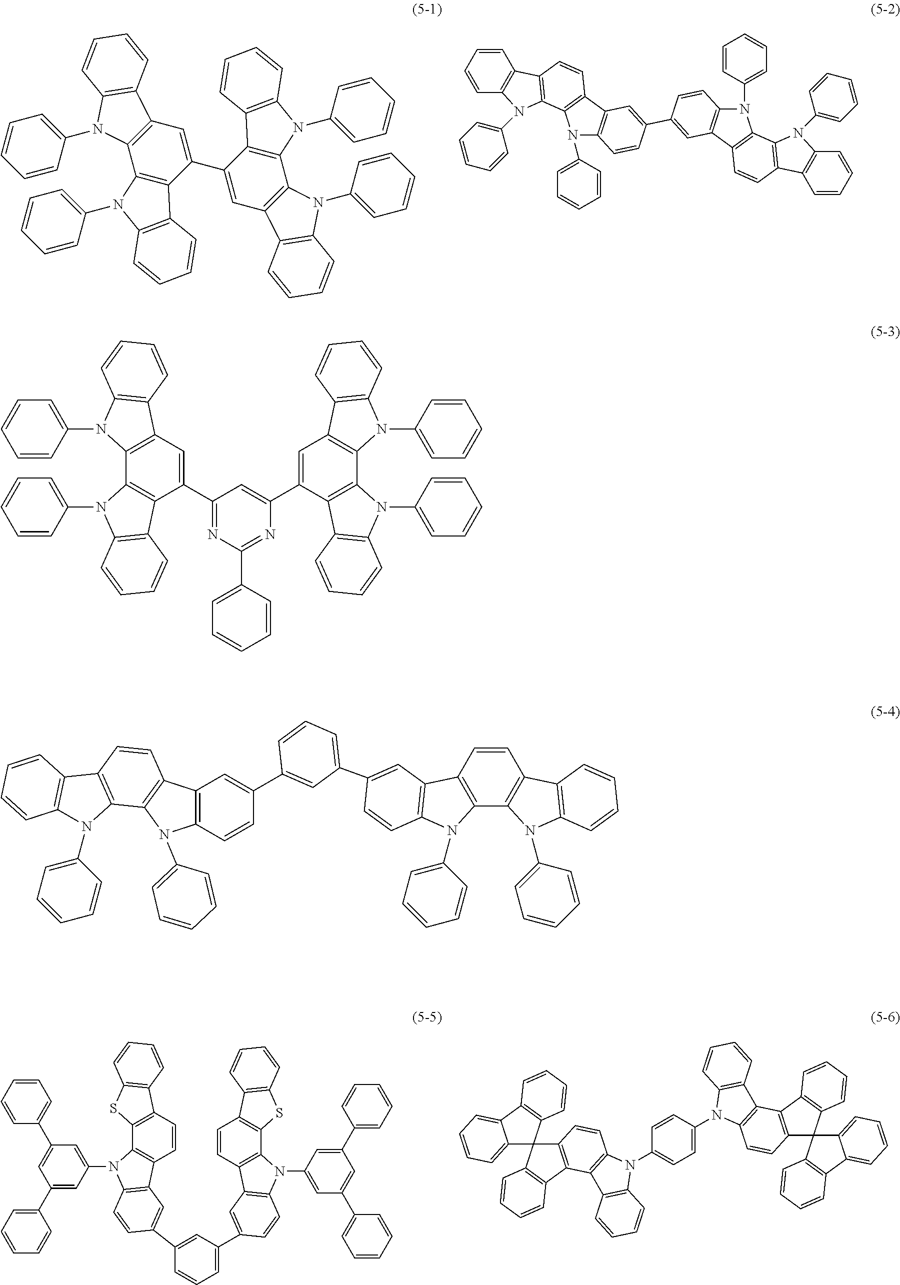

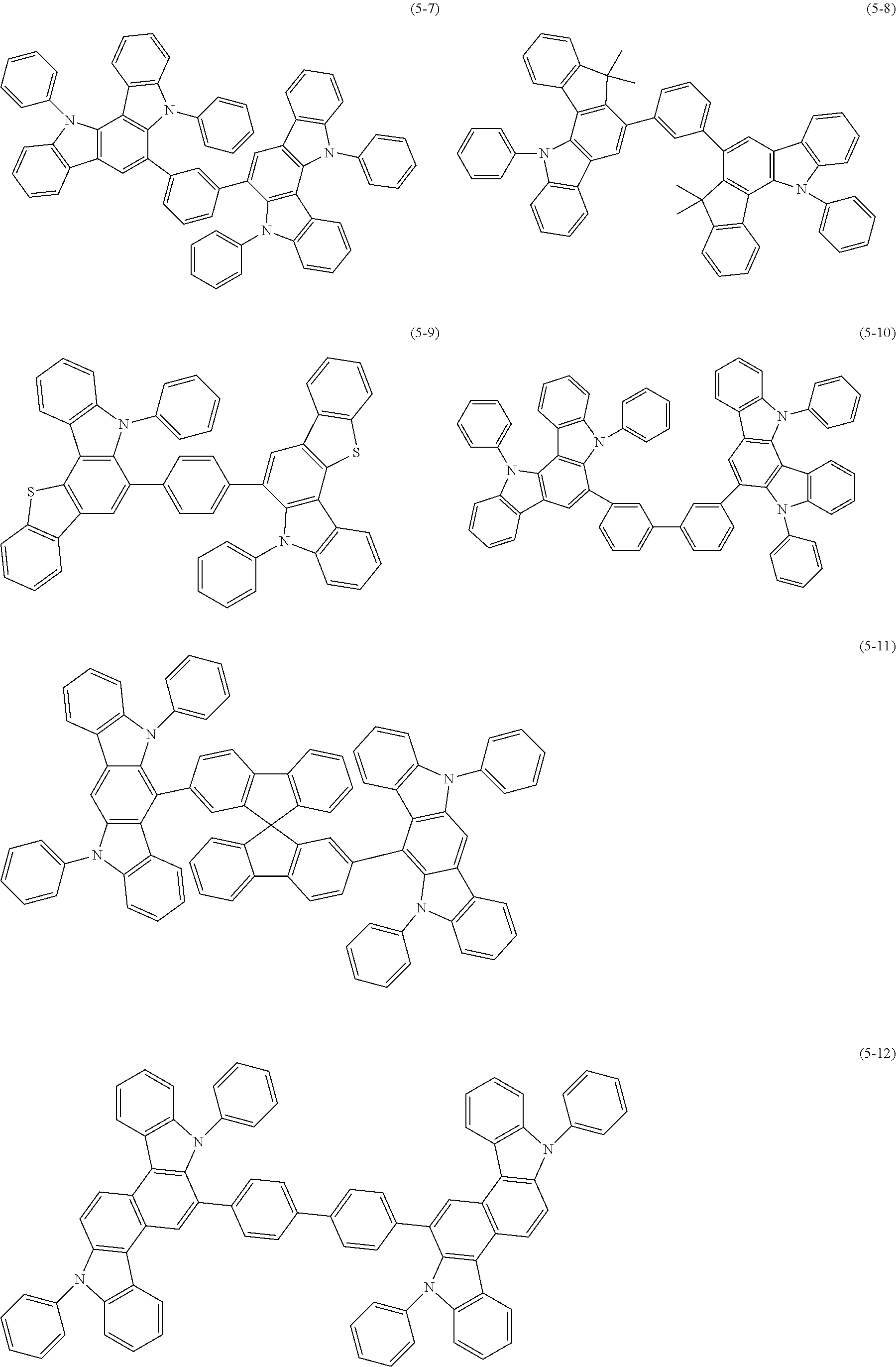

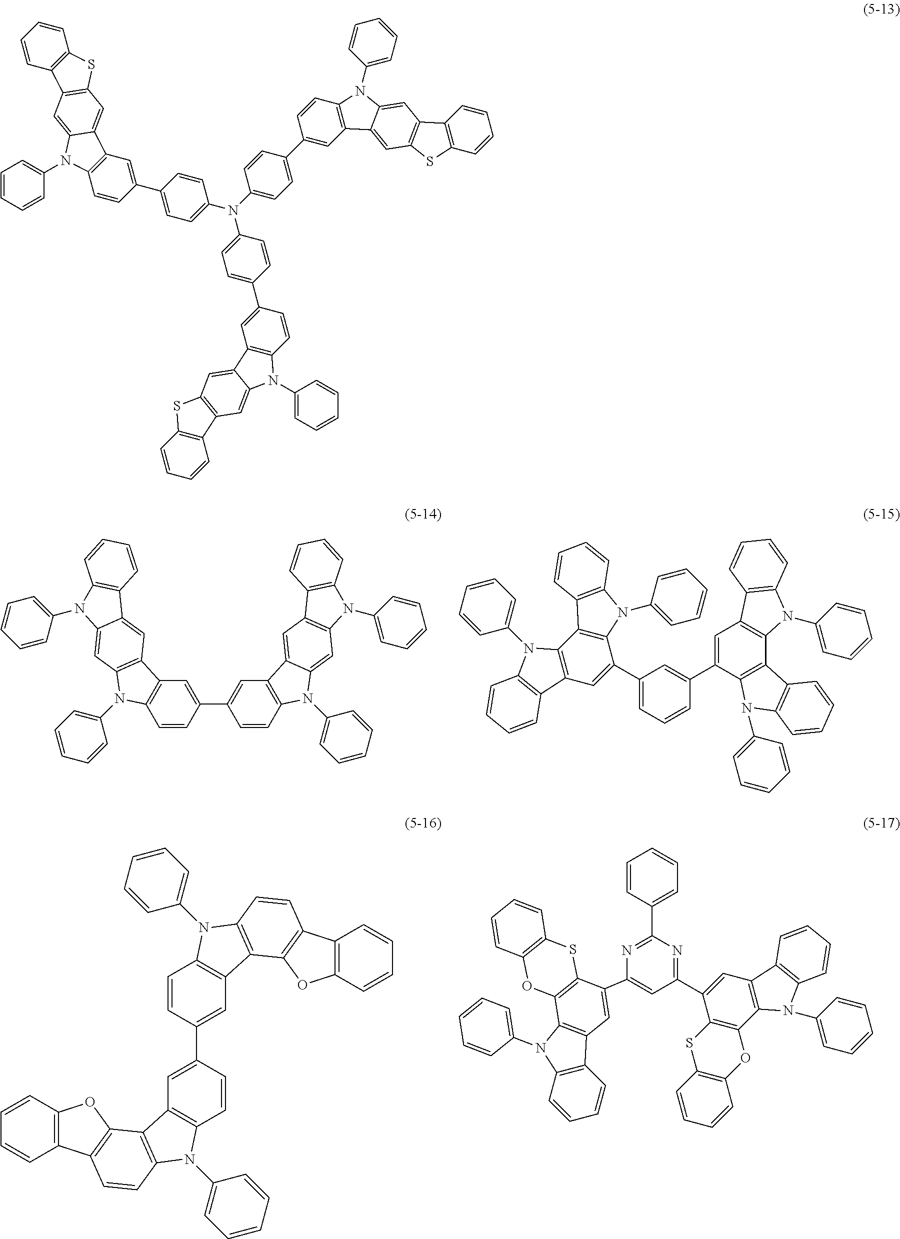

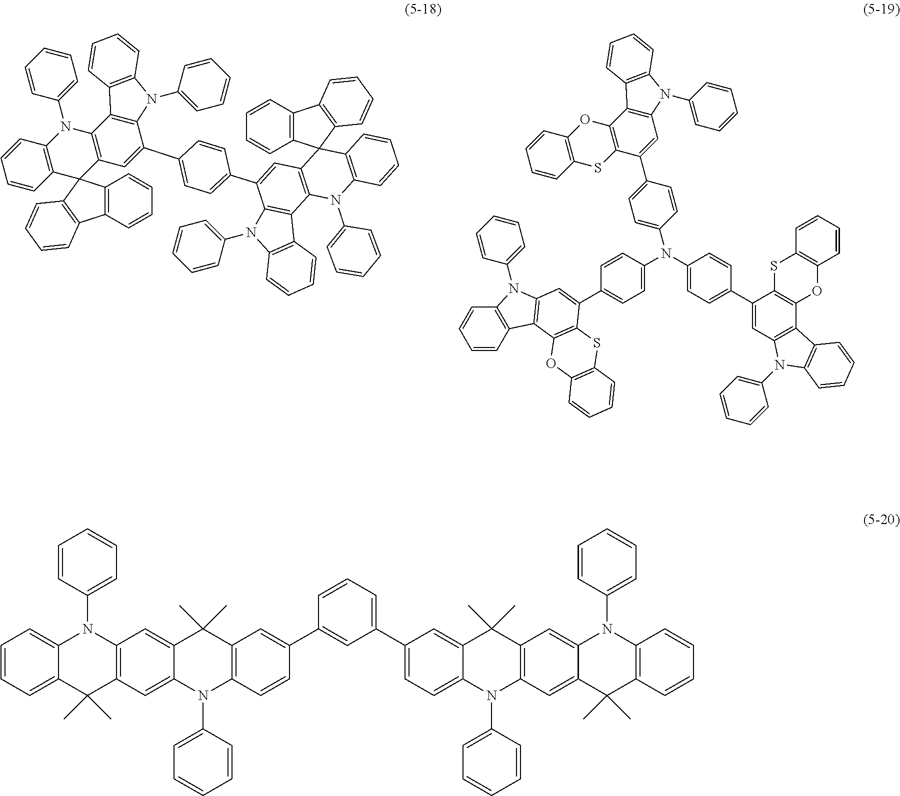

Further, the organic mixture further includes an organic functional material selected from the group consisting of a hole (also called electron hole) injection material (HIM), a hole transport material (HTM), a hole blocking material (HBM), an electron injection material (EIM), an electron transport material (ETM), an electron blocking material (EBM), an organic host material and a light-emitting material. Wherein, the light-emitting material is selected from a singlet emitter (fluorescent emitter), a triplet emitter (phosphorescent emitter) and a thermally activated delayed fluorescent material (TADF material). The organic host material referred to herein can be clearly used as an organic host material.

The organic functional material may be a small molecule material or a polymer material. Specifically, the organic functional material may be those disclosed in WO2010135519A1, US20090134784A1 and WO2011110277A1.

Herein, polymer includes homopolymer, copolymer, and block copolymer. In the present embodiment, the polymer also includes dendrimer which may be those disclosed in document [Dendrimers and Dendrons, Wiley-VCH Verlag GmbH & Co. KGaA, 2002, Ed. George R. Newkome, Charles N. Moorefield, Fritz Vogtle.], or may be synthesized by a synthesis method in the above document.

When the organic functional material is a phosphorescent emitter, the first organic compound and the second organic compound serve as a host material, and the ratio of the sum of the mass of the first organic compound and the second organic compound to the mass of the organic functional material is 100:30 or more; further, the ratio of the sum of the mass of the first organic compound and the second organic compound to the mass of the organic functional material is 100:25 or more; and still further, the ratio of the sum of the mass of the first organic compound and the second organic compound to the mass of the organic functional material is 100:20 or more.

When the organic functional material includes a phosphorescent emitter and an organic host material, the first organic compound and the second organic compound serve as an auxiliary light-emitting material, and the ratio of the sum of the weights of the first organic compound and the second organic compound to the weight of the phosphorescent emitter is 1:2 to 2:1. At this time, the energy level of the exciplex formed by the mixture is higher than that of the phosphorescent emitter.

When the organic functional material is a fluorescent emitter, the first organic compound and the second organic compound serve as a host material, and the ratio of the sum of the mass of the first organic compound and the second organic compound to the mass of the organic functional material is 100:15 or more; Further, the ratio of the sum of the mass of the first organic compound and the second organic compound to the mass of the organic functional material is 100:10 or more; and still further, the ratio of the sum of the mass of the first organic compound and the second organic compound to the mass of the organic functional material is 100:8 or more.

Alternatively, when the organic functional material is a fluorescent host material, the first organic compound and the second organic compound serve as a fluorescent emitting material, and the ratio of the sum of the mass of the first organic compound and the second organic compound to the mass of the organic functional material is 100:15 or more; Further, the ratio of the sum of the mass of the first organic compound and the second organic compound to the mass of the organic functional material is 100:10 or more; and still further, the ratio of the sum of the mass of the first organic compound and the second organic compound to the mass of the organic functional material is 100:8 or more.

When the organic functional material is a TADF material, the first organic compound and the second organic compound serve as a host material, and the ratio of the sum of the weights of the first organic compound and the second organic compound to the weight of the TADF material is 100:15 or more; further, the ratio of the sum of the weights of the first organic compound and the second organic compound to the weight of the TADF material is 100:10 or more; and still further, the ratio of the sum of the weights of the first organic compound and the second organic compound to the weight of the TADF material is 100:8 or more.

The fluorescent emitting material (singlet emitter), the phosphorescent emitting material (triplet emitter), and the TADF material are described in more detail below (but not limited thereto).

(1) Singlet Emitter

Singlet emitter tends to have a longer conjugated π-electron system. To date, there have been many examples, such as, styrylamine and derivatives thereof disclosed in JP2913116B and WO2001021729A1, and indenofluorene and derivatives thereof disclosed in WO2008/006449 and WO2007/140847.

In an embodiment, the singlet emitter is one selected from the group consisting of mono-styrylamine, di-styrylamine, tri-styrylamine, tetra-styrylamine, styrene phosphine, styrene ether and arylamine.

Wherein, a mono-styrylamine is a compound which comprises an unsubstituted or substituted styryl group and at least one amine, and amine is particularly an aromatic amine. A di-styrylamine is a compound which comprises two unsubstituted or substituted styryl group and at least one amine, and amine is particularly an aromatic amine. A tri-styrylamine is a compound which comprises three unsubstituted or substituted styryl group and at least one amine, and amine is particularly an aromatic amine. A tetra-styrylamine is a compound which comprises four unsubstituted or substituted styryl group and at least one amine, and amine is particularly an aromatic amine. Wherein, styrene herein is stilbene, which may be further substituted.

The definitions of styrene phosphine and styrene ether are similar to those of the above amines and will not be described herein.

An aryl amine (aromatic amine) refers to a compound which comprises three unsubstituted or substituted aromatic cyclic or heterocyclic systems directly attached to nitrogen. At least one of these aromatic or heterocyclic ring systems is a fused ring system and particularly has a carbon atom number of no less than 14.

Specifically, the aryl amine is one selected from the group consisting of aromatic anthramine, aromatic anthradiamine, aromatic pyrene amine, aromatic pyrene diamine, aromatic chrysene amine and aromatic chrysene diamine. Aromatic anthracene amine refers to a compound in which a diarylamino group is directly attached to anthracene; further, the diarylamino group is attached to anthracene at position 9. Aromatic anthradiamine refers to a compound in which two diarylamino group is directly attached to anthracene; further, the two diarylamino group is attached to anthracene at positions 9 and 10, respectively. Wherein, the definitions of aromatic pyrene amine, aromatic pyrene diamine, aromatic chrysene amine and aromatic chrysene diamine are similar to that of aromatic anthracene amine. Wherein, the diarylarylamino groups of aromatic pyrene amine and aromatic pyrene diamine are especially attached to pyrene at position 1, alternatively, the diarylarylamino groups of aromatic pyrene amine and aromatic pyrene diamine are attached to pyrene at positions 1 and 6, respectively.

Wherein, singlet emitters based on styrylamine and arylamine may be those disclosed in WO2006/000388, WO2006/058737, WO2006/000389, WO2007/065549, WO2007/115610, U.S. Pat. No. 7,250,532B2, DE102005058557A1, CN1583691A, JP08053397A, U.S. Pat. No. 6,251,531 B1, US2006/210830 A, EP1957606 A1 and US2008/0113101 A1.

Singlet emitters based on styrylamine and derivatives thereof may be those disclosed in U.S. Pat. No. 5,121,029.

Further, the singlet emitters may be one selected from indenofluorene-amine and indenofluorene-diamine, such as, benzoindenofluorene-amine or benzoindenofluorene-diamine disclosed in WO 2006/122630, dibenzoindenofluorene-amine or dibenzoindenofluorene-diamine disclosed in WO 2008/006449, and indenofluorene-amine or indenofluorene-diamine disclosed in WO2007/140847.

Wherein, the singlet emitters may be polycyclic aromatic hydrocarbon compounds, such as the following compounds and derivatives thereof: anthracenes such as 9,10-di(2-naphthylanthracene), naphthalene, tetraphenyl, oxyanthene, phenanthrene, perylene (e.g., 2,5,8,11-tetra-t-butylperylene), indenoperylene, phenylene (e.g., 4,4′-(bis (9-ethyl-3-carbazovinylene)-1,1′-biphenyl), diindenoperylene, decacyclene, coronene, fluorene, spirobifluorene, arylperylene (e.g., those disclosed in US20060222886), arylenevinylene (e.g., those disclosed in U.S. Pat. Nos. 5,121,029 and 5,130,603), cyclopentadiene such as tetraphenylcyclopentadiene, rubrene, coumarine, rhodamine, quinacridone, pyrane such as 4 (dicyanoethylene)-6-(4-p-dimethylaminostyryl-2-methyl)-4H-pyrane (DCM), thiapyran, bis (azinyl) imine-boron compounds (those disclosed in US 2007/0092753 A1), bis (azinyl) methene compounds, carbostyryl compounds, oxazone, benzoxazole, benzothiazole, benzimidazole and diketopyrrolopyrrole. The singlet emitters may be those disclosed in US20070252517 A1, U.S. Pat. Nos. 4,769,292, 6,020,078, US2007/0252517 A1 and US2007/0252517 A1.

In the present embodiment, the singlet emitters include, but are not limited to, the following compounds:

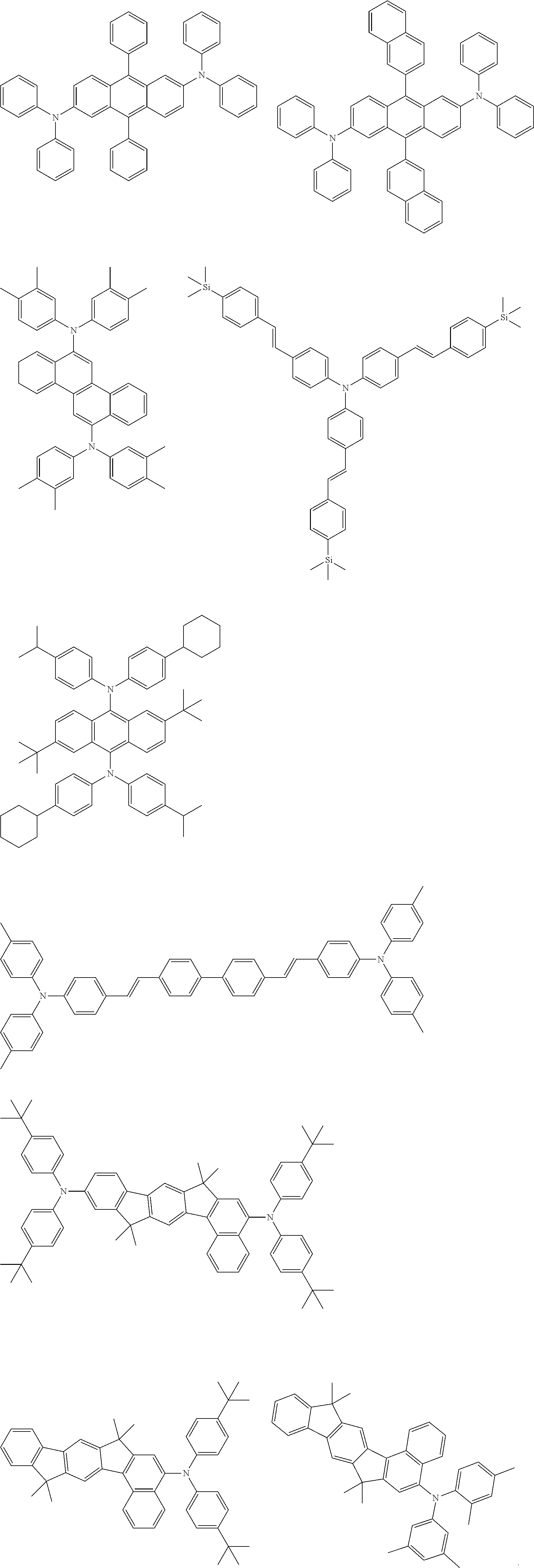

(2) Thermally Activated Delayed Fluorescent Materials (TADF):

Traditional organic fluorescent materials can only emit light using 25% singlet excitons formed by electrical excitation, and the devices have relatively low internal quantum efficiency (up to 25%). Since the intersystem crossing is enhanced due to the strong spin-orbital coupling of the heavy atom center, the phosphorescent material can emit light using the singlet and triplet excitons formed by the electric excitation effectively, to achieve 100% internal quantum efficiency of the device. However, the application of phosphorescent material in OLEDs is limited by the problems such as high cost, poor material stability and serious roll-off of the device efficiency, etc. Thermally-activated delayed fluorescent materials are the third generation of organic light-emitting materials developed after organic fluorescent materials and organic phosphorescent materials. This type of material generally has a small singlet-triplet excited state energy level difference (ΔEₛₜ), and triplet excitons can be converted to singlet excitons by anti-intersystem crossing to emit light. Thus, singlet excitons and triplet excitons formed under electric excitation can be fully utilized and the device can achieve 100% internal quantum efficiency.

TADF materials need to have a smaller singlet-triplet excited state energy level difference, typically ΔEₛₜ<0.3 eV, further ΔEₛₜ<0.2 eV, further ΔEₛₜ<0.1 eV, and further ΔEₛₜ<0.05 eV. In addition, TADF materials have a good fluorescence quantum efficiency. The TADF materials may be those disclosed in the following documents: CN103483332(A), TW201309696(A), TW201309778(A), TW201343874(A), TW201350558(A), US20120217869(A1), WO2013133359(A1), WO2013154064(A1), Adachi, et. al. Adv. Mater., 21, 2009, 4802, Adachi, et. al. Appl. Phys. Lett., 98, 2011, 083302, Adachi, et. al. Appl. Phys. Lett., 101, 2012, 093306, Adachi, et. al. Chem. Commun., 48, 2012, 11392, Adachi, et. al. Nat. Photon., 6, 2012, 253, Adachi, et. al. Nature, 492, 2012, 234, Adachi, et. al. J. Am. Chem. Soc, 134, 2012, 14706, Adachi, et. al. Angew. Chem. Int. Ed, 51, 2012, 11311, Adachi, et. al. Chem. Commun., 48, 2012, 9580, Adachi, et. al. Chem. Commun., 48, 2013, 10385, Adachi, et. al. Adv. Mater., 25, 2013, 3319, Adachi, et. al. Adv. Mater., 25, 2013, 3707, Adachi, et. al. Chem. Mater., 25, 2013, 3038, Adachi, et. al. Chem. Mater., 25, 2013, 3766, Adachi, et. al. J. Mater. Chem. C., 1, 2013, 4599, Adachi, et. al. J. Phys. Chem. A., 117, 2013, 5607.

In the present embodiment, the TADF light-emitting materials include, but are not limited to, the following compounds:

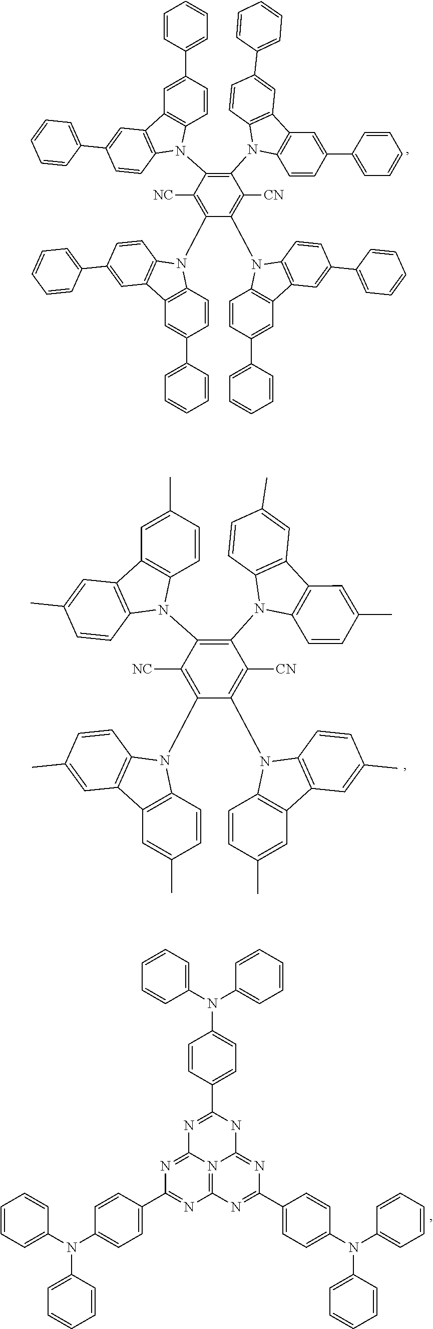

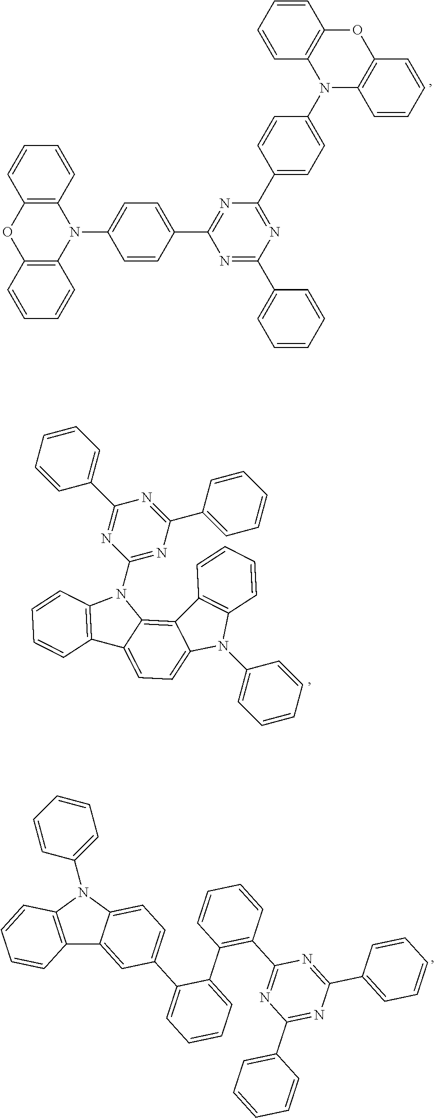 [SMILES: c1ccc(-c2nc(-c3ccc(N4c5ccccc5Oc5ccccc54)cc3)nc(-c3ccc(N4c5ccccc5Oc5ccccc54)cc3)n2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-c3ccccc3-c3ccccc3-c3ccc4c(c3)c3ccccc3n4-c3ccccc3)n2)cc1]

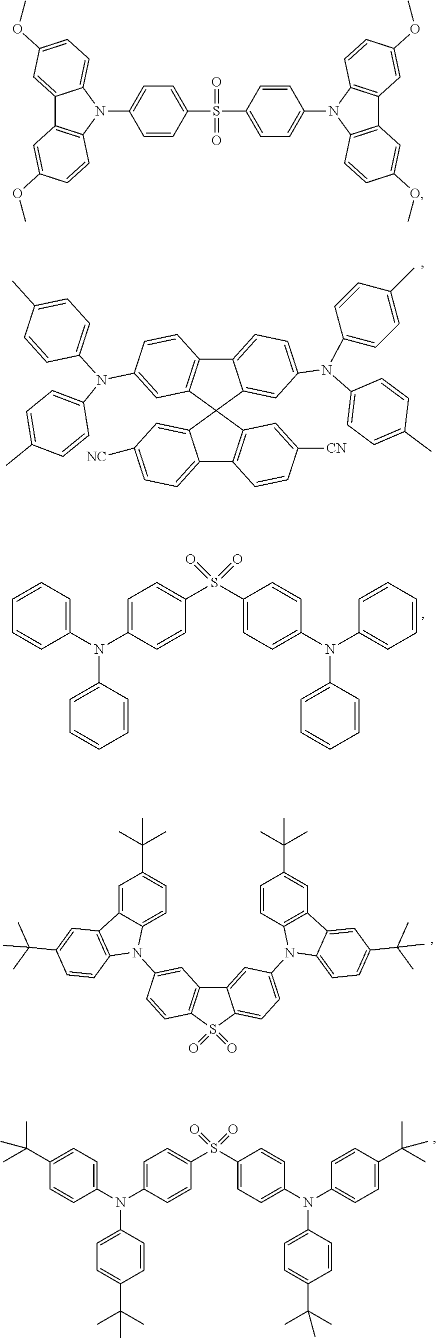

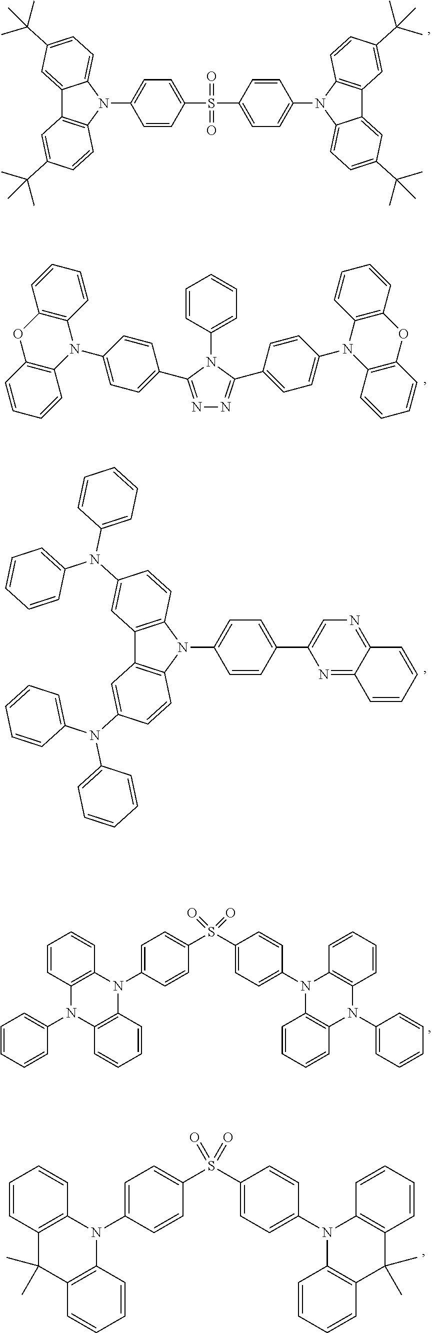

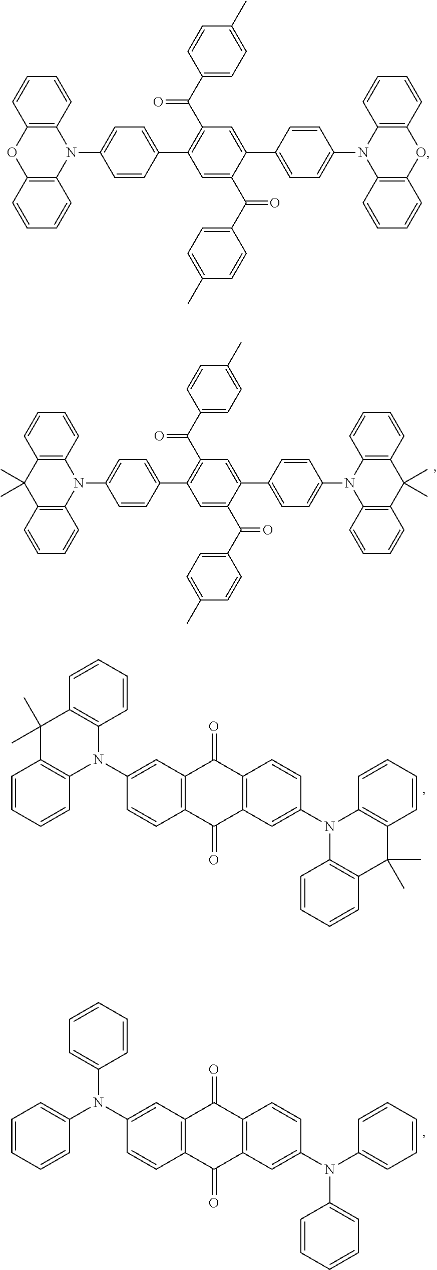

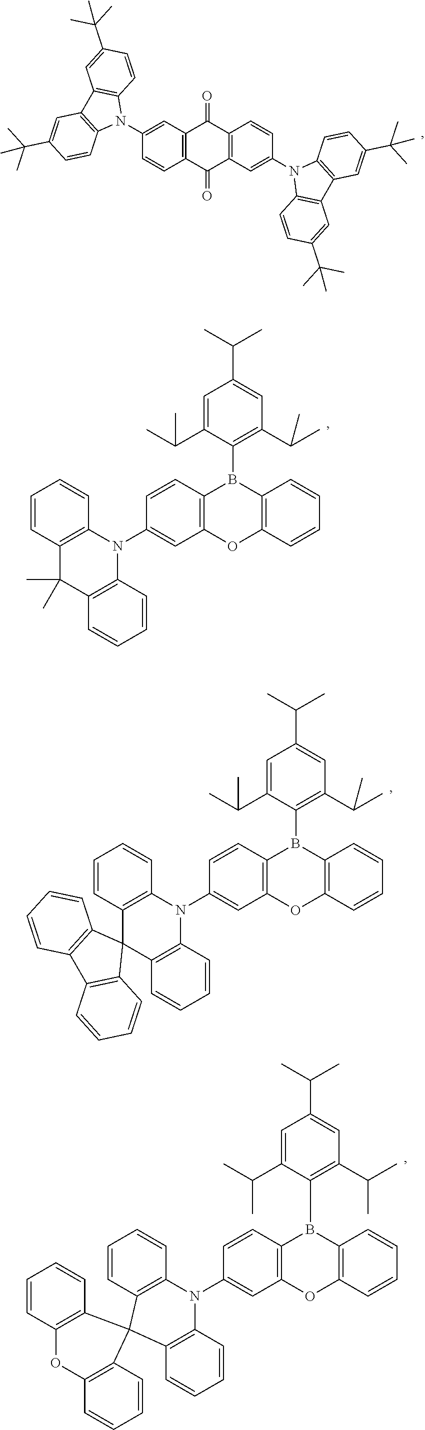

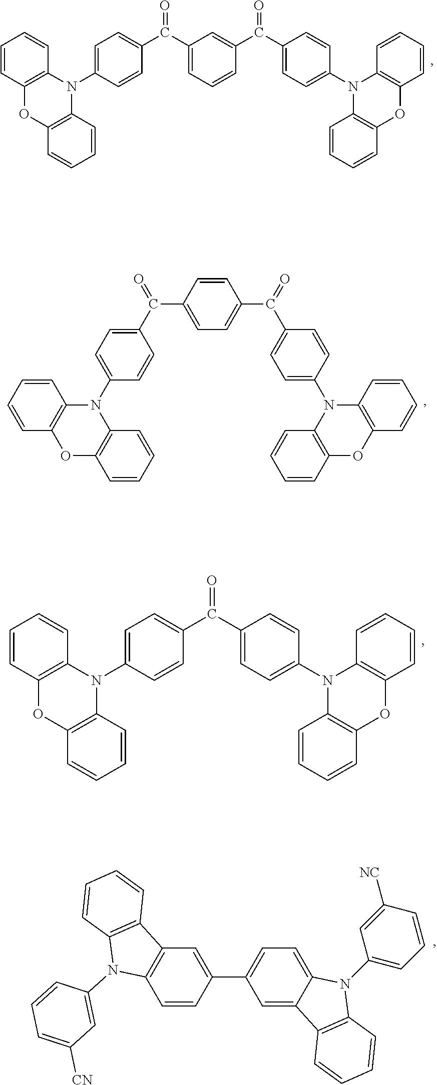 [SMILES: O=C(c1ccc(N2c3ccccc3Oc3ccccc32)cc1)c1cccc(C(=O)c2ccc(N3c4ccccc4Oc4ccccc43)cc2)c1 ; O=C(c1ccc(C(=O)c2ccc(N3c4ccccc4Oc4ccccc43)cc2)cc1)c1ccc(N2c3ccccc3Oc3ccccc32)cc1 ; O=C(c1ccc(N2c3ccccc3Oc3ccccc32)cc1)c1ccc(N2c3ccccc3Oc3ccccc32)cc1 ; N#Cc1cccc(-n2c3ccccc3c3cc(-c4ccc5c(c4)c4ccccc4n5-c4cccc(C#N)c4)ccc32)c1]

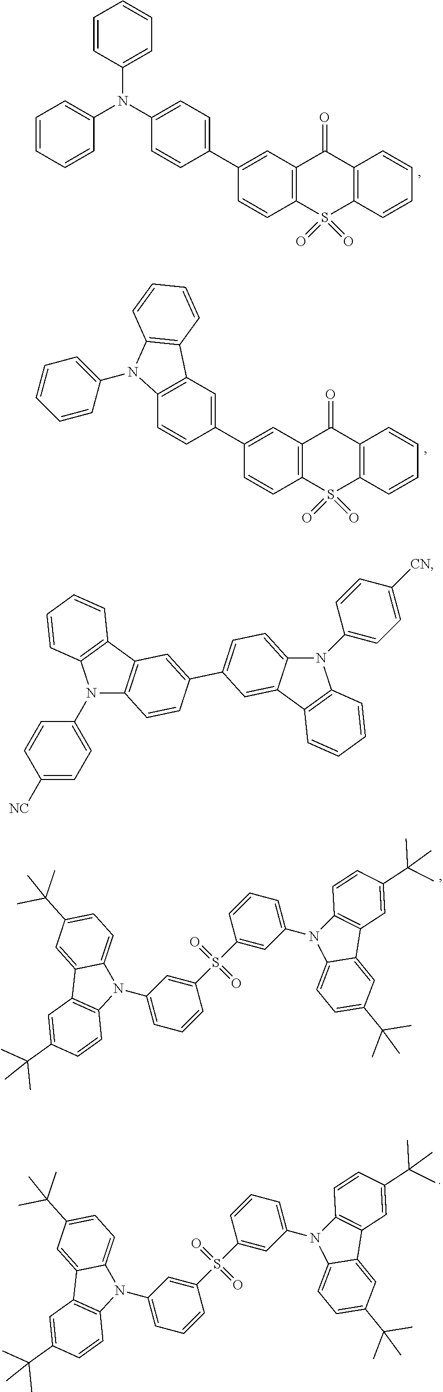

(3) Triplet Emitter

Triplet emitters are also called phosphorescent emitters. Specifically, the triplet emitter is a metal complex with general formula M(L)ₙ. Wherein, M is a metal atom, and each occurrence of L may be the same or different and is an organic ligand which is bonded or coordinated to the metal atom M through one or more positions; n is an integer greater than 1, and further n is 1, 2, 3, 4, 5 or 6. Further, the metal complex is attached to a polymer through one or more positions; and further, the metal complex is attached to a polymer through organic ligands.

Further, the metal atom M is one selected from a transitional metal element, a lanthanide element and an actinide element; furthermore, the metal atom M is one selected from Ir, Pt, Pd, Au, Rh, Ru, Os, Sm, Eu, Gd, Tb, Dy, Re, Cu and Ag; and still further, the metal atom M is one selected from Os, Ir, Ru, Rh, Re, Pd and Pt.

Further, L is a chelating ligand (i.e., a ligand) that coordinates with the metal via at least two binding sites; and still further, L has two bidentate ligands, three bidentate ligands, two multidentate ligands, or three multidentate ligands. Wherein, the bidentate ligands may be identical or different, and the multidentate ligands may be identical or different. The chelating ligands are helpful to improve the stability of the metal complexes.

Specifically, the organic ligand may be one selected from the group consisting of derivatives of phenylpyridine, derivatives of 7,8-benzoquinoline, derivatives of 2-(2-thienyl)pyridine, derivatives of 2-(1-naphthyl)pyridine and derivatives of 2-phenylquinoline. Furthermore, the organic ligands may be substituted, for example, by fluoromethyl or trifluoromethyl. Auxiliary ligands may be especially selected from acetylacetone or picric acid.

Further, the metal complex of the triplet emitter has the following structural formula:

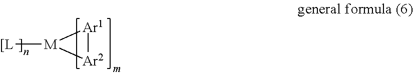

in the general formula (6), M is described above; Ar₁ is a cyclic group, which may be the same or different at each occurrence, and each Ar¹ contains at least one donor atom (i.e., an atom containing a lone pair of electrons, such as nitrogen or phosphorus) through which the cyclic group is coordinately coupled with metal; Ar₂ is a cyclic group, which may be the same or different at each occurrence, and Ar₂ contains at least one C atom through which the cyclic group is coupled with metal; Ar₁ and Ar₂ are covalently bonded together, and each of them may carry one or more substituent groups, and they may be coupled together by the substituent groups again; L may be the same or different at each occurrence, and L is an auxiliary ligand, particularly a bidentate chelating ligand, further a monoanionic bidentate chelating ligand; m is selected from 1, 2 or 3; n is selected from 0, 1 or 2, further, n is 0 or 1, and still further, n is 0.

Specifically, the triplet emitters may be those disclosed in following patents: WO 200070655, WO 200141512, WO 200202714, WO 200215645, EP 1191613, EP 1191612, EP 1191614, WO 2005033244, WO 2005019373, US 2005/0258742, WO 2009146770, WO 2010015307, WO 2010031485, WO 2010054731, WO 2010054728, WO 2010086089, WO 2010099852, WO 2010102709, US 20070087219 A1, US 20090061681 A1, US 20010053462 A1, US 2007/0252517 A1, U.S. Pat. Nos. 6,824,895, 7,029,766, 6,835,469, U.S. Pat. No. 6,830,828, US 20010053462 A1, WO 2007095118 A1, US 2012004407A1, WO 2012007088A1, WO2012007087A1, WO 2012007086A1, US 2008027220A1, WO 2011157339A1, CN 102282150A and WO 2009118087A1, and may be those disclosed in following documents: Baldo, Thompson et al. Nature 403, (2000), 750-753, Adachi et al. Appl. Phys. Lett. 78(2001), 1622-1624, J. Kido et al. Appl. Phys. Lett. 65(1994), 2124, Kido et al. Chem. Lett. 657, 1990, Johnson et al., JACS 105, 1983, 1795, Wrighton, JACS 96, 1974, 998 and Ma et al., Synth. Metals 94, 1998, 245.

In the present embodiment, the triplet emitters include, but are not limited to, the following compounds:

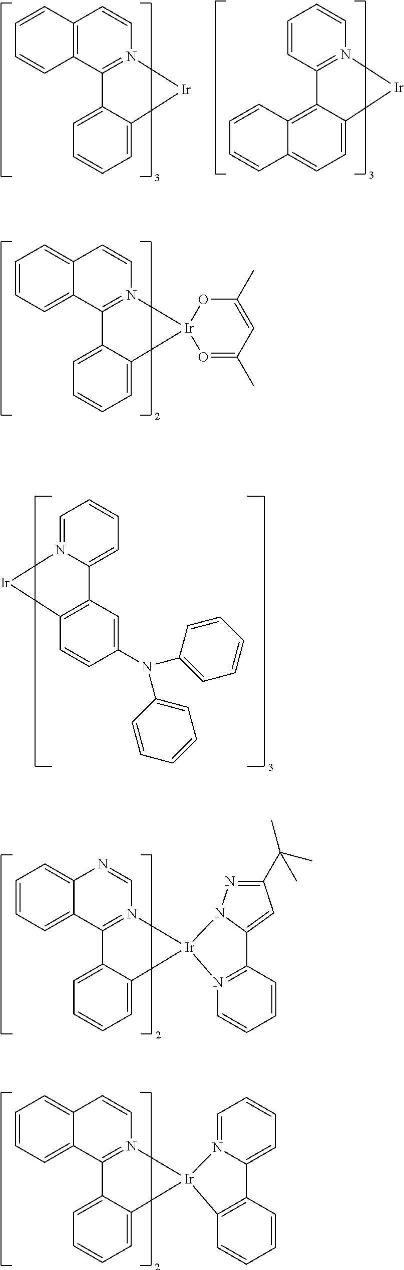

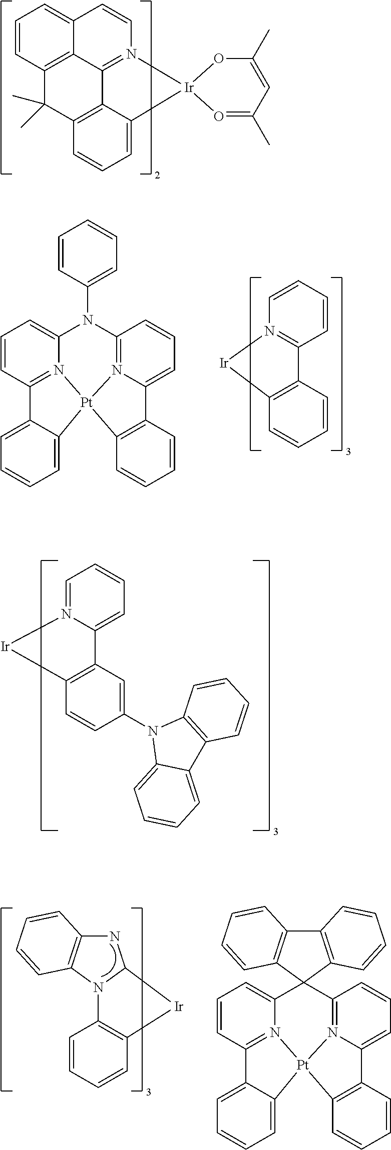

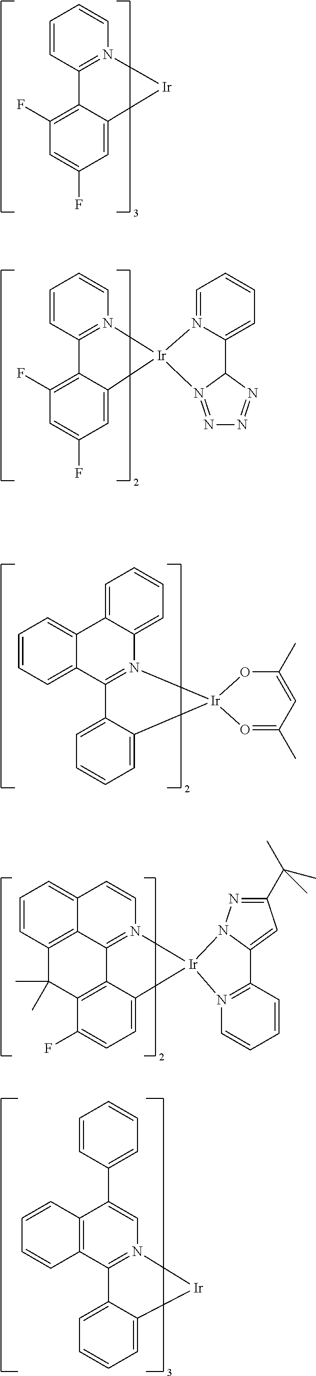

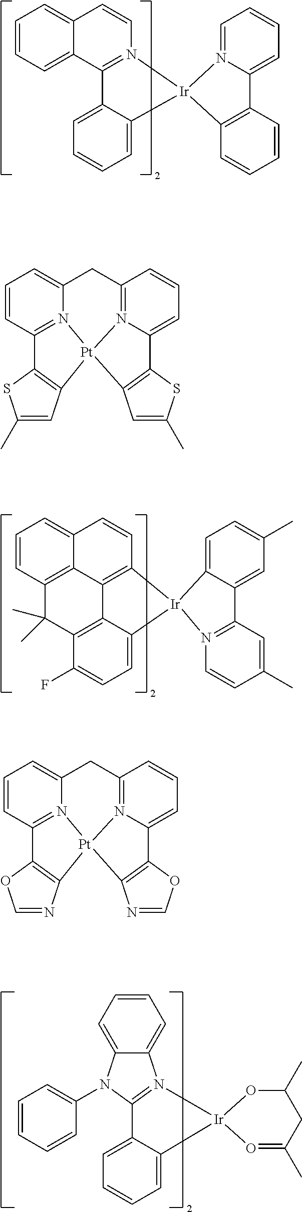

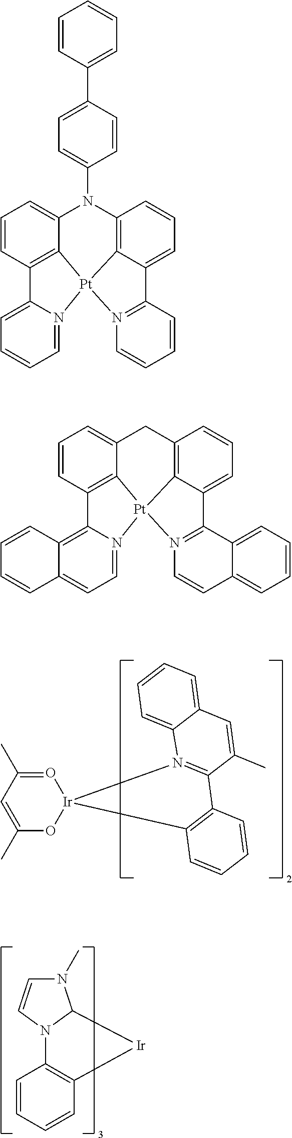

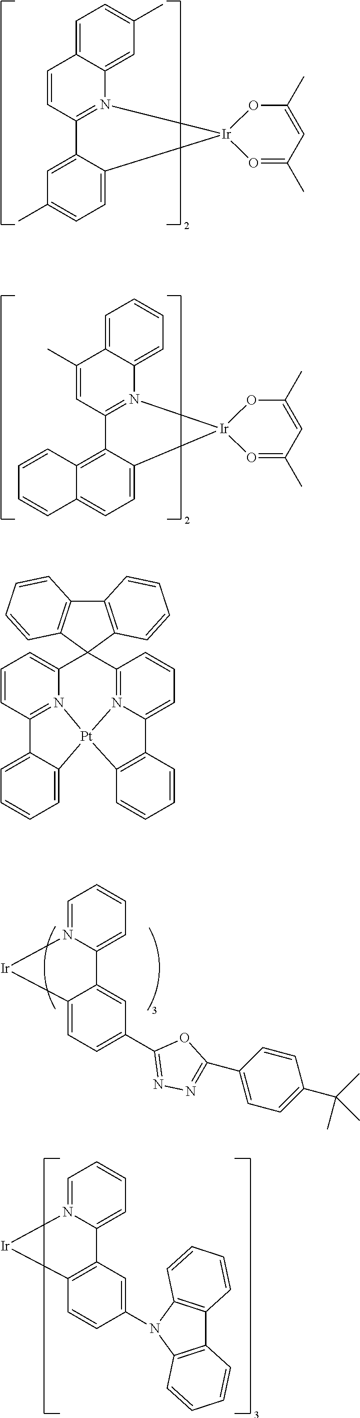

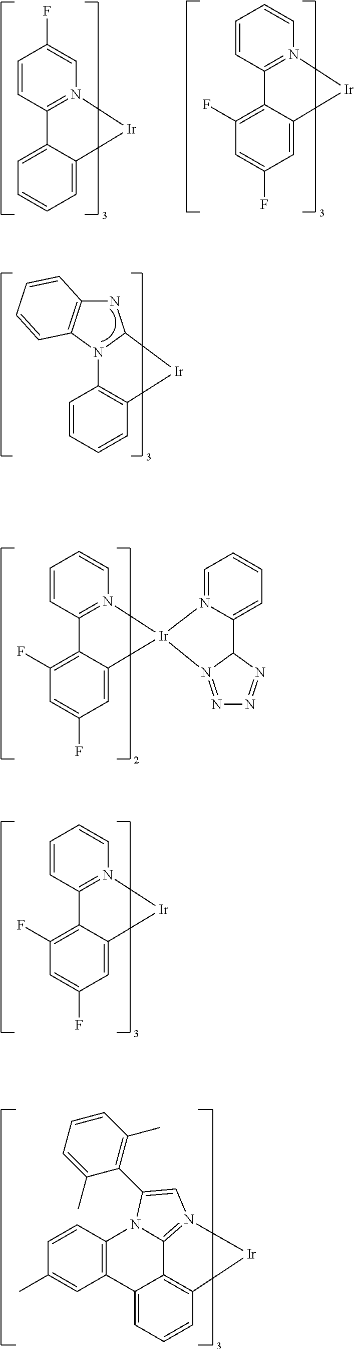

The above organic mixture has at least the following advantages: since the first organic compound of the above organic mixture is an aromatic compound containing a triphenylboron heterocycle, and the second organic compound is a compound containing an aromatic fused heterocycle, and min((LUMO_{H1}−HOMO_{H2}, LUMO_{H2}−HOMO_{H1})≤min(E_{T}(H1), E_{T}(H2))+0.1 eV, when the organic mixture is applied as a co-host material to an organic electronic device, higher emitting efficiency and lifetime of the device can be achieved. The possible reasons are as follows, but are not limited thereto: both of the aromatic compound containing a triphenylboron heterocycle and the compound containing an aromatic fused heterocycle have suitable HOMO and LUMO energy levels, which facilitate the injection and transmission of electron and hole; and since the intermediate state of exciplex with suitable energy level is formed between the two host materials, energy transfer can be more fully achieved, thereby the efficiency and lifetime of the device can be effectively improved.

The formulation of an embodiment can be used as a coating ink and can be applied to prepare an organic electronic device. The organic electronic device may be one selected from the group consisting of an organic light-emitting diode (OLED), an organic photovoltaic cell (OPV), an organic light-emitting electrochemical cell (OLEEC), an organic field effect transistor (OFET), an organic light-emitting field effect transistor, an organic laser, an organic spintronic device, an organic sensor and an organic plasmon emitting diode. Specifically, the formulation can be used to prepare the light-emitting layer of the OLED. The formulation may be a solution or a suspension. Wherein, the formulation comprises an organic mixture and an organic solvent.

The organic mixture is substantially the same as the organic mixture described above, except that in the organic mixture of the present embodiment, at least one of the first organic compound and the second organic compound has a molar mass of no less than 700 g/mmol; further, at least one of the first organic compound and the second organic compound has a molar mass of no less than 800 g/mmol; further, at least one of the first organic compound and the second organic compound has a molar mass of no less than 900 g/mmol; further, at least one of the first organic compound and the second organic compound has a molar mass of no less than 1000 g/mmol; and still further, at least one of the first organic compound and the second organic compound has a molar mass of no less than 1100 g/mmol.

Further, in the present embodiment, the solubility of the organic mixture in toluene at 25° C. is 10 mg/ml, further the solubility of the organic mixture in toluene at 25° C. is 15 mg/ml, and still further the solubility of the organic mixture in toluene at 25° C. is 20 mg/ml.

Since the formulation of the present embodiment is used as a printing ink, the viscosity and surface tension of the formulation are important parameters. Only formulations with appropriate parameters can be suitable for specific substrates and specific printing methods.

Specifically, the surface tension of the formulation of the present embodiment at working temperature or at 25° C. is in the range of about 19 dyne/cm to 50 dyne/cm, further in the range of 22 dyne/cm to 35 dyne/cm, and still further in the range of 25 dyne/cm to 33 dyne/cm.

Specifically, the viscosity of the formulation of the present embodiment at working temperature or at 25° C. is in the range of about 1 cps to 100 cps, further in the range of 1 cps to 50 cps, furthermore in the range of 1.5 cps to 20 cps, and still further in the range of 4.0 cps to 20 cps. In this case, the formulation is more suitable for inkjet printing.

Wherein, the viscosity can be adjusted by different methods, such as by selecting proper solvent and concentration of the organic mixture in the formulation. The formulation comprising the metal organic complex or polymer according to the present disclosure can facilitate to adjust the viscosity of the formulation in an appropriate range and to be printed according to the printing method used.

Specifically, the weight percentage of the organic functional material in the formulation of the present embodiment is in the range of 0.3% to 30%, further in the range of 0.5% to 20%, further in the range of 0.5% to 1%; further in the range of 0.5% to 10%; and further in the range of 1% to 5%.

Specifically, the solvent comprises a first organic solvent. Wherein, the first solvent is at least one selected from an aromatic solvent, a heteroaromatic solvent, a ketone solvent, and an ether solvent.

Further, the aromatic solvent is at least one selected from an aliphatic chain substituted aromatic compound and a cyclic aliphatic substituted aromatic compound.

Specifically, the aromatic solvent and the heteroaromatic solvent are at least one selected from the group consisting of p-diisopropylbenzene, pentylbenzene, tetrahydronaphthalene, cyclohexyl benzene, chloronaphthalene, 1,4-dimethylnaphthalene, 3-isopropylbiphenyl, p-cymene, dipentylbenzene, tripentylbenzene, pentyltoluene, o-xylene, m-xylene, p-xylene, o-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, dihexylbenzene, dibutylbenzene, p-diisopropylbenzene, 1-methoxynaphthalene, cyclohexylbenzene, dimethylnaphthalene, 3-isopropylbiphenyl, p-cymene, 1-methylnaphthalene, 1,2,4-trichlorobenzene, 1,3-dipropoxybenzene, 4,4-difluorodiphenylmethane, 1,2-dimethoxy-4-(1-propenyl)benzene, diphenylmethane, 2-phenylpyridine, 3-phenylpyridine, N-methyldiphenylamine 4-isopropylbiphenyl, α,α-dichlorodiphenylmethane, 4-(3-phenylpropyl)pyridine, benzylbenzoate, 1,1-di(3,4-dimethylphenyl)ethane, 2-isopropylnaphthalene and dibenzylether.

Specifically, the ketone solvent is at least one selected from the group consisting of 1-tetralone, 2-tetralone, 2-(phenylepoxy)tetralone, 6-(methoxyl)tetralone, acetophenone, phenylacetone, benzophenone, derivatives of 1-tetralone, derivatives of 2-tetralone, derivatives of 2-(phenylepoxy)tetralone, derivatives of 6-(methoxyl)tetralone, derivatives of acetophenone, derivatives of phenylacetone, and derivatives of benzophenone. Wherein, derivatives of 1-tetralone, derivatives of 2-tetralone, derivatives of 2-(phenylepoxy)tetralone, derivatives of 6-(methoxyl)tetralone, derivatives of acetophenone, derivatives of phenylacetone, and derivatives of benzophenone may be 4-methylacetophenone, 3-methylacetophenone, 2-methylacetophenone, 4-methylphenylacetone, 3-methylphenylacetone, 2-methylphenylacetone, isophorone, 2,6,8-trimethyl-4-nonanone, fenchone, 2-nonanone, 3-nonanone, 5-nonanone, 2-decanone, 2,5-hexanedione, phorone, 6-undecanone, and the like.

Specifically, the ether solvent is at least one selected from the group consisting of 3-phenoxytoluene, butoxybenzene, benzylbutylbenzene, p-anisaldehyde dimethyl acetal, tetrahydro-2-phenoxy-2H-pyran, 1,2-dimethoxy-4-(1-propenyl)benzene, 1,4-benzodioxane, 1,3-dipropylbenzene, 2,5-dimethoxytoluene, 4-ethylphenetole, 1,2,4-trimethoxybenzene, 4-(1-propenyl)-1,2-dimethoxybenzene, 1,3-dimethoxybenzene, glycidyl phenyl ether, dibenzyl ether, 4-tert-butylanisole, trans-p-propenylanisole, 1,2-dimethoxybenzene, 1-methoxynaphthalene, diphenyl ether, 2-phenoxymethyl ether, 2-phenoxytetrahydrofuran, ethyl-2-naphthyl ether, pentyl ether, hexyl ether, dicaprylyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethyl ether butyl methyl ether, tripropylene glycol dimethyl ether, and tetraethylene glycol dimethyl ether.

Specifically, the ester solvent is at least one selected from the group consisting of alkyl octoate, alkyl sebacate, alkyl stearate, alkyl benzoate, alkyl phenylacetate, alkyl cinnamate, alkyl oxalate, alkyl maleate, alkyl lactone and alkyl oleate.

Further, the first solvent is at least one selected from an aliphatic ketone and an aliphatic ether. Specifically, the aliphatic ketone is at least one selected from the group consisting of 2-nonanone, 3-nonanone, 5-nonanone, 2-decanone, 2,5-hexanedione, 2,6,8-trimethyl-4-nonanone, phorone and 6-undecanone. The aliphatic ether is at least one selected from the group consisting of amyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether and tetraethylene glycol dimethyl ether.

Further, the organic solvent comprises a second solvent at least one selected from the group consisting of methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 4-dioxane, acetone, methyl ethyl ketone, 1,2-dichloroethane, 3-phenoxy toluene, 1,1,1-trichloroethane, 1,1,2,2-tetrachloroethane, ethyl acetate, butyl acetate, dimethylformamide, dimethylacetamide, dimethyl sulfoxide, tetrahydronaphthalene, decalin and indene.

Further, the weight percentage of the organic mixture in the formulation is 0.01 to 20 wt %, further 0.1 to 15 wt %, further 0.2 to 10 wt %, and still further 0.25 to 5 wt %.

The formulation of the present embodiment can be used to prepare an organic electronic device by a printing or coating method.

Wherein, the printing method may be inkjet printing or nozzle printing. The coating method may be typography, screen printing, dip coating, spin coating, gravure, blade coating, roller printing, twist roller printing, lithography, flexography, rotary printing, spray coating, brush coating or pad printing, slot die coating, etc. Further, the coating method is gravure printing, and the printing method is nozzle printing or inkjet printing.

Further, the formulation may further include at least one of a surfactant compound, a lubricant, a wetting agent, a dispersant, a hydrophobic agent and a binder, to adjust the viscosity and film forming property of the formulation and to improve the adhesion property. The printing technology, solvent, concentration and viscosity of the formulation may be adjusted according to Handbook of Print Media: Technologies and Production Methods, Helmut Kipphan, ISBN 3-540-67326-1.

The organic electronic device of an embodiment is an organic light-emitting diode and comprises a substrate, an anode, a functional layer and a cathode.

Wherein, the functional layer comprises a light-emitting layer, and the material of the light-emitting layer comprises the above organic mixture. In this case, the organic functional material in the organic mixture is a light-emitting material, i.e., the above-described fluorescent emitter, phosphorescent emitter, TADF material or light-emitting quantum dot.

Specifically, the first organic compound and the second organic compound in the organic mixture of the light-emitting layer may be evaporated as two sources separately; alternatively, the organic mixture may be directly evaporated as a source.

Further, the functional layer further comprises at least one of a hole injection layer (HIL), a hole transport layer (HTL), an electron blocking layer (EBL), an electron injection layer (EIL), an electron transport layer (ETL), and a hole blocking layer (HBL). The material of such functional layers may be the organic mixture described above. In this case, the organic functional material in the organic mixture is a material having corresponding function as described above, for example, the organic functional material in the organic mixture of the hole transport layer is the hole transport material described above. Alternatively, the materials of the above functional layers may be those disclosed in WO2010135519A1, US20090134784A1 and WO2011110277A1.

The substrate can be opaque or transparent. A transparent substrate can be used to fabricate a transparent light-emitting device. For example, the transparent substrate may be that disclosed in Bulovic et al. Nature 1996, 380, p 29 and Gu et al. Appl. Phys. Lett. 68, 68, p 2606. The substrate may be a rigid substrate or an elastic substrate.

Specifically, the substrate may be plastic, metal, semiconductor wafer or glass. Further, the substrate has a smooth surface. The substrate with no surface defects is an ideal option.

Further, the substrate is flexible. The substrate is a polymer thin film or a plastic; the substrate has a glass transition temperature T_{g} of 150° C. or more, further larger than 200° C., further larger than 250° C., and further larger than 300° C. Specifically, the substrate is one selected from poly(ethylene terephthalate) (PET) and polyethylene(2,6-naphthalate) (PEN).

The anode material comprises one of a conductive metal, a metallic oxide and a conductive polymer. The anode can inject holes easily into the light-emitting layer, hole injection layer (HIL), or the hole transport layer (HTL).

Specifically, the absolute value of the difference between the work function of the anode and the HOMO energy level or the valence band energy level of the organic functional material (light-emitting material) in the light-emitting layer, p-type semiconductor material in the hole injection layer, p-type semiconductor material in the hole transport layer or p-type semiconductor material in the electron blocking layer is less than 0.5 eV, further less than 0.3 eV, and still further less than 0.2 eV.

Specifically, the anode material is one selected from the group consisting of Al, Cu, Au, Ag, Mg, Fe, Co, Ni, Mn, Pd, Pt, ITO and aluminum-doped zinc oxide (AZO). The anode material may be prepared by physical vapor deposition. Wherein, the physical vapor deposition specifically refers to radio frequency magnetron sputtering, vacuum thermal evaporation, electron beam (e-beam) evaporation, and the like.

It should be noted that the anode material is not limited to the above materials and may also be patterned ITO.

The cathode material is one selected from a conductive metal and a metal oxide. The cathode material can inject electrons easily into the electron injection layer (EIL), the electron transport layer (ETL), or the light-emitting layer.

Further, the absolute value of the difference between the work function of the cathode and the HUMO energy level or the valence band energy level of the organic functional material (light-emitting material) in the light-emitting layer, n-type semiconductor material in the electron injection layer, n-type semiconductor material in the electron transport layer or n-type semiconductor material in the hole blocking layer is less than 0.5 eV, further less than 0.3 eV, and still further less than 0.2 eV. In principle, all materials capable of using as the cathode of the OLED may be used as the cathode material of the organic electronic device according to the present embodiment.

Further, the cathode material is one selected from the group consisting of Al, Au, Ag, Ca, Ba, Mg, LiF/Al, Mg/Ag alloy, BaF₂/Al, Cu, Fe, Co, Ni, Mn, Pd, Pt and ITO. The cathode material may be prepared by physical vapor deposition. Wherein, the physical vapor deposition specifically refers to radio frequency magnetron sputtering, vacuum thermal evaporation, electron beam (e-beam) evaporation, and the like.

Wherein, the emission wavelength of the organic electronic device according to the present embodiment is between 300 and 1000 nm, further between 350 and 900 nm, and further between 400 and 800 nm.

The above organic electronic device can be applied in various electronic equipments, such as display equipments, lighting equipments, light sources or sensors.

The following are examples.

### Example 1

The preparation process of the first organic compound (1-4) of the present embodiment is as follows:

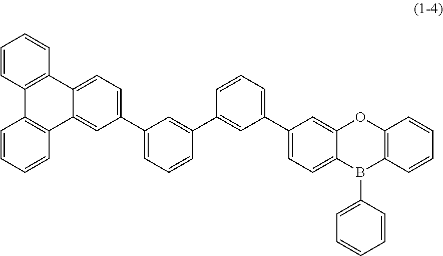 [SMILES: c1ccc(B2c3ccccc3Oc3cc(-c4cccc(-c5cccc(-c6ccc7c8ccccc8c8ccccc8c7c6)c5)c4)ccc32)cc1]

(1) Compound 1-4-1 (16.3 g, 60 mmol) and Compound 1-4-2 (18.7 g, 60 mmol), tetrakis(triphenylphosphine)palladium (3.45 g, 3 mmol), tetrabutylammonium bromide (2.6 g, 8 mmol), sodium hydroxide (3.2 g, 80 mmol), water (20 mL) and toluene (150 mL) were added to a 250 mL three-necked flask under nitrogen atmosphere, and the solution was heated to 80° C. and reacted under stirring for 12 hours at this temperature, and then the reaction was ended. The reaction solution was rotary evaporated to remove most of the solvent, and then dissolved with dichloromethane and washed with water for 3 times. The organic solution was collected, mixed with silica gel, and then purified by column chromatography to obtain compound 1-4-3, with a yield of 70%.

Synthetic route of the compound 1-4-3 is as follows:

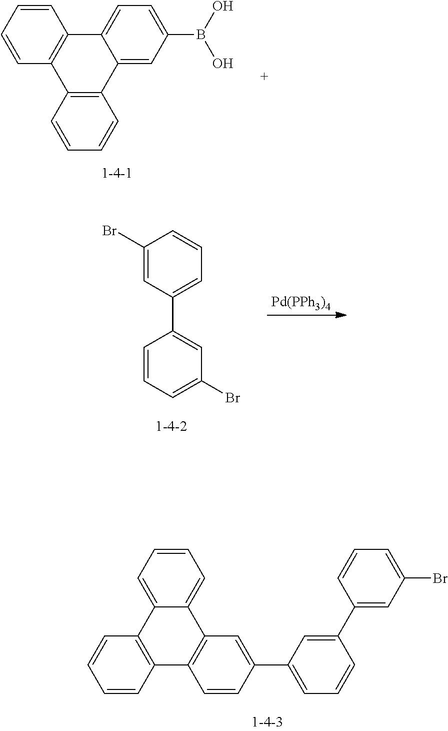 [SMILES: OB(O)c1ccc2c3ccccc3c3ccccc3c2c1 ; Brc1cccc(-c2cccc(Br)c2)c1 ; Brc1cccc(-c2cccc(-c3ccc4c5ccccc5c5ccccc5c4c3)c2)c1 ; "Pd(PPh3)4"]

(2) Compound 1-4-3 (13.8 g, 30 mmol) and 150 mL of anhydrous tetrahydrofuran were added to a 300 mL three-necked flask under nitrogen atmosphere, cooled to −78° C., and 35 mmol of n-butyllithium was slowly added dropwise, the solution was reacted for 2 hours. Then 40 mmol of isopropoxyboronic acid pinacol ester was added one time to allow the reaction temperature to rise to room temperature naturally. The reaction was further performed for 12 hours and then quenched by addition of pure water. The reaction solution was rotary evaporated to remove most of the solvent, and then extracted with dichloromethane and washed with water for 3 times. The organic phase was collected, spin dried, and then recrystallized to obtain compound 1-4-4, with a yield of 90%.

Synthetic route of the compound 1-4-4 is as follows:

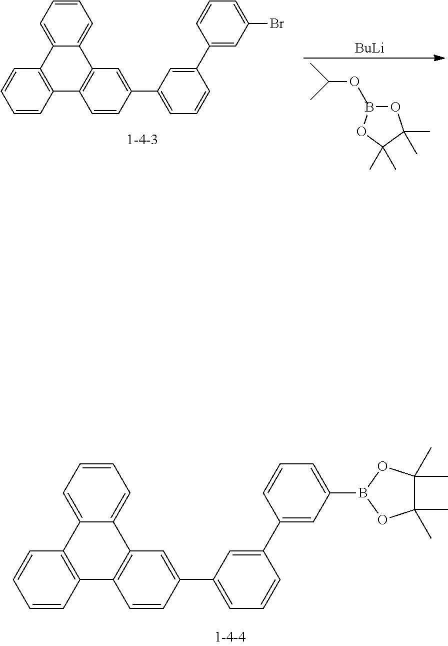

(3) Compound 1-4-5 (12.7 g, 50 mmol), Compound 1-4-6 (8.7 g, 50 mmol), potassium carbonate (100 mmol) and triglyme (80 mL) were added to a 150 mL three-necked flask under nitrogen atmosphere, and the solution was heated to 135° C. and reacted under stirring for 12 hours at this temperature, and then the reaction was ended when completed. The reaction solution was poured into 300 mL of water, and filter residue was filtered with suction, then recrystallized with the mixture solvent of ethanol and dichloromethane to obtain compound 1-4-7, with a yield of 95%.

Synthetic route of the compound 1-4-7 is as follows:

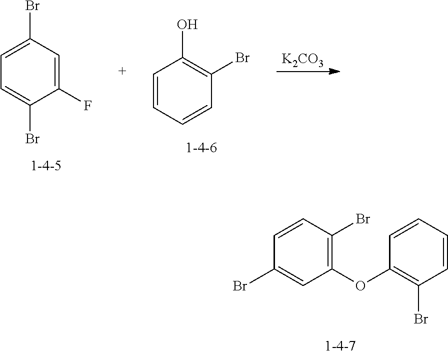

(4) Compound 1-4-7 (12.2 g, 30 mmol) and 150 mL of anhydrous tetrahydrofuran were added to a 300 mL three-necked flask under nitrogen atmosphere, cooled to 0° C., and 60 mmol of n-butyllithium was slowly added dropwise, the mixture was reacted for 1 hours. Then 30 mmol of anhydrous tetrahydrofuran solution of methyl 4-boronobenzoate was added one time to allow the reaction temperature to rise to room temperature naturally. The reaction was further performed for 12 hours and then quenched by addition of pure water. The reaction solution was rotary evaporated to remove most of the solvent, and then extracted with dichloromethane and washed with water for 3 times. The organic phase was collected, spin dried, and then recrystallized to obtain compound 1-4-8, with a yield of 80%.

Synthetic route of the compound 1-4-8 is as follows:

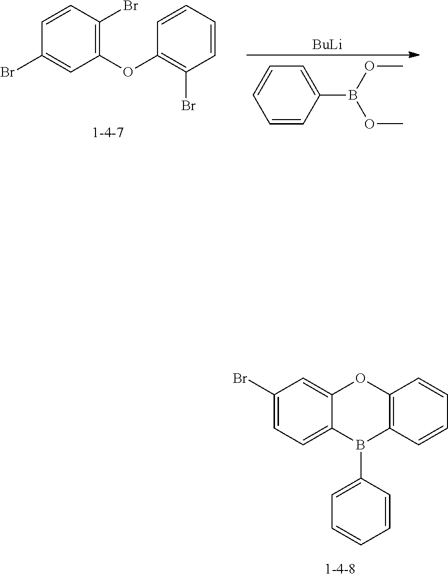

(5) Compound 1-4-4 (10.1 g, 20 mmol) and Compound 1-4-8 (6.7 g, 20 mmol), tetrakis(triphenylphosphine)palladium (1.15 g, 1 mmol), tetrabutylammonium bromide (1.3 g, 4 mmol), sodium hydroxide (1.6 g, 40 mmol), water (10 mL) and toluene (60 mL) were added to a 150 mL three-necked flask under nitrogen atmosphere, and the solution was heated to 80° C. and reacted under stirring for 12 hours, and then the reaction was ended. The reaction solution was rotary evaporated to remove most of the solvent, and then dissolved with dichloromethane and washed with water for 3 times. The organic solution was collected, mixed with silica gel, and then purified by column chromatography to obtain compound 1-4, with a yield of 85%.

Synthetic route of the compound 1-4 is as follows:

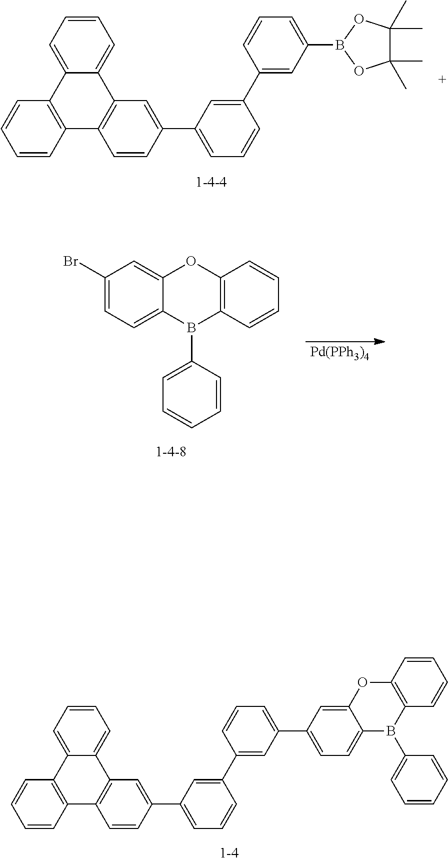 [SMILES: CC1(C)OB(c2cccc(-c3cccc(-c4ccc5c6ccccc6c6ccccc6c5c4)c3)c2)OC1(C)C ; Brc1ccc2c(c1)Oc1ccccc1B2c1ccccc1 ; c1ccc(B2c3ccccc3Oc3cc(-c4cccc(-c5cccc(-c6ccc7c8ccccc8c8ccccc8c7c6)c5)c4)ccc32)cc1 ; "Pd(PPh3)4"]

### Example 2

The preparation process of the first organic compound (1-23) of the present embodiment is as follows:

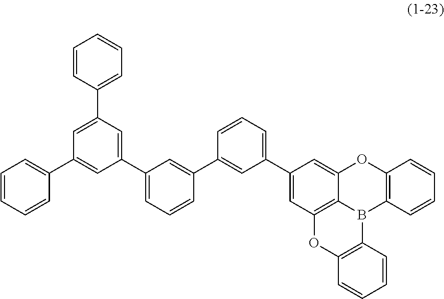 [SMILES: c1ccc(-c2cc(-c3ccccc3)cc(-c3cccc(-c4cccc(-c5cc6c7c(c5)Oc5ccccc5B7c5ccccc5O6)c4)c3)c2)cc1]

(1) Compound 1-23-1 (16.4 g, 60 mmol) and Compound 1-23-2 (18.7 g, 60 mmol), tetrakis(triphenylphosphine)palladium (3.45 g, 3 mmol), tetrabutylammonium bromide (2.6 g, 8 mmol), sodium hydroxide (3.2 g, 80 mmol), water (20 mL) and toluene (150 mL) were added to a 250 mL three-necked flask under nitrogen atmosphere, and the solution was heated to 80° C. and reacted under stirring for 12 hours at this temperature, and then the reaction was ended. The reaction solution was rotary evaporated to remove most of the solvent, and then dissolved with dichloromethane and washed with water for 3 times. The organic solution was collected, mixed with silica gel, and then purified by column chromatography to obtain compound 1-23-3, with a yield of 75%.

Synthetic route of the compound 1-23-3 is as follows:

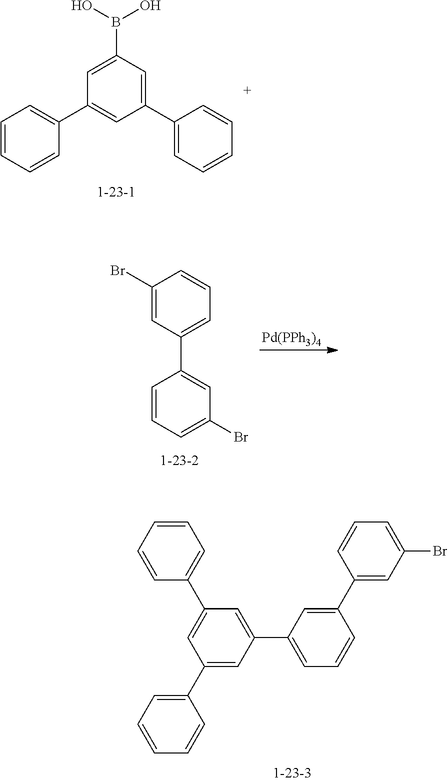 [SMILES: OB(O)c1cc(-c2ccccc2)cc(-c2ccccc2)c1 ; Brc1cccc(-c2cccc(Br)c2)c1 ; Brc1cccc(-c2cccc(-c3cc(-c4ccccc4)cc(-c4ccccc4)c3)c2)c1 ; "Pd(PPh3)4"]

(2) Compound 1-23-3 (18.4 g, 40 mmol) and 150 mL of anhydrous tetrahydrofuran were added to a 300 mL three-necked flask under nitrogen atmosphere, cooled to −78° C., and 45 mmol of n-butyllithium was slowly added dropwise, the solution was reacted for 2 hours. Then 50 mmol of isopropoxyboronic acid pinacol ester was added one time to allow the reaction temperature to rise to room temperature naturally. The reaction was further performed for 12 hours and then quenched by addition of pure water. The reaction solution was rotary evaporated to remove most of the solvent, and then extracted with dichloromethane and washed with water for 3 times. The organic phase was collected, spin dried, and then recrystallized to obtain compound 1-23-4, with a yield of 90%.

Synthetic route of the compound 1-23-4 is as follows:

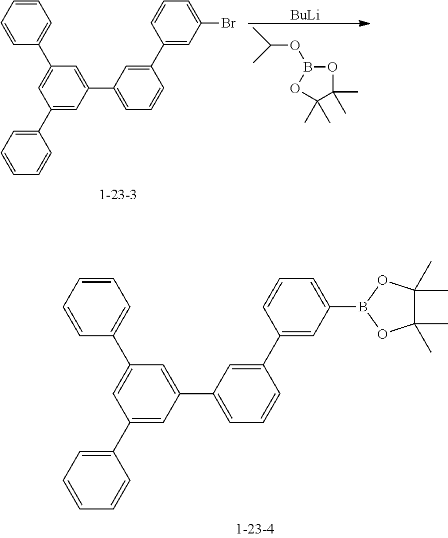

(3) Compound 1-23-5 (9.65 g, 50 mmol), Compound 1-23-6 (9.4 g, 100 mmol), potassium carbonate (150 mmol) and triglyme (80 mL) were added to a 150 mL three-necked flask under nitrogen atmosphere, and the solution was heated to 135° C. and reacted under stirring for 12 hours at this temperature, and then the reaction was ended when completed. The reaction solution was poured into 300 mL of water, and filter residue was filtered with suction, then recrystallized with the mixture solvent of ethanol and dichloromethane to obtain compound 1-23-7, with a yield of 90%.

Synthetic route of the compound 1-23-7 is as follows:

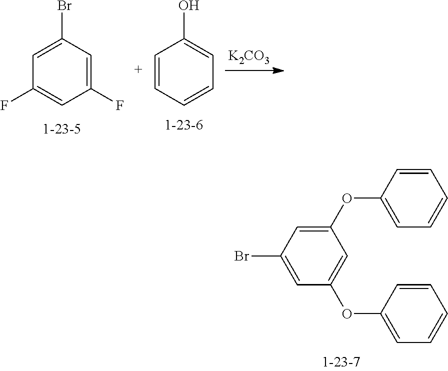

(4) Compound 1-23-4 (10.2 g, 20 mmol) and Compound 1-23-7 (6.8 g, 20 mmol), tetrakis(triphenylphosphine)palladium (1.15 g, 1 mmol), tetrabutylammonium bromide (1.3 g, 4 mmol), sodium hydroxide (1.6 g, 40 mmol), water (10 mL) and toluene (60 mL) were added to a 150 mL three-necked flask under nitrogen atmosphere, and the mixture was heated to 80° C. and reacted under stirring for 12 hours at this temperature, and then the reaction was ended. The reaction solution was rotary evaporated to remove most of the solvent, and then dissolved with dichloromethane and washed with water for 3 times. The organic solution was collected, mixed with silica gel, and then purified by column chromatography to obtain compound 1-23-8, with a yield of 90%.

Synthetic route of the compound 1-23-8 is as follows:

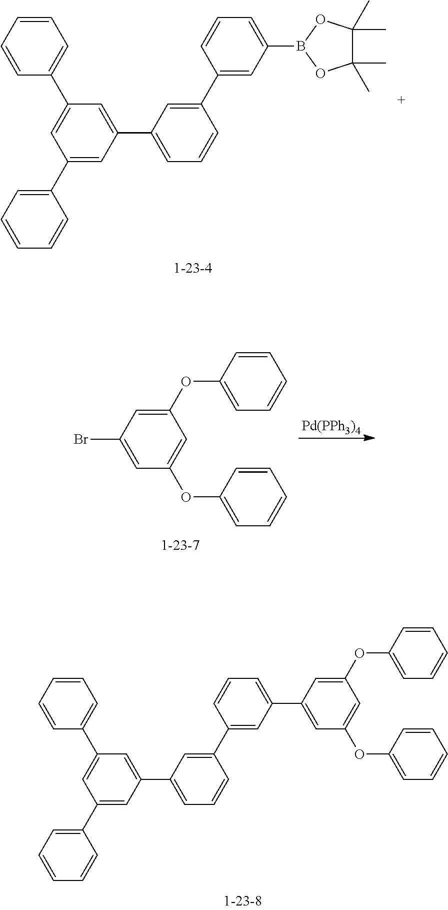 [SMILES: CC1(C)OB(c2cccc(-c3cccc(-c4cc(-c5ccccc5)cc(-c5ccccc5)c4)c3)c2)OC1(C)C ; Brc1cc(Oc2ccccc2)cc(Oc2ccccc2)c1 ; c1ccc(Oc2cc(Oc3ccccc3)cc(-c3cccc(-c4cccc(-c5cc(-c6ccccc6)cc(-c6ccccc6)c5)c4)c3)c2)cc1 ; "Pd(PPh3)4"]

(5) Compound 1-23-8 (9.3 g, 15 mmol) and 50 mL of anhydrous toluene were added to a 150 mL three-necked flask under nitrogen atmosphere, cooled to −78° C., and 20 mmol of n-butyllithium was slowly added dropwise, the solution was reacted for 2 hours. Then 20 mmol of boron tribromide was added dropwise at −20° C., the solution was reacted for 1 hour at room temperature. The reaction solution was cooled to 0° C., and 25 mmol of N,N-diisopropylethylamine was added dropwise. The reaction solution was heated to 110° C. and reacted for 12 hours. After the reaction was ended, the reaction solution was rotary evaporated to remove most of the solvent, and then extracted with dichloromethane and washed with water for 3 times. The organic phase was collected, spin dried, and then purified by column chromatography to obtain compound 1-23, with a yield of 90%.

Synthetic route of the compound 1-23 is as follows:

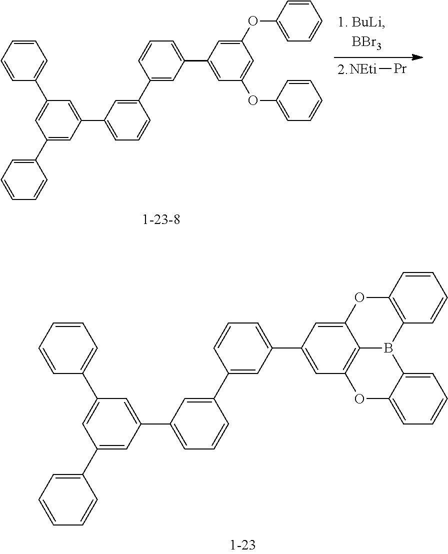

### Example 3

The preparation process of the first organic compound (1-130) of the present embodiment is as follows:

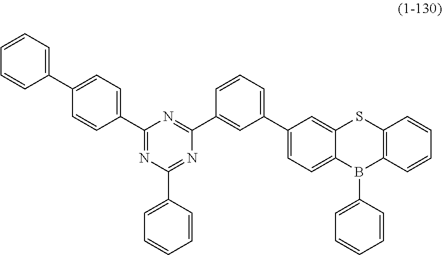 [SMILES: c1ccc(B2c3ccccc3Sc3cc(-c4cccc(-c5nc(-c6ccccc6)nc(-c6ccc(-c7ccccc7)cc6)n5)c4)ccc32)cc1]

(1) Compound 1-130-1 (11.9 g, 60 mmol) and Compound 1-130-2 (13.6 g, 60 mmol), tetrakis(triphenylphosphine)palladium (3.45 g, 3 mmol), tetrabutylammonium bromide (2.6 g, 8 mmol), sodium hydroxide (3.2 g, 80 mmol), water (20 mL) and toluene (150 mL) were added to a 250 mL three-necked flask under nitrogen atmosphere, and the solution was heated to 80° C. and reacted under stirring for 12 hours at this temperature, and then the reaction was ended. The reaction solution was rotary evaporated to remove most of the solvent, and then dissolved with dichloromethane and washed with water for 3 times. The organic solution was collected, mixed with silica gel, and then purified by column chromatography to obtain compound 1-130-3, with a yield of 70%.

Synthetic route of the compound 1-130-3 is as follows:

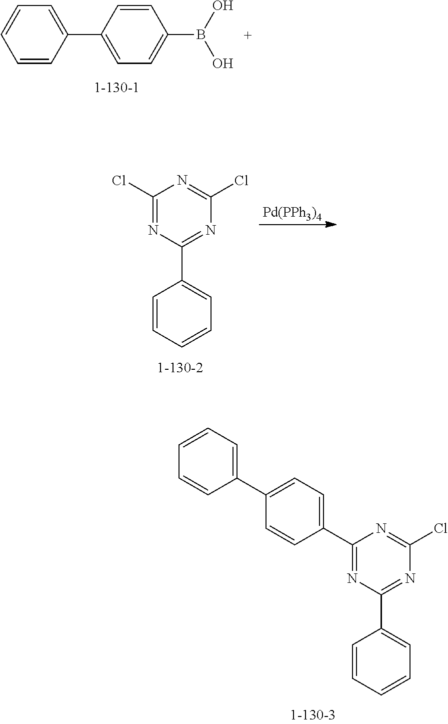

(2) Compound 1-4-5 (25.4 g, 100 mmol), Compound 1-130-4 (19 g, 100 mmol), potassium carbonate (200 mmol) and triglyme (160 mL) were added to a 300 mL three-necked flask under nitrogen atmosphere, and the solution e was heated to 135° C. and reacted under stirring for 12 hours at this temperature, and then the reaction was ended when completed. The reaction solution was poured into 500 mL of water, and filter residue was filtered with suction, then recrystallized with the mixture solvent of ethanol and dichloromethane to obtain compound 1-130-5, with a yield of 90%.

Synthetic route of the compound 1-130-5 is as follows:

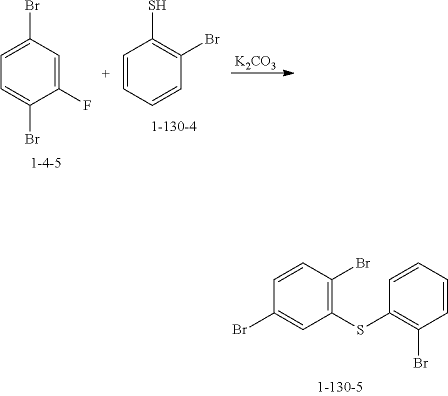

(3) Compound 1-130-5 (25.4 g, 60 mmol) and 300 mL of anhydrous tetrahydrofuran were added to a 500 mL three-necked flask under nitrogen atmosphere, cooled to 0° C., and 120 mmol of n-butyllithium was slowly added dropwise, the solution was reacted for 1 hours. Then 60 mmol of anhydrous tetrahydrofuran solution of methyl 4-boronobenzoate was added one time to allow the reaction temperature to rise to room temperature naturally. The reaction was further performed for 12 hours and then quenched by addition of pure water. The reaction solution was rotary evaporated to remove most of the solvent, and then extracted with dichloromethane and washed with water for 3 times. The organic phase was collected, spin dried, and then recrystallized to obtain compound 1-130-6, with a yield of 75%.

Synthetic route of the compound 1-130-6 is as follows:

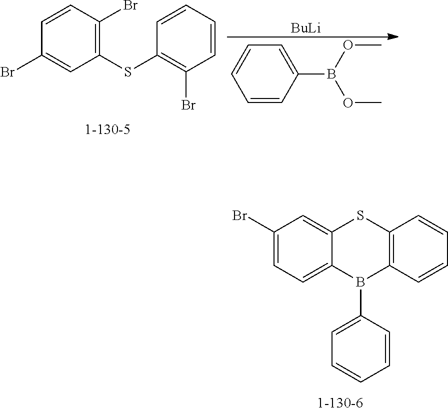

(4) Compound 1-130-6 (14 g, 40 mmol) and 150 mL of anhydrous tetrahydrofuran were added to a 300 mL three-necked flask under nitrogen atmosphere, cooled to −78° C., and 45 mmol of n-butyllithium was slowly added dropwise, the solution was reacted for 2 hours. Then 50 mmol of isopropoxyboronic acid pinacol ester was added one time to allow the reaction temperature to rise to room temperature naturally. The reaction was further performed for 12 hours and then quenched by addition of pure water. The reaction solution was rotary evaporated to remove most of the solvent, and then extracted with dichloromethane and washed with water for 3 times. The organic phase was collected, spin dried, and then recrystallized to obtain compound 1-130-7, with a yield of 90%.

Synthetic route of the compound 1-130-7 is as follows:

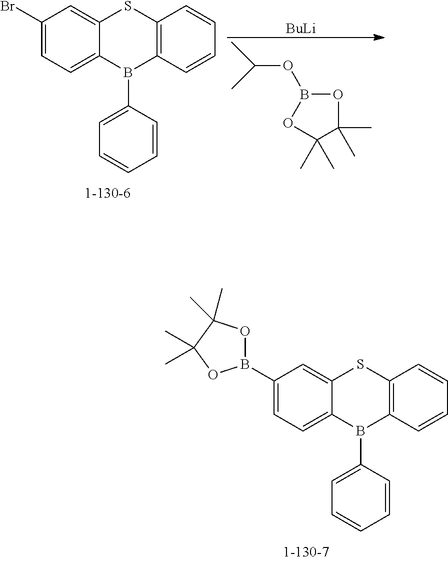

(5) Compound 1-130-7 (11.9 g, 30 mmol) and Compound 1-130-8 (8.5 g, 30 mmol), tetrakis(triphenylphosphine)palladium (1.73 g, 1.5 mmol), tetrabutylammonium bromide (1.3 g, 4 mmol), sodium hydroxide (1.6 g, 40 mmol), water (10 mL) and toluene (80 mL) were added to a 250 mL three-necked flask under nitrogen atmosphere, and the solution was heated to 80° C. and reacted under stirring for 12 hours at this temperature, and then the reaction was ended. The reaction solution was rotary evaporated to remove most of the solvent, and then dissolved with dichloromethane and washed with water for 3 times. The organic solution was collected, mixed with silica gel, and then purified by column chromatography to obtain compound 1-130-9, with a yield of 80%.

Synthetic route of the compound 1-130-9 is as follows:

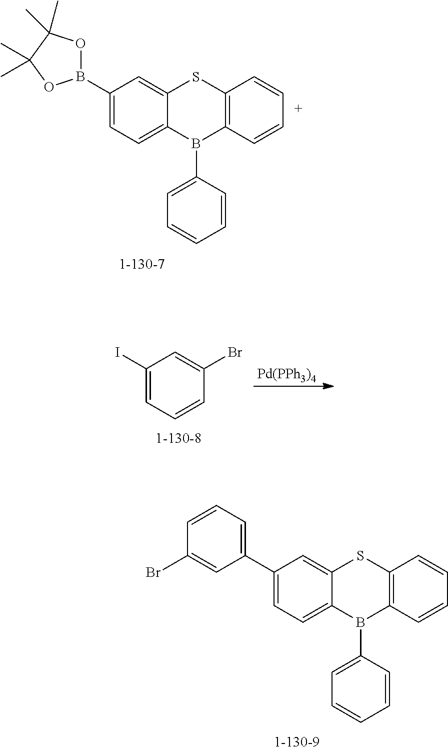 [SMILES: CC1(C)OB(c2ccc3c(c2)Sc2ccccc2B3c2ccccc2)OC1(C)C ; Brc1cccc(I)c1 ; Brc1cccc(-c2ccc3c(c2)Sc2ccccc2B3c2ccccc2)c1 ; "Pd(PPh3)4"]

(6) Compound 1-130-9 (8.5 g, 20 mmol) and 60 mL of anhydrous tetrahydrofuran were added to a 150 mL three-necked flask under nitrogen atmosphere, cooled to −78° C., and 25 mmol of n-butyllithium was slowly added dropwise, the solution was reacted for 2 hours. Then 30 mmol of isopropoxyboronic acid pinacol ester was added one time to allow the reaction temperature to rise to room temperature naturally. The reaction was further performed for 12 hours and then quenched by addition of pure water. The reaction solution was rotary evaporated to remove most of the solvent, and then extracted with dichloromethane and washed with water for 3 times. The organic phase was collected, spin dried, and then recrystallized to obtain compound 1-130-10, with a yield of 85%.

Synthetic route of the compound 1-130-10 is as follows:

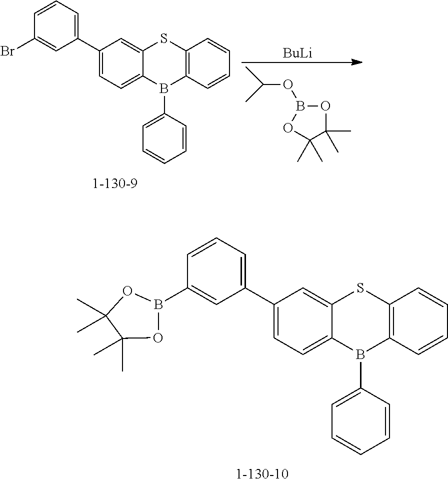

(7) Compound 1-130-3 (3.4 g, 10 mmol) and Compound 1-130-10 (4.7 g, 10 mmol), tetrakis(triphenylphosphine)palladium (0.57 g, 0.5 mmol), tetrabutylammonium bromide (0.7 g, 2 mmol), sodium hydroxide (0.8 g, 20 mmol), water (50 mL) and toluene (50 mL) were added to a 150 mL three-necked flask under nitrogen atmosphere, and the solution was heated to 80° C. and reacted under stirring for 12 hours at this temperature, and then the reaction was ended. The reaction solution was rotary evaporated to remove most of the solvent, and then dissolved with dichloromethane and washed with water for 3 times. The organic solution was collected, mixed with silica gel, and then purified by column chromatography to obtain compound 1-130, with a yield of 80%.

Synthetic route of the compound 1-130 is as follows:

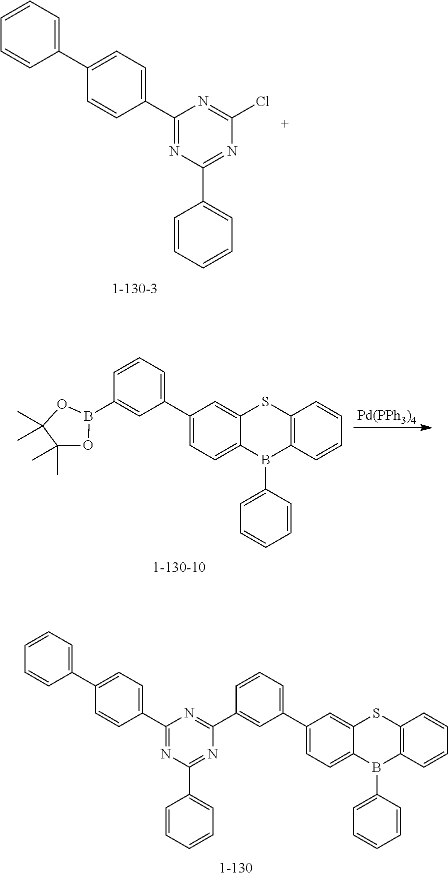

### Example 4

The preparation process of the second organic compound (2-40) of the present embodiment is as follows:

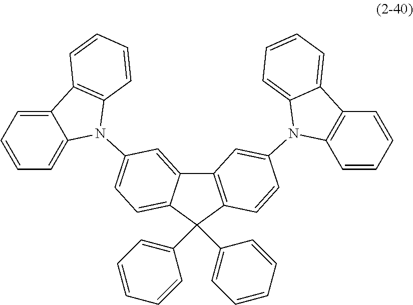 [SMILES: c1ccc(C2(c3ccccc3)c3ccc(-n4c5ccccc5c5ccccc54)cc3-c3cc(-n4c5ccccc5c5ccccc54)ccc32)cc1]

Compound 2-40-1 (10 g, 60 mmol) and Compound 2-40-2 (28.6 g, 60 mmol), copper powder (0.39 g, 6 mmol), potassium carbonate (8.28 g, 60 mmol) and 18-crown-6 (2.65 g, 5 mmol) and o-dichlorobenzene (150 mL) were added to a 300 mL two-necked flask under nitrogen atmosphere, and the solution was heated to 150° C. and reacted under stirring for 24 hours at this temperature, and then the reaction was ended. The reaction solution was distilled under reduced pressure to remove most of the solvent, and then dissolved with dichloromethane and washed with water for 3 times. The organic solution was collected, mixed with silica gel, and purified by column chromatography to obtain compound 2-40, with a yield of 80%.

Synthetic route of the compound 2-40 is as follows:

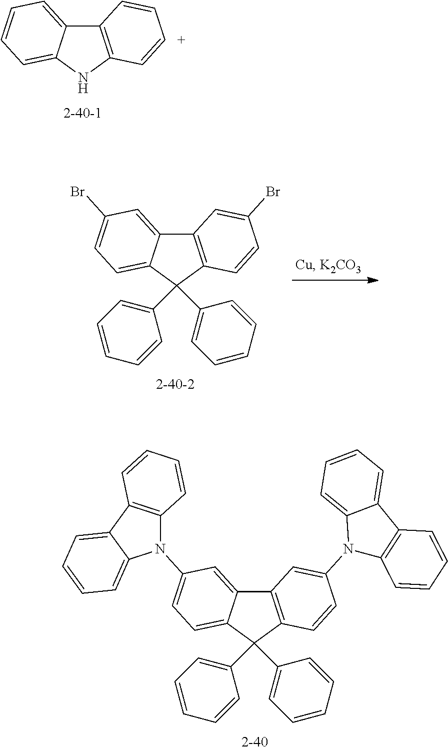 [SMILES: c1ccc2c(c1)[nH]c1ccccc12 ; Brc1ccc2c(c1)-c1cc(Br)ccc1C2(c1ccccc1)c1ccccc1 ; c1ccc(C2(c3ccccc3)c3ccc(-n4c5ccccc5c5ccccc54)cc3-c3cc(-n4c5ccccc5c5ccccc54)ccc32)cc1]

### Example 5

The preparation process of the second organic compound (3-2) of the present embodiment is as follows:

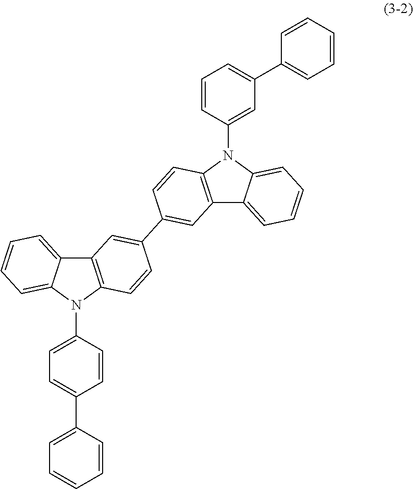 [SMILES: c1ccc(-c2ccc(-n3c4ccccc4c4cc(-c5ccc6c(c5)c5ccccc5n6-c5cccc(-c6ccccc6)c5)ccc43)cc2)cc1]

(1) Compound 3-2-1 (15.9 g, 40 mmol) and 300 mL of anhydrous tetrahydrofuran were added to a 500 mL three-necked flask under nitrogen atmosphere, cooled to −78° C., and 50 mmol of n-butyllithium was slowly added dropwise, the solution was reacted for 2 hours. Then 55 mmol of isopropoxyboronic acid pinacol ester was added one time to allow the reaction temperature to rise to room temperature naturally. The reaction was further performed for 12 hours and then quenched by addition of pure water. The reaction solution was rotary evaporated to remove most of the solvent, and then extracted with dichloromethane and washed with water for 3 times. The organic phase was collected, spin dried, and then recrystallized to obtain compound 3-2-2, with a yield of 80%.

Synthetic route of the compound 3-2-2 is as follows:

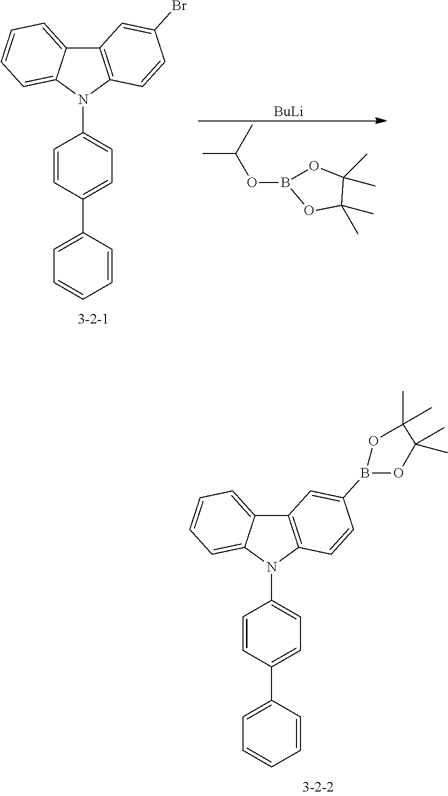

(2) Compound 3-2-2 (4.45 g, 20 mmol) and Compound 3-2-3 (3.98 g, 20 mmol), tetrakis(triphenylphosphine)palladium (1.15 g, 1 mmol), tetrabutylammonium bromide (2.6 g, 8 mmol), sodium hydroxide (3.2 g, 80 mmol), water (10 mL) and toluene (100 mL) were added to a 250 mL three-necked flask under nitrogen atmosphere, and the solution was heated to 80° C. and reacted under stirring for 12 hours, and then the reaction was ended. The reaction solution was rotary evaporated to remove most of the solvent, and then dissolved with dichloromethane and washed with water for 3 times. The organic solution was collected, mixed with silica gel, and then purified by column chromatography to obtain compound 3-2, with a yield of 80%.

Synthetic route of the compound 3-2 is as follows:

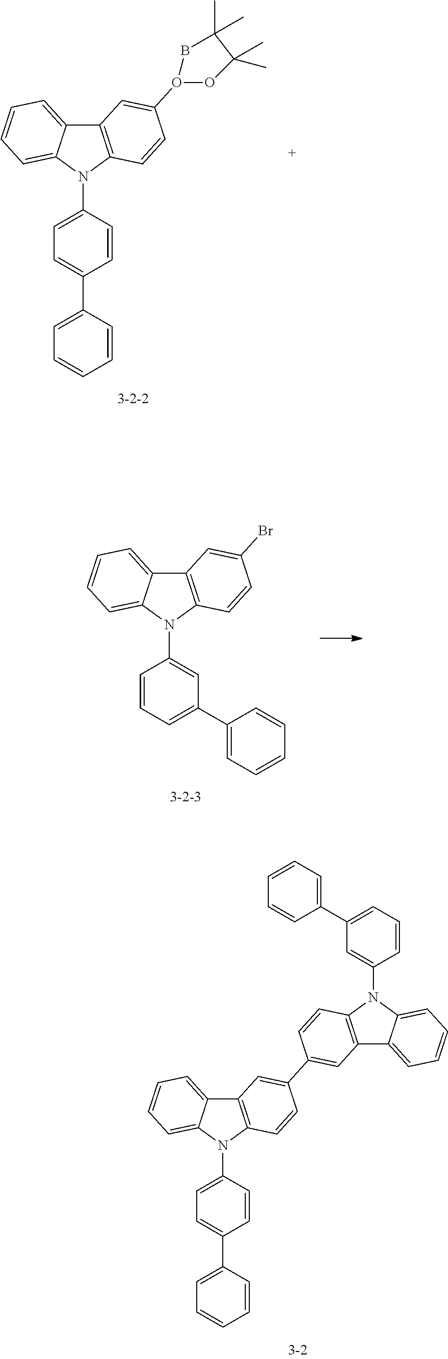

### Example 6

The preparation process of the second organic compound (3-23) of the present embodiment is as follows:

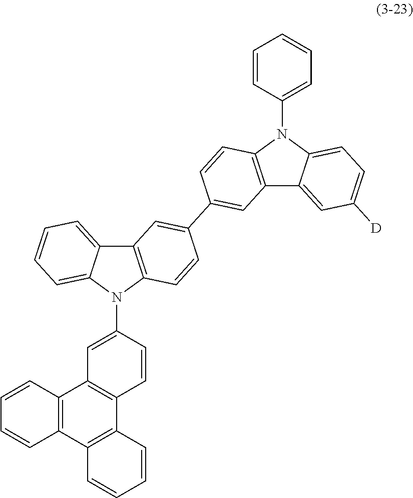 [SMILES: [2H]c1ccc2c(c1)c1cc(-c3ccc4c(c3)c3ccccc3n4-c3ccc4c5ccccc5c5ccccc5c4c3)ccc1n2-c1ccccc1]

(1) Compound 3-23-1 (9.8 g, 40 mmol) and 100 mL of N,N-dimethylformamide were added into a 250 mL single-necked flask, and 40 mmol of N,N-dimethylformamide solution of NBS was added dropwise in an ice bath. The solution was reacted under stirring for 12 hours in the dark, and then the reaction was ended. The reaction solution was poured into 500 mL of water, filtered with suction, and the filter residue was recrystallized to obtain compound 3-23-2, with a yield 90%.

Synthetic route of the compound 3-23-2 is as follows:

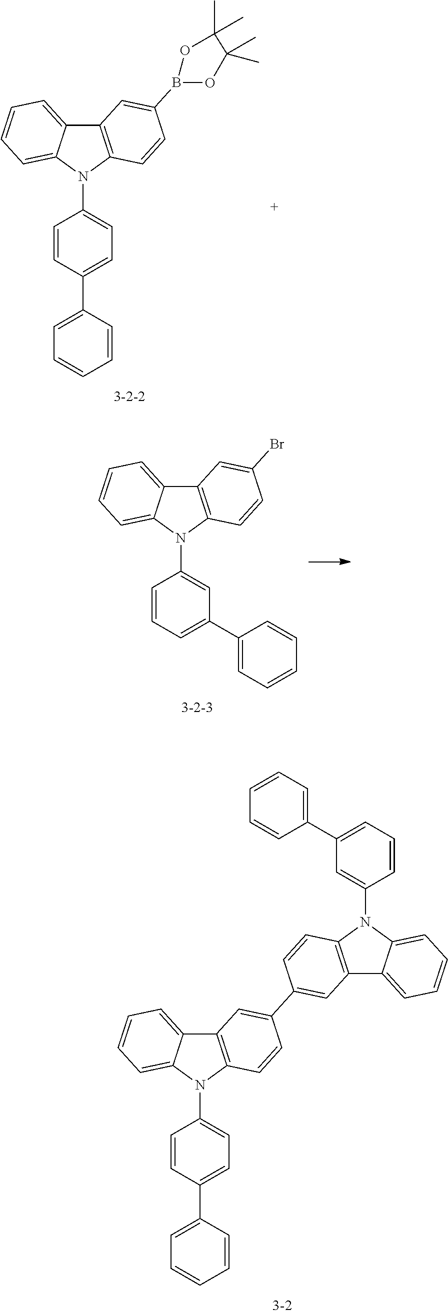 [SMILES: CC1(C)OB(c2ccc3c(c2)c2ccccc2n3-c2ccc(-c3ccccc3)cc2)OC1(C)C ; Brc1ccc2c(c1)c1ccccc1n2-c1cccc(-c2ccccc2)c1 ; c1ccc(-c2ccc(-n3c4ccccc4c4cc(-c5ccc6c(c5)c5ccccc5n6-c5cccc(-c6ccccc6)c5)ccc43)cc2)cc1]

(2) Compound 3-23-2 (9.69 g, 30 mmol) and 150 mL of anhydrous tetrahydrofuran were added to a 300 mL three-necked flask under nitrogen atmosphere, cooled to −78° C., and 35 mmol of n-butyllithium was slowly added dropwise, the mixture was reacted for 2 hours. Then 40 mmol of isopropoxyboronic acid pinacol ester was added one time to allow the reaction temperature to rise to room temperature naturally. The reaction was further performed for 12 hours and then quenched by addition of pure water. The reaction solution was rotary evaporated to remove most of the solvent, and then extracted with dichloromethane and washed with water for 3 times. The organic phase was collected, spin dried, and then recrystallized to obtain compound 3-23-3, with a yield of 90%.

Synthetic route of the compound 3-23-3 is as follows:

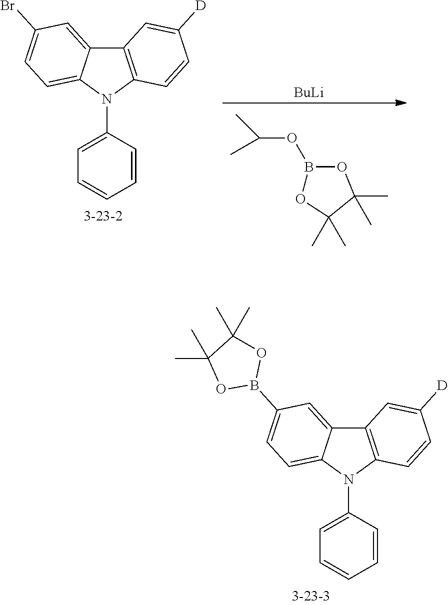

(3) Compound 3-23-4 (10 g, 60 mmol) and Compound 3-23-5 (18.4 g, 60 mmol), copper powder (0.39 g, 6 mmol), potassium carbonate (8.28 g, 60 mmol) and 18-crown-6 (2.65 g, 5 mmol) and o-dichlorobenzene (150 mL) were added to a 300 mL two-necked flask under nitrogen atmosphere, and the solution was heated to 150° C. and reacted under stirring for 24 hours at this temperature, and then the reaction was ended. The reaction solution was distilled under reduced pressure to remove most of the solvent, and then dissolved with dichloromethane and washed with water for 3 times. The organic solution was collected, mixed with silica gel, and purified by column chromatography to obtain compound 3-23-6, with a yield of 80%.

Synthetic route of the compound 3-23-6 is as follows:

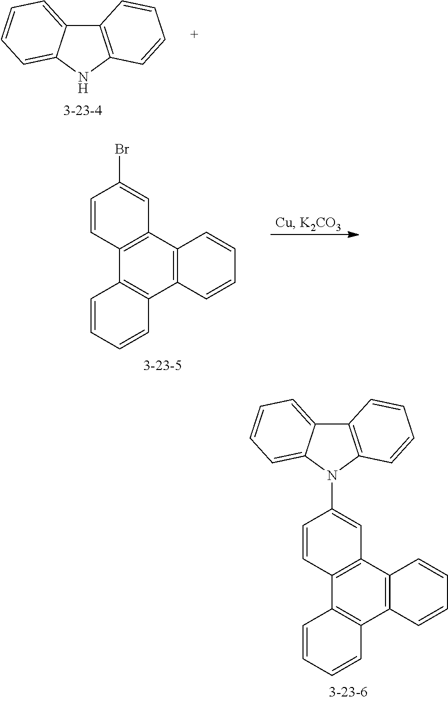

(4) Compound 3-23-6 (15.7 g, 40 mmol) and 100 mL of N,N-dimethylformamide were added into a 250 mL single-necked flask, and 40 mmol of N,N-dimethylformamide solution of NBS was added dropwisely in an ice bath. The solution was reacted under stirring for 12 hours in the dark, and then the reaction was ended. The reaction solution was poured into 500 mL of water, filtered with suction, and the filter residue was recrystallized to obtain compound 3-23-7, with a yield 92%.

Synthetic route of the compound 3-23-7 is as follows:

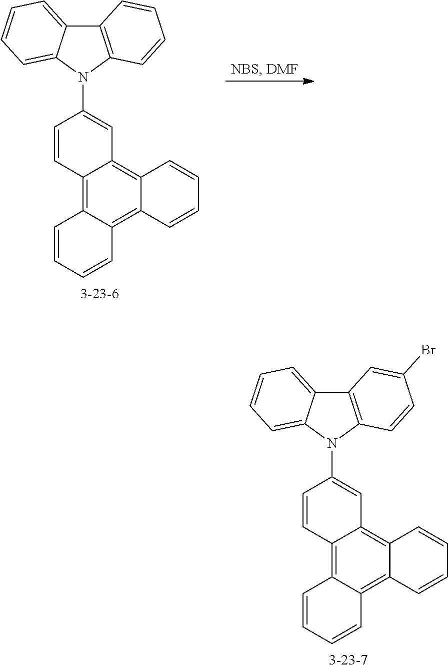

(5) Compound 3-23-3 (14 g, 20 mmol) and Compound 3-23-7 (9.4 g, 20 mmol), tetrakis(triphenylphosphine)palladium (2.3 g, 2 mmol), tetrabutylammonium bromide (2.6 g, 8 mmol), sodium hydroxide (3.2 g, 80 mmol), water (10 mL) and toluene (100 mL) were added to a 250 mL three-necked flask under nitrogen atmosphere, and the solution was heated to 80° C. and reacted under stirring for 12 hours, and then the reaction was ended. The reaction solution was rotary evaporated to remove most of the solvent, and then dissolved with dichloromethane and washed with water for 3 times. The organic solution was collected, mixed with silica gel, and then purified by column chromatography to obtain compound 3-23, with a yield of 85%.

Synthetic route of the compound 3-23 is as follows:

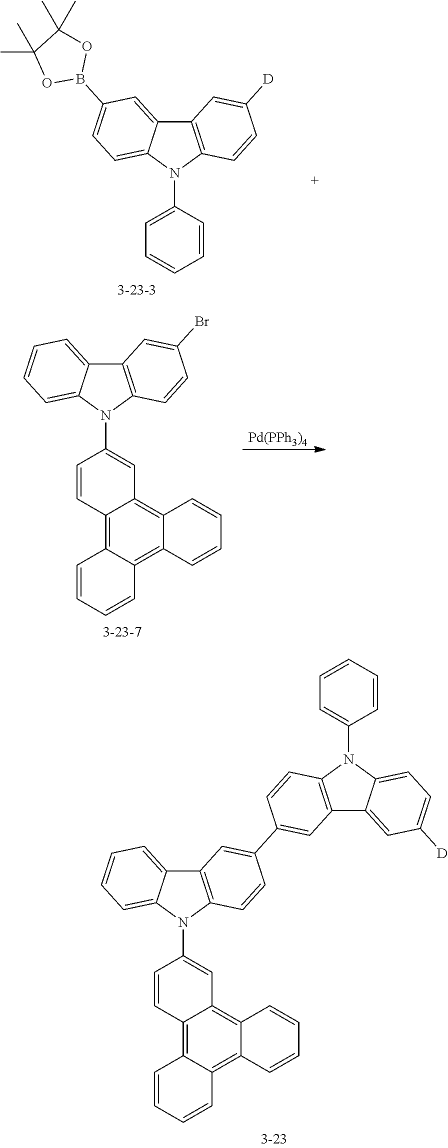 [SMILES: [2H]c1ccc2c(c1)c1cc(B3OC(C)(C)C(C)(C)O3)ccc1n2-c1ccccc1 ; Brc1ccc2c(c1)c1ccccc1n2-c1ccc2c3ccccc3c3ccccc3c2c1 ; [2H]c1ccc2c(c1)c1cc(-c3ccc4c(c3)c3ccccc3n4-c3ccc4c5ccccc5c5ccccc5c4c3)ccc1n2-c1ccccc1 ; "Pd(PPh3)4"]

### Example 7

The preparation process of the second organic compound (4-18) of the present embodiment is as follows:

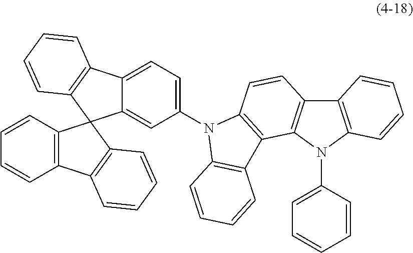 [SMILES: c1ccc(-n2c3ccccc3c3ccc4c(c5ccccc5n4-c4ccc5c(c4)C4(c6ccccc6-c6ccccc64)c4ccccc4-5)c32)cc1]

(1) Compound 4-18-1 (20 g, 120 mmol) and Compound 4-18-2 (41.4 g, 120 mmol), copper powder (0.78 g, 12 mmol), potassium carbonate (16.6 g, 120 mmol) and 18-crown-6 (5.3 g, 10 mmol) and o-dichlorobenzene (300 mL) were added to a 500 mL two-necked flask under nitrogen atmosphere, and the solution was heated to 150° C. and reacted under stirring for 24 hours at this temperature, and then the reaction was ended. The reaction solution was distilled under reduced pressure to remove most of the solvent, and then dissolved with dichloromethane and washed with water for 3 times. The organic solution was collected, mixed with silica gel, and purified by column chromatography to obtain compound 4-18-3, with a yield of 80%.

Synthetic route of the compound 4-18-3 is as follows:

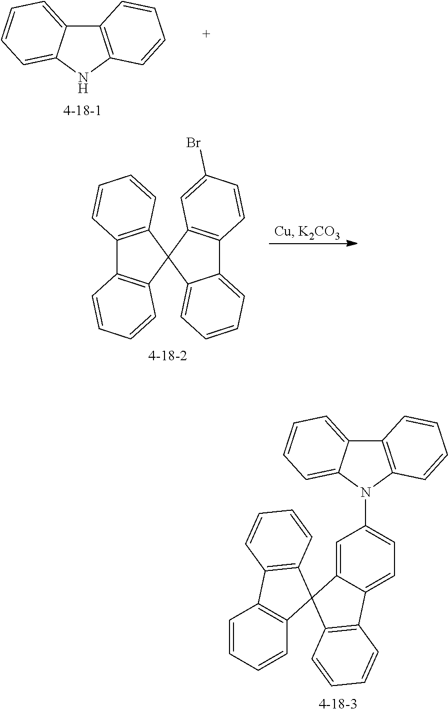

(2) Compound 4-18-3 (38.6 g, 80 mmol) and 200 mL of N,N-dimethylformamide were added into a 500 mL single-necked flask, and 80 mmol of N,N-dimethylformamide solution of NBS was added dropwise in an ice bath. The mixture was reacted under stirring for 12 hours in the dark, and then the reaction was ended. The reaction solution was poured into 500 mL of water, filtered with suction, and the filter residue was recrystallized to obtain compound 4-18-4, with a yield 90%.

Synthetic route of the compound 4-18-4 is as follows:

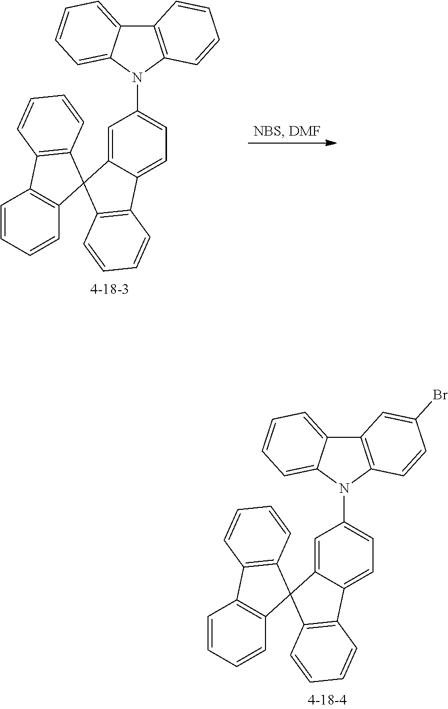

(3) Compound 4-18-4 (33.6 g, 60 mmol) and 200 mL of anhydrous tetrahydrofuran were added to a 500 mL three-necked flask under nitrogen atmosphere, cooled to −78° C., and 65 mmol of n-butyllithium was slowly added dropwise, the solution was reacted for 2 hours. Then 70 mmol of isopropoxyboronic acid pinacol ester was added one time to allow the reaction temperature to rise to room temperature naturally. The reaction was further performed for 12 hours and then quenched by addition of pure water. The reaction solution was rotary evaporated to remove most of the solvent, and then extracted with dichloromethane and washed with water for 3 times. The organic phase was collected, spin dried, and then recrystallized to obtain compound 4-18-5, with a yield of 85%.

Synthetic route of the compound 4-18-5 is as follows:

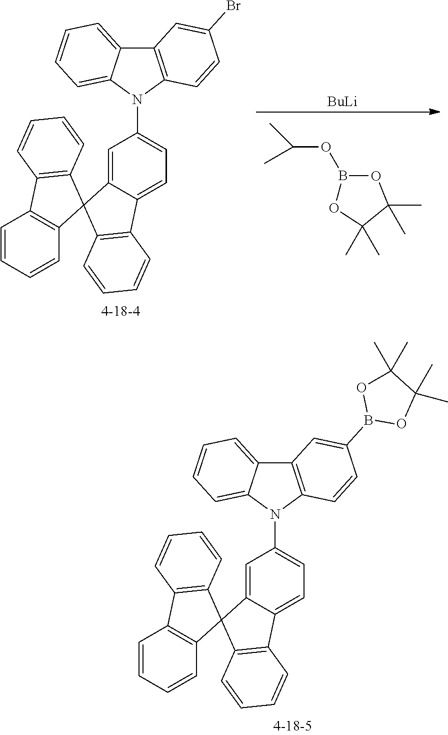

(4) Compound 4-18-5 (24.2 g, 40 mmol) and Compound 4-18-6 (8.1 g, 40 mmol), tetrakis(triphenylphosphine)palladium (2.3 g, 2 mmol), tetrabutylammonium bromide (6.5 g, 20 mmol), sodium hydroxide (3.2 g, 80 mmol), water (10 mL) and toluene (60 mL) were added to a 150 mL three-necked flask under nitrogen atmosphere, and the solution was heated to 80° C. and reacted under stirring for 12 hours at this temperature, and then the reaction was ended. The reaction solution was rotary evaporated to remove most of the solvent, and then dissolved with dichloromethane and washed with water for 3 times. The organic solution was collected, mixed with silica gel, and then purified by column chromatography to obtain compound 4-18-7, with a yield of 75%.

Synthetic route of the compound 4-18-7 is as follows:

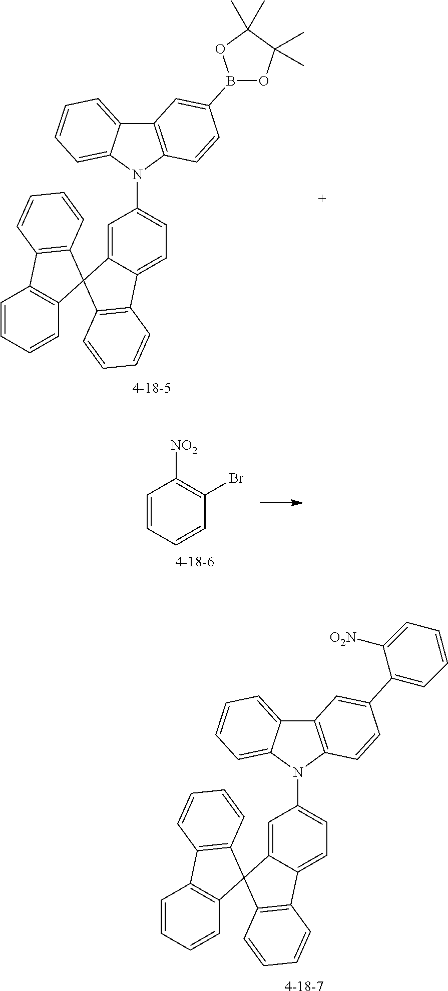

(5) Compound 4-18-7 (15 g, 25 mmol) and triethylphosphine (8.3 g, 50 mmol) were added to a 150 mL two-necked flask under nitrogen atmosphere, and the solution was heated to 190° C. and reacted under stirring for 12 hours at this temperature, and then the reaction was ended. The reaction solution was distilled under reduced pressure to remove most of the solvent, and then dissolved with dichloromethane and washed with water for 3 times. The organic solution was collected, mixed with silica gel, and then purified by column chromatography to obtain compound 4-18-9, with a yield of 85%.

Synthetic route of the compound 4-18-9 is as follows:

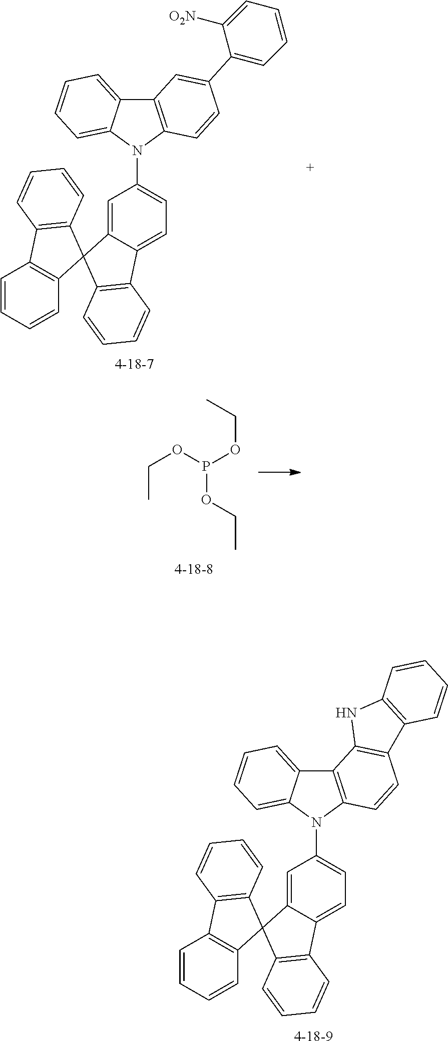

(6) Compound 4-18-9 (8.6 g, 15 mmol) and Compound 4-18-10 (3.1 g, 15 mmol), copper powder (0.16 g, 2 mmol), potassium carbonate (2.8 g, 20 mmol) and 18-crown-6 (2.65 g, 5 mmol) and o-dichlorobenzene (60 mL) were added to a 150 mL two-necked flask under nitrogen atmosphere, and the solution was heated to 150° C. and reacted under stirring for 24 hours at this temperature, and then the reaction was ended. The reaction solution was distilled under reduced pressure to remove most of the solvent, and then dissolved with dichloromethane and washed with water for 3 times. The organic solution was collected, mixed with silica gel, and purified by column chromatography to obtain compound 4-18, with a yield of 80%.

Synthetic route of the compound 4-18 is as follows:

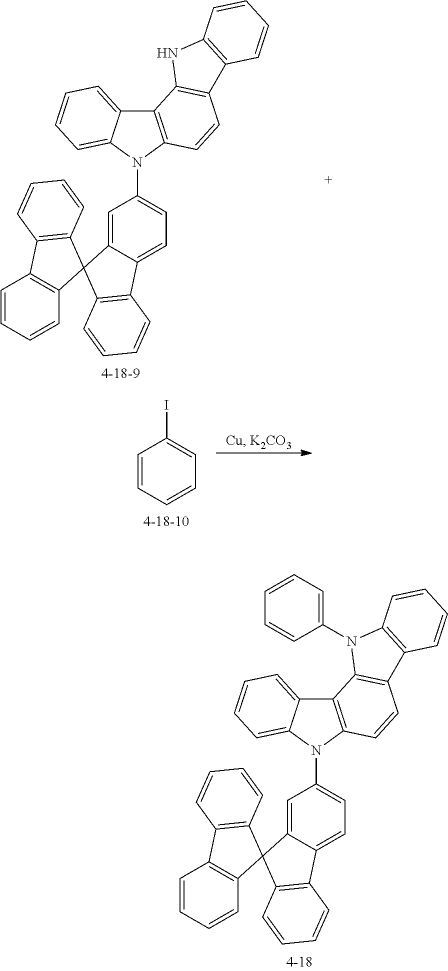 [SMILES: c1ccc2c(c1)-c1ccccc1C21c2ccccc2-c2ccc(-n3c4ccccc4c4c5[nH]c6ccccc6c5ccc43)cc21 ; Ic1ccccc1 ; c1ccc(-n2c3ccccc3c3ccc4c(c5ccccc5n4-c4ccc5c(c4)C4(c6ccccc6-c6ccccc64)c4ccccc4-5)c32)cc1]

### Example 8

The preparation process of the second organic compound (4-18) of the present embodiment is as follows:

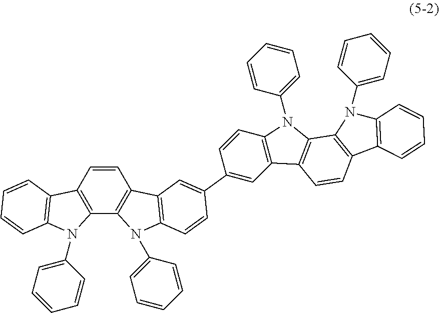 [SMILES: c1ccc(-n2c3ccccc3c3ccc4c5cc(-c6ccc7c(c6)c6ccc8c9ccccc9n(-c9ccccc9)c8c6n7-c6ccccc6)ccc5n(-c5ccccc5)c4c32)cc1]

(1) Compound 5-2-1 (6.2 g, 15 mmol), Compound 4-18-10 (3.1 g, 15 mmol), copper powder (0.16 g, 2 mmol), potassium carbonate (2.8 g, 20 mmol) and 18-crown-6 (2.65 g, 5 mmol) and o-dichlorobenzene (60 mL) were added to a 150 mL two-necked flask under nitrogen atmosphere, and the solution was heated to 150° C. and reacted under stirring for 24 hours at this temperature, and then the reaction was ended. The reaction solution was distilled under reduced pressure to remove most of the solvent, and then dissolved with dichloromethane and washed with water for 3 times. The organic solution was collected, mixed with silica gel, and purified by column chromatography to obtain compound 4-18, with a yield of 70%.

Synthetic route of the compound 5-2-2 is as follows:

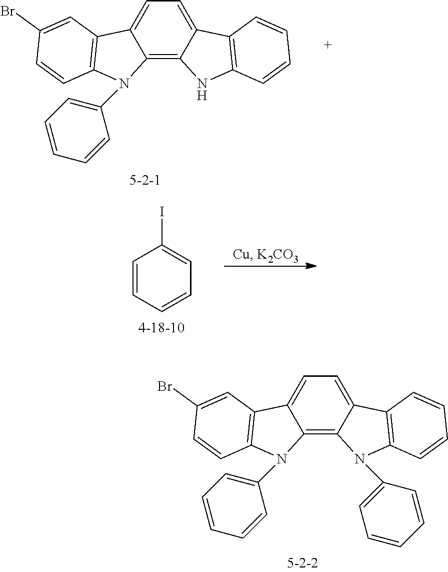 [SMILES: Brc1ccc2c(c1)c1ccc3c4ccccc4[nH]c3c1n2-c1ccccc1 ; Ic1ccccc1 ; Brc1ccc2c(c1)c1ccc3c4ccccc4n(-c4ccccc4)c3c1n2-c1ccccc1]

(2) Compound 5-2-2 (4.9 g, 10 mmol) and 60 mL of anhydrous tetrahydrofuran were added to a 150 mL three-necked flask under nitrogen atmosphere, cooled to −78° C., and 12 mmol of n-butyllithium was slowly added dropwise, the solution was reacted for 2 hours. Then 15 mmol of isopropoxyboronic acid pinacol ester was added one time to allow the reaction temperature to rise to room temperature naturally. The reaction was further performed for 12 hours and then quenched by addition of pure water. The reaction solution was rotary evaporated to remove most of the solvent, and then extracted with dichloromethane and washed with water for 3 times. The organic phase was collected, spin dried, and then recrystallized to obtain compound 5-2-3, with a yield of 80%.

Synthetic route of the compound 5-2-3 is as follows:

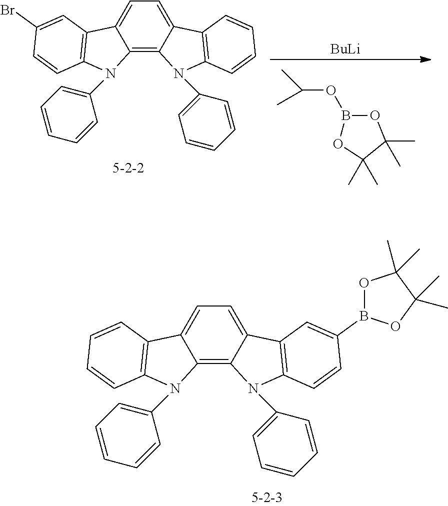

(4) Compound 5-2-2 (4.1 g, 6 mmol) and Compound 5-2-3 (3.0 g, 6 mmol), tetrakis(triphenylphosphine)palladium (0.35 g, 0.3 mmol), tetrabutylammonium bromide (3.3 g, 10 mmol), sodium hydroxide (0.8 g, 20 mmol), water (5 mL) and toluene (40 mL) were added to a 150 mL three-necked flask under nitrogen atmosphere, and the solution was heated to 80° C. and reacted under stirring for 12 hours at this temperature, and then the reaction was ended. The reaction solution was rotary evaporated to remove most of the solvent, and then dissolved with dichloromethane and washed with water for 3 times. The organic solution was collected, mixed with silica gel, and then purified by column chromatography to obtain compound 5-2, with a yield of 80%.

Synthetic route of the compound 5-2 is as follows:

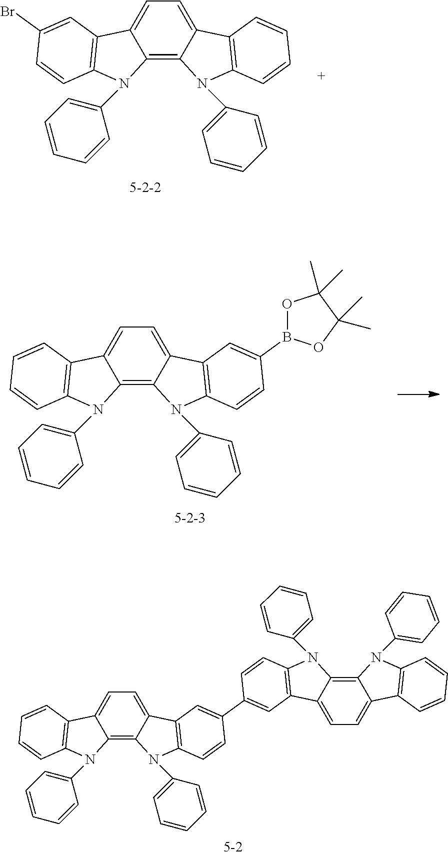 [SMILES: Brc1ccc2c(c1)c1ccc3c4ccccc4n(-c4ccccc4)c3c1n2-c1ccccc1 ; CC1(C)OB(c2ccc3c(c2)c2ccc4c5ccccc5n(-c5ccccc5)c4c2n3-c2ccccc2)OC1(C)C ; c1ccc(-n2c3ccccc3c3ccc4c5cc(-c6ccc7c(c6)c6ccc8c9ccccc9n(-c9ccccc9)c8c6n7-c6ccccc6)ccc5n(-c5ccccc5)c4c32)cc1]

### Examples 9 to 23

The structures of the organic light-emitting diodes (OLED devices) of Examples 9 to 23 are all ITO/HATCN/HTL/host material: Ir(p-ppy)₃/NaTzF₂: Liq/Liq/Al, wherein “/” means layered structure:

The first organic compound (1-23) prepared in Example 2 and the second organic compound (2-40) prepared in Example 4 were used as the host material at a mass ratio of 1:1 in the organic light-emitting diodes of Examples 9 to 11; The first organic compound (1-4) prepared in Example 1 and the second organic compound (3-23) prepared in Example 6 were used as the host material at a mass ratio of 1:1 in the organic light-emitting diodes of Examples 12 to 14; The first organic compound (1-23) prepared in Example 2 and the second organic compound (3-2) prepared in Example 5 were used as the host material at a mass ratio of 1:1 in the organic light-emitting diodes of Examples 15 to 17; The first organic compound (1-23) prepared in Example 2 and the second organic compound (4-18) prepared in Example 7 were used as the host material at a mass ratio of 1:1 in the organic light-emitting diodes of Examples 18 to 20; The first organic compound (1-23) prepared in Example 2 and the second organic compound (5-2) prepared in Example 8 were used as the host material at a mass ratio of 1:1 in the organic light-emitting diodes of Examples 21 to 23.

In Examples 9 to 23, Ir(p-ppy)₃ as shown below was used as the light-emitting material to form the light-emitting layer with the mass ratio of the host material to the light-emitting material being 90:10, HATCN with following structure as the hole injection material, SFNFB as the hole transport material, NaTzF₂ as the electron transport material, and Liq as the electron injection material, to prepare organic light-emitting diodes with the above structure.

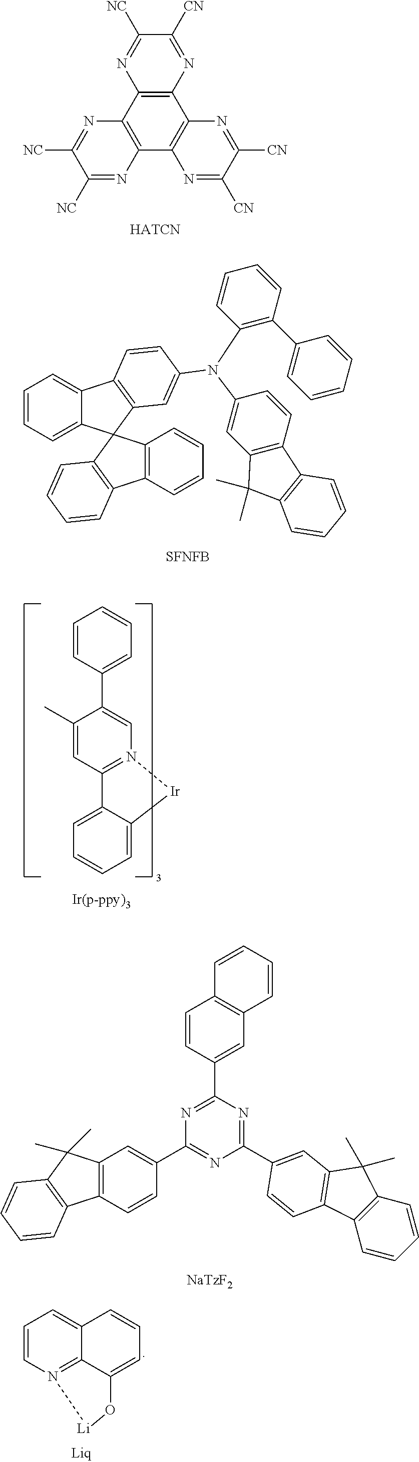

The above materials such as HATCN, SFNFB, Ir(p-ppy)₃, NaTzF₂ and Liq are all commercially available, such as from Jilin OLED Material Tech Co., Ltd (www.jl-oled.com), and will not described herein.

The specific preparation process of the organic light-emitting diodes of Examples 9 to 23 is as follows:

a. Cleaning of ITO (Indium Tin Oxide) conductive glass substrate: the substrate was cleaned with a variety of solvents (such as one or more of chloroform, acetone or isopropanol), and then treated with ultraviolet and ozone;

b. HATCN (30 nm), SFNFB (50 nm), NaTzF₂: Liq (30 nm), Liq (1 nm), Al (100 nm) was formed by thermal evaporation in high vacuum (1×10⁻⁶ mbar); host material: 10% Ir(p-ppy)₃ (40 nm) was prepared by the method as shown in Table 3.

Wherein, the host material may be prepared by the following three ways: (1) Vacuum co-evaporation, i.e., the two host materials were respectively placed in two different sources, and the doping ratio of the two host materials was controlled by controlling the respective evaporation rates. (2) Simple blending, i.e., the two host materials were weighed according to a certain ratio, doped together, ground at room temperature, and the resulting mixture was placed in an organic source for evaporation. (3) Organic alloy, i.e., the two host materials were weighed according to a certain ratio, doped together, heated and stirred until the mixture was melted under a vacuum lower than 10⁻³ torr. The mixture was cooled and then ground, and the resulting mixture was placed in an organic source for evaporation.

d. Encapsulating: the device was encapsulated with UV-curable resin in a nitrogen glove box.

### Comparative Example 1

The structure of the organic light-emitting diode of Comparative Example 1 was substantially the same as that of the organic light-emitting diode of Example 9, except that the host material of the light-emitting layer of Comparative Example 1 was mCP having the following structural formula (wherein, mCP was purchased from Jilin OLED Material Tech Co., Ltd):

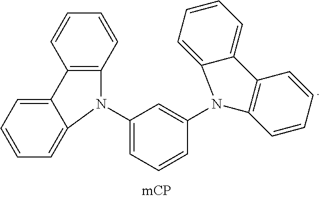 [SMILES: c1cc(-n2c3ccccc3c3ccccc32)cc(-n2c3ccccc3c3ccccc32)c1]

Test:

(1) Energy level tests of various materials used in Examples 9 to 23 and Comparative Example 1:

The energy levels of organic materials can be obtained by quantum calculations, such as using TD-DFT (Time Dependent-Density Functional Theory) by Gaussian03W (Gaussian Inc.), and the specific simulation methods can be found in WO2011141110. Firstly, the molecular geometry is optimized by semi-empirical method “Ground State/Semi-empirical/Default Spin/AM1” (Charge 0/Spin Singlet), and then the energy structure of organic molecules is calculated by TD-DFT (time-density functional theory) “TD-SCF/DFT/Default Spin/B3PW91” and the basis set “6-31G (d)” (Charge 0/Spin Singlet). The HOMO and LUMO energy levels are calculated according to the following calibration formulas, S1 and T1 are used directly.

HOMO(eV)=((HOMO(G)×27.212)−0.9899)/1.1206

LUMO(eV)=((LUMO(G)×27.212)−2.0041)/1.385

wherein, HOMO(G) and LUMO(G) in the unit of Hartree are the direct calculation results of Gaussian 03W. The results of various materials used in Examples 9 to 23 and Comparative Example 1 are shown in Table 1:

TABLE 1

| Materials | HOMO [eV] | LUMO [eV] | T1 [eV] | S1 [eV] |
|---|---|---|---|---|
| HATCN | −9.04 | −5.08 | 2.32 | 3.17 |
| SFNFB | −5.26 | −2.19 | 2.59 | 3.22 |
| The first organic | −6.09 | −2.75 | 2.71 | 3.22 |
| compound (1-4) | | | | |
| The first organic | −6.08 | −2.78 | 2.91 | 3.30 |
| compound (1-23) | | | | |
| The second organic | −5.54 | −2.34 | 2.83 | 3.20 |
| compound (2-40) | | | | |
| The second organic | −5.44 | −2.22 | 2.92 | 3.12 |
| compound (3-2) | | | | |
| The second organic | −5.44 | −2.36 | 2.69 | 3.17 |
| compound (3-23) | | | | |
| The second organic | −5.50 | −2.20 | 2.81 | 3.18 |
| compound (4-18) | | | | |
| The second organic | −5.49 | −2.25 | 2.79 | 3.18 |
| compound (5-2) | | | | |
| mCP | −5.82 | −2.12 | 3.11 | 3.39 |
| Ir(p-ppy)₃ | −5.17 | −2.32 | 2.67 | 2.90 |
| NaTzF₂ | −6.19 | −2.82 | 2.55 | 3.52 |

According to the results in Table 1, values of Δ((HOMO−(HOMO−1)), Δ((LUMO+1)−LUMO), min((LUMO(H1)−HOMO(H2), LUMO(H2)−HOMO(H1)) and min(E_{T}(H1), E_{T}(H2))[eV] of the first organic compound (1-4), the first organic compound (1-23), the second organic compound (2-40), the second organic compound (3-2), the second organic compound (3-23), the second organic compound (4-18) and the second organic compound (5-2) in Table 2 were calculated.

TABLE 2

| | | | | min((LUMO(H1) − | |
|---|---|---|---|---|---|
| | | Δ((HOMO − | | HOMO(H2), | |
| | | (HOMO − | Δ((LUMO + 1) − | LUMO(H2) − | min(ET(H1), |
| | | 1))[eV] | LUMO)[eV] | HOMO(H1))[eV] | E_{T}(H2))[eV] |
| Organic | The first | 0.23 | 0.58 | 2.79 | 2.83 |
| mixture | organic | | | | |
| 1 | compound | | | | |
| | (1-23) | | | | |
| | The second | 0.14 | 0.32 | | |
| | organic | | | | |
| | compound | | | | |
| | (2-40) | | | | |
| Organic | The first | 0.17 | 0.36 | 2.69 | 2.69 |
| mixture | organic | | | | |
| 2 | compound | | | | |
| | (1-4) | | | | |
| | The second | 0.42 | 0.03 | | |
| | organic | | | | |
| | compound | | | | |
| | (3-23) | | | | |
| Organic | The first | 0.23 | 0.58 | 2.66 | 2.91 |
| mixture | organic | | | | |
| 3 | compound | | | | |
| | (1-23) | | | | |
| | The second | 0.42 | 0.05 | | |
| | organic | | | | |
| | compound | | | | |
| | (3-2) | | | | |
| Organic | The first | 0.23 | 0.58 | 2.72 | 2.79 |
| mixture | organic | | | | |
| 4 | compound | | | | |
| | (1-23) | | | | |
| | The second | 0.12 | 0.09 | | |
| | organic | | | | |
| | compound | | | | |
| | (4-18) | | | | |
| Organic | The first | 0.23 | 0.58 | 2.71 | 2.79 |
| mixture | organic | | | | |
| 5 | compound | | | | |
| | (1-23) | | | | |
| | The second | 0.26 | 0.66 | | |
| | organic | | | | |
| | compound | | | | |
| | (5-2) | | | | |

(2) The current-voltage (J-V) characteristics of the organic light-emitting diodes in Examples 9 to 23 and Comparative Example 1 were characterized by characterization equipment while important parameters such as efficiency, lifetime (see Table 3, wherein, T90@1000 nits refers to the time it takes for the brightness to decay to 90% under the initial brightness of 1000 nit.) and external quantum efficiency (see Table 3, operating current density is 10 mA/cm².) were recorded. In Table 3, the lifetimes of the organic light-emitting diodes of Examples 9 to 23 all refer to a multiple of that of the organic light-emitting diode of Comparative Example 1. For example, the lifetime of the organic light-emitting diode of Comparative Example 1 is 1, and the lifetime of the organic light-emitting diode of Example 9 in Table 3 is 3.8, which means that the lifetime of the organic light-emitting diode of Example 9 is 3.8 times that of the organic light-emitting diode of Comparative Example 1, which also applies to Examples 10 to 23, and details are not described herein. It can be seen that the light-emitting efficiency and lifetime of the organic light-emitting diode based on the organic mixture are the highest among the same types of devices, wherein the lifetime of the device based on the organic mixture of Example 17 is 8 times or more that of the device of Comparative Example 1. It can be seen that the lifetimes of the devices prepared by using the above organic mixtures have been greatly improved.

TABLE 3

| | | external quantum | |
|---|---|---|---|
| OLED | | efficiency at | T90@ |
| devices | Host materials | 10 mA/cm² | 1000 nits |
| Example 9 | (1-23): (2-40) = 1:1 | 14.0% | 3.8 |
| | Vacuum co-evaporation | | |
| Example 10 | (1-23): (2-40) = 1:1 | 13.3% | 3.2 |
| | Simple blending | | |
| Example 11 | (1-23): (2-40) = 1:1 | 14.1% | 4.6 |
| | Organic alloy | | |
| Example 12 | (1-4): (3-23) = 1:1 | 15.3% | 5.3 |
| | Vacuum co-evaporation | | |
| Example 13 | (1-4): (3-23) = 1:1 | 14.8% | 4.8 |
| | Vacuum co-evaporation | | |
| Example 14 | (1-4): (3-23) = 1:1 | 16.0% | 6.2 |
| | Vacuum co-evaporation | | |
| Example 15 | (1-23): (3-2) = 1:1 | 17.0% | 7.2 |
| | Vacuum co-evaporation | | |
| Example 16 | (1-23): (3-2) = 1:1 | 16.2% | 6.5 |
| | Simple blending | | |
| Example 17 | (1-23): (3-2) = 1:1 | 18.1% | 8.7 |
| | Organic alloy | | |
| Example 18 | (1-23): (4-18) = 1:1 | 14.6% | 4.4 |
| | Vacuum co-evaporation | | |
| Example 19 | (1-23): (4-18) = 1:1 | 14.0% | 3.8 |
| | Simple blending | | |
| Example 20 | (1-23): (4-18) = 1:1 | 15.0% | 5.1 |
| | Organic alloy | | |
| Example 21 | (1-23): (5-2) = 1:1 | 14.7% | 4.9 |
| | Vacuum co-evaporation | | |
| Example 22 | (1-23): (5-2) = 1:1 | 14.2% | 4.3 |
| | Simple blending | | |
| Example 23 | (1-23): (5-2) = 1:1 | 15.5% | 5.6 |
| | Organic alloy | | |
| Comparative | mCP | 10.1% | 1 |
| Example 1 | | | |

The technical features of the above-described embodiments may be combined arbitrarily. To simplify the description, all the possible combinations of the technical features in the above embodiments are not described. However, all of the combinations of these technical features should be considered as within the scope of the present disclosure, as long as such combinations do not contradict with each other.

The above-described embodiments merely represent several embodiments of the present disclosure, and the description thereof is more specific and detailed, but it should not be construed as limiting the scope of the present disclosure. It should be noted that, for those skilled in the art, several variations and improvements may be made without departing from the concept of the present disclosure, and these are all within the protection scope of the present disclosure. Therefore, the scope of the present disclosure shall be defined by the appended claims.

1. An organic mixture comprising a first organic compound and a second organic compound that forms an exciplex with the first organic compound, the first organic compound is an aromatic compound containing a triphenylboron heterocycle, the second organic compound is a compound containing an aromatic fused heterocycle, and LUMO_{H1} is defined as the lowest unoccupied molecular orbital energy level of the first organic compound, HOMO_{H1} is defined as the highest occupied molecular orbital energy level of the first organic compound, E_{T}(H1) is defined as the triplet excited state energy level of the first organic compound, and LUMO_{H2} is defined as the lowest unoccupied molecular orbital energy level of the second organic compound, HOMO_{H2} is defined as the highest occupied molecular orbital energy level of the second organic compound, E_{T}(H2) is defined as the triplet excited state energy level of the second organic compound, wherein, min((LUMO_{H1}−HOMO_{H2}, LUMO_{H2}−HOMO_{H1})≤min(E_{T}(H1), E_{T}(H2))+0.1 eV.

2. The organic mixture of claim 1, wherein, the first organic compound has the following structural formulas: 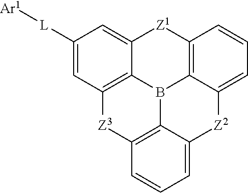
wherein, -L- is one selected from a single bond, a double bond and a triple bond, or L is one selected from the group consisting of an aromatic group with a ring atom number of 5 to 30 and a heteroaromatic group with a ring atom number of 5 to 30;
Ar¹ is one selected from the group consisting of an aromatic group with a ring atom number of 5 to 60 and a heteroaromatic group with a ring atom number of 5 to 60;
—Z¹—, —Z²— and —Z³— are each independently selected from the group consisting of none, —N(R)—, —C(R)₂—, —Si(R)₂—, —O—, —C═N(R)—, —C═C(R)₂—, —P(R)—, —P(═O)R—, —S—, —S═O— and —SO₂—, and at most two of —Z¹—, —Z²— and —Z³— are none; R is one selected from the group consisting of H, D, F, CN, alkenyl, alkynyl, nitrile group, amino, nitro, acyl, alkoxy, carbonyl, sulfonyl, C_{1˜30} alkyl, C_{3˜30} cycloalkyl, an aromatic hydrocarbyl with a ring atom number of 5 to 60, and an aromatic heterocyclic group with a ring atom number of 5 to 60.

3. The organic mixture of claim 2, wherein, the structural formula of Ar¹ is one selected from the group consisting of 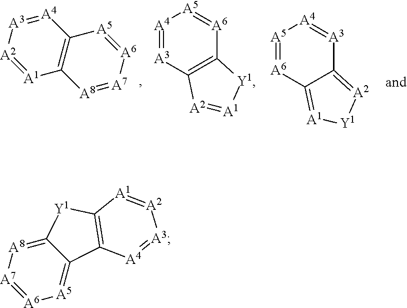
wherein, A¹, A², A³, A⁴, A⁵, A⁶, A⁷ and A⁸ are each independently selected from CR³ and N;
Y¹ and Y² are each independently selected from the group consisting of CR⁴R⁵, SiR⁴R⁵, NR³, C(═O), S and O;
R³, R⁴ and R⁵ are each independently selected from the group consisting of H, D, a linear alkyl with a carbon atom number of 1 to 20, a branched alkyl with a carbon atom number of 1 to 20, a cyclic alkyl with a carbon atom number of 1 to 20, an alkoxy with a carbon atom number of 1 to 20, a thioalkoxy with a carbon atom number of 1 to 20 and a silyl with a carbon atom number of 1 to 20, a ketone group with a carbon atom number of 1 to 20, an alkoxycarbonyl group with a carbon atom number of 1 to 20, an aryloxycarbonyl with a carbon atom number of 7 to 20, cyano, carbamoyl, haloformyl, formyl, isocyano, an isocyanate group, a thiocyanate group, an isothiocyanate group, hydroxyl, nitro, CF₃, Cl, Br, F, a crosslinkable group, an aryl containing 5 to 40 ring atoms, a heteroaryl containing 5 to 40 ring atoms, an aryloxy containing 5 to 40 ring atoms and a heteroaryloxy containing 5 to 40 ring atoms.

4. The organic mixture of claim 3, wherein, Ar¹ is one selected from the group consisting of 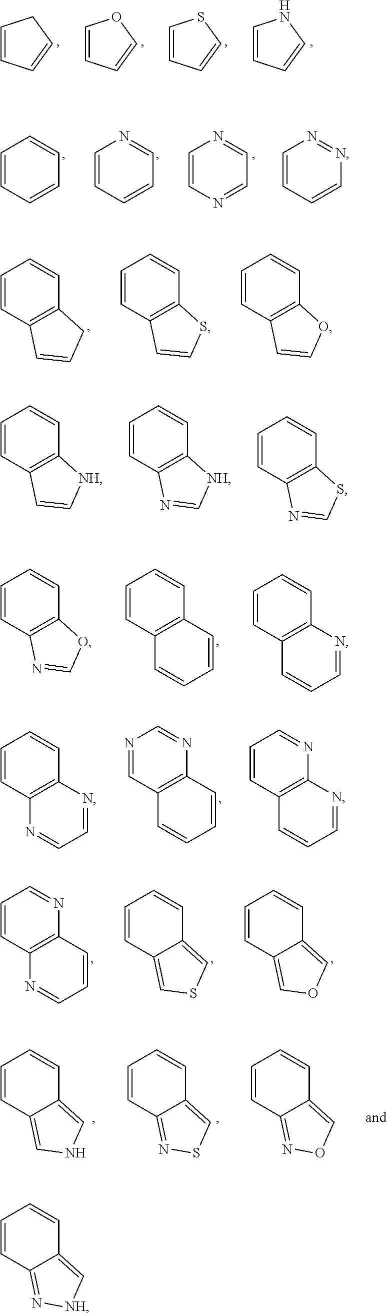
wherein, H on the ring may be arbitrarily substituted.

5. The organic mixture of claim 2, wherein, the L is one selected from the group consisting of 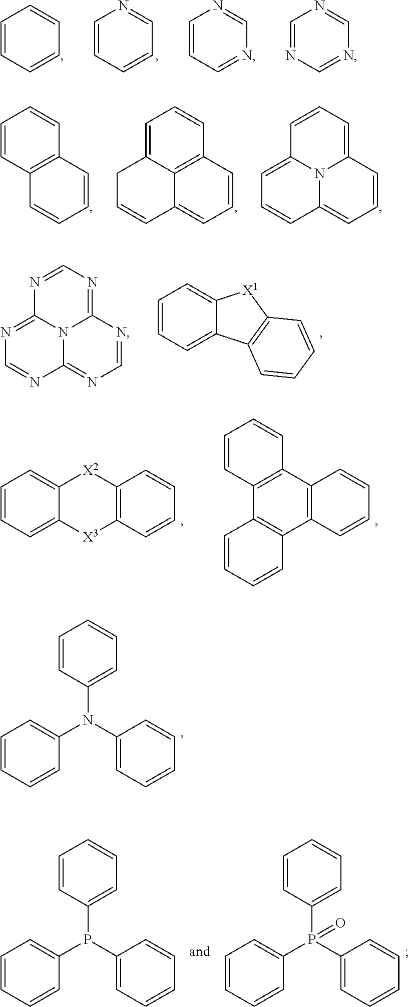
wherein, C—X¹—C, C—X²—C and C—X³—C are each independently selected from the group consisting of C—N(R)—C, C—C(R)₂—C, C—Si(R)₂—C, C—O—C, C—C═N(R)—C, C—C═C(R)₂—C, C—P(R)—C, C—P(═O)R—C, C—S—C, C—S═O—C, C—SO₂—C and C—C, and at most one of C—X²—C and C—X³—C is C—C.

6. The organic mixture of claim 2, wherein, the first organic compound is one selected from the group consisting of

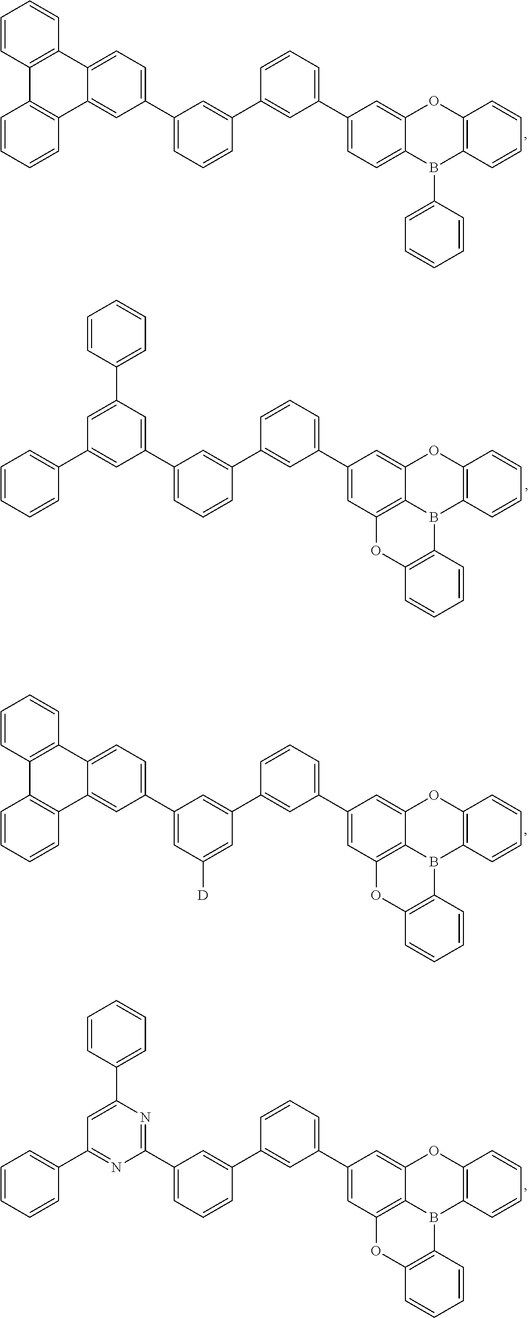 [SMILES: c1ccc(B2c3ccccc3Oc3cc(-c4cccc(-c5cccc(-c6ccc7c8ccccc8c8ccccc8c7c6)c5)c4)ccc32)cc1 ; c1ccc(-c2cc(-c3ccccc3)cc(-c3cccc(-c4cccc(-c5cc6c7c(c5)Oc5ccccc5B7c5ccccc5O6)c4)c3)c2)cc1 ; [2H]c1cc(-c2cccc(-c3cc4c5c(c3)Oc3ccccc3B5c3ccccc3O4)c2)cc(-c2ccc3c4ccccc4c4ccccc4c3c2)c1 ; c1ccc(-c2cc(-c3ccccc3)nc(-c3cccc(-c4cccc(-c5cc6c7c(c5)Oc5ccccc5B7c5ccccc5O6)c4)c3)n2)cc1]

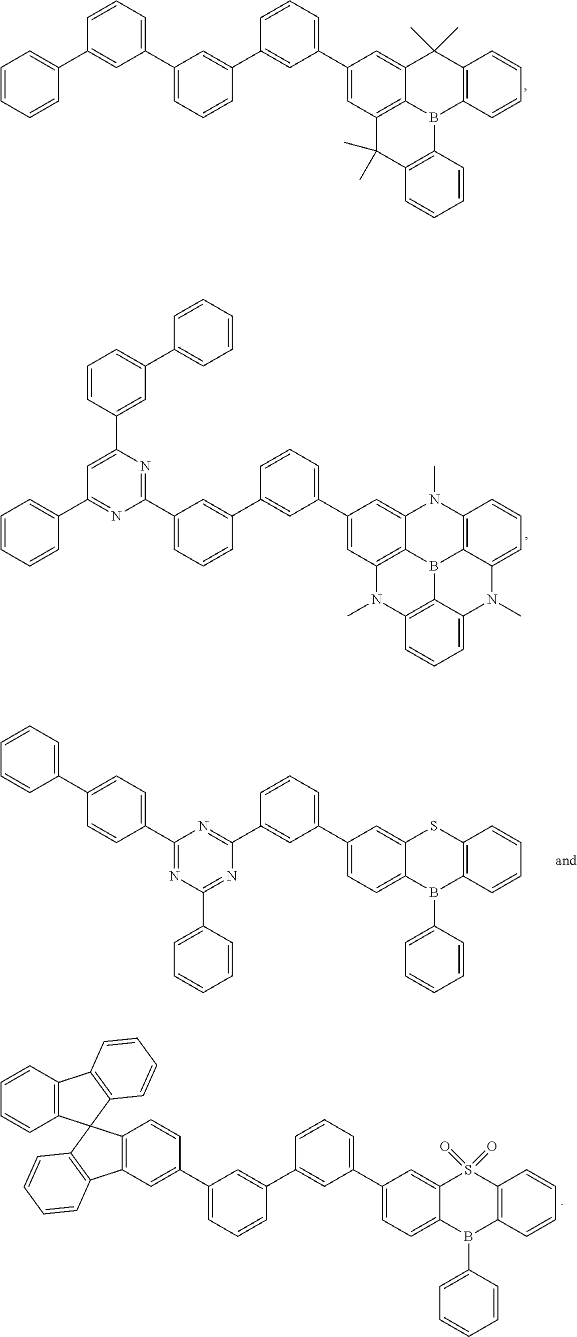 [SMILES: CC1(C)c2ccccc2B2c3ccccc3C(C)(C)c3cc(-c4cccc(-c5cccc(-c6cccc(-c7ccccc7)c6)c5)c4)cc1c32 ; CN1c2cccc3c2B2c4c1cccc4N(C)c1cc(-c4cccc(-c5cccc(-c6nc(-c7ccccc7)cc(-c7cccc(-c8ccccc8)c7)n6)c5)c4)cc(c12)N3C ; c1ccc(B2c3ccccc3Sc3cc(-c4cccc(-c5nc(-c6ccccc6)nc(-c6ccc(-c7ccccc7)cc6)n5)c4)ccc32)cc1 ; O=S1(=O)c2ccccc2B(c2ccccc2)c2ccc(-c3cccc(-c4cccc(-c5ccc6c(c5)-c5ccccc5C65c6ccccc6-c6ccccc65)c4)c3)cc21]

7. The organic mixture of claim 2, wherein, the Ar¹ is one selected from the group consisting of an aromatic group with a ring atom number of 5 to 60 containing an electron-accepting group, and a heteroaromatic group with a ring atom number of 5 to 60 containing an electron-accepting group.

8. The organic mixture of claim 7, wherein, the electron-accepting group is one selected from the group consisting of F, CN, 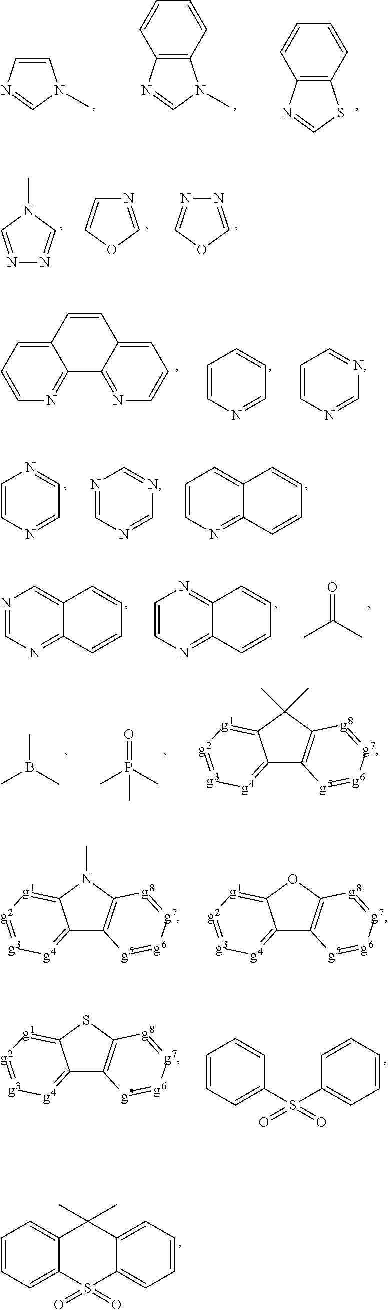 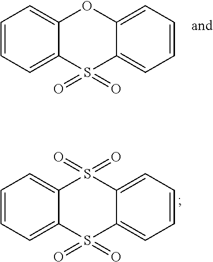
wherein, g¹, g², g³, g⁴, g⁵, g⁶, g⁷ and g⁸ are each independently one selected from C and N, and at least one of g¹, g², g³, g⁴, g⁵, g⁶, g⁷ and g⁸ is N.

9. The organic mixture of claim 1, wherein, the structural formula of the second organic compound is one selected from 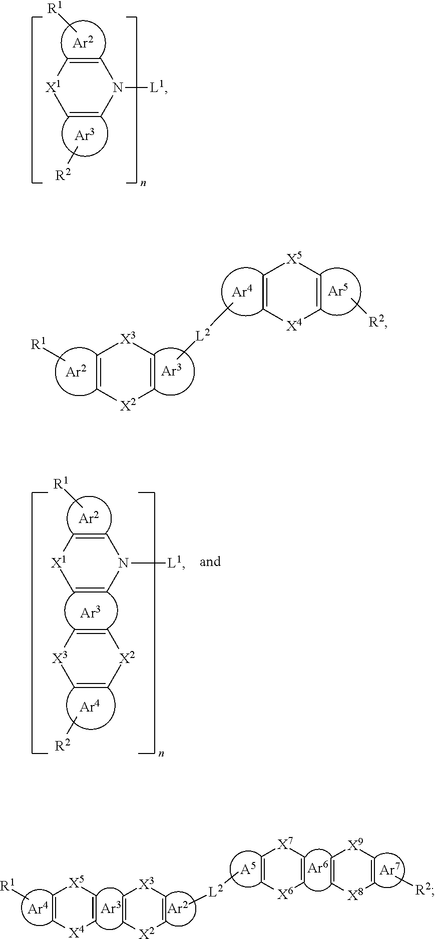
wherein, L¹ is one selected from the group consisting of an aromatic group with a ring atom number of 5 to 60, and a heteroaromatic group with a ring atom number of 5 to 60;
-L²- is a single bond, or L² is one selected from the group consisting of an aromatic group with a ring atom number of 5 to 30, and a heteroaromatic group with a ring atom number of 5 to 30; 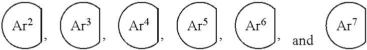
are each independently selected from the group consisting of an aromatic group with a ring atom number of 5 to 30, and a heteroaromatic group with a ring atom number of 5 to 30;
—X¹— is a single bond, or one selected from the group consisting of N(R), C(R)₂, Si(R)₂, O, C═N(R), C═C(R)₂, P(R), P(═O)R, S, S═O or SO₂;
—X²—, —X³—, —X⁴—, —X⁵—, —X⁶—, —X⁷—, —X⁸— and —X⁹— are each independently selected from the group consisting of a single bond, —N(R)—, —C(R)₂—, —Si(R)₂—, —O—, —(C═N(R⁶))—, —(C═C(R⁶)₂)—, —P(R⁶)—, —(P(═O)R⁶)—, —S—, —(S═O)— and —(SO₂)—, and at most one of —X²— and —X³— is a single bond, at most one of —X⁴— and —X⁵— is a single bond, at most one of —X⁶— and —X⁷— is a single bond, and at most one of —X⁸— and —X⁹— is a single bond;
R¹, R² and R⁶ are each independently selected from the group consisting of H, D, F, CN, alkenyl, alkynyl, nitrile group, amino, nitro, acyl, alkoxy, carbonyl, sulfonyl, C_{1˜30} alkyl, C_{3˜30} cycloalkyl, an aromatic hydrocarbyl with a ring atom number of 5 to 60, and an aromatic heterocyclic group with a ring atom number of 5 to 60;
m is any integer from 1 to 4.

10. The organic mixture of claim 9, wherein, 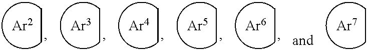
are each independently selected from the group consisting of 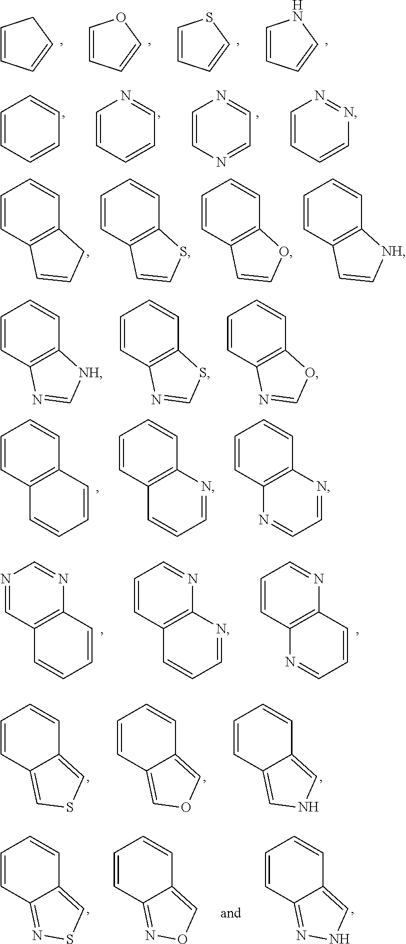
wherein, H on the ring may be arbitrarily substituted.

11. The organic mixture of claim 9, wherein, the L¹ and L² are each independently selected from the group consisting of 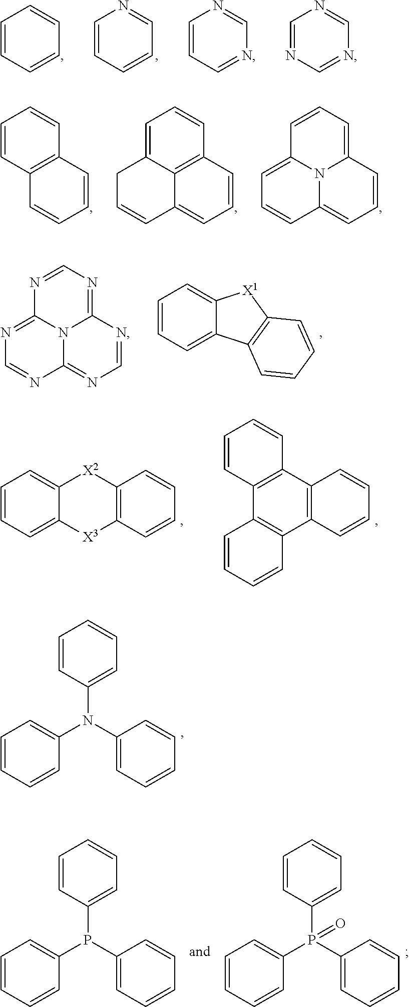
wherein, C—X¹—C, C—X²—C and C—X³—C are each independently selected from the group consisting of C—N(R)—C, C—C(R)₂—C, C—Si(R)₂—C, C—O—C, C—C═N(R)—C, C—C═C(R)₂—C, C—P(R)—C, C—P(═O)R—C, C—S—C, C—S═O—C, C—SO₂—C and C—C, and at most one of C—X²—C and C—X³—C is C—C.

12. The organic mixture of claim 9, wherein, the second organic compound is one selected from the group consisting of

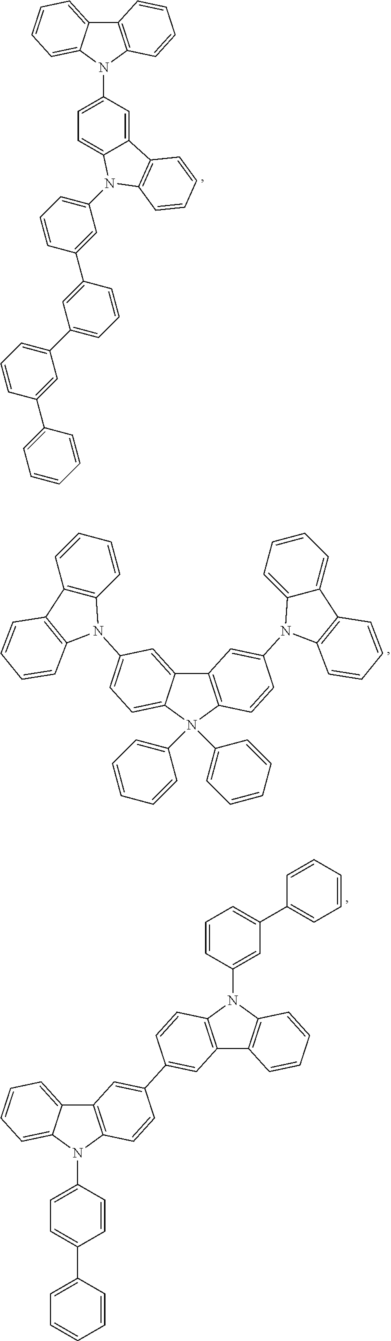

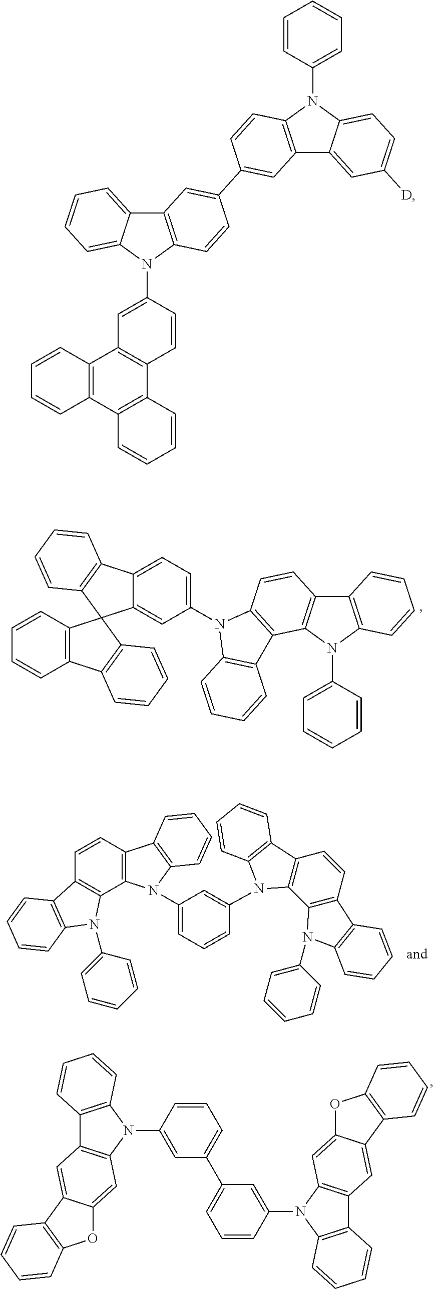

13. The organic mixture of claim 1, wherein, at least one of the first organic compound and the second organic compound satisfies ((HOMO−(HOMO−1))≥0.2 eV, (HOMO−1) is defined as the second highest occupied molecular orbital energy level.

14. The organic mixture of claim 1, wherein, the difference between the sublimation temperature of the first organic compound and that of the second organic compound does not exceed 30 K.

15. The organic mixture of claim 1, wherein, the organic mixture further comprises an organic functional material selected from the group consisting of a hole injection material, a hole transport material, a hole blocking material, an electron injection material, an electron transport material, an electron blocking material, an organic host material and a light-emitting material.

16. A formulation comprising the organic mixture of claim 1 and an organic solvent.

17. An organic electronic device comprising a functional layer, wherein the material of the functional layer comprises the organic mixture of claim 1.

18. The organic electronic device of claim 17, wherein, the organic electronic device is one selected from the group consisting of an organic light-emitting diode, an organic photovoltaic cell, an organic light-emitting electrochemical cell, an organic field effect transistor, an organic light-emitting field effect transistor, an organic laser, and an organic spintronic device, an organic sensor and an organic plasmon emitting diode.

19. The organic electronic device of claim 17, wherein, the organic electronic device is an organic light-emitting diode, and the functional layer is a light-emitting layer.

20. (canceled)

21. The organic mixture of claim 2, wherein the first organic compound is one selected from the group consisting of [chemical structure]
